(19) United States
(12) Patent Application Publication
(10) Pub. No.: US 2020/0397807 A1
(43) Pub. Date: Dec. 24, 2020

(54) NICOTINYL RIBOSIDE COMPOUNDS AND THEIR USES

(71) Applicant: MitoPower, LLC, Palo Alto, CA (US)

(72) Inventors: Gautham Tumkur PRANESH, Bengaluru (IN); Gangadhara GANAPATI, Palo Alto, CA (US); Atignal Shankara Rao ARVIND, Bangalore (IN); G. Mani SUBRAMANIAN, Los Altos Hills, CA (US)

(21) Appl. No.: 16/904,270

(22) Filed: Jun. 17, 2020

Related U.S. Application Data
(60) Provisional application No. 62/863,239, filed on Jun. 18, 2019.

(51) Int. Cl. A61K 31/706; A61K 31/06

(52) U.S. Cl. CPC A61K 31/706; A61K 45/06; A61K 31/06

(57) ABSTRACT

The disclosure provides nicotinamide riboside (NR), the reduced form of NR (NRH), nicotinic acid riboside (NAR), the reduced form of NAR (NARH), derivatives thereof, compositions thereof and uses thereof. The NR and NAR derivatives have improved stability and bioavailability compared to NR and NAR. NR, NRH, NAR, NARH, and derivatives thereof can increase cellular NAD⁺ levels and enhance mitochondrial and cellular function and cell viability. Therefore, NR, NRH, NAR, NARH, and derivatives thereof, whether alone or in combination with one or more additional therapeutic agents (e.g., a mitochondrial uncoupler or/and a PARP inhibitor), are useful for treating mitochondrial diseases, mitochondria-related diseases and conditions, metabolic disorders, and other disorders and conditions.

### CROSS-REFERENCE TO RELATED APPLICATION

The present application claims priority to/from and the benefit of U.S. Provisional Application No. 62/863,239 filed on Jun. 18, 2019.

### FIELD OF THE DISCLOSURE

The disclosure relates to nicotinamide riboside (NR), the reduced form of NR (NRH), nicotinic acid riboside (NAR), the reduced form of NAR (NARH), derivatives thereof, compositions thereof and uses thereof, including to increase NAD⁺ levels, to enhance mitochondrial and cellular function and cell viability, and to treat or prevent mitochondrial diseases, mitochondria-related diseases, metabolic disorders and other disorders.

### BACKGROUND OF THE DISCLOSURE

Nicotinamide adenine dinucleotide (NAD) is a coenzyme that is critical for cellular function. It serves two major functions. First, NAD serves as a carrier for redox functions. These are chemical reactions involving the transfer of electrons and form the basis for energy production in every cell [Croteau et al. (2017); Fang et al. (2017); Chini et al. (2016); and Yang et al. (2016)]. The oxidized form of NAD is abbreviated NAD⁺, and the reduced form of NAD is abbreviated NADH. NAD⁺ is an oxidizing agent that accepts electrons from other molecules to form NADH, which in turn is a reducing agent that donates electrons to other molecules. Such electron-transfer reactions are the main function of NAD.

Second, NAD is an essential cofactor in several non-redox reactions by providing ADP-ribose to catalyze the enzymatic function of two key protein families—the sirtuins (SIRTs) and the poly(ADP-ribose) polymerases (PARPs). SIRTs are deacetylases involved in the maintenance of nuclear, mitochondrial and cytoplasmic or metabolic homeostasis (references 1-3). PARPs are involved in DNA repair and play a broad role in the maintenance of chromatin structure and function [Croteau et al. (2017); Fang et al. (2017); Chini et al. (2016); and Yang et al. (2016)].

When NAD⁺ levels are depleted, cellular functioning is impaired due to both reduced level of energy production and disruption of cellular homeostasis. Reduction in NAD⁺ levels is observed in physiological states such as in aging, and across a wide range of pathological states ranging from acute injury to chronic metabolic and inflammatory conditions [Bonkowski et al. (2016); Frederick et al. (2016); Zang et al. (2016); and Imai et al. (2014)]. In particular, given the central role of the mitochondria in energy production, tissues and organs with higher numbers of mitochondria such as the liver, heart, skeletal muscle, brain and kidneys are most susceptible to NAD⁺ depletion and thus are most amenable to therapies that can enhance NAD⁺ levels [Bonkowski et al. (2016); Frederick et al. (2016); Zang et al. (2016); and Imai et al. (2014)]. In addition, efficient mitochondrial activity is critical for immune cell function, and mitochondrial dysfunction is associated with poor immune surveillance (impaired antigen recognition and immune exhaustion) and immune cell senescence [Bonkowski et al. (2016); Frederick et al. (2016); Zang et al. (2016); and Imai et al. (2014)].

There are several approaches to enhancing NAD⁺ levels based on the different ways NAD is synthesized in the body. In each such instance, the starting point is usually a compound obtained from the diet. Such compounds include dietary tryptophan, and derivatives of vitamin B₃ that include nicotinic acid (NA), nicotinamide (NAM), nicotinamide riboside (NR) and nicotinamide mononucleotide (NMN). While NAD can also be obtained in the diet, it is rapidly broken down into NAM or NR by extracellular hydrolases such as CD38 and CD73 [Camacho-Pereira et al. (2016)].

Nicotinamide, nicotinic acid and nicotinamide riboside are natural compounds that are currently available as nutritional supplements. NMN is a nucleotide derivative of NAM that is considered to be a biochemical precursor of NAD⁺. There is in vitro data showing that NR and NMN in particular can elevate NAD⁺ levels. Further, in animal models NR and NMN elevate NAD⁺ levels and improve organ function [Fang et al. (2016); Mills et al. (2016); de Picciotto et al. (2016); and Zang et al. (2016)], disease pathology and longevity [Fang et al. (2016); Mills et al. (2016); de Picciotto et al. (2016); and Zang et al. (2016)].

While NR and NMN are useful as precursors of NAD⁺ and can potentially elevate levels of NAD⁺ and thus promote cellular health and mitochondrial function, the bioavailability of these molecules is not optimal for their use as pharmacological and nutritional agents [Ratajczak et al. (2016) and Trammell et al. (2016)]. The reasons for their poor bioavailability include pH-dependent stability, degradation due to hydrolysis, and the need for enzymatic conversion within the cell to NAD for biological effects.

### SUMMARY OF THE DISCLOSURE

The disclosure relates to nicotinamide riboside (NR), the reduced form of NR (NRH), nicotinic acid riboside (NAR), the reduced form of NAR (NARH), derivatives thereof, compositions thereof and uses thereof, including to increase NAD⁺ levels, to enhance mitochondrial and cellular function and cell viability, to provide cytoprotection, and to treat or prevent mitochondrial diseases, mitochondria-related diseases, metabolic disorders and other disorders. The nicotinyl riboside compounds can be used alone or in combination with one or more additional therapeutic agents, such as a PARP inhibitor or/and a mitochondrial uncoupler.

The disclosure provides compounds of Formulas I and II:

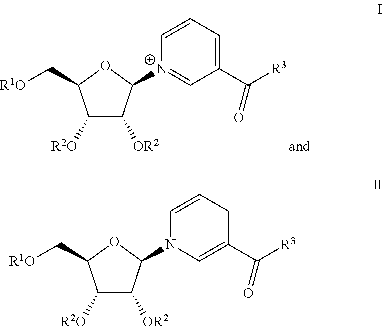

wherein R¹, R² and R³ are defined elsewhere herein.

The disclosure further provides compounds of Formulas III and IV:

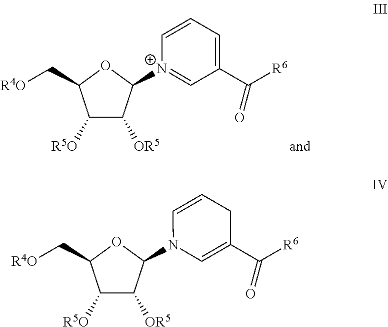

wherein R⁴, R⁵ and R⁶ are defined elsewhere herein.

The compounds of Formulas I, II, III and IV can increase NAD⁺ levels in the mitochondria, the cytoplasm or/and the nucleus of cells (e.g., total cellular NAD⁺ level) and can enhance mitochondrial and cellular function and cell viability, and have suitable bioavailability and stability in intracellular and extracellular environments. Therefore, the compounds are useful for treating mitochondrial diseases, mitochondria-related diseases and conditions, diseases and conditions characterized by acute NAD⁺ depletion due to DNA damage, metabolic disorders, and other disorders and conditions.

### BRIEF DESCRIPTION OF THE FIGURES

FIG. 1 illustrates an exemplary process for making compounds of Formulas I and II, which can be adapted to make compounds of Formulas III and IV.

FIG. 2 shows a process for synthesizing MP-05 and MP-06.

FIG. 3 shows a process for synthesizing MP-07 and MP-08.

FIG. 4 shows a process for synthesizing MP-09 and MP-10.

FIG. 5 shows a process for synthesizing MP-14 and MP-16.

FIG. 6 shows a process for synthesizing MP-12 and MP-15.

FIG. 7 shows a process for synthesizing MP-17, MP-20, MP-23 and MP-24.

FIG. 8 shows a process for synthesizing MP-18, MP-19, MP-21 and MP-22.

FIG. 9 shows a process for synthesizing MP-17 and MP-20.

FIG. 10 shows a process for synthesizing MP-41.

FIG. 11 shows a process for synthesizing MP-42.

FIG. 12 shows % recovery of NAD⁺ level depleted by the DNA-alkylating mutagen N-methyl-N-nitroso-N′-nitroguanidine (MNNG) in Jurkat cells with varying concentrations of MP-17.

FIG. 13 shows % reduction of MNNG-induced cytotoxicity in Jurkat cells with varying concentrations of MP-17.

FIG. 14 shows % recovery of NAD⁺ level depleted by MNNG in Jurkat cells with varying concentrations of MP-41.

FIG. 15 shows % reduction of MNNG-induced cytotoxicity in Jurkat cells with varying concentrations of MP-41.

FIG. 16 shows synergistic repletion of NAD⁺ level in MNNG-treated Jurkat cells by a combination of nicotinamide riboside (“MP2” in FIG. 16) and a very low concentration (5 nM) of the PARP inhibitor olaparib.

FIG. 17 shows synergistic cytoprotection (reduction of cytotoxicity) in MNNG-treated Jurkat cells by a combination of nicotinamide riboside (“MP2” in FIG. 17) and a very low concentration (5 nM) of olaparib.

FIG. 18A shows the effects of NRH (MP04), nitazoxanide (NTZ) or olaparib (Ola) alone, and combinations thereof, on NAD⁺ level in Jurkat cells not treated with MNNG (n=3 for each experiment). FIG. 18B shows the effects of NR (MP2), nitazoxanide or olaparib alone, and combinations thereof, on NAD⁺ level in Jurkat cells not treated with MNNG (n=3 for each experiment).

FIG. 19A shows the effects of NRH (MP04), nitazoxanide (NTZ) or olaparib (Ola) alone, and combinations thereof, on NAD⁺ level in Jurkat cells treated with MNNG (n=3 for each experiment). FIG. 19B shows the effects of NR (MP2), nitazoxanide or olaparib alone, and combinations thereof, on NAD⁺ level in Jurkat cells treated with MNNG (n=3 for each experiment).

FIG. 20 shows the effects of NRH (MP04) or nitazoxanide (NTZ) alone, and a combination of both, on NAD⁺/NADH ratio in Jurkat cells not treated or treated with MNNG (n=3 for each experiment).

FIG. 21 shows the effects of NRH (MP04) alone, and combinations of NRH plus nitazoxanide (NTZ) or NRH plus nitazoxanide and olaparib (Ola), on ATP level in Jurkat cells not treated with MNNG (n=3 for each experiment).

FIG. 22 shows the effects of NRH (MP04), nitazoxanide (NTZ) at various concentrations or olaparib (Ola) alone, and combinations thereof, on NAD⁺ level in HepG2 cells not treated with MNNG (n=3 for each experiment).

FIG. 23 shows the effects of NRH (MP04), nitazoxanide (NTZ) at various concentrations or olaparib (Ola) alone, and combinations thereof, on NAD⁺ level in HepG2 cells treated with MNNG (n=3 for each experiment).

FIG. 24 shows the effects of NRH (MP04) or 0.5 or 5 μM nitazoxanide (NTZ) alone, and combinations of both, on NAD⁺/NADH ratio in HepG2 cells not treated or treated with MNNG (n=3 for each experiment).

FIG. 25 shows the effects of NRH (MP04) or 0.5 or 5 μM nitazoxanide (NTZ) alone, and combinations of both, on ATP level in HepG2 cells not treated or treated with MNNG (n=3 for each experiment).

FIG. 26 shows the % viability of HepG2 cells untreated or treated with 50 μM DNP alone, 100 μM NRH (MP04) alone, or 50 μM DNP plus 100 μM NRH.

FIG. 27 shows the activity (in international units per liter [IU/L]) of lactate dehydrogenase (LDH) released into the media from HepG2 cells untreated or treated with 50 μM DNP alone, 100 μM NRH (MP04) alone, or 50 μM DNP plus 100 μM NRH.

FIG. 28 shows the concentration (mmol/L) of lactate released into the media from HepG2 cells untreated or treated with 50 μM DNP alone, 100 μM NRH (MP04) alone, or 50 μM DNP plus 100 μM NRH.

FIG. 29 shows the concentration (mmol/L) of glucose in the media containing HepG2 cells untreated or treated with 50 μM DNP alone, 100 μM NRH (MP04) alone, or 50 μM DNP plus 100 μM NRH.

### GENERAL STATEMENTS

While various embodiments of the present disclosure are described herein, it will be obvious to those skilled in the art that such embodiments are provided by way of example only. Numerous modifications and changes to, and variations and substitutions of, the embodiments described herein will be apparent to those skilled in the art without departing from the disclosure. It is understood that various alternatives to the embodiments described herein can be employed in practicing the disclosure. It is also understood that every embodiment of the disclosure can optionally be combined with any one or more of the other embodiments described herein which are consistent with that embodiment.

Where elements are presented in list format (e.g., in a Markush group), it is understood that each possible subgroup of the elements is also disclosed, and any one or more elements can be removed from the list or group.

It is further understood that the disclosure of a numerical range is a specific disclosure of all the possible subranges and all the possible individual numbers (whether whole numbers or fractions) within that range regardless of the breadth of that range.

It is also understood that, unless clearly indicated to the contrary, in any method described or claimed herein that includes more than one act or step, the order of the acts or steps of the method is not necessarily limited to the order in which the acts or steps of the method are recited, but the disclosure encompasses embodiments in which the order is so limited.

It is further understood that, in general, where an embodiment in the description or the claims is referred to as comprising one or more features, the disclosure also encompasses embodiments that consist of, or consist essentially of, such feature(s).

It is also understood that any embodiment of the disclosure, e.g., any embodiment or compound found within the prior art, can be explicitly excluded from the claims, regardless of whether or not the specific exclusion is recited in the specification.

It is further understood that the present disclosure encompasses salts, solvates, hydrates, clathrates and polymorphs of all of the compounds disclosed herein. The specific recitation of “salts”, “solvates”, “hydrates”, “clathrates” or “polymorphs” with respect to a compound or a group of compounds in certain instances of the disclosure shall not be interpreted as an intended omission of any of these forms in other instances of the disclosure where the compound or the group of compounds is mentioned without recitation of any of these forms, unless stated otherwise or the context clearly indicates otherwise.

All patent literature and all non-patent literature cited herein are incorporated herein by reference in their entirety to the same extent as if each patent literature or non-patent literature were specifically and individually indicated to be incorporated herein by reference in its entirety.

### Definitions

Unless defined otherwise or clearly indicated otherwise by their use herein, all technical and scientific terms used herein have the same meaning as commonly understood by those of ordinary skill in the art to which this application belongs.

As used in the specification and the appended claims, the indefinite articles “a” and “an” and the definite article “the” can include plural referents as well as singular referents unless specifically stated otherwise or the context clearly indicates otherwise.

The term “exemplary” as used herein means “serving as an example, instance or illustration”. Any embodiment or feature characterized herein as “exemplary” is not necessarily to be construed as preferred or advantageous over other embodiments or features.

In some embodiments, the term “about” or “approximately” means within ±10% or 5% of the given value. Whenever the term “about” or “approximately” precedes the first numerical value in a series of two or more numerical values or in a series of two or more ranges of numerical values, the term “about” or “approximately” applies to each one of the numerical values in that series of numerical values or in that series of ranges of numerical values.

Whenever the term “at least” or “greater than” precedes the first numerical value in a series of two or more numerical values, the term “at least” or “greater than” applies to each one of the numerical values in that series of numerical values.

Whenever the term “no more than” or “less than” precedes the first numerical value in a series of two or more numerical values, the term “no more than” or “less than” applies to each one of the numerical values in that series of numerical values.

The term “agent” denotes a chemical compound, a mixture of chemical compounds, a biological macromolecule (such as a nucleic acid, a polypeptide or a portion thereof, or an antibody or a fragment thereof), a mixture of biological macromolecules, or an extract of a biological material such as an animal (particularly a mammalian) cell or tissue, a plant, a bacterium or a fungus. The term “polypeptides” includes peptides (e.g., polypeptides containing no more than about 50 amino acid residues) and proteins (which are larger polypeptides).

A “modulator” of, e.g., a receptor or enzyme can be an activator or inhibitor of that receptor or enzyme, and can increase or reduce the activity or/and the level of that receptor or enzyme. For example, a “sirtuin-modulating compound” can be an activator or inhibitor of a sirtuin, and can increase or reduce the activity or/and the level of a sirtuin.

The term “therapeutic agent” refers to any biologically, physiologically or pharmacologically active substance that acts locally or systemically in or/and on a subject and is administered to a subject for purposes of diagnosis, treatment, mitigation, cure or prevention of a medical condition or enhancement of a desired physical or mental development or condition.

The term “therapeutically effective amount” refers to an amount of an agent that, when administered to a subject, is sufficient to prevent, reduce the risk of developing, delay the onset of, slow the progression of or cause regression of the medical condition being treated, or to alleviate to some extent the medical condition or one or more symptoms or complications of that condition, at least in some fraction of the subjects taking that agent. The term “therapeutically effective amount” also refers to an amount of an agent that is sufficient to elicit the biological or medical response of a cell, tissue, organ, system, animal or human which is sought by a researcher, veterinarian, medical doctor or clinician.

The terms “treat”, “treating” and “treatment” include alleviating, ameliorating or reducing the severity or frequency of, inhibiting the progress of, reversing or abrogating a medical condition or one or more symptoms or complications associated with the condition, and alleviating, ameliorating or eradicating one or more causes of the condition. Reference to “treatment” of a medical condition includes prevention of the condition. The terms “prevent”, “preventing” and “prevention” include precluding, reducing the risk of developing and delaying the onset of a medical condition or one or more symptoms or complications associated with the condition.

The term “medical conditions” (or “conditions” for short) includes diseases and disorders. The terms “diseases” and “disorders” are used interchangeably herein.

“Diabetes mellitus” (or “diabetes” for short) is a metabolic disorder characterized by high blood sugar level over a prolonged period, and can include complications such as ketoacidosis. Diabetes is characterized by chronic, general metabolic abnormalities resulting from prolonged high blood sugar level or/and a decrease in glucose tolerance. The main types of diabetes include type 1 diabetes (T1D), type 2 diabetes (T2D) and gestational diabetes.

“Mitochondrial diseases” are disorders caused by dysfunctional mitochondria or malfunction or failure in mitochondrial homeostasis, and occur when the mitochondria of the cell, e.g., fail to produce enough energy for cell or organ function, or produce excessive amounts of reactive oxygen species (ROS) that cause oxidative damage to the cell or components thereof or lead to other pathological effects. Mitochondrial homeostasis includes, e.g., autophagy of defective mitochondria (mitophagy) and mitochondrial biogenesis, and mitochondrial fission and fusion. A mitochondrial disease can be due to, e.g., a congenital genetic deficiency or defect (e.g., a mutation or deletion in mitochondrial DNA resulting in defective mitochondria), or an acquired deficiency or defect. A mitochondrial disease can be caused by, e.g., oxidative damage during aging, excessive mitochondrial calcium level, excessive exposure of affected cells to nitric oxide, ischemia, hypoxia, microtubule-associated deficit in axonal transport of mitochondria, or excessive expression of mitochondrial uncoupling proteins. Congenital mitochondrial diseases result from, e.g., hereditary mutations, deletions or other defects in mitochondrial DNA or in nuclear genes encoding proteins (e.g., those regulating mitochondrial DNA function or integrity). Acquired mitochondrial defects can be caused by, e.g., damage to mitochondrial DNA due to oxidative processes or aging, mitochondrial dysfunction, inhibition of respiratory chain complexes, mitochondrial respiration deficiencies and defects, oxygen deficiency, impaired nuclear-mitochondrial interactions, and excessive expression of mitochondrial uncoupling proteins in response to, e.g., lipids, oxidative damage or inflammation.

Mitochondrial diseases encompass primary mitochondrial diseases (PMDs) and secondary mitochondrial dysfunctions (SMDs). PMDs are inherited, whereas SMDs can be inherited or acquired. A PMD is caused by a pathogenic defect (e.g., a mutation or deletion) in a germline mitochondrial DNA (mtDNA) or nuclear DNA (nDNA) gene before conception (inherited) which encodes a protein involved in the oxidative phosphorylation (oxphos) process in the electron transport chain (ETC) or affects oxphos performance by impacting production or function of the machinery for running the oxphos process. A pathogenic defect in a wild-type germline mtDNA or nDNA gene after conception (acquired) which encodes an ETC protein or affects the production or function of the ETC machinery, such as due to alteration of such a mtDNA or nDNA gene by oxidative stress, or a pathogenic defect in such a somatic mtDNA or nDNA gene before or after conception, results in an SMD. An SMD can also be caused by a pathogenic defect in a germline or somatic mtDNA or nDNA gene before or after conception which neither encodes an ETC protein nor affects the production or function of the ETC machinery (e.g., a gene encoding a protein involved in a non-ETC mitochondrial process such as fatty acid oxidation or the citric acid/Krebs cycle). Moreover, an SMD can be due to a non-genetic cause such as an environmental insult that causes oxidative stress, which can, e.g., alter mitochondrial or non-mitochondrial protein(s) and adversely impact mitochondria, and which can occur as a result of, e.g., aging, an inflammatory response or a mitotoxic agent. An SMD can also be associated with a hereditary or acquired mitochondria-related disease or condition.

“Mitochondria-related diseases and conditions” are diseases and conditions that are associated with (e.g., are caused by or result in) mitochondrial dysfunction or malfunction or failure in mitochondrial homeostasis which may be secondary to or accompany other pathologies or pathophysiologies, and can be inherited or acquired.

The term “subject” refers to an animal, including but not limited to a mammal, such as a primate (e.g., a human, a chimpanzee or a monkey), a rodent (e.g., a rat, a mouse, a guinea pig, a gerbil or a hamster), a lagomorph (e.g., a rabbit), a bovine (e.g., a cattle), a suid (e.g., a pig), a caprine (e.g., a sheep), an equine (e.g., a horse), a canine (e.g., a dog) or a feline (e.g., a cat). The terms “subject” and “patient” are used interchangeably herein in reference, e.g., to a mammalian subject, such as a human subject.

The term “bioavailable”, when referring to an agent, refers to the extent to which the agent is taken up by a cell, tissue or organ, or is otherwise physiologically available to the subject after administration.

The term “parenteral” refers to a route of administration other than through the alimentary canal, such as by injection, infusion or inhalation. Parenteral administration includes without limitation subcuticular, intradermal, subcutaneous, intravascular, intravenous, intra-arterial, intramuscular, intracardiac, intraperitoneal, intracavitary, intra-articular, intracapsular, subcapsular, intra-orbital, transtracheal, intrasternal, intrathecal, intramedullary, intraspinal, subarachnoid and topical administrations. Topical administration includes without limitation dermal/epicutaneous, transdermal, mucosal, transmucosal, intranasal (e.g., by nasal spray or drop), ocular (e.g., by eye drop), pulmonary (e.g., by oral or nasal inhalation), buccal, sublingual, rectal (e.g., by suppository), and vaginal (e.g., by suppository).

The term “pharmaceutically acceptable” refers to a substance (e.g., an active ingredient or an excipient) that is suitable for use in contact with the tissues and organs of a subject without excessive irritation, allergic response, immunogenicity and toxicity, is commensurate with a reasonable benefit/risk ratio, and is effective for its intended use. A “pharmaceutically acceptable” excipient or carrier of a pharmaceutical composition is also compatible with the other ingredients of the composition.

The term “nicotinyl (or nicotinoyl or nicotinic) riboside compounds” as used herein includes nicotinamide riboside (NR), the reduced form of NR (NRH), nicotinic acid riboside (NAR), the reduced form of NAR (NARH), and derivatives thereof. As used herein, the term “nicotinamide riboside (NR) derivatives” includes derivatives of both the oxidized form and the reduced form of NR, and the term “nicotinic acid riboside (NAR) derivatives” includes derivatives of both the oxidized form and the reduced form of NAR.

The disclosure encompasses salts, solvates, hydrates, clathrates and polymorphs of the compounds described herein. A “solvate” of a compound includes a stoichiometric or non-stoichiometric amount of a solvent (e.g., water, acetone or an alcohol [e.g., ethanol]) bound non-covalently to the compound. A “hydrate” of a compound includes a stoichiometric or non-stoichiometric amount of water bound non-covalently to the compound. A “clathrate” of a compound contains molecules of a substance (e.g., a solvent) enclosed in a crystal structure of the compound. A “polymorph” of a compound is a crystalline form of the compound.

The term “alkyl” refers to a linear (straight chain) or branched, saturated monovalent hydrocarbon radical, which can optionally be substituted with one or more substituents. The term “lower alkyl” refers to a linear C₁-C₆ or branched C₃-C₆ alkyl group. Lower alkyl groups include without limitation methyl, ethyl, propyl (including n-propyl and isopropyl), butyl (including all isomeric forms, such as n-butyl, isobutyl, sec-butyl and tert-butyl), pentyl (including all isomeric forms, such as n-pentyl and isopentyl), and hexyl (including all isomeric forms, such as n-hexyl).

The term “alkenyl” refers to an alkyl group having one or more C═C double bonds. An alkenyl group can optionally be substituted with one or more substituents.

The term “cycloalkyl” refers to a cyclic saturated, bridged or non-bridged monovalent hydrocarbon radical, which can optionally be substituted with one or more substituents. C₃-C₁₀ cycloalkyl groups include without limitation cyclopropyl, cyclobutyl, cyclopentyl, cyclohexyl, cycloheptyl, cyclo-octyl, norbornyl and adamantyl.

The term “heterocyclyl” or “heterocyclic” refers to a monocyclic non-aromatic group or a multicyclic group that contains at least one non-aromatic ring, wherein at least one non-aromatic ring contains one or more heteroatoms independently selected from O, N and S. The non-aromatic ring containing one or more heteroatoms may be attached or fused to one or more saturated, partially unsaturated or aromatic rings. A heterocyclyl or heterocyclic group can optionally be substituted with one or more substituents. 3- to 8-membered, nitrogen-containing heterocyclic rings include without limitation aziridinyl, azetidinyl, pyrrolidinyl, piperidinyl, piperazinyl, morpholinyl, azepanyl and azocanyl. 3- to 8-membered, oxygen-containing heterocyclic rings include without limitation oxiranyl, oxetanyl, tetrahydrofuranyl, tetrahydropyranyl, morpholinyl, oxepanyl and oxocanyl.

The term “aryl” refers to a monocyclic aromatic hydrocarbon group or a multicyclic group that contains at least one aromatic hydrocarbon ring. An aryl group can optionally be substituted with one or more substituents. Aryl groups include without limitation phenyl, naphthyl, azulenyl, fluorenyl, anthryl, phenanthryl, biphenyl and terphenyl. The aromatic hydrocarbon ring of an aryl group may be attached or fused to one or more saturated, partially unsaturated or aromatic rings—e.g., dihydronaphthyl, indenyl, indanyl and tetrahydronaphthyl (tetralinyl).

The term “heteroaryl” refers to a monocyclic aromatic group or a multicyclic group that contains at least one aromatic ring, wherein at least one aromatic ring contains one or more heteroatoms independently selected from O, N and S. The heteroaromatic ring may be attached or fused to one or more saturated, partially unsaturated or aromatic rings that may contain only carbon atoms or that may contain one or more heteroatoms. A heteroaryl group can optionally be substituted with one or more substituents. Monocyclic heteroaryl groups include without limitation pyrrolyl, pyrazolyl, pyrazolinyl, imidazolyl, oxazolyl, isoxazolyl, thiazolyl, thiadiazolyl, isothiazolyl, furanyl, thienyl (thiophenyl), oxadiazolyl, triazolyl, tetrazolyl, pyridyl, pyridonyl, pyrazinyl, pyrimidinyl, pyridazinyl, pyridazinonyl and triazinyl. Bicyclic heteroaryl groups include without limitation indolyl, benzothiazolyl, benzothiadiazolyl, benzoxazolyl, benzisoxazolyl, benzothienyl (benzothiophenyl), quinolinyl, tetrahydroisoquinolinyl, isoquinolinyl, benzimidazolyl, benzotriazolyl, indolizinyl, benzofuranyl, isobenzofuranyl, chromonyl, coumarinyl, cinnolinyl, quinazolinyl, quinoxalinyl, indazolyl, naphthyridinyl, phthalazinyl, quinazolinyl, purinyl, pyrrolopyridinyl, furopyridinyl, thienopyridinyl, dihydroisoindolyl and tetrahydroquinolinyl.

### DETAILED DESCRIPTION OF THE DISCLOSURE

The disclosure provides nicotinamide riboside (NR), the reduced form of NR (NRH), nicotinic acid riboside (NAR), the reduced form of NAR (NARH), derivatives thereof, compositions thereof and uses thereof. The NR and NAR derivatives described herein can act as precursors or prodrugs of NR/NRH and NAR/NARH and thereby serve as sources of NR/NRH and NAR/NARH with improved stability and bioavailability. Both the oxidized form and the reduced form of both NR and NAR can be converted within the body to NMN and then to NAD⁺. Alternatively, without intending to be bound by theory, NRH and NARH may be converted to a reduced form of NMN and NAMN (NMNH and NAMNH), which may then be converted to NADH, which functions as a reducing agent in redox reactions and becomes oxidized to NAD⁺ in the process. By increasing NAD⁺ levels, the NR and NAR derivatives can enhance mitochondrial and cellular function and health and provide cytoprotection, and thus are useful for treating mitochondrial diseases, mitochondria-related diseases and conditions, diseases and conditions associated with acute NAD⁺ depletion induced by DNA damage, and other disorders.

NR and NAR Derivatives In some embodiments, NR and NAR derivatives have Formulas I and II:

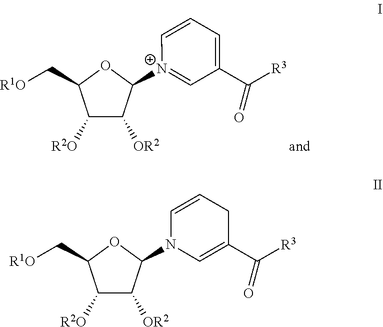

wherein:

R¹ is hydrogen,

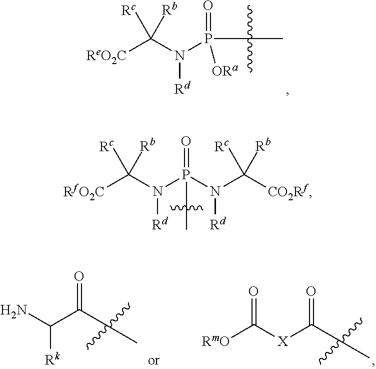

wherein:
R^{a} is hydrogen, a counterion, linear or branched C₁-C₆ alkyl, C₃-C₆ cycloalkyl, phenyl, 1-naphthyl or 2-naphthyl, wherein the phenyl is optionally substituted with F, Cl, —NO₂, linear or branched C₁-C₄ alkyl, —CF₃ or —O-(linear or branched C₁-C₄ alkyl);
R^{b} and R^{c} at each occurrence independently are hydrogen, linear or branched C₁-C₅ alkyl, —CH₂-phenyl, —CH₂-3-indole or —CH₂-5-imidazole, wherein the alkyl is optionally substituted with —OH, —OR^{j}, —SH, —SR^{j}, —NH₂, —NHR^{j}, —N(R^{j})₂, —NHC(═O)R^{j}, —NHC(═NH)NH₂, —C(═O)NH₂, —CO₂H or —C(═O)OR^{j}, and the phenyl is optionally substituted with —OH or —OR^{j}, wherein R^{j} at each occurrence independently is linear or branched C₁-C₄ alkyl;
R^{d} at each occurrence independently is hydrogen, methyl or linear or branched C₂-C₄ alkyl;
R^{e} and R^{f} at each occurrence independently are hydrogen, a counterion, linear or branched C₁-C₅ alkyl, C₃-C₆ cycloalkyl, —CH₂—(C₃-C₆ cycloalkyl), phenyl or —CH₂-phenyl, wherein the phenyl is optionally substituted with F, Cl, —NO₂, linear or branched C₁-C₄ alkyl, —CF₃ or —O-(linear or branched C₁-C₄ alkyl);
R^{k} is hydrogen, linear or branched C₁-C₆ alkyl, —CH₂-phenyl, —CH₂-3-indole or —CH₂-5-imidazole, wherein the alkyl is optionally substituted with —OH, —OR^{j}, —SH, —SR^{j}, —NH₂, —NHR^{j}, —N(R^{j})₂, —NHC(═O)R^{j}, —NHC(═NH)NH₂, —C(═O)NH₂, —CO₂H or —C(═O)OR^{j}, and the phenyl is optionally substituted with —OH or —OR^{j}, wherein R^{j} at each occurrence independently is linear or branched C₁-C₄ alkyl;
R^{m} is hydrogen, a counterion, linear or branched C₁-C₆ alkyl, C₃-C₆ cycloalkyl, phenyl, —CH₂-phenyl or

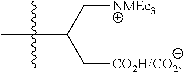

wherein the phenyl is optionally substituted with F, Cl, —NO₂, linear or branched C₁-C₄ alkyl, —CF₃ or —O-(linear or branched C₁-C₄ alkyl); and
X is cis or trans —HC═CH— or —(CH₂)— optionally substituted with —OH, —OR^{j} or —OC(═O)R^{j}, wherein R^{j} is linear or branched C₁-C₄ alkyl and n is 1, 2, 3, 4, 5 or 6;

R² at each occurrence independently is hydrogen

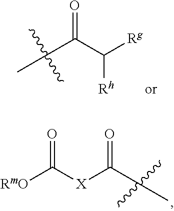

wherein:
R^{g} is hydrogen, linear or branched C₁-C₅ alkyl, —CH₂-phenyl, —CH₂-3-indole or —CH₂-5-imidazole, wherein the alkyl is optionally substituted with —OH, —OR^{j}, —SH, —SR^{j}, —NH₂, —NHR^{j}, —N(R^{j})₂, —NHC(═O)R^{j}, —NHC(═NH)NH₂, —C(═O)NH₂, —CO₂H or —C(═O)OR^{j}, and the phenyl is optionally substituted with —OH or —OR, wherein R^{j} at each occurrence independently is linear or branched C₁-C₄ alkyl;
R^{h} is hydrogen, methyl or —NH₂;
or R^{g} and R^{h} together with the carbon atom to which they are connected form a C₃-C₆ cycloalkyl or phenyl ring, wherein the phenyl ring is optionally substituted with F, Cl, —NO₂, linear or branched C₁-C₄ alkyl, —CF₃ or —O-(linear or branched C₁-C₄ alkyl); and
R^{m} and X are as defined above; and

R³ is —NH₂, —NHR^{j}, —N(Rⁿ)₂, —OH, —OR^{o} or

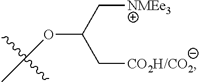

wherein:
Rⁿ at each occurrence independently is linear or branched C₁-C₆ alkyl or allyl, wherein the alkyl is optionally substituted with —OH or —O-(linear or branched C₁-C₃ alkyl), or both occurrences of Rⁿ and the nitrogen atom to which they are connected form a 3- to 6-membered heterocyclic ring; and
R^{o} is a counterion, linear or branched C₁-C₆ alkyl, C₃-C₆ cycloalkyl, phenyl or —CH₂-phenyl, wherein the phenyl is optionally substituted with F, Cl, —NO₂, linear or branched C₁-C₄ alkyl, —CF₃ or —O-(linear or branched C₁-C₄ alkyl);
or pharmaceutically acceptable salts, solvates, hydrates, clathrates, polymorphs or stereoisomers thereof;
with the proviso that:

1) R¹ and both occurrences of R² all are not hydrogen except when R³ is

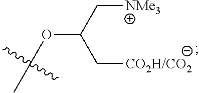

and

2) the compounds of Formulas I and II are not:

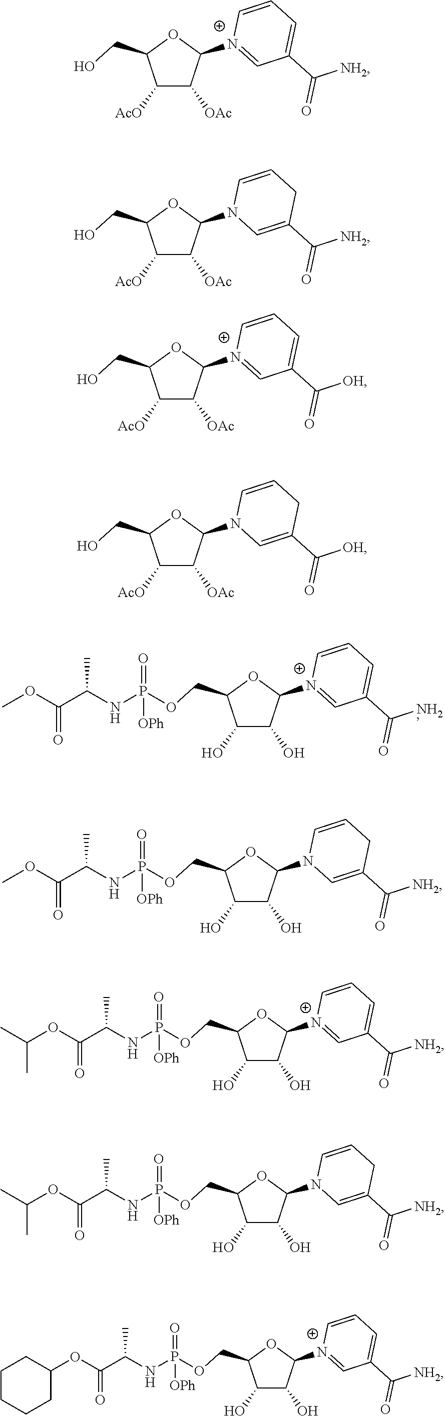

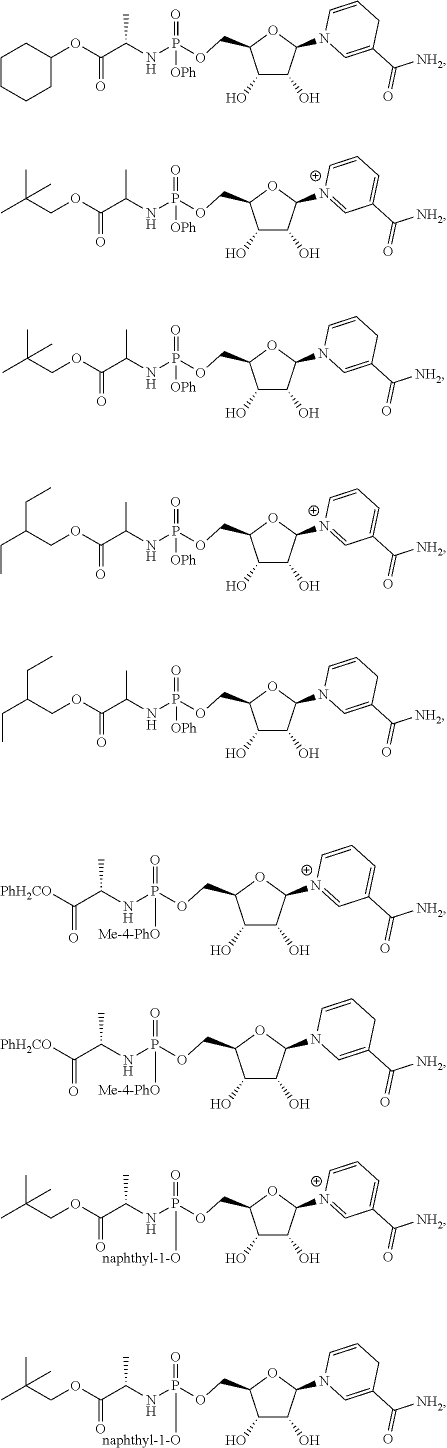

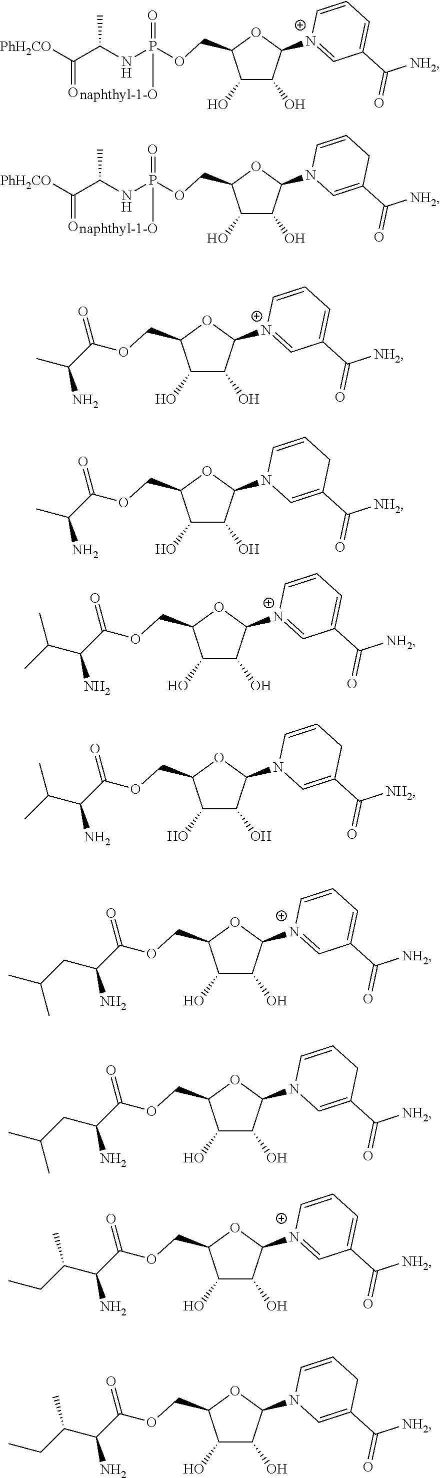

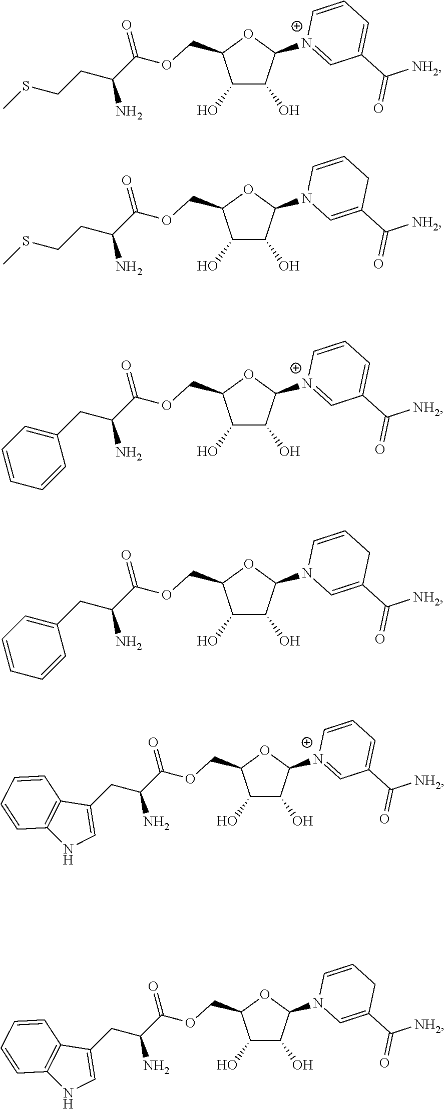 [SMILES: CSCC[C@H](N)C(=O)OC[C@H]1O[C@@H]([n+]2cccc(C(N)=O)c2)[C@H](O)[C@@H]1O ; CSCC[C@H](N)C(=O)OC[C@H]1O[C@@H](N2C=CCC(C(N)=O)=C2)[C@H](O)[C@@H]1O ; NC(=O)c1ccc[n+]([C@@H]2O[C@H](COC(=O)[C@@H](N)Cc3ccccc3)[C@@H](O)[C@H]2O)c1 ; NC(=O)C1=CN([C@@H]2O[C@H](COC(=O)[C@@H](N)Cc3ccccc3)[C@@H](O)[C@H]2O)C=CC1 ; NC(=O)c1ccc[n+]([C@@H]2O[C@H](COC(=O)[C@@H](N)Cc3c[nH]c4ccccc34)[C@@H](O)[C@H]2O)c1 ; NC(=O)C1=CN([C@@H]2O[C@H](COC(=O)[C@@H](N)Cc3c[nH]c4ccccc34)[C@@H](O)[C@H]2O)C=CC1]

or salts or stereoisomers thereof.

However, the compounds excluded from Formulas I and II above can be included within the scope of NR/NAR derivatives generally, or within the scope of Formulas I and II more specifically, in some embodiments of pharmaceutical compositions and therapeutic or medical uses.

In some embodiments, when both occurrences of R² are acetyl:

1) R¹ is not hydrogen; or
2) R³ is not —NH₂ or —OH or a salt thereof; or
3) R¹ is not hydrogen and R³ is not —NH₂ or —OH or a salt thereof.

In certain embodiments, when R¹ is

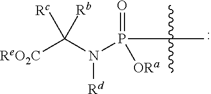

1) both occurrences of R² are not hydrogen; or
2) R³ is not —NH₂ or —OH or a salt thereof; or
3) both occurrences of R² are not hydrogen and R³ is not —NH₂ or —OH or a salt thereof.

In some embodiments, when R¹ is

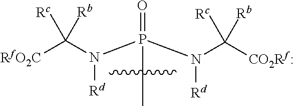

1) both occurrences of R² are not hydrogen; or
2) R³ is not —NH₂ or —OH or a salt thereof; or
3) both occurrences of R² are not hydrogen and R³ is not —NH₂ or —OH or a salt thereof.

In certain embodiments, when R¹ is

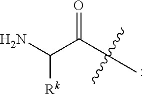

1) both occurrences of R² are not hydrogen; or
2) R³ is not —NH₂ or —OH or a salt thereof; or
3) both occurrences of R² are not hydrogen and R³ is not —NH₂ or —OH or a salt thereof.

In some embodiments, R¹ is

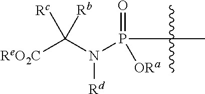

(phosphoramidate). In certain embodiments, R¹ is

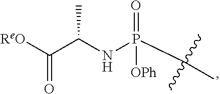

and R^{e} is linear or branched C₁-C₆ alkyl. In certain embodiments, R^{e} is methyl, ethyl or isopropyl.

In further embodiments, R¹ is

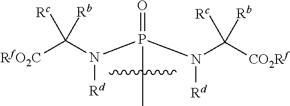

(phosphorodiamidate/bisphosphoramidate). In certain embodiments, R¹ is

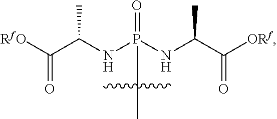

and both occurrences of R^{f} are linear or branched C₁-C₆ alkyl. In certain embodiments, both occurrences of R^{f} are methyl, ethyl or isopropyl.

In other embodiments, R¹ is

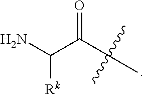

In certain embodiments, R¹ is

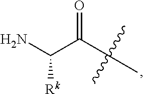

and R^{k} is linear or branched C₁-C₆ alkyl. In certain embodiments, R^{k} is methyl, ethyl or isopropyl. An amino acid group can facilitate penetration of an NR/NAR derivative through membrane barriers via peptide transporters, such as peptide transporter 1 in the intestinal epithelium.

In additional embodiments, R¹, or/and R² at either occurrence or at both occurrences, is/are

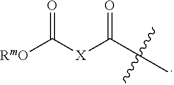

In some embodiments, X is trans —HC═CH—, —CH₂CH₂— or —CH(OH)CH₂—, and R^{m} is hydrogen, a counterion, linear or branched C₁-C₆ alkyl (e.g., methyl, ethyl or isopropyl) or

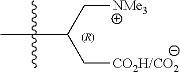

(an L-carnitine group). The —CH(OH)CH₂— portion can have the S-stereochemistry or a mixture (e.g., an approximately 1:1 ratio) of S/R-stereochemistry. In certain embodiments, R¹, or/and R² at either occurrence or at both occurrences, is/are selected from:

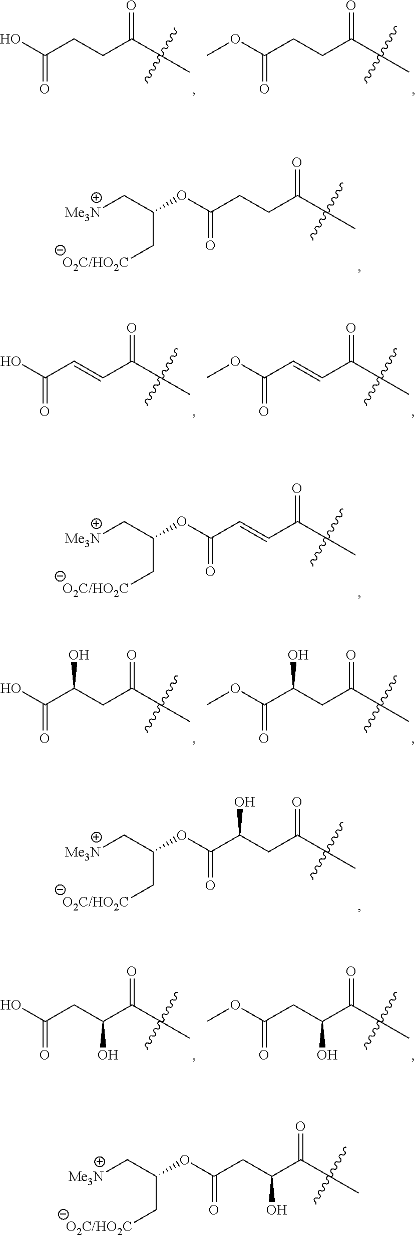

and salts thereof. A carnitine group can facilitate transport of an NR/NAR derivative into the mitochondria.

In some embodiments, R² at each occurrence independently, or at both occurrences, is hydrogen, —C(═O)-(linear or branched C₁-C₆ alkyl),

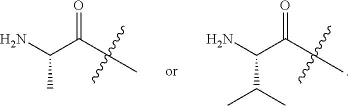

In certain embodiments, R² at each occurrence independently, or at both occurrences, is hydrogen, acetyl or propanoyl.

If a compound of Formula I or II comprises an amino acid group at R¹ or/and at either occurrence or both occurrences of R², including an amino acid group in a phosphoramidate moiety at R¹ or two amino acid groups in a phosphorodiamidate/isphosphoramidate moiety at R¹, the amino acid group can independently be a natural amino acid or an unnatural amino acid. In some embodiments, an amino acid group is glycine, alanine, valine, leucine, isoleucine, methionine, proline, tryptophan, phenylalanine, tyrosine, serine, threonine, cysteine, asparagine, glutamine, aspartic acid, glutamic acid, lysine, arginine or histidine, or a derivative thereof. In other embodiments, an amino acid group is an unnatural or non-proteinogenic amino acid, such as ornithine, citrulline or homoarginine. In certain embodiments, an amino acid group is glycine, alanine or valine. An amino acid group can be the L-isomer or the D-isomer, or can be a D/L (e.g., racemic) mixture. In certain embodiments, an amino acid group is the L-isomer.

In some embodiments, R³ is —NH₂, —OH or a salt thereof, or

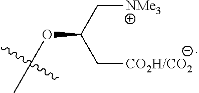

In certain embodiments, R³ is

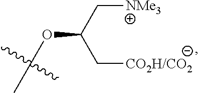

an L-carnitine moiety. The carnitine moiety can exist as a zwitterion.

In some embodiments of compounds of Formulas I and II:

### 1) R¹ is

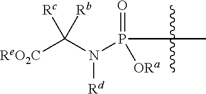

and both occurrences of R² are acetyl or propanoyl; or

### 2) R¹ is

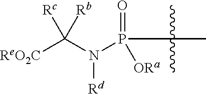

and R³ is —OH or a salt thereof; or

### 3) R¹ is

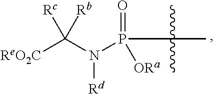

both occurrences of R² are acetyl or propanoyl, and R³ is —OH or a salt thereof.
In certain embodiments, R¹ is

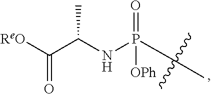

and R^{e} is linear or branched C₁-C₆ alkyl. In certain embodiments, R^{e} is methyl, ethyl or isopropyl.

In further embodiments of compounds of Formulas I and II:

R¹ is

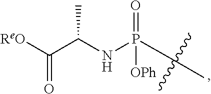

wherein R^{e} is linear or branched C₁-C₆ alkyl;

R² at both occurrences is —C(═O)-(linear or branched C₁-C₆ alkyl); and

R³ is —NH₂ or —OH or a salt thereof.

In certain embodiments, R^{e} of the R¹ moiety is methyl, ethyl or isopropyl, and both occurrences of R² are acetyl or propanoyl.

In other embodiments of compounds of Formulas I and II:

R¹ is

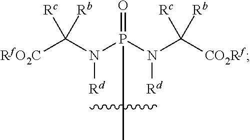

R² at each occurrence independently, or at both occurrences, is hydrogen, acetyl or propanoyl; and

R³ is —NH₂ or —OH or a salt thereof.

In some embodiments:

R^{b} and R^{c} at each occurrence independently are hydrogen or linear or branched C₁-C₅ alkyl, or each pair of R^{b} and R^{c} is hydrogen and linear or branched C₁-C₅ alkyl;

R^{d} at both occurrences is hydrogen; and

R^{f} at both occurrences is linear or branched C₁-C₆ alkyl.

In certain embodiments, R¹ is

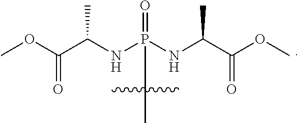

In further embodiments of compounds of Formulas I and II:

R¹ is

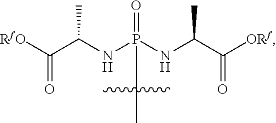

wherein both occurrences of R^{f} are linear or branched C₁-C₆ alkyl;

R² at each occurrence independently, or at both occurrences, is hydrogen or —C(═O)-(linear or branched C₁-C₆ alkyl); and

R³ is —NH₂ or —OH or a salt thereof.

In certain embodiments, both occurrences of R^{f} of the R¹ moiety are methyl, ethyl or isopropyl, and R² at each occurrence independently, or at both occurrences, is hydrogen, acetyl or propanoyl.

In additional embodiments of compounds of Formulas I and II:

R¹ is

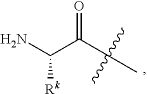

wherein R^{k} is linear or branched C₁-C₆ alkyl;

R² at each occurrence independently, or at both occurrences, is hydrogen or —C(═O)-(linear or branched C₁-C₆ alkyl); and

R³ is —NH₂ or —OH or a salt thereof.

In certain embodiments, R^{k} of the R¹ moiety is methyl, ethyl or isopropyl, and R² at each occurrence independently, or at both occurrences, is hydrogen, acetyl or propanoyl.

In other embodiments of compounds of Formulas I and II:

R¹ is

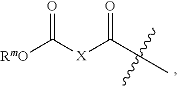

wherein:
X is cis or trans —HC═CH— or —(CH₂)ₙ— optionally substituted with —OH, —OR^{j} or —OC(═O)R^{j}, wherein R^{j} is linear or branched C₁-C₄ alkyl and n is 1, 2, 3, 4, 5 or 6; and
R^{m} is hydrogen, a counterion, linear or branched C₁-C₆ alkyl or

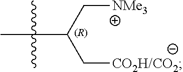

R² at each occurrence independently, or at both occurrences, is hydrogen or —C(═O)-(linear or branched C₁-C₆ alkyl); and

R³ is —NH₂ or —OH or a salt thereof.

In certain embodiments:

for the R¹ moiety, X is trans —HC═CH—, —CH₂CH₂— or —CH(OH)CH₂—, and R^{m} is hydrogen, a counterion, methyl, ethyl, isopropyl or

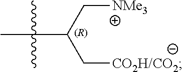

R² at each occurrence independently, or at both occurrences, is hydrogen, acetyl or propanoyl; and

R³ is —NH₂.

In some embodiments, the compounds of Formulas I and II are selected from:

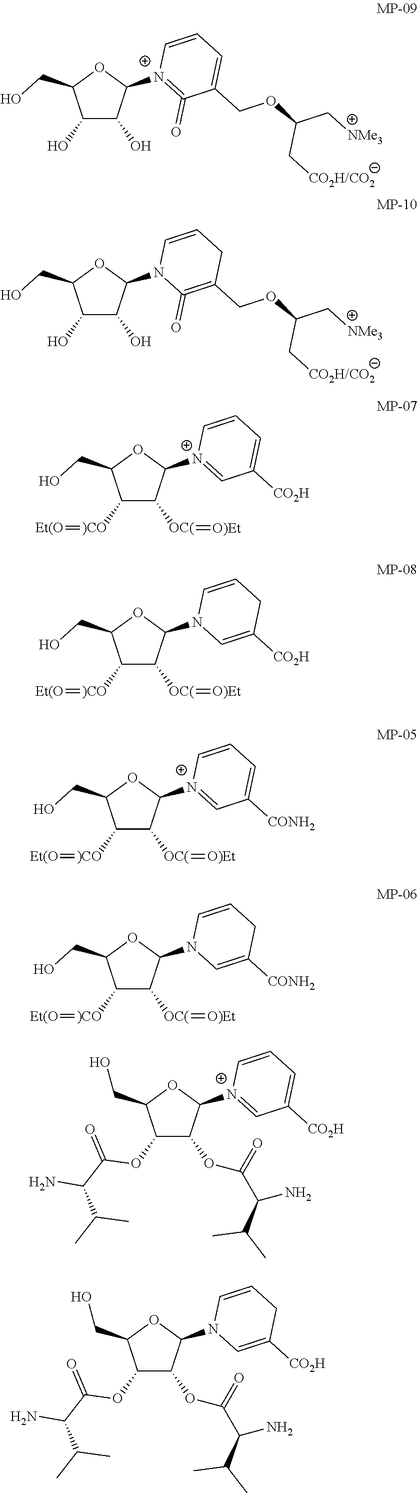

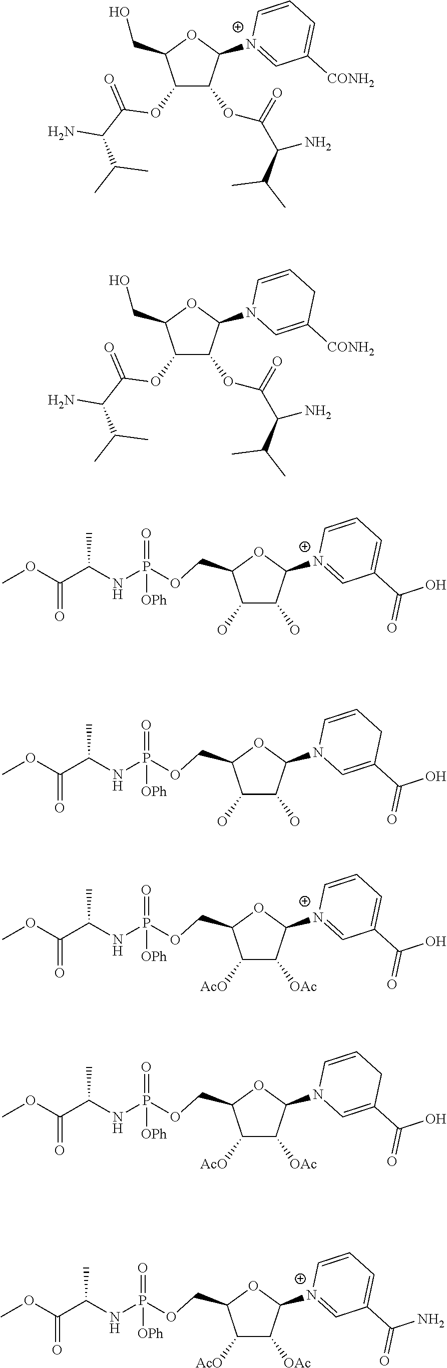

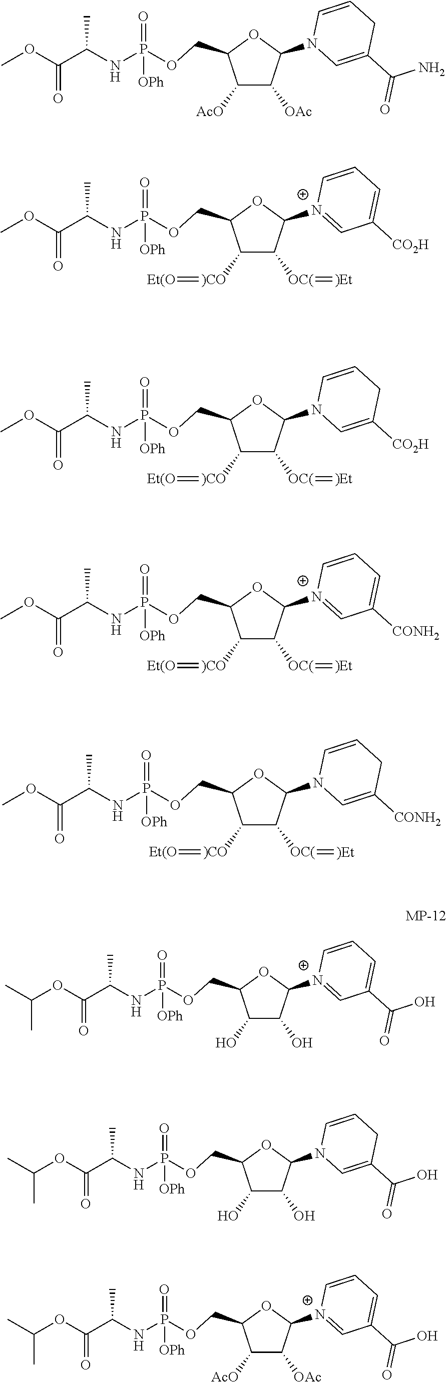

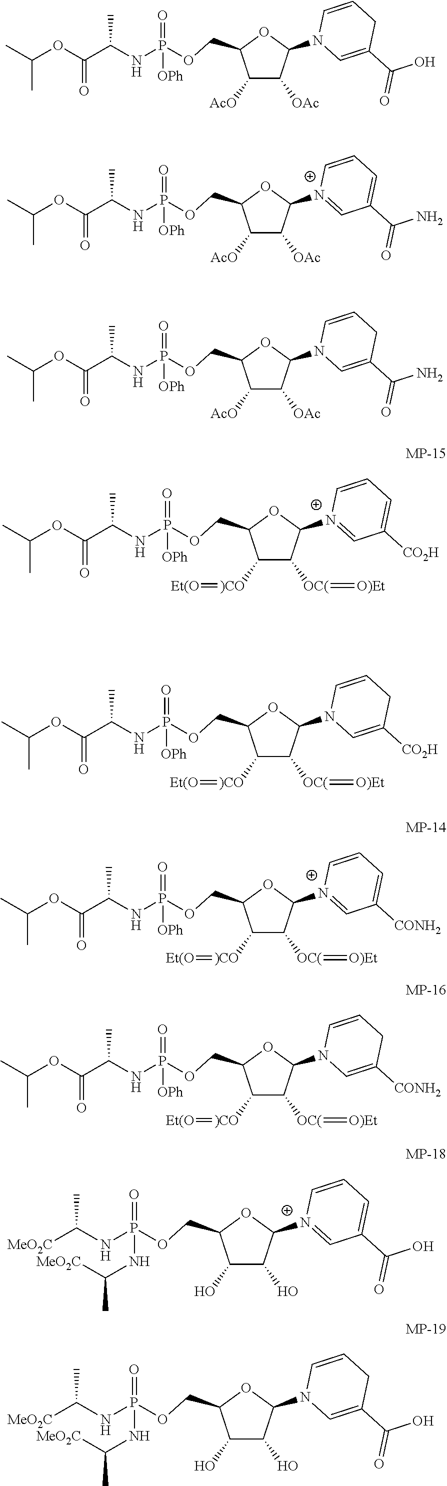

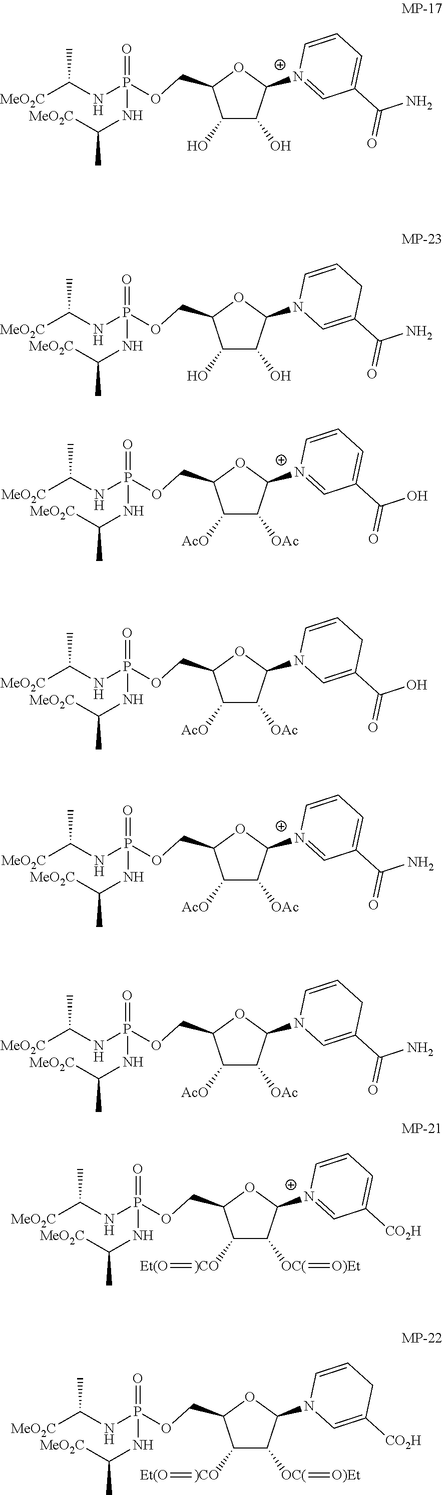

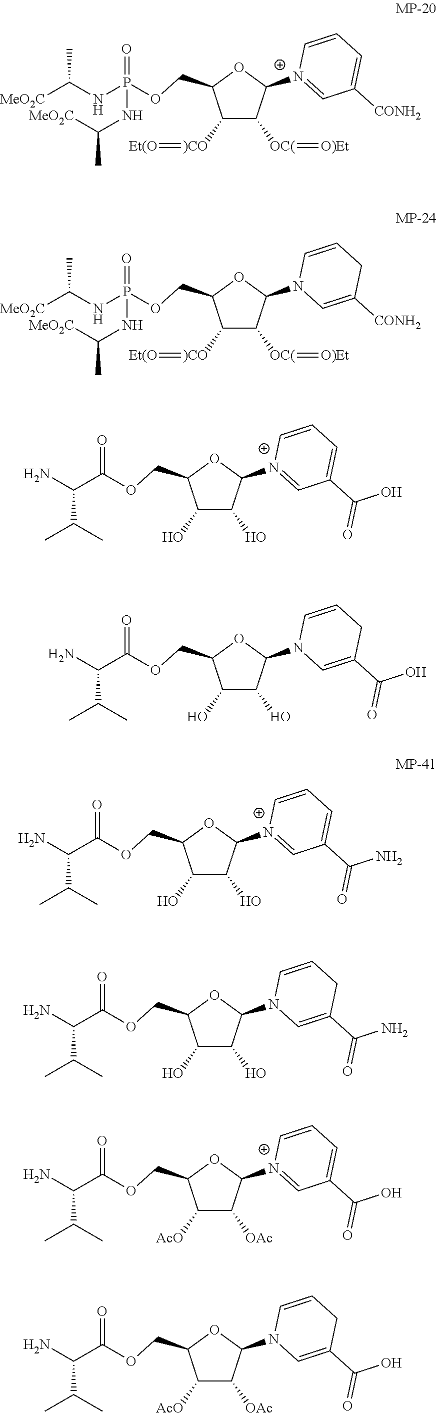

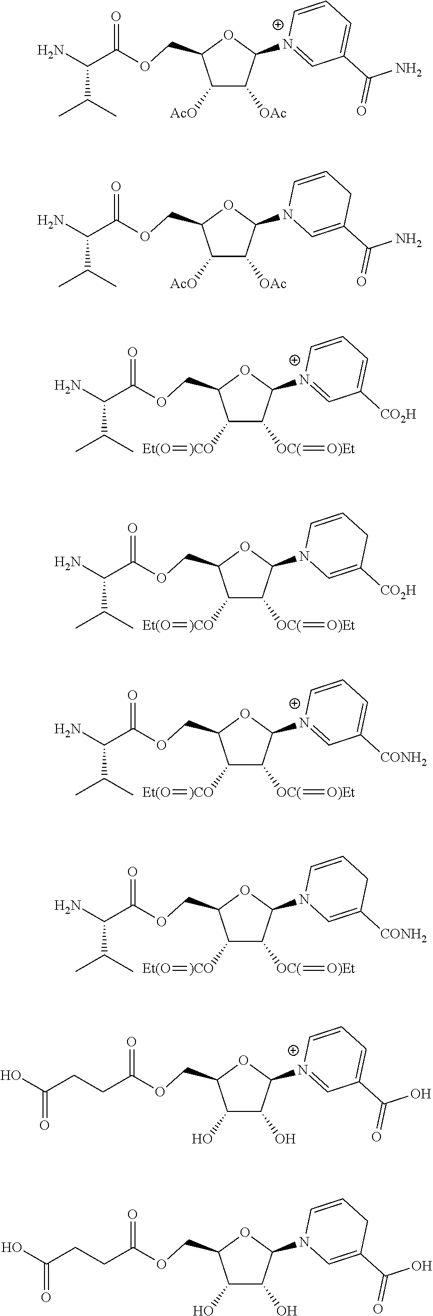

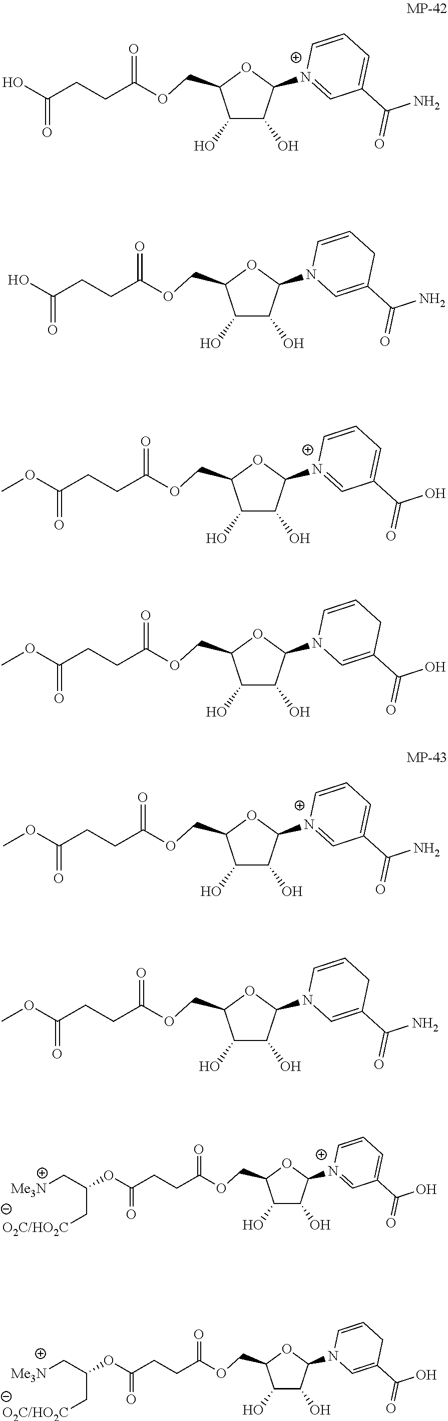

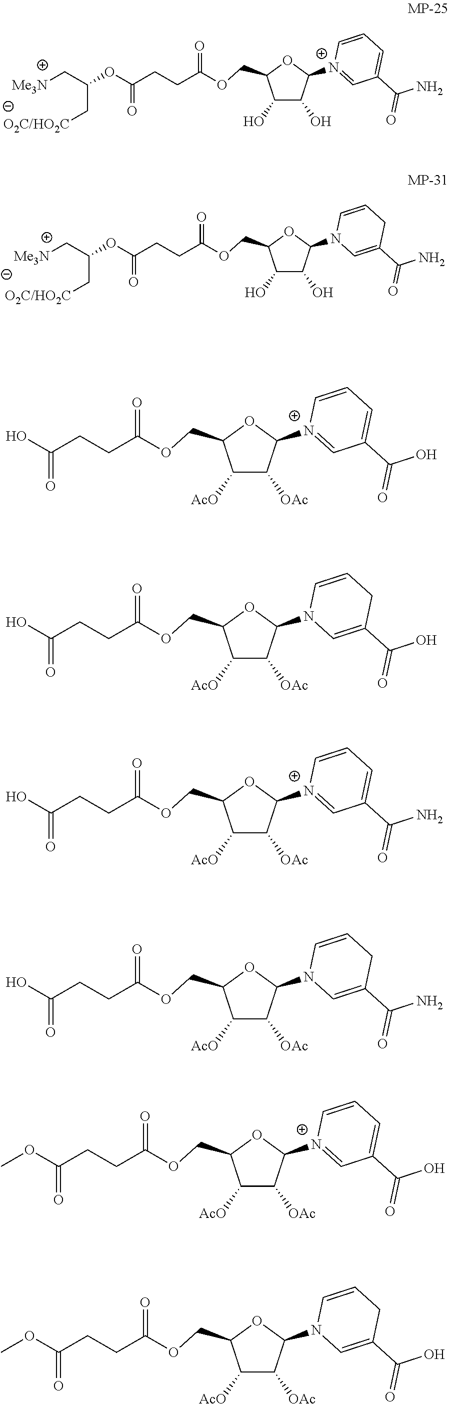

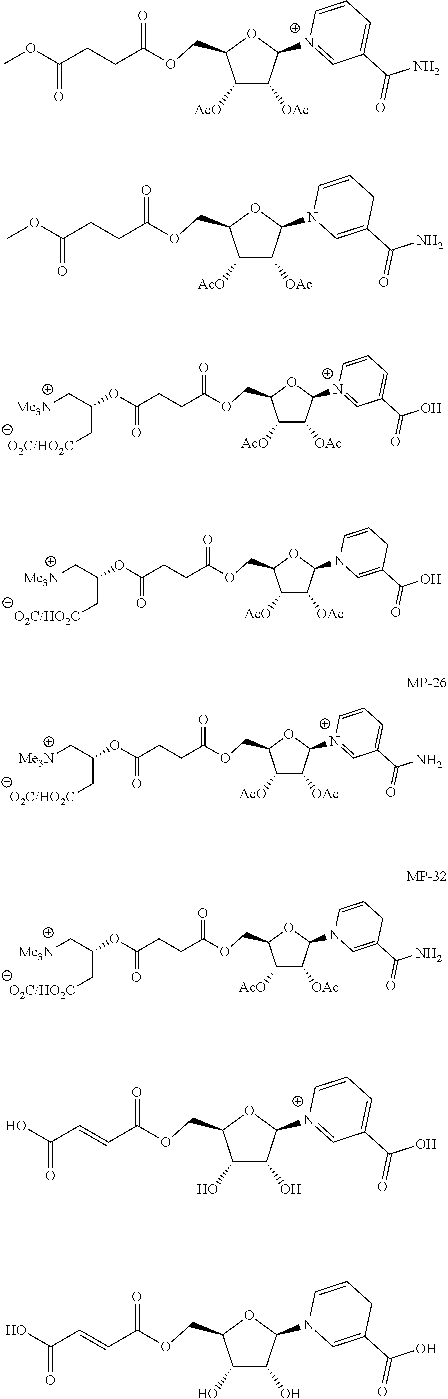

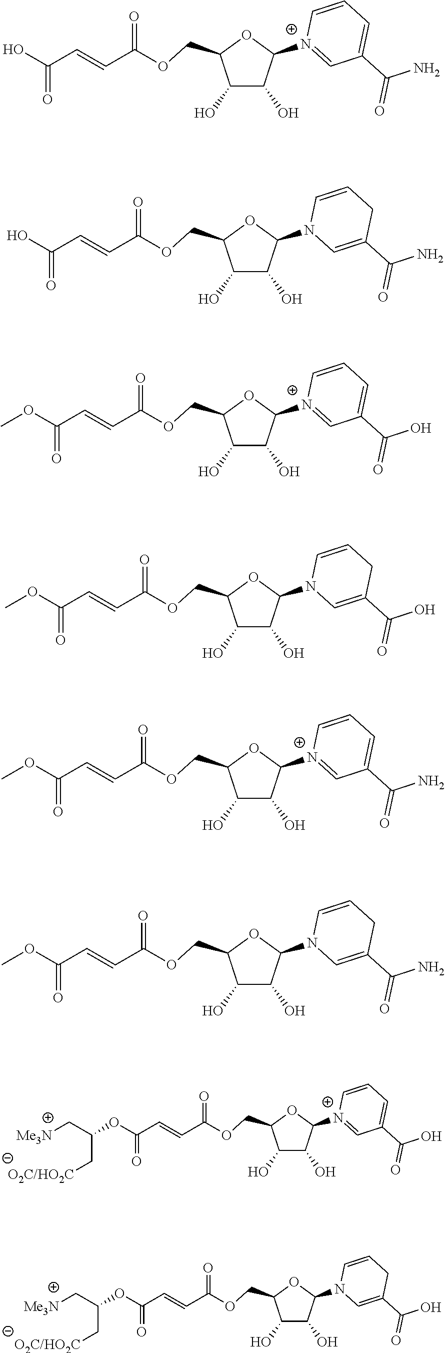

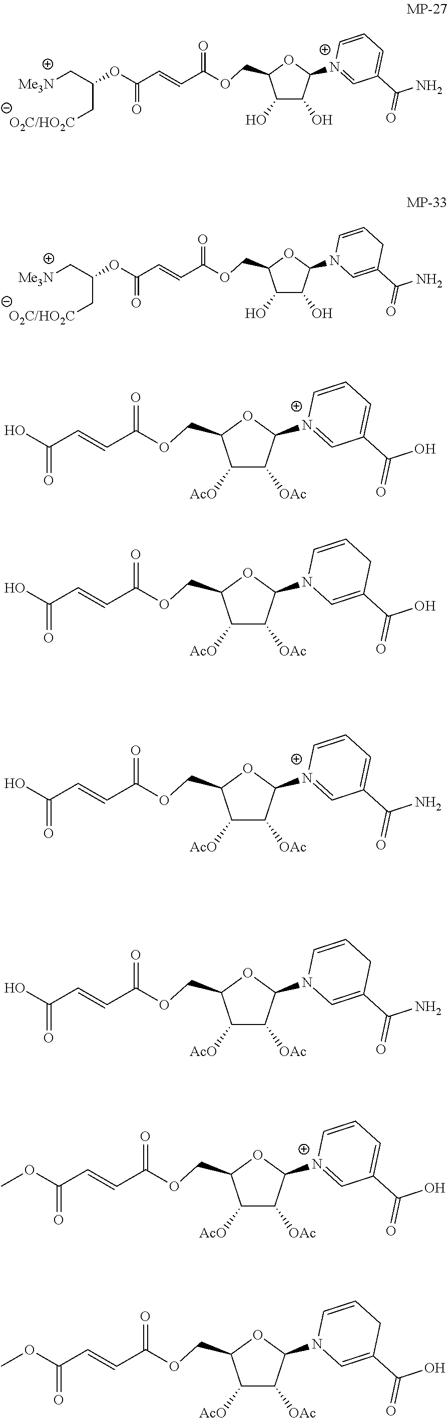

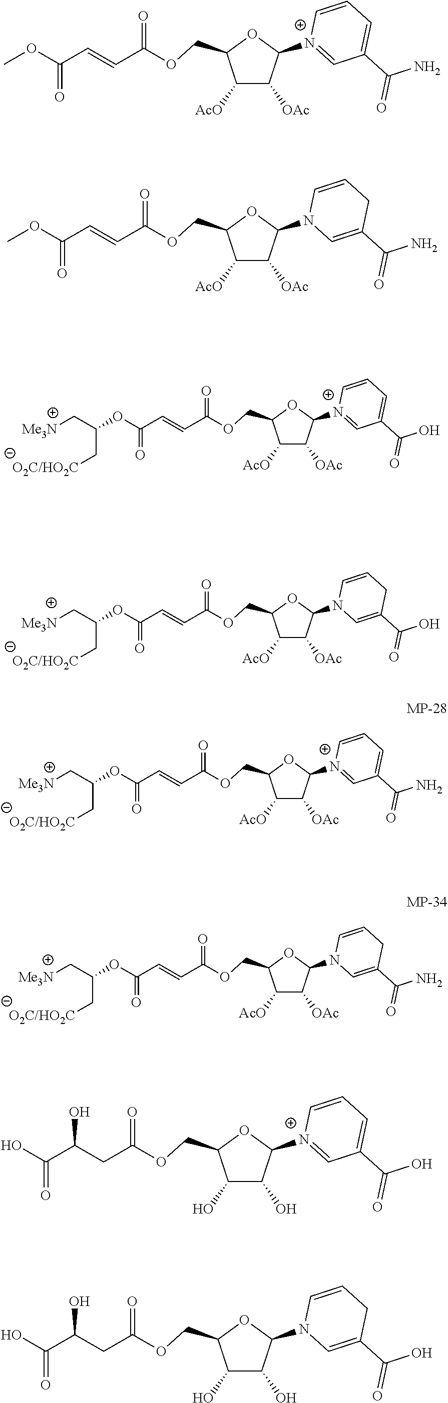

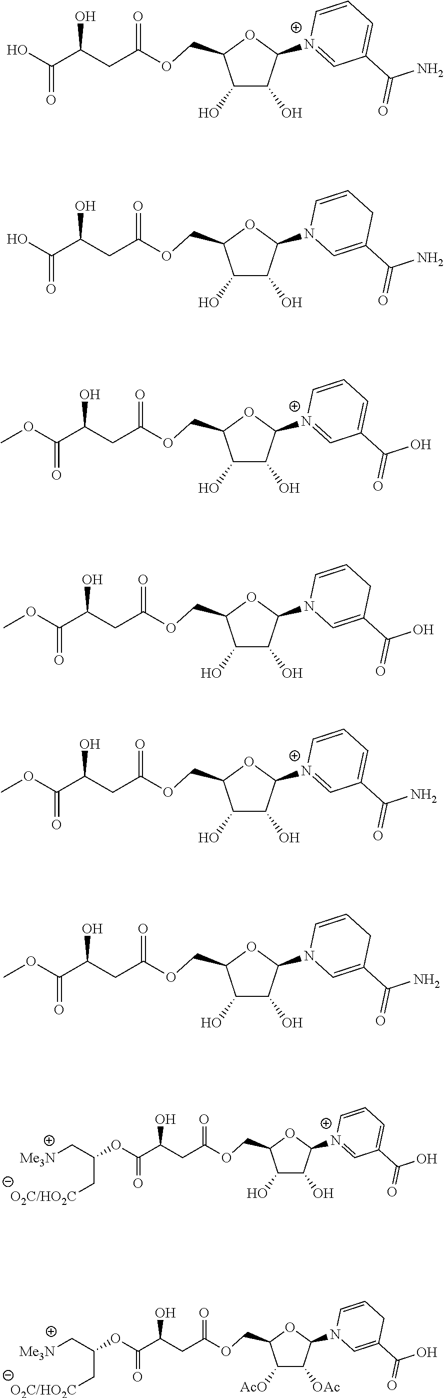

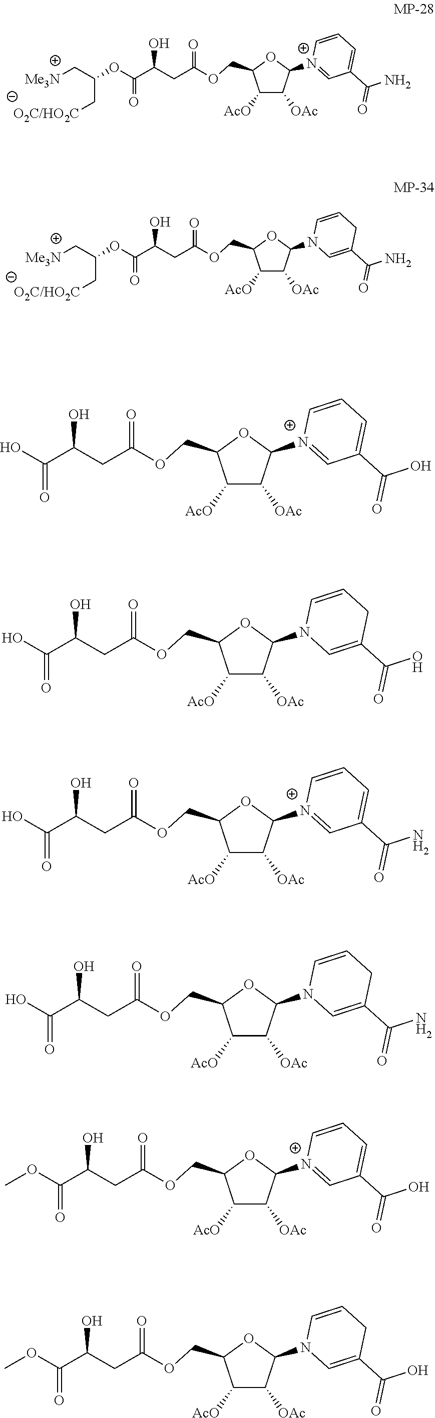

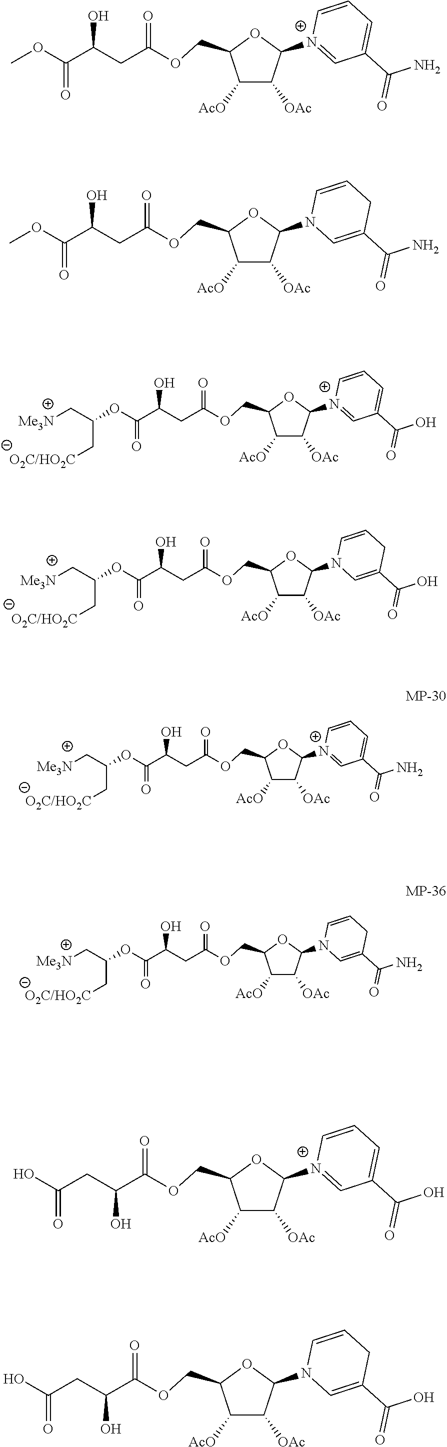

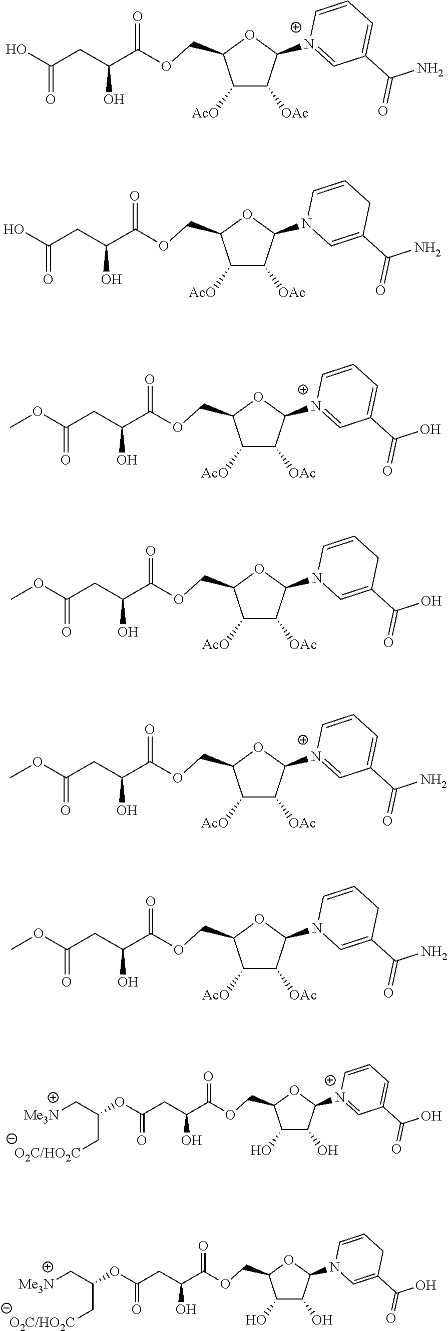

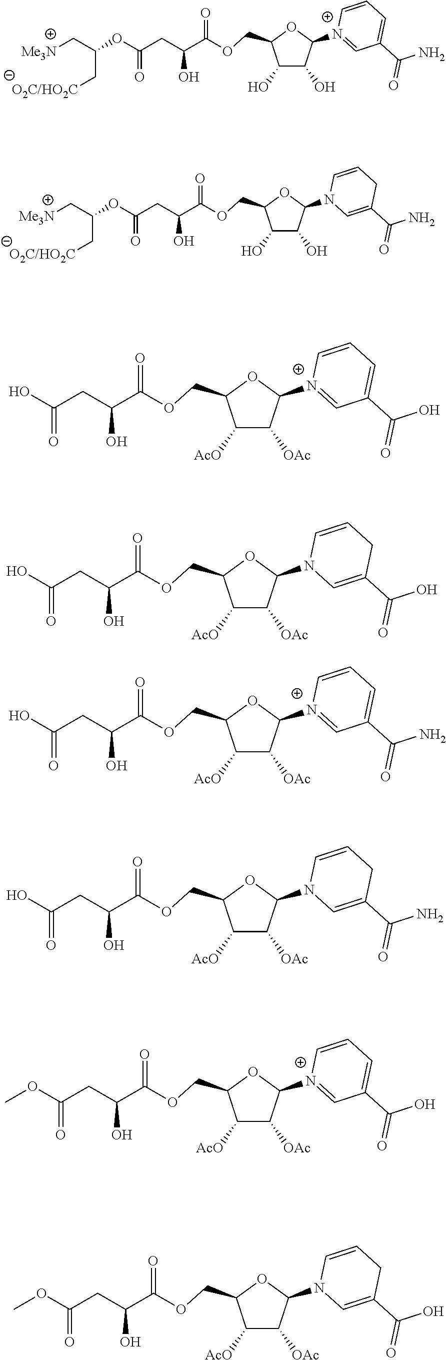

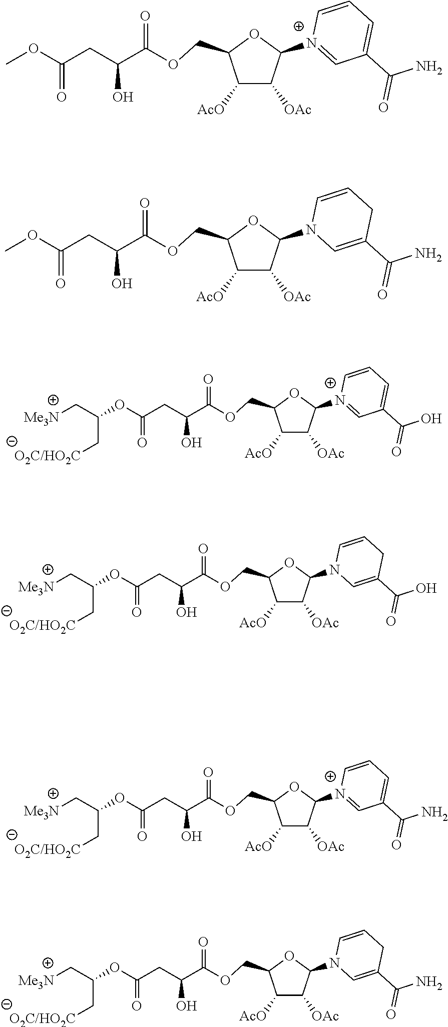

and pharmaceutically acceptable salts, solvates, hydrates, clathrates, polymorphs and stereoisomers thereof.

Other embodiments of the disclosure relate to NAR derivatives of Formulas III and IV:

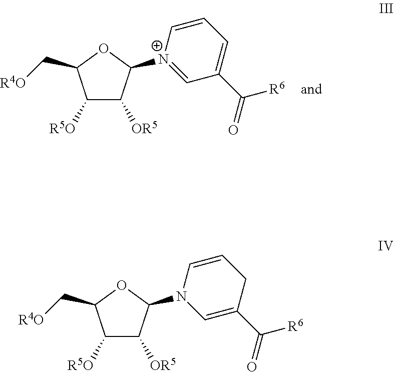

wherein:

R⁴ is hydrogen or —C(═O)R⁷, wherein R⁷ is linear or branched C₁-C₆ alkyl, C₃-C₆ cycloalkyl, or phenyl optionally substituted with F, Cl, —NO₂, linear or branched C₁-C₄ alkyl, —CF₃ or —O-(linear or branched C₁-C₄ alkyl);

R⁵ at each occurrence independently is hydrogen or —C(═O)R⁸, wherein R⁸ has the same definition as R⁷; and

R⁶ is

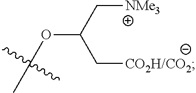

and pharmaceutically acceptable salts, solvates, hydrate, clathrates, polymorphs and stereoisomers thereof.

In some embodiments of compounds of Formulas III and IV:

R⁴ is hydrogen or —C(═O)R⁷, wherein R⁷ is linear or branched C₁-C₆ alkyl; and

R⁵ at each occurrence independently, or at both occurrences, is hydrogen or —C(═O)R⁸, wherein R⁸ is linear or branched C₁-C₆ alkyl.

In certain embodiments, R⁴ is hydrogen, acetyl or propanoyl, and R⁵ at each occurrence independently, or at both occurrences, is hydrogen, acetyl or propanoyl. In referred embodiments, the carnitine moiety of R⁶ is the L-isomer

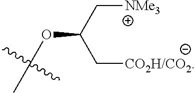

In certain embodiments, the compounds of Formulas III and IV are selected from:

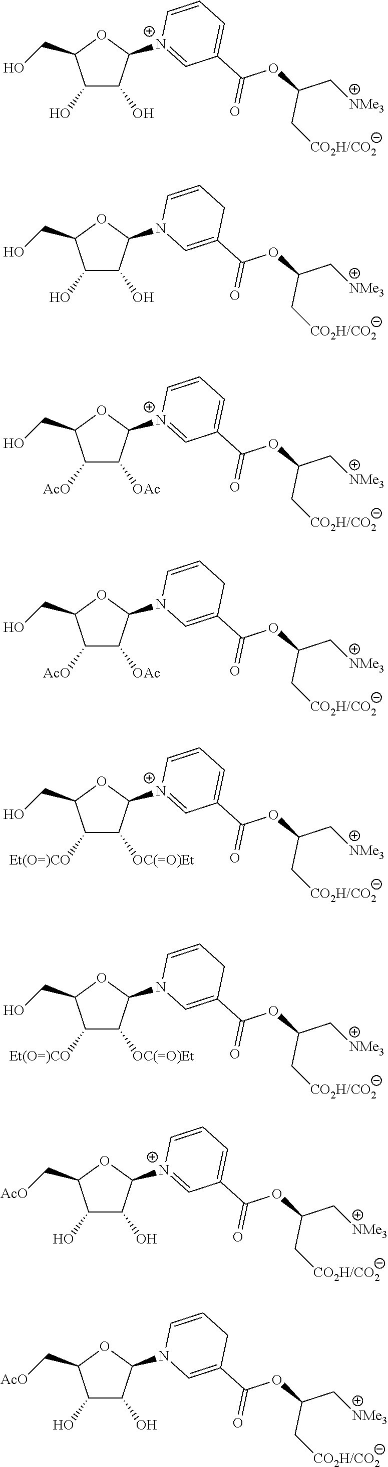

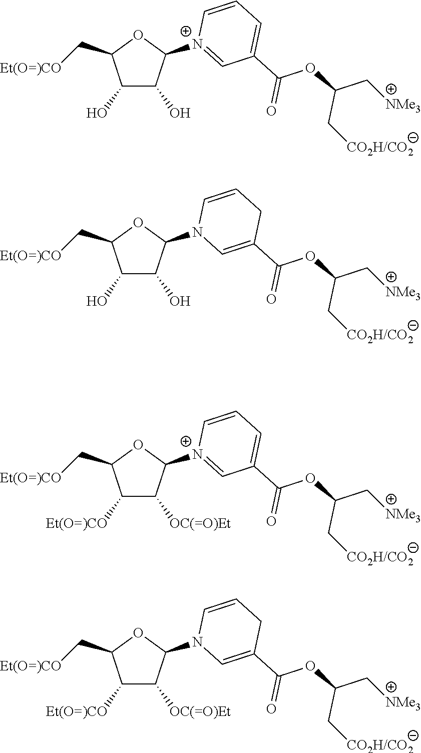

and pharmaceutically acceptable salts, solvates, hydrates, clathrates, polymorphs and stereoisomers thereof.

The compounds of Formulas I. II, III and IV can comprise a hydrophobic/lipophilic group at R¹/R⁴, at either occurrence or both occurrences of R²/R⁵, or at R³, or any combination thereof. One or more hydrophobic groups can facilitate permeation of an NR/NAR derivative through membrane barriers, including the cell membrane. In certain embodiments, a hydrophobic group contains 6-20 or 8-20 carbon atoms. In some embodiments, a hydrophobic group is a linear or branched, saturated (e.g., acyl or alkyl) group containing 6-20 or 8-20 carbon atoms, such as a linear saturated (e.g., acyl or alkyl) group containing 6, 8, 10, 12, 14, 16, 18 or 20 carbon atoms. In other embodiments, a hydrophobic group is a linear unsaturated (e.g., acyl or alkenyl) group containing 8-20 (e.g., 8, 10, 12, 14, 16, 18 or 20) carbon atoms and having 1, 2, 3 or 4 C═C double bonds, each of which can independently be cis or trans. In some embodiments, for the compounds of Formulas I and II:

R^{a} can be linear or branched C₁-C₂₀ alkyl or alkenyl;

R^{b} or R^{c} can be linear or branched C₁-C₂₀ alkyl or alkenyl for a phosphoramidate moiety:

R^{b} or R^{c} at either occurrence or both occurrences can be linear or branched C₁-C₂₀ alkyl or alkenyl for a phosphorodiamidate/bisphosphoramidate moiety;

R^{e} can be linear or branched C₁-C₂ alkyl or alkenyl;

R^{f} at either occurrence or both occurrences can be linear or branched C₁-C₂₀ alkyl or alkenyl;

R^{g} can be linear or branched C₁-C₂) alkyl or alkenyl;

R^{k} can be linear or branched C₁-C₂₀ alkyl or alkenyl;

R^{m} at any occurrence can be linear or branched C₁-C₂₀ alkyl or alkenyl;

Rⁿ at any occurrence can be linear or branched C₁-C₂₀ alkyl or alkenyl;

R^{o} can be linear or branched C₁-C₂₀ alkyl or alkenyl; or

n for —(CH₂)ₙ— for X at any occurrence can be an integer from 1 to 20, and —(CH₂)ₙ— for X can have one or more C═C double bonds; or

any combination or all of the above.

In some embodiments, for the compounds of Formulas III and IV:

R⁷ can be linear or branched C₁-C₂₀ alkyl or alkenyl; or/and

R⁸ at either occurrence or both occurrences can be linear or branched C₁-C₂₀ alkyl or alkenyl.

In some embodiments, the NR and NAR derivatives are the reduced form—i.e., have Formula II or IV.

The disclosure also encompasses isotopologues of the compounds of Formulas I, II, III and IV. Isotopically enriched forms of the NR and NAR derivatives described herein include without limitation those enriched in the content of ²H (deuterium), ³C, ¹⁵N, ¹⁷O or ¹⁸O, or any combination thereof, at one or more, or all, positions of the corresponding atom(s).

### Isomers of Compounds

The present disclosure encompasses all possible stereoisomers, including both enantiomers and all possible diastereomers in substantially pure form and mixtures of both enantiomers in any ratio (including a racemic mixture of enantiomers) and mixtures of two or more diastereomers in any ratio, of the compounds described herein, and not only the specific stereoisomers as indicated by drawn structure or nomenclature. In preferred embodiments, the disclosure relates to the specific stereoisomers indicated by drawn structure or nomenclature, including the beta-anomer of nicotinamide and nicotinic acid D-riboside derivatives. The specific recitation of the phrase “or stereoisomers thereof” or the like with respect to a compound in certain instances of the disclosure shall not be interpreted as an intended omission of any of the other possible stereoisomers of the compound in other instances of the disclosure where the compound is mentioned without recitation of the phrase “or stereoisomers thereof” or the like, unless stated otherwise or the context clearly indicates otherwise.

In some embodiments, the NR and NAR derivatives are stereoisomerically pure. In some embodiments, at least about 90%, 95%, 98% or 99% of the compounds of Formulas I, II, III and IV have the stereochemistry indicated by drawn structure or nomenclature, including the beta-D-riboside configuration. In similar embodiments, the compounds of Formulas I, II, III and IV have the beta-D-riboside configuration and an enantiomeric excess of at least about 80%, 90% or 95%.

In other embodiments, the compounds of Formulas I, II, III and IV are mixtures of enantiomers or mixtures of two or more diastereomers. In certain embodiments, the compounds of Formulas I, II, III and IV are racemic mixtures. In other embodiments, the compounds of Formulas I, II, III and IV have the D-riboside configuration and a mixture of beta-/alpha-anomers. In certain embodiments, the compounds of Formulas I, II, III and IV have the D-riboside configuration and an approximately 1:1 ratio of beta-/alpha-anomers.

The description and all of the embodiments relating to isomers of the NR and NAR derivatives disclosed herein also apply to isomers of other NR and NAR derivatives (e.g., nicotinamide riboside triacetate [NRTA, i.e., NR having an acetate group at each of the C-2, C-3 and C-5 positions of riboside], the reduced form of NRTA [NRHTA], nicotinic acid riboside triacetate [NARTA], and the reduced form of NARTA [NARHTA]) and to isomers of NR, NRH, NAR and NARH.

### Salt Forms of Compounds

The NR and NAR derivatives described herein can exist as salts, in particular their oxidized form—i.e., NR and NAR derivatives of Formulas I and III. The disclosure encompasses all pharmaceutically acceptable salts of NR and NAR derivatives. Examples of counteranions of salts of NR and NAR derivatives, including those of Formulas I and III, include without limitation internal salt, fluoride, chloride, bromide, iodide, nitrate, sulfate, sulfite, phosphate, bicarbonate, carbonate, thiocyanate, formate, acetate, trifluoroacetate, glycolate, lactate, gluconate, ascorbate, benzoate, oxalate, malonate, succinate, citrate, methanesulfonate (mesylate), ethanesulfonate, propanesulfonate, benzenesulfonate (bezylate), p-toluenesulfonate (tosylate) and trifluoromethanesulfonate (triflate). In certain embodiments, the NR and NAR derivatives, including those of Formulas I and III, are chloride, formate, acetate, trifluoroacetate or triflate salts.

If an NR or NAR derivative has an acidic group, such as a carboxylic acid or phosphoric acid group, it may form a salt with the acidic group. The countercation can be, e.g., Li⁺, Na⁺, K⁺, Ca⁺², Mg⁺², ammonium, a protonated organic amine (e.g., diethanolamine) or a quaternary ammonium compound (e.g., choline).

The description and all of the embodiments relating to salt forms of the NR and NAR derivatives disclosed herein also apply to salt forms of other NR and NAR derivatives (e.g., NRTA, NRHTA, NARTA and NARHTA) and to salt forms of NR, NRH, NAR and NARH.

### Pharmaceutical Compositions

The disclosure provides pharmaceutical compositions comprising one or more NR/NAR derivatives described herein, or a pharmaceutically acceptable salt, solvate, hydrate, clathrate, polymorph or stereoisomer thereof, and one or more pharmaceutically acceptable excipients or carriers. The compositions can optionally contain an additional therapeutic agent. In some embodiments, a pharmaceutical composition comprises a compound of Formula II or a compound of Formula IV. In further embodiments, a pharmaceutical composition comprises a compound of Formula I and a compound of Formula II, or a compound of Formula III and a compound of Formula IV. A pharmaceutical composition generally contains a therapeutically effective amount of the active ingredient, but can contain an appropriate fraction thereof. For purposes of the content of a pharmaceutical composition, the term “active ingredient”, “active agent”, “therapeutic agent” or “drug” encompasses a prodrug. For brevity, the term “pharmaceutical composition” encompasses a cosmetic composition, a cosmeceutical composition and a nutricosmetic composition.

A pharmaceutical composition contains an NR or NAR derivative in substantially pure form. In some embodiments, the purity of the NR or NAR derivative is at least about 95%, 96%, 97%, 98% or 99%. In certain embodiments, the purity of the NR or NAR derivative is at least about 98% or 99%. In addition, a pharmaceutical composition is substantially free of contaminants or impurities. In some embodiments, the level of contaminants or impurities other than residual solvent in a pharmaceutical composition is no more than about 5%, 4%, 3%, 2% or 1% relative to the combined weight of the intended active and inactive ingredients. In certain embodiments, the level of contaminants or impurities other than residual solvent in a pharmaceutical composition is no more than about 2% or 1% relative to the combined weight of the intended active and inactive ingredients.

Pharmaceutical compositions/formulations can be prepared in sterile form. For example, pharmaceutical compositions/formulations for parenteral administration by injection or infusion generally are sterile. Sterile pharmaceutical compositions/formulations are compounded or manufactured according to pharmaceutical-grade sterilization standards known to those of skill in the art, such as those disclosed in or required by the United States Pharmacopeia Chapters 797, 1072 and 1211, and 21 Code of Federal Regulations 211.

Pharmaceutically acceptable excipients and carriers include pharmaceutically acceptable substances, materials and vehicles. Non-limiting examples of types of excipients include liquid and solid fillers, diluents, binders, lubricants, glidants, surfactants, dispersing agents, disintegration agents, emulsifying agents, wetting agents, suspending agents, thickeners, solvents, isotonic agents, buffers, pH adjusters, absorption-delaying agents, stabilizers, antioxidants, preservatives, antimicrobial agents, antibacterial agents, antifungal agents, chelating agents, adjuvants, sweetening agents, flavoring agents, coloring agents, encapsulating materials and coating materials. The use of such excipients in pharmaceutical formulations is known in the art. For example, conventional vehicles and carriers include without limitation oils (e.g., vegetable oils such as olive oil and sesame oil), aqueous solvents {e.g., saline, buffered saline (e.g., phosphate-buffered saline [PBS]) and isotonic solutions (e.g., Ringer's solution)}, and organic solvents (e.g., dimethyl sulfoxide [DMSO] and alcohols [e.g., ethanol, glycerol and propylene glycol]). Except insofar as any conventional excipient or carrier is incompatible with the active ingredient, the disclosure encompasses the use of conventional excipients and carriers in formulations containing one or more NR/NAR derivatives. See, e.g., Remington: The Science and Practice of Pharmacy, 21st Ed., Lippincott Williams & Wilkins (Philadelphia, Pa.) (2005); Handbook of Pharmaceutical Excipients, 5th Ed., Rowe et al., Eds., The Pharmaceutical Press and the American Pharmaceutical Association (2005); Handbook of Pharmaceutical Additives, 3rd Ed., Ash and Ash, Eds., Gower Publishing Co. (2007); and Pharmaceutical Pre-formulation and Formulation, Gibson, Ed., CRC Press (Boca Raton, Fla.) (2004).

Appropriate formulation can depend on various factors, such as the route of administration chosen. Potential routes of administration of pharmaceutical compositions containing one or more NR/NAR derivatives include without limitation oral, parenteral (including intradermal, subcutaneous, intramuscular, intravascular, intravenous, intra-arterial, intraperitoneal, intracavitary, intramedullary, intrathecal and topical), and topical (including dermal/epicutaneous, transdermal, mucosal, transmucosal, intranasal [e.g., by nasal spray or drop], ocular [e.g., by eye drop], pulmonary [e.g., by oral or nasal inhalation], buccal, sublingual, rectal [e.g., by suppository], and vaginal [e.g., by suppository]). Topical formulations can be designed to produce a local or systemic therapeutic effect.

As an example, formulations of NR/NAR derivatives suitable for oral administration can be presented as, e.g., boluses; capsules (including push-fit capsules and soft capsules), tablets, pills, cachets or lozenges; as powders or granules; as semisolids, electuaries, pastes or gels; as solutions or suspensions in an aqueous liquid or/and a non-aqueous liquid; or as oil-in-water liquid emulsions or water-in-oil liquid emulsions.

Push-fit capsules or two-piece hard gelatin capsules can contain one or more NR/NAR derivatives in admixture with, e.g., a filler or inert solid diluent (e.g., calcium carbonate, calcium phosphate, kaolin or lactose), a binder (e.g., a starch), a glidant or lubricant (e.g., talc or magnesium stearate), and a disintegrant (e.g., crospovidone), and optionally a stabilizer or/and a preservative. For soft capsules or single-piece gelatin capsules, one or more NR/NAR derivatives can be dissolved or suspended in a suitable liquid (e.g., liquid polyethylene glycol or an oil medium, such as a fatty oil, peanut oil, olive oil or liquid paraffin), and the liquid-filled capsules can contain one or more other liquid excipients or/and semi-solid excipients, such as a stabilizer or/and an amphiphilic agent (e.g., a fatty acid ester of glycerol, propylene glycol or sorbitol).

Tablets can contain one or more NR/NAR derivatives in admixture with, e.g., a filler or inert diluent (e.g., calcium carbonate, calcium phosphate, lactose, mannitol or microcrystalline cellulose [MCC]), a binding agent (e.g., a starch, gelatin, acacia, alginic acid or a salt thereof, or MCC), a lubricating agent (e.g., stearic acid, magnesium stearate, talc or silicon dioxide), and a disintegrating agent (e.g., crospovidone, croscarmellose sodium or colloidal silica), and optionally a surfactant (e.g., sodium lauryl sulfate). The tablets can be uncoated or can be coated with, e.g., an enteric coating (e.g., Opadryo Enteric [94 Series]) that protects the active ingredient from the acidic environment of the stomach, or/and with a material that delays disintegration and absorption of the active ingredient in the gastrointestinal (GI) tract and thereby provides a sustained action over a longer time period.

Compositions for oral administration can also be formulated as solutions or suspensions in an aqueous liquid or/and a non-aqueous liquid, or as oil-in-water liquid emulsions or water-in-oil liquid emulsions. Dispersible powder or granules of one or more NR/NAR derivatives can be mixed with any suitable combination of an aqueous liquid, an organic solvent or/and an oil and any suitable excipients (e.g., any combination of a dispersing agent, a wetting agent, a suspending agent, an emulsifying agent or/and a preservative) to form a solution, suspension or emulsion.

NR and NAR derivatives can also be formulated for parenteral administration by, e.g., injection or infusion to circumvent GI absorption and first-pass metabolism. An exemplary parenteral route is intravenous. Additional advantages of intravenous administration include direct administration of a therapeutic agent into systemic circulation to achieve a rapid systemic effect, and the ability to administer the agent continuously or/and in a large volume if desired. Formulations for injection or infusion can be in the form of, e.g., solutions, suspensions or emulsions in oily or aqueous vehicles, and can contain excipients such as suspending agents, dispersing agents or/and stabilizing agents. For example, aqueous (e.g., saline) or non-aqueous (e.g., oily) sterile injection solutions can contain one or more NR/NAR derivatives along with excipients such as an antioxidant, a buffer, a bacteriostat and solutes that render the formulation isotonic with the blood of the subject. Aqueous or non-aqueous sterile suspensions can contain one or more NR/NAR derivatives along with excipients such as a suspending agent and a thickening agent, and optionally a stabilizer and an agent that increases the solubility of the NR/NAR derivative(s) to allow for the preparation of a more concentrated solution or suspension. As another example, a sterile aqueous solution for injection or infusion (e.g., subcutaneously or intravenously) can contain one or more NR/NAR derivatives, sodium chloride, a buffering agent (e.g., sodium citrate), a preservative (e.g., meta-cresol), and optionally a base (e.g., NaOH) or/and an acid (e.g., HCl) to adjust pH.

In some embodiments, a composition for parenteral (e.g., intravenous) administration comprises a complex of an NR or NAR derivative with a dendrimer [e.g., a poly(amidoamine) (PAMAM) or poly(ethylene glycol) (PEG) dendrimer], which can be, e.g., in an aqueous solution or a colloidal liposomal formulation. As an illustrative example, an NR or NAR derivative can be combined with a dendrimer (e.g., a PAMAM or PEG dendrimer) by encapsulation (e.g., the dendrimer forms a nanoparticle or micelle encapsulating the NR or NAR derivative), electrostatic or ionic interaction or other non-covalent association, or covalent conjugation using, e.g., an enzyme-cleavable linker (e.g., Gly-Phe-Leu-Gly). The dendrimer can optionally have one or more (e.g., ten or more) moieties (e.g., attached to the surface of a dendrimer core) that target the dendrimer-NR/NAR derivative complex to specific organ(s), tissue(s), cell type(s) or organelle(s), such as the liver, tumor/cancer cells or mitochondria. For example, the dendrimer can optionally have one or more N-acetylgalactosamine (GaNAc) moieties, which can target the dendrimer-containing composition to the liver by binding to asialoglycoprotein receptors on hepatocytes for treatment of, e.g., a liver or metabolic disorder. Such a dendrimer-containing composition can also be formulated for oral administration or other modes of parenteral administration (e.g., subcutaneous, intramuscular, intrathecal or topical).

For topical administration, one or more NR/NAR derivatives can be formulated as, e.g., a buccal or sublingual tablet or pill. Advantages of a buccal or sublingual tablet or pill include avoidance of GI absorption and first-pass metabolism, and rapid absorption into systemic circulation. A buccal or sublingual tablet or pill can be designed to provide faster release of the NR/NAR derivative(s) for more rapid uptake into systemic circulation. A buccal or sublingual tablet or pill can contain suitable excipients, including without limitation any combination of fillers and diluents (e.g., mannitol and sorbitol), binding agents (e.g., sodium carbonate), wetting agents (e.g., sodium carbonate), disintegrants (e.g., crospovidone and croscarmellose sodium), lubricants (e.g., silicon dioxide [including colloidal silicon dioxide] and sodium stearyl fumarate), stabilizers (e.g., sodium bicarbonate), flavoring agents (e.g., spearmint flavor), sweetening agents (e.g., sucralose), and coloring agents (e.g., yellow iron oxide).

For topical administration, NR and NAR derivatives can also be formulated for intranasal administration. The nasal mucosa provides a big surface area, a porous endothelium, a highly vascular subepithelial layer and a high absorption rate, and hence allows for high bioavailability. Moreover, intranasal administration avoids first-pass metabolism and can introduce a significant concentration of the active ingredient to the central nervous system (CNS). An intranasal formulation can comprise one or more NR/NAR derivatives along with excipients, such as a solubility enhancer (e.g., propylene glycol), a humectant (e.g., mannitol or sorbitol), a buffer and water, and optionally a preservative (e.g., benzalkonium chloride), a mucoadhesive agent (e.g., hydroxyethylcellulose) or/and a penetration enhancer. An intranasal solution or suspension formulation can be administered to the nasal cavity by any suitable means, including but not limited to a dropper, a pipette, or spray using, e.g., a metering atomizing spray pump.

An additional mode of topical administration of NR and NAR derivatives is pulmonary, including by oral inhalation and nasal inhalation. The lungs serve as a portal to the systemic circulation. Advantages of pulmonary drug delivery include, for example: 1) avoidance of first-pass hepatic metabolism; 2) fast drug action; 3) large surface area of the alveolar region for absorption, high permeability of the lungs (thin air-blood barrier), and profuse vasculature of the airways; 4) reduced extracellular enzyme levels compared to the GI tract due to the large alveolar surface area; and 5) smaller doses to achieve equivalent therapeutic effect compared to other oral routes, and hence reduced systemic side effects. Oral inhalation can also enable more rapid action of a drug in the CNS. An advantage of oral inhalation over nasal inhalation includes deeper penetration/deposition of the drug into the lungs. Oral or nasal inhalation can be achieved by means of, e.g., a metered-dose inhaler, a dry powder inhaler or a nebulizer, as is known in the art. In certain embodiments, a sterile aqueous solution for oral inhalation contains one or more NR/NAR derivatives, sodium chloride, a buffering agent (e.g., sodium citrate), optionally a preservative (e.g., meta-cresol), and optionally a base (e.g., NaOH) or/and an acid (e.g., HCl) to adjust pH.

Topical formulations for application to the skin or mucosa can be useful for transdermal or transmucosal administration of a drug into the underlying tissue or/and the blood for systemic distribution. Advantages of topical administration can include circumvention of GI absorption and first-pass metabolism, delivery of a drug with a short half-life and low oral bioavailability, more controlled and sustained release of the drug, a more uniform plasma dosing or delivery profile of the drug, less frequent dosing of the drug, less side effects, minimal or no invasiveness, ease of self-administration, and increased patient compliance.

In general, compositions suitable for topical administration include without limitation liquid or semi-liquid preparations such as sprays, gels, liniments and lotions, oil-in-water or water-in-oil emulsions such as creams, foams, ointments and pastes, and solutions or suspensions such as drops (e.g., eye drops, nose drops and ear drops). In some embodiments, a topical composition comprises a drug dissolved, dispersed or suspended in a carrier. The carrier can be in the form of, e.g., a solution, a suspension, an emulsion, an ointment or a gel base, and can contain, e.g., petrolatum, lanolin, a wax (e.g., bee wax), mineral oil, a long-chain alcohol, polyethylene glycol or polypropylene glycol, or a diluent (e.g., water or/and an alcohol [e.g., ethanol or propylene glycol]), or any combination thereof. A solvent such as an alcohol can be used to solubilize the drug. A topical composition can contain any of a variety of excipients, such as a gelling agent, an emulsifier, a thickening agent, a buffer, a stabilizer, an antioxidant, a preservative, a chemical permeation enhancer (CPE) or an irritation-mitigating agent, or any combination thereof. A topical composition can include, or a topical formulation can be administered by means of, e.g., a transdermal or transmucosal delivery device, such as a transdermal patch, a microneedle patch or an iontophoresis device. A topical composition can deliver a drug transdermally or transmucosally via a concentration gradient (with or without the use of a CPE) or an active mechanism (e.g., iontophoresis or microneedles).

For transdermal or transmucosal administration, in some embodiments a topical composition comprises a chemical penetration enhancer (CPE) that increases permeation of a drug across the skin or mucosa into the underlying tissue or/and systemic circulation. Examples of CPEs include without limitation alcohols and fatty alcohols (e.g., methanol, ethanol, isopropyl alcohol, pentanol, lauryl alcohol, oleyl alcohol, menthol, benzyl alcohol, diethylene glycol mono-ethyl ether, propylene glycol, dipropylene glycol, polyethylenc glycol and glycerol); ethers (e.g., eucalyptol); fatty acids (e.g., capric acid, lauric acid, myristic acid, oleic acid, linoleic acid and linolenic acid); esters, fatty alcohol esters and fatty acid esters (e.g., ethyl acetate, methyl laurate, isopropyl myristate, isopropyl palmitate, methyl oleate, ethyl oleate, propylene glycol mono-oleate, glycerol mono-oleate, triacetin and pentadecalactone); hydroxyl-containing esters, fatty alcohol esters and fatty acid esters (e.g., lauryl lactate, glyceryl/glycerol monolaurate, glycerol monoleate [mono-olein], sorbitan oleate, octyl salicylate and fatty acid esters of saccharides [e.g., sucrose fatty acid esters such as sucrose laurate]); amides, fatty amine amides and fatty acid amides (e.g., urea, dimethylformamide, dimethylacetamide, diethylacetamide, diethyltoluamide, N-lauroyl sarcosine, 1-dodecylazacycloheptane-2-one [laurocapram or Azoneo], Azone-related compounds, and pyrrolidone compounds [e.g., 2-pyrrolidone and N-methyl-2-pyrrolidone]); and ionic and non-ionic surfactants (e.g., cetyltrimethylammonium bromide, sodium laurate, sodium laureth sulfate [sodium lauryl ether sulfate], sodium cholate, sodium lauroyl sarcosinate. N-lauroyl sarcosine, sorbitan monolaurate, Brij® surfactants, Pluronic® surfactants, Tween® surfactants, saponins, alkyl glycosides, and fatty ether and fatty ester saccharides). US 2007/0269379 provides an extensive list of CPEs.

In some embodiments, the CPE includes a surfactant. In certain embodiments, the CPE includes two or more surfactants, such as a non-ionic surfactant (e.g., sorbitan monolaurate or N-lauroyl sarcosine) and an ionic surfactant (e.g., an anionic surfactant such as sodium lauroyl sarcosinate). In other embodiments, the CPE includes a surfactant (e.g., an anionic surfactant such as sodium laureth sulfate) and an aromatic compound (e.g., 1-phenylpiperazine). Such combinations of CPEs can greatly enhance permeation of a drug through the skin with a low potential for skin irritation.

For transmucosal administration, in certain embodiments the CPE is or includes an alkyl glycoside (e.g., a 1-O or S—C₈-C₂₀ alkyl glycoside such as the corresponding glucoside, galactoside, mannoside, lactoside, maltoside [e.g., dodecyl, tridecyl or tetradecyl maltoside], melibioside or sucroside [e.g., dodecyl sucrose]), or a fatty ether or fatty ester saccharide (e.g., a C₈-C₂₀ alkyl ether or ester saccharide such as the corresponding glucoside, galactoside, mannoside, lactoside, maltoside, melibioside, sucroside [e.g., sucrose mono-, di- and tri-dodecanoate and mixtures thereof such as J-1205 and J-1216] or trehaloside).

In some embodiments, one or more NR/NAR derivatives are administered via a transdermal patch. In certain embodiments, a transdermal patch is a reservoir-type patch comprising an impermeable backing layer/film, a liquid- or gel-based drug reservoir, a semi-permeable membrane that controls drug release, and a skin-contacting adhesive layer. The semi-permeable membrane can be composed of, e.g., a suitable polymeric material such as cellulose nitrate or acetate, polyisobutene, polypropylene, polyvinyl acetate or a polycarbonate. In other embodiments, a transdermal patch is a drug-in-adhesive patch comprising an impermeable backing layer/film and a skin-contacting adhesive layer incorporating the drug in a polymeric or viscous adhesive. The adhesive of the drug-loaded, skin-contacting adhesive layer can be, e.g., a pressure-sensitive adhesive (PSA), such as a PSA composed of an acrylic polymer (e.g., polyacrylate), a polyalkylene (e.g., polyisobutylene) or a silicone-based polymer (e.g., silicone-2675 or silicone-2920). Transdermal drug-delivery systems, including patches, can be designed to provide controlled and prolonged release of a drug over a period of about 1 week, 2 weeks, 3 weeks, 1 month or longer.

In some embodiments, one or more NR/NAR derivatives are delivered from a sustained-release composition. As used herein, the term “sustained-release composition” encompasses sustained-release, prolonged-release, extended-release, delayed-release and slow-release compositions, systems and devices. A sustained-release composition can also be designed to be controlled-release. Advantages of a sustained-release composition include without limitation a more uniform blood level of the drug (e.g., avoidance of wide peak-to-trough fluctuations), delivery of a therapeutically effective amount of the drug over a prolonged time period, reduced frequency of administration, and reduced side effects (e.g., avoidance of a drug overdose). In certain embodiments, a sustained-release composition delivers one or more NR/NAR derivatives over a period of at least about 12 hours, 1 day, 2 days, 3 days, 1 week, 2 weeks, 3 weeks, 1 month, 2 months, 3 months or longer. In some embodiments, a sustained-release composition is a drug-encapsulation system, such as nanoparticles, microparticles or a capsule made of, e.g., a lipid, a biodegradable polymer or/and a hydrogel. In certain embodiments, a sustained-release composition comprises a hydrogel. Non-limiting examples of polymers of which a hydrogel can be composed include polyvinyl alcohol, acrylate polymers (e.g., sodium polyacrylate), and other homopolymers and copolymers having a relatively large number of hydrophilic groups (e.g., hydroxyl or/and carboxylate groups). In other embodiments, a sustained-release drug-encapsulation system comprises a membrane-enclosed reservoir, wherein the reservoir contains a drug and the membrane is permeable to the drug. Such a drug-delivery system can be in the form of, e.g., a transdermal patch.

In certain embodiments, a sustained-release composition is an oral dosage form, such as a tablet or capsule. For example, a drug can be embedded in an insoluble porous matrix such that the dissolving drug must make its way out of the matrix before it can be absorbed through the GI tract. Alternatively, a drug can be embedded in a matrix that swells to form a gel through which the drug exits. Sustained release can also be achieved by way of a single-layer or multi-layer osmotic controlled-release oral delivery system (OROS). An OROS is a tablet with a semi-permeable outer membrane and one or more small laser-drilled holes in it. As the tablet passes through the body, water is absorbed through the semi-permeable membrane via osmosis, and the resulting osmotic pressure pushes the drug out through the hole(s) in the tablet and into the GI tract where it can be absorbed.

In further embodiments, a sustained-release composition is formulated as polymeric nanoparticles or microparticles, which can be delivered, e.g., by injection or inhalation or as an implant (e.g., a depot). In some embodiments, the polymeric implant or polymeric nanoparticles or microparticles are composed of a biodegradable polymer. In certain embodiments, the biodegradable polymer comprises lactic acid or/and glycolic acid [e.g., an L-lactic acid-based copolymer, such as poly(L-lactide-co-glycolide) or poly(L-lactic acid-co-D,L-2-hydroxyoctanoic acid)]. For instance, biodegradable polymeric nano-/microspheres composed of polylactic acid or/and polyglycolic acid can serve as sustained-release pulmonary drug-delivery systems. The biodegradable polymer of the polymeric implant or polymeric nanoparticles or microparticles can be selected so that the polymer substantially completely degrades around the time the period of treatment is expected to end, and so that the byproducts of the polymer's degradation, like the polymer, are biocompatible.

In some embodiments, a sustained-release composition comprises a water-soluble polymer [e.g., poly(DL-lactide)] encapsulating an NR or NAR derivative complexed with or conjugated to a dendrimer (e.g., a PAMAM or/and PEG dendrimer). In other embodiments, a sustained-release composition is a nanoparticle composed of a dendrimer (e.g., a PAMAM or/and PEG dendrimer) and encapsulating an NR or NAR derivative. The dendrimer (e.g., the surface of a nanoparticle composed of a dendrimer) can optionally have or bear one or more moieties for targeting to specific organ(s), tissue(s), cell type(s) or organelle(s), such as one or more N-acetylgalactosamine (GalNAc) moieties for targeting to the liver for treatment of, e.g., a liver or metabolic disorder, or one or more RGD-containing moieties for targeting to tumor/cancer cells with upregulated cell-membrane integrins for treatment of a tumor or cancer. A dendrimer can have good cell membrane permeability.

In other embodiments, a sustained-release composition is in the form of nanoparticles or microparticles composed of one or more lipids (e.g., solid lipid nanoparticles [SLNs]) and encapsulating an NR or NAR derivative. The one or more lipids composing the nanoparticles or microparticles (e.g., the lipid core of SLNs) can be, e.g., physiological lipid(s) (thereby avoiding biotoxicity) and can be selected from, e.g., triglycerides (e.g. tristearin and Miglyol® 812), diglycerides (e.g. glycerol behenate), monoglycerides (e.g. glycerol monostearate), fatty acids (e.g. stearic acid), steroids (e.g. cholesterol), and waxes (e.g. cetyl palmitate). The lipid core of SLNs can be stabilized by one or more surfactants or emulsifiers. Lipid nanoparticles or microparticles can incorporate a lipophilic or hydrophilic drug. For example, a lipid core composed of stearic acid can incorporate a hydrophilic drug in SLNs. Relatively slow or slow degradation of the lipid(s) can provide controlled, slow or sustained release of the NR or NAR derivative. Furthermore, the lipid nanoparticles or microparticles can increase the oral bioavailability of the NR or NAR derivative by improving gastrointestinal absorption, can increase penetration of the NR or NAR derivative into cells (including target cells) after oral or parenteral administration by improving cell membrane permeability, and can increase the stability and half-life of the NR or NAR derivative by protecting the compound from the chemical environments and degradative enzymes of the body. The lipid nanoparticles or microparticles can be conjugated to a polymer, such as a hydrophilic polymer (e.g., PEG) to increase the aqueous solubility of the lipid particles. Moreover, the lipid nanoparticles or microparticles can be conjugated to one or more targeting moieties, such as one or more GaNAc moieties for targeting to the liver for treatment of, e.g., a liver or metabolic disorder.

For a delayed or sustained release of one or more NR/NAR derivatives, a composition can also be formulated as a depot that can be implanted in or injected into a subject, e.g., intramuscularly, intracutaneously or subcutaneously. A depot formulation can be designed to deliver an NR or NAR derivative over a longer period of time, e.g., over a period of at least about 1 week, 2 weeks, 3 weeks, 1 month, 6 weeks, 2 months, 3 months or longer. For example, an NR or NAR derivative can be formulated with a polymeric material (e.g., polyethylene glycol [PEG], polylactic acid [PLA] or polyglycolic acid [PGA], or a copolymer thereof [e.g., PLGA]), with a hydrophobic material (e.g., as an emulsion in an oil) or/and an ion-exchange resin, as a more lipophilic derivative (e.g., as an ester of or a salt with a fatty acid such as a C₈-C₂₀ fatty acid [e.g., decanoic acid]), or as a sparingly soluble derivative (e.g., a sparingly soluble salt). A depot can also be formed from liposomes, micelles, cholestosomes, nano-/microparticles or nano-/microspheres encapsulating one or more NR/NAR derivatives as described herein. As an illustrative example, an NR or NAR derivative can be incorporated or embedded in sustained-release nano-/microparticles composed of PLGA and formulated as a monthly depot.

In some embodiments, a pharmaceutical composition containing one or more NR/NAR derivatives is a controlled-release composition. A controlled-release composition can deliver a drug in a controlled time-dependent manner, and can be designed to deliver the drug, e.g., with delay after administration or/and for a prolonged time period. A controlled-release composition can also be designed to achieve particular profiles of dissolution of the drug in particular environments (e.g., in the GI tract) and to improve pharmacokinetics (e.g., bioavailability) of the drug. In certain embodiments, a controlled-release composition is administered once daily, once every two or three days, twice weekly or once weekly. In certain embodiments, a controlled-release composition is enterically coated for oral administration.

In some embodiments, a capsule for oral administration contains a plurality of pellets, each pellet comprising a pellet core containing one or more NR/NAR derivatives and a controlled-release coating surrounding the pellet core. The one or more NR/NAR derivatives can be, e.g., dispersed in a solid or semi-solid pellet core or in a drug layer coating the pellet core. In certain embodiment, the controlled-release coating comprises a polymer such as ethyl cellulose or/and hydroxypropyl cellulose, optionally povidone or/and hydroxypropyl methyl cellulose, and optionally a plasticizer (e.g., dibutyl sebacate).

In addition, pharmaceutical compositions comprising one or more NR/NAR derivatives can be formulated as, e.g., liposomes, micelles, cholestosomes, nano-/microparticles or nano-/microspheres encapsulating the compound(s), whether or not designed for controlled, slow or sustained release. The nano-/microparticles or nano/-microspheres can be composed of, e.g., a lipid, a biodegradable polymer or/and a non-degradable polymer, or a hydrogel. For example, liposomes can be used as a sustained-release pulmonary drug-delivery system that delivers a drug to the alveolar surface for treatment of a lung disorder or a systemic disorder. Such liposomes, micelles, cholestosomes, nano-/microparticles and nano-/microspheres can be formulated for oral or parenteral (e.g., intravenous, subcutaneous, intramuscular, intrathecal or topical) administration.

In some embodiments, liposomes or micelles are composed of one or more phospholipids. Phospholipids include without limitation phosphatidic acids (e.g., DEPA, DLPA, DMPA, DOPA, DPPA and DSPA), phosphatidylcholines (e.g., DDPC, DEPC, DLPC, DLOPC, DMPC, DOPC, DPPC, DSPC, MPPC, MSPC, PLPC, PMPC, POPC, PSPC, SMPC, SOPC and SPPC), phosphatidylethanolamines (e.g., DEPE, DLPE, DMPE, DOPE, DPPE, DSPE and POPE), phosphatidylglycerols (e.g., DEPG, DLPG, DMPG, DOPG, DPPG, DSPG and POPG), phosphatidylserines (e.g., DLPS, DMPS, DOPS, DPPS and DSPS), and salts (e.g., sodium and ammonium salts) thereof. In certain embodiments, liposomes or micelles are composed of one or more phosphatidylcholines. Liposomes have a hydrophilic core, so liposomes are particularly suited for delivery of more hydrophilic drugs, whereas micelles have a hydrophobic core, so micelles are particularly suited for delivery of more hydrophobic drugs. Liposomes and micelles can permeate across biological membranes. Liposomes and micelles composed of a fusogenic lipid (e.g., DPPG) can fuse with the plasma membrane of cells and thereby deliver a drug into those cells. Liposomes and micelles can provide controlled, slow or sustained release of a drug based in part on the rate of extracellular degradation of the liposomes and micelles.

In other embodiments, micelles are composed of biodegradable natural or/and synthetic polymer(s), such as lactosomes. In certain embodiments, micelles are lactosomes composed of a block copolymer, such as that containing two or three poly(sarcosine) blocks and a poly(lactic acid) block, where lactic acid can be L-lactic acid, D-lactic acid or D,L-lactic acid. In further embodiments, micelles are composed of an amphiphilic block copolymer, such as an amphiphilic di-, tri- or tetra-block copolymer containing hydrophilic block(s) and hydrophobic block(s). In additional embodiments, micelles are composed of one or more surfactants.

Cholestosomes are lipid particles (e.g., nanoparticles or microparticles) composed of one or more naturally occurring (and thus non-toxic) lipids or/and lipid esters and encapsulating a drug. They are typically neutral. Orally administered cholestosomes are resistant to degradation in the stomach, are absorbed through the intestines into the bloodstream (or into the lymphatic system if incorporated into chylomicrons), are taken up by cells (e.g., via endocytosis or permeation), escape lysosomal trapping, and degrade in the cells to release the drug. Cholestosomes can provide controlled, slow or sustained release of the drug based in part on the rate of extracellular degradation of the cholestosomes.

In some embodiments, one or more NR/NAR derivatives are encapsulated in nano-/microparticles or nano-/microspheres composed of a biodegradable synthetic or natural polymer, such as PLA, PGA, PLGA, poly(ε-caprolactone) (PCL) or a polysaccharide (e.g., chitosan), where lactic acid can be L-lactic acid, D-lactic acid or D,L-lactic acid. In other embodiments, one or more NR/NAR derivatives are encapsulated in nano-/microparticles or nano-/microspheres composed of a substantially non-degradable polymer, such as PEG. In further embodiments, one or more NR/NAR derivatives are encapsulated in nano-/microparticles or nano-/microspheres composed of a mixture or blend of a biodegradable polymer (e.g., PLA, PGA, PLGA or PCL) and a substantially non-degradable polymer (e.g., PEG). In still further embodiments, one or more NR/NAR derivatives are encapsulated in nano-/microparticles or nano-/microspheres composed of a copolymer or block copolymer containing a biodegradable polymer (e.g., PLA, PGA, PLGA or PCL) and a substantially non-degradable polymer (e.g., PEG). In yet further embodiments, one or more NR/NAR derivatives are encapsulated in nano-/microparticles or nano-/microspheres composed of a dendrimer, such as a PAMAM or/and PEG dendrimer. Such compositions can provide controlled, slow or sustained release of the NR/NAR derivative(s) based in part on the rate of degradation of the polymer or dendrimer or/and the rate of diffusion of the NR/NAR derivative(s) through the polymer or dendrimer (e.g., through pores formed by the polymer or dendrimer).

In some embodiments, liposomes, micelles, cholestosomes, nano-/microparticles or nano-/microspheres encapsulating one or more NR/NAR derivatives are conjugated to or coated with a biodegradable or non-degradable polymer. In certain embodiments, the surface-conjugating/coating polymer is a hydrophilic polymer, such as PEG. In some embodiments, the surface-conjugating/coating polymer (e.g., PEG) has a molecular weight of about 0.5-1 kDa, 1-2 kDa, 2-5 kDa or higher. Conjugation or coating of the surface of such compositions with a polymer can have various benefits, including minimizing aggregation and immunogenicity of the compositions, and shielding the compositions from the degradative environments of the body, opsonization and phagocytosis, thereby increasing their half-life.

In further embodiments, liposomes, micelles, cholestosomes, nano-/microparticles or nano-/microspheres encapsulating one or more NR/NAR derivatives are conjugated to one or more targeting moieties. In certain embodiments, the targeting moieties are GaNAc moieties for targeting of the compositions to the liver for treatment of, e.g., a liver or metabolic disorder. In other embodiments, the targeting moieties are RGD-containing moieties for targeting of the compositions to tumor/cancer cells with upregulated cell-membrane integrins for treatment of a tumor or cancer.

Pharmaceutical compositions can be manufactured in any suitable manner known in the art, such as by means of conventional mixing, dissolving, suspending, granulating, dragee-making, levigating, emulsifying, encapsulating, entrapping or compressing processes, or any combination thereof.

A pharmaceutical composition can be presented in unit dosage form as a single dose wherein all active and inactive ingredients are combined in a suitable system, and components do not need to be mixed to form the composition to be administered. A unit dosage form generally contains a therapeutically effective dose of the drug, but can contain an appropriate fraction thereof so that taking multiple unit dosage forms achieves the therapeutically effective dose. Representative examples of a unit dosage form include a tablet, capsule or pill for oral uptake; a solution in a pre-filled syringe of a single-use pen or a pen with a dose counter for parenteral (e.g., intravenous, subcutaneous or intramuscular) injection; a capsule, cartridge or blister pre-loaded in or manually loaded into an inhaler; and a reservoir-type transdermal patch or a drug-in-adhesive patch.

Alternatively, a pharmaceutical composition can be presented as a kit in which the drug, excipients and carriers (e.g., solvents) are provided in two or more separate containers (e.g., ampules, vials, tubes, bottles or syringes) and need to be combined to form the composition to be administered. The kit can contain instructions for storing, preparing and administering the composition (e.g., a solution to be injected parenterally).

A kit can contain all active and inactive ingredients in unit dosage form or the active ingredient and inactive ingredients in two or more separate containers, and can contain instructions for administering or using the pharmaceutical composition to treat a medical condition. A kit can further contain a device for delivering the composition, such as an injection pen, an inhaler or a transdermal patch.

In some embodiments, a kit contains one or more NR/NAR derivatives or a pharmaceutical composition comprising the same, and instructions for administering or using the one or more NR/NAR derivatives or the pharmaceutical composition comprising the same to treat a disease, disorder or condition described herein. In certain embodiments, a kit contains a compound of Formula II or a compound of Formula IV, or a pharmaceutical composition comprising the same. In further embodiments, a kit contains a compound of Formula I and a compound of Formula II, or a compound of Formula III and a compound of Formula IV, or a pharmaceutical composition comprising the same. A kit comprising one or more NR/NAR derivatives or a pharmaceutical composition comprising the same can further comprise one or more additional therapeutic agents (e.g., a mitochondrial uncoupler or/and a PARP inhibitor).

The description and all of the embodiments relating to pharmaceutical compositions and kits comprising the NR and NAR derivatives disclosed herein also apply to pharmaceutical compositions and kits comprising other NR and NAR derivatives (e.g., NRTA, NRHTA, NARTA and NARHTA), to pharmaceutical compositions and kits comprising NR, NRH, NAR or/and NARH, to pharmaceutical compositions and kits comprising any other therapeutic agents described herein (e.g., a mitochondrial uncoupler or/and a PARP inhibitor), and to pharmaceutical compositions and kits comprising nicotinyl riboside compounds and any other therapeutic agents described herein (e.g., a mitochondrial uncoupler or/and a PARP inhibitor).

### Uses of NR and NAR Derivatives

The NR and NAR derivatives described herein can increase NAD⁺ levels in a subject, including in cells, tissues, organs and the blood. By increasing NAD⁺ levels, the NR and NAR derivatives can improve mitochondrial function and cellular function (e.g., oxidative metabolism and DNA repair) in target cells, tissues and organs and can improve cell viability. Benefits of improved mitochondrial function include without limitation enhanced mitochondrial oxidative metabolism, mitochondrial respiration, ATP production, mitochondrial membrane potential, mitophagy (autophagy of defective mitochondria) and mitochondrial biogenesis, and reduced levels of reactive oxygen species (ROS). For example, higher NAD⁺ levels increase the activity of the mitochondrial NAD-dependent deacetylases sirtuin-1 (SIRT1) and sirtuin-3 (SIRT3). SIRT1 promotes autophagy of defective mitochondria, stimulates mitochondrial biogenesis, inhibits the pro-inflammatory transcription factor NF-κB, increases insulin sensitivity, and mimics the effects of calorie restriction. Stimulation of SIRT3 activity increases mitochondrial biogenesis, increases cellular respiration and energy production, reduces ROS levels (e.g., by stimulating mitochondrial superoxide dismutase 2 [SOD2]), promotes cell survival during genotoxic stress, functions as a mitochondrial tumor suppressor, increases insulin sensitivity and sensitizes cells to glucose uptake, and mimics calorie restriction and exercise. Improved DNA repair reduces cell damage and enhances cell function, health and lifespan. In addition, prevention of NAD⁺ depletion protects neurons in excitotoxic or ischemic conditions.

Therefore, the NR and NAR derivatives are useful for treating pellagra, mitochondrial diseases, mitochondria-related diseases and conditions, diseases and conditions associated with acute NAD⁺ depletion resulting from DNA damage, aging-related disorders and conditions, skin disorders and conditions, and other types of disorders and conditions. In some embodiments, a single NR or NAR derivative (e.g., a compound of Formula II or IV) is used to treat a disease/disorder or condition disclosed herein or to bring about a biological effect disclosed herein (e.g., increase NAD⁺ level, enhance mitochondrial or cellular function, improve metabolic health or cell viability, or provide cytoprotection). In other embodiments, a compound of Formula I and a compound of Formula II, or a compound of Formula III and a compound of Formula IV, are used to treat a disease/disorder or condition disclosed herein or to bring about a biological effect disclosed herein. The use of both an oxidized form of an NR or NAR derivative (Formula I or III) and a reduced form of an NR or NAR derivative (Formula II or IV) can have an additive effect or potentially a synergistic effect. In further embodiments, one or more NR/NAR derivatives disclosed herein are used in conjunction with NR, NRH, NAR or NARH, or any combination thereof, to treat a disease/disorder or condition disclosed herein or to bring about a biological effect disclosed herein. The use of an NR or NAR derivative plus NR, NRH, NAR or NARH can have an additive effect or potentially a synergistic effect. A single NR or NAR derivative can be administered in the form of, e.g., a pharmaceutical or cosmetic composition. If, e.g., two NR/NAR derivatives are utilized, they can be administered in the same composition or in different compositions.

The NR and NAR derivatives have other beneficial effects. For example, they can enhance immune function of peripheral blood mononuclear cells (PBMCs, such as T-cells, B-cells, macrophages [e.g., lymph node- and tissue-resident macrophages] and natural killer [NK] cells) based on reversal of immune exhaustion and improved antigen recognition and antigen-specific immune reactivity as a function of immune surveillance. For such an application, one or more NR/NAR derivatives can be employed alone, as a component of a vaccine, as a component of an ex vivo therapy (e.g., a CAR-T cell therapy), or as a component of some other therapy.

Mitochondrial diseases include without limitation mitochondrial myopathies; limb-girdle distribution weakness; mitochondrial transcription factor A (TFAM) deficiency; Kearns-Sayre syndrome (KSS); Pearson syndrome; Leigh syndrome; Barth syndrome; Friedreich's ataxia; ataxia neuropathy syndrome/spectrum (ANS, including mitochondrial recessive ataxia syndrome [MIRAS] and sensory ataxia neuropathy, dysarthria and ophthalmoplegia [SANDO]); neuropathy, ataxia and retinitis pigmentosa (NARP); mitochondrial DNA depletion syndrome (MDDS. Alper's disease or Alpers-Huttenlocher syndrome); mitochondrial neurogastrointestinal encephalopathy (MNGIE) syndrome; mitochondrial encephalopathy, lactic acidosis and stroke-like episodes (MELAS) syndrome; myoclonic epilepsy with ragged red fibers (MERRF or Fukuhara syndrome); chronic progressive external ophthalmoplegia (CPEO); Leber's hereditary optic neuropathy (LHON); inherited forms of blindness and deafness (e.g., diabetes mellitus and deafness [MIDD], and aminoglycoside-induced non-syndromic deafness); acquired forms of reversible or permanent hearing loss (e.g., type 2 diabetes-associated hearing loss and hearing loss induced by ototoxic chemicals (e.g., heavy metals [e.g., lead], solvents [e.g., styrene and toluene] and asphyxiants [e.g., carbon monoxide]) and medications (e.g., loop diuretics [e.g., bumetanide and furosemide], NSAIDs [e.g., aspirin, celecoxib, diclofenac, ibuprofen and naproxen], PDE5 inhibitors, macrolide antibiotics, aminoglycosides [e.g., gentamicin], platinum-based chemotherapeutics [e.g., carboplatin and cisplatin], paracetamol and quinine)); and disorders (e.g., myopathy, neuropathy, lactic acidosis and lipodystrophy) resulting from mitochondrial toxicity due to, e.g., medications {e.g., antiviral drugs (including antiretroviral drugs [e.g., nucleoside analog reverse transcriptase inhibitors {NRTIs})] against HIV and nucleoside and nucleotide analogs against HBV, HCV and CMV)}.

Primary mitochondrial diseases include, but are not limited to, Keams-Sayre syndrome, Pearson syndrome, Leigh syndrome, NARP, ataxia neuropathy syndrome/spectrum. MDDS (Alpers-Huttenlocher syndrome), MNGIE, MELAS, MERRF, LHON, and aminoglycoside-induced non-syndromic deafness.

Mitochondria-related diseases and conditions include, but are not limited to, neurodegenerative disorders, neuronal activation disorders, muscle disorders (including eye muscle disorders), metabolic disorders, fatty acid/beta oxidation disorders, disorders associated with abnormal or ectopic lipid accumulation or storage, lysosomal storage diseases (including lipid storage disorders), disorders associated with oxidative stress, inflammatory disorders, immune-related disorders, vascular disorders (including ocular vascular disorders), renal disorders, liver disorders, proliferative disorders (including tumors and cancers), male and female infertility, and aging-related disorders.

Neurodegenerative disorders include without limitation dementias (e.g., Alzheimer's disease [AD], vascular dementia, dementia with Lewy bodies and frontotemporal dementia [Pick's disease]), motor neuron disorders (e.g., Parkinson's disease, amyotrophic lateral sclerosis [ALS or Lou Gehrig's disease], primary lateral sclerosis [PLS], spinal muscular atrophy [SMA] and hereditary spastic paraplegia [HSP, including types 1-79 and HSNSP]), ataxia (e.g., spinocerebellar ataxia/degeneration, Friedreich's ataxia, ataxia-telangiectasia [Louis-Bar syndrome] and fragile X-associated tremor/ataxia syndrome [FXTAS]), dyskinesias (e.g., cerebral palsy, chorea, dystonias and essential tremor), cognitive-motor disorders (e.g., corticobasal degeneration, Huntington's disease [HD] and Parkinson-plus syndromes), chorea-acanthocytosis, retinal neuronal degeneration, Batten disease, DNA-repair syndromes (e.g., Cockayne syndrome), and prion diseases (e.g., Creutzfeldt-Jakob disease).

Neuronal activation disorders include without limitation neurodegenerative disorders (e.g., ALS), neuronal injuries (including traumatic and mechanical injuries to the brain, the spinal cord and the peripheral nervous system [PNS], and excitotoxic neuronal injuries such as those associated with seizures and ischemia), nerve lesions, neuropathies (e.g., peripheral neuropathies [e.g., Charcot-Marie-Tooth disease and drug-induced peripheral neuropathies], mononeuropathies [e.g., those caused by compression, traumatic injury, cumulative trauma, ischemia, inflammation, connective tissue disorders and neoplasms], polyneuropathies [e.g., chronic inflammatory demyelinating polyneuropathy], brachial plexus neuropathies, diabetic neuropathies [e.g. third nerve palsy, mononeuropathy, mononeuropathy multiplex, autonomic neuropathy, thoracoabdominal neuropathy and diabetic amyotrophy], and chemotherapy-induced neuropathies), autoimmune nerve disorders (e.g., multiple sclerosis, Guillain-Barré syndrome. Lambert-Eaton myasthenic syndrome and myasthenia gravis), neuroinflammation, tardy ulnar nerve palsy, and toxic myoneural disorder.

Muscle disorders include, but are not limited to, muscle structure disorders, muscle mass disorders and muscle fatigue disorders. Muscle structure disorders include without limitation myopathies (e.g., fatal infantile myopathy, later-onset myopathy, Bethlem myopathy, cardiomyopathy, hyaline body myopathy, myotubular myopathy and inflammatory myopathies), neuromuscular degeneration, muscular dystrophies (e.g., congenital MD, distal MD, Duchenne MD, Becker MD, Emery-Dreifuss MD, limb-girdle MD, myotonic MD, facioscapulohumeral MD and oculopharyngeal MD), myotonic dystrophy, myotonic chondrodystrophy, central core disease, congenital fiber-type disproportion, muscle sodium channel disorders, nemaline body disease, myositis, sarcopenia, rhabdomyolysis, and stress urinary incontinence. Muscle mass disorders include without limitation muscle atrophy, cachexia, cartilage degeneration, cerebral palsy, compartment syndrome, critical illness myopathy, inclusion body myositis, sarcopenia, steroid myopathy, and systemic lupus erythematosus (SLE). Muscle fatigue disorders include without limitation chronic fatigue syndrome, fibromyalgia, thyrotoxic myopathy, lipid-storage myopathy, Friedreich's ataxia, glycogen storage diseases (e.g., Pompe disease), intermittent claudication, MELAS, and mucopolysaccharidosis.

Eye muscle disorders include, but are not limited to, disorders of refraction, disorders of accommodation, disorders of refraction and accommodation, strabismus, progressive external ophthalmoplegia, internal ophthalmoplegia, esotropia, exotropia, hypermetropia, myopia, astigmatism, anisometropia, and presbyopia.

Metabolic disorders include without limitation lipodystrophy (including congenital/genetic and acquired, partial and generalized, and severe), metabolic syndrome, hyperglycemia, impaired glucose tolerance (including prediabetes and diabetes), insulin resistance, hyperinsulinism, diabetes mellitus (including types 1 and 2), diabetic complications (e.g., diabetic nephropathy, diabetic neuropathy and diabetic retinopathy), obesity, dyslipidemia (inherited and acquired), hyperlipidemia, hypercholesterolemia, familial hypercholesterolemia (homozygous and heterozygous), non-high-density lipoprotein (non-HDL) hypercholesterolemia, low-density lipoprotein (LDL) hypercholesterolemia, HDL hypocholesterolemia, hypertriglyceridemia, fatty acid/beta oxidation disorders (infra), lysosomal storage diseases (infra, including lipid storage disorders such as lysosomal acid lipase deficiency [including Wolman disease and cholesteryl ester storage disease] and lipid storage droplet disorders such as CGI-58 deficiency [Chanarin-Dorfman syndrome], MTP deficiency and apolipoprotein B [ApoB] deficiency), hyperphagia-associated disorders (e.g., Alstrom syndrome. Bardet-Biedl syndrome and Prader-Willi syndrome), dyslipoproteinemia, very low-density lipoprotein (VLDL) hyperproteinemia, apolipoprotein A-I hypoproteinemia, hypertension, cardiovascular diseases (e.g., cardiomyopathy [e.g., metabolic cardiomyopathy], cardiac insufficiency, myocardial infarction, atherosclerosis, thrombotic disorders and peripheral vascular diseases), inflammatory disorders (e.g., arthritis, asthma and pancreatitis), liver disorders (e.g., non-alcoholic fatty liver disease [NAFLD], non-alcoholic steatohepatitis [NASH], alcoholic liver disease [ALD], alcoholic steatohepatitis [ASH] and primary biliary cholangitis/cirrhosis [PBC]), kidney disorders (e.g., chronic kidney disease [CKD]), gastrointestinal (GI) disorders (e.g., Crohn's disease, hypersensitive intestine syndrome, ulcerative colitis and dyspepsia), thyroid disorders (e.g., hypothyroidism), neurodegenerative disorders (e.g., Alzheimer disease), demyelinating disorders (e.g., multiple sclerosis and leukodystrophies [e.g., metachromatic leukodystrophy, Krabbe disease, X-linked adrenoleukodystrophy, Canavan disease and Alexander disease]), proliferative disorders (e.g., tumors and cancers), metabolic acidosis (e.g., ketoacidosis), organic acidemias/acidurias (e.g., isovaleric acidemia, methylmalonic acidemia, propionic acidemia, and maple syrup urine disease), urea cycle disorders (e.g., argininosuccinic aciduria), purine/pyrimidine synthesis disorders (e.g., Lesch-Nyhan syndrome), edema, sexual (e.g., erectile) dysfunction, skin disorders (e.g., acne, dermatitis, psoriasis and skin aging), and trichosis.

Lipodystrophy is a group of genetic/congenital or acquired disorders in which the body is unable to produce and maintain healthy fat tissue. Lipodystrophy is characterized by abnormal or degenerative conditions of adipose tissue in certain areas of the body (partial, such as the arms, the legs or the face) or throughout most of the body (generalized), such as impaired ability of adipose tissue to store lipids and loss or absence of adipose tissue (lipoatrophy) under the skin (loss or absence of subcutaneous fat). Lipodystrophy can lead to fat accumulation elsewhere in the body such as in vital organs (e.g., the liver, kidneys and heart) and muscles (e.g., skeletal muscles) and severe metabolic complications, and is associated with disorders that are also associated with obesity, such as metabolic syndrome, hypertriglyceridemia, insulin resistance, diabetes (e.g., type 2 diabetes), cardiovascular diseases (e.g., coronary artery disease [CAD]), and NAFLD (e.g., NASH). Lipodystrophy severity often correlates with the severity of metabolic complications such as insulin resistance. Patients with generalized lipodystrophy typically have little or no adipose tissue, including subcutaneous adipose tissue for storing fat, so fat is deposited in non-adipose tissues and organs, leading to lipotoxicity (and hence cellular dysfunction and death), hypertriglyceridemia, insulin resistance and severe fatty liver disease, with liver failure being the usual cause of death at about 30 years of age. In contrast to high leptin levels often seen in NAFLD associated with obesity, leptin levels are typically low in patients with generalized forms of lipodystropy or severe lipodystropy, who thus have hyperphagia. Genetic/congenital lipodystrophy disorders include without limitation congenital generalized lipodystrophy (Berardinelli-Seip syndrome), familial partial lipodystrophy (Köbberling-Dunnigan syndrome), Marfanoid-progeroid-lipodystrophy syndrome (Marfan lipodystrophy syndrome), and CANDLE syndrome. Acquired lipodystrophy disorders include without limitation acquired generalized lipodystrophy (Lawrence syndrome), acquired partial lipodystrophy (Barraquer-Simons syndrome), centrifugal abdominal lipodystrophy, lipoatrophia annularis (Ferreira-Marques lipoatrophy), localized lipodystrophy (e.g., localized to sites of insulin injection), HIV-associated lipodystrophy (in the presence or absence of antiretroviral therapy [ART]), and drug-induced (e.g., ART-induced) lipodystrophy.

HIV-associated lipodystrophy is characterized by loss of subcutaneous fat, commonly in the face, buttocks, arms and legs. Fat accumulates in various parts of the body, including the upper back and abdomen. HIV-associated lipodystrophy can be caused by HIV infection which may interfere with some key genes of adipocyte differentiation and mitochondrial function in patients who have not taken ART, or by ART. ART-induced lipodystrophy can occur with antiretroviral HIV-1 protease inhibitors which may interfere with lipid metabolism, or with NRTIs which may cause mitochondrial toxicity. ART-induced lipodystrophy typically does not reverse after stoppage of ART. Patients with HIV-associated lipodystrophy typically do not have low leptin levels.

In some embodiments, the NR and NAR derivatives are used to treat hyperglycemia, impaired glucose tolerance and insulin resistance and disorders and conditions related thereto, including prediabetes, types 1 and 2 diabetes, and obesity-related disorders and conditions. The NR and NAR derivatives stimulate SIRT1 and SIRT3 activity, either of which increases insulin sensitivity, sensitizes cells to glucose uptake and mimics calorie restriction. Increased insulin sensitivity can reduce insulin production. Hyperinsulinemia promotes differentiation of preadipocytes into adipocytes. Therefore, reduction of high blood insulin level can inhibit fat cell differentiation and adipogenesis and thus can have therapeutic effects on obesity-related disorders and conditions, including but not limited to dyslipogenesis, hyperlipidemia, hypercholesterolemia, atherosclerosis, metabolic syndrome, lipodystrophy and hypertension.

Fatty acid/beta oxidation disorders include without limitation systemic carnitine transporter deficiency, carnitine palmitoyl transferase (CPT) II deficiency, very long-chain acyl-CoA dehydrogenase (LCHAD or VLCAD) deficiency, medium-chain acyl-CoA dehydrogenase (MCAD) deficiency, short-chain acyl-CoA dehydrogenase (SCAD) deficiency, comparative gene identification-58 (CGI-58) deficiency (Chanarin-Dorfman syndrome), trifunctional enzyme deficiency (mitochondrial trifunctional protein [MTP] deficiency), and riboflavin-responsive disorders of β-oxidation (RR-MADD).

Disorders associated with abnormal or ectopic lipid accumulation or storage are described in the section on combination therapies with mitochondrial uncouplers.

Lysosomal storage diseases include without limitation lipid storage disorders {including sphingolipidoses (e.g., acid sphingomyelinase deficiency, Farber disease, Fabry disease [Anderson-Fabry disease], Gaucher disease [including types I, II and III], Krabbe disease, metachromatic leukodystrophy, multiple sulfatase deficiency, Niemann-Pick disease [NPD, including types A. B. C and D] and Tay-Sachs disease), GM1 gangliosidoses. GM2 gangliosidoses (e.g., Sandhoff disease, Tay-Sachs disease and GM2-gangliosidosis, AB variant), gangliosidoses (e.g., mucolipidosis IV), fucosidosis, lysosomal acid lipase (LAL) deficiency (including Wolman disease and cholesteryl ester storage disease [CESD]). Schindler disease (Kanzaki disease), xanthomatoses (including xanthelasma, eruptive xanthoma [e.g., xanthoma diabeticorum], tuberoeruptive xanthoma, plane xanthoma, palmar xanthoma, tendinous xanthoma [e.g., cerebrotendineous xanthomatosis], tuberous xanthoma and xanthoma disseminatum), histiocytoses (e.g., NPD, sea-blue histiocytosis and verrucous xanthoma [histiocytosis Y]), and lipid storage droplet disorders (infra)}, neuronal ceroid lipofuscinoses (including types 1 [Santavuori disease], 2 [Jansky-Bielschowsky disease], 3 [Batten disease], 4 [Kufs disease] and 5-10), glycoprotein storage disorders {including mucolipidoses (e.g., mucolipidosis I [sialidosis], mucolipidosis II [1-cell disease] and mucolipidosis III [pseudo-Hurler polydystrophy]), aspartylglucosaminuria, fucosidosis, galactosialidosis and mannosidoses (including alpha- and beta-mannosidoses)}, glycogen storage disorders (e.g., GSD II [Pompe disease] and GSD IIb [Danon disease]), sialic acid storage disorders (e.g., infantile free sialic acid storage disease and Salla disease), mucopolysaccharidoses (e.g., Hurler syndrome [MPS I H], Hurler-Scheie syndrome [MPS I H-S], Scheie syndrome [MPS I S], Hunter syndrome [MPS II], Sanfilippo syndrome [MPS III, including types A-D], Morquio syndrome [MPS IV, including types A and B], Maroteaux-Lamy syndrome [MPS VI], Sly syndrome [MPS VII] and Natowicz syndrome [MPS IX, hyaluronidase deficiency]), cystinosis and pycnodysostosis.

Lipid droplets (LDs) are lipid-rich organelles that regulate storage and hydrolysis of neutral lipids and are found primarily in adipose tissues, storing a large portion of lipids in adipocytes. LDs have a hydrophobic core composed mostly of the neutral lipids triacylglycerol (TAG)/triglycerides (TG) and esterified cholesterol (CE) surrounded by a phospholipid monolayer membrane bearing proteins that regulate LD dynamics. The main cells involved in lipid-imbalance disorders such as adipocytes, hepatocytes and myocytes have mostly TAG LDs. Degradation of LDs, primarily by lipolysis and autophagy, provides lipids (mainly cholesterol and acyl-glycerols) and metabolic energy for a variety of cellular processes such as membrane synthesis and molecular signaling. LDs play an important role in lipid homeostasis through intracellular lipid storage, lipid synthesis, lipid metabolism, and lipid transportation. LDs are also involved in cell signaling. In non-adipocytes, LDs can protect from lipotoxicity by storing fatty acids in the form of TAG. Alternatively, fatty acids can be converted to lipid intermediates such as diacylglycerol (DAG), ceramides and fatty acyl-CoA. Such lipid intermediates can cause lipotoxicity and impair insulin signaling (lipid-induced insulin resistance). LDs are also associated with inflammation through synthesis and metabolism of eicosanoids. Consequently, LDs are implicated in a wide range of disorders affected by lipid imbalances such as metabolic disorders, dyslipidemia, obesity, lipodystrophy, fatty liver diseases, type 2 diabetes, cardiovascular diseases, atherosclerosis, inflammatory disorders, tumors and cancers, and Alzheimer's disease. Examples of lipid storage droplet disorders include CGI-58 deficiency (Chanarin-Dorfman syndrome), MTP deficiency and ApoB deficiency.

Dysfunctional mitochondria can produce a high level of ROS and oxidative stress. Disorders associated with oxidative stress are described in the section on combination therapies with mitochondrial uncouplers, and also include diseases and conditions characterized by acute NAD⁺ depletion due to DNA damage (described below).

ROS incite inflammation, in part by activating transcriptions factors such as NF-κB that increase the expression of pro-inflammatory cytokines. The NR and NAR derivatives disclosed herein can reduce ROS levels by, e.g., stimulating SIRT3 activity. Moreover, the NR and NAR derivatives can increase the activity of NAD-dependent deacetylase sirtuin-1 (SIRT1), which inhibits NF-κB. NF-κB is the main promoter of the transcription of genes encoding pro-inflammatory cytokines. Thus, the NR and NAR derivatives are useful for treating inflammatory disorders. Inflammatory disorders include without limitation neuroinflammation (e.g., neuritis [e.g., ocular neuritis and peripheral neuritis], encephalomyelitis [e.g., autoimmune encephalomyelitis], Alzheimer's disease and multiple sclerosis), muscle disorders (e.g., myositis), GI disorders {e.g., gastritis, colitis (e.g., mucous colitis, ulcerative colitis [UC] and necrotizing enterocolitis), inflammatory bowel disease (IBD, including UC and Crohn's disease), irritable bowel syndrome, and celiac disease}, peritonitis, pancreatitis (acute and chronic), glomerulonephritis, liver disorders (e.g., hepatitis, non-alcoholic and alcoholic steatohepatitis, cirrhosis and chronic liver disease), multiple organ dysfunction syndrome (e.g., secondary to septicemia or trauma), metabolic disorders (e.g., diabetes [e.g., types 1 and 2 diabetes and juvenile-onset diabetes] and metabolic syndrome), cardiac disorders (e.g., myocarditis, non-ischemic cardiomyopathy and myocardial infarction), vascular disorders (e.g., vasculitis, atherosclerosis, stroke, peripheral artery disease and shock), reperfusion injury (e.g., due to myocardial ischemia, cerebral ischemia, cardiopulmonary bypass or kidney dialysis), airway disorders (e.g., rhinitis [e.g., allergic rhinitis], esophagitis, asthma, acute respiratory distress syndrome, bronchitis [e.g., chronic bronchitis], pneumonitis and chronic obstructive pulmonary disease [COPD]), rheumatic disorders {e.g., arthritis (e.g., osteoarthritis [degenerative joint disease], rheumatoid arthritis, juvenile arthritis, psoriatic arthritis, gout, axial spondyloarthritis and ankylosing spondylitis) and diffuse connective tissue disorders (e.g., SLE, Sjögren syndrome, and localized and systemic scleroderma)}, skin disorders (e.g., dermatitis/eczema, pemphigoid, psoriasis, urticaria, dermatosis with acute inflammatory components, and sunburn), eye disorders (e.g., conjunctivitis, retinitis, uveitis and AMD), hypertension and dysmenorrhea (menstrual cramps).

Immune-related disorders include without limitation inflammatory disorders, autoimmune disorders, and disorders associated with overactivation of the immune system. Disorders associated with overactivation of the immune system are described in the section on combination therapies with mitochondrial uncouplers.

Autoimmune responses generally incite or are induced by an inflammatory reaction. Thus, many inflammatory disorders are also autoimmune disorders. Autoimmune disorders include without limitation nervous system disorders (e.g., multiple sclerosis and Guillain-Barré syndrome [GBS]), GI disorders (e.g., ulcerative colitis and celiac disease), liver disorders (e.g., autoimmune hepatitis), metabolic disorders (e.g., type 1 diabetes, Grave's disease [which causes hyperthyroidism], and Hashimoto's thyroiditis [which causes hypothyroidism]), rheumatic disorders (e.g., arthritis [e.g, rheumatoid arthritis and juvenile arthritis] and diffuse connective tissue disorders [e.g., SLE, Sjögren syndrome, and localized and systemic scleroderma]), and skin disorders (e.g., pemphigus, pemphigoid and psoriasis).

Inflammation is a major stimulant of fibrosis. In part by reducing inflammation, the NR and NAR derivatives disclosed herein are useful for treating fibrotic disorders. Fibrotic disorders include without limitation cardiomyopathy (e.g., ischemic and non-ischemic cardiomyopathy, diabetic cardiomyopathy and uremic cardiomyopathy), cardiac fibrosis, myocardial fibrosis, collagen-vascular diseases (e.g., arterial stiffness and vascular fibrosis), atherosclerosis, chronic heart failure, diabetic nephropathy, renal fibrosis (e.g., renal tubulointerstitial fibrosis), chronic kidney disease (e.g., chronic renal failure), liver fibrosis, cirrhosis. NASH, chronic liver disease, liver failure (e.g., chronic liver failure), pulmonary fibrosis (e.g., idiopathic pulmonary fibrosis [IPF], connective tissue disease-related pulmonary fibrosis and radiation-induced pulmonary fibrosis), cystic fibrosis, and scleroderma (e.g., localized scleroderma and systemic scleroderma/systemic sclerosis).

Vascular disorders include, but are not limited to, cardiovascular diseases (e.g., myocardial ischemia, ischemia-reperfusion injury [IRI], atherosclerosis and arteriosclerosis), cerebrovascular diseases (e.g., cerebral ischemia and IRI), peripheral vascular diseases (e.g., peripheral vascular insufficiency, peripheral artery disease, intermittent/vascular claudication, critical limb ischemia, peripheral artery occlusive disease, and peripheral obliterative arteriopathy), thrombotic/blood clotting/hemostatic disorders (e.g., disseminated intravascular coagulation, deep vein thrombosis, thrombophilia [e.g., due to anti-thrombin III deficiency, protein S deficiency, protein C deficiency or resistance to activated protein C], thrombotic thrombocytopenic purpura, heparin-induced thrombocytopenia, dysfibrinogenemia, atherosclerosis, arteriosclerosis, myocardial ischemia/infarction, angina [e.g., unstable angina], ischemic stroke, sickle cell disease, myeloproliferative neoplasms, cancer metastasis, homocystinuria, and miscarriage), and embolism (e.g., thromboembolism, fat embolism, arterial embolism [e.g., myocardial ischemia, ischemic stroke and acute limb ischemia], and venous embolism [e.g., pulmonary embolism]). As an illustrative example, one or more NR/NAR derivatives can be used to treat or prevent thrombosis or a thrombotic disorder, including to reduce or prevent thrombotic events or re-occlusion during or/and after a clot-clearing intervention (e.g., a surgery such as angioplasty).

Ocular vascular disorders include without limitation retinopathy (e.g., hypertensive retinopathy and diabetic retinopathy), macular degeneration (e.g., age-related macular degeneration [AMD]), Stargardt disease, retinal hemorrhage and glaucoma.

Renal disorders include without limitation glomerular diseases, tubular diseases, acute nephritis, chronic nephritis, rapidly progressive nephritis, glomerulonephritis, glomerulosclerosis, hypertensive nephrosclerosis, renal ischemia, IRI, Bartter syndrome, diabetic nephropathy, acute renal failure (acute kidney injury), chronic renal failure/CKD, nephrotic syndrome, recurrent hematuria and persistent hematuria.

Liver disorders include without limitation NAFLD, NASH, ALD (which encompasses liver manifestations of alcohol overconsumption, including fatty liver, alcoholic hepatitis, and chronic hepatitis with liver fibrosis or cirrhosis), ASH, hepatitis (e.g., autoimmune hepatitis, hepatitis B and hepatitis C), cholestatic disorders (e.g., cholestasis, PBC and primary sclerosing cholangitis [PSC]), liver injury, liver fibrosis, chronic liver disease {including CLD caused by, e.g., a virus (e.g., hepatitis B [HBV], hepatitis C [HCV], cytomegalovirus [CMV] or Epstein-Barr virus [EBV]), a parasite (e.g., schistosomiasis), a hepatotoxic agent (e.g., alcohol) or drug (e.g., methotrexate), or a metabolic disorder (e.g., NAFLD, NASH, hemochromatosis or Wilson's disease)}, liver failure (acute and chronic), cirrhosis, and liver cancer (e.g., hepatocellular carcinoma [HCC]).

Tumors (benign and malignant) and cancers include without limitation brain tumors, spinal cord tumors, germ cell tumors, neuroendocrine tumors, carcinoid tumors, tumors and cancers associated with viral infections (e.g., HIV and HTLV-1), carcinomas, sarcomas, and cancers of the digestive/gastrointestinal system, gynecological organs (e.g., the breast), genitourinary system, musculoskeletal system, respiratory system, head and neck, eye, skin (e.g., melanomas), blood (e.g., leukemias, multiple myeloma, Hodgkin's lymphomas and non-Hodgkin's lymphomas), endocrine system (e.g., hormone-dependent cancers such as breast, ovarian, prostate and testicular cancers), neuroendocrine system, neurological system, and germ cells. In some embodiments, one or more NR/NAR derivatives are used to treat a cancer of the breast, ovary, colon/large intestine, rectum, pancreas, liver, kidney, lung, prostate, brain or skin. In further embodiments, one or more NR/NAR derivatives are used to treat a hematological malignancy, such as acute lymphoblastic leukemia (ALL), acute myeloid leukemia (AML), chronic lymphocytic leukemia (CLL), chronic myelogenous leukemia (CML), non-Hodgkin lymphoma or multiple myeloma.

Disorders relating to female infertility include without limitation polycystic ovarian syndrome (PCOS), diminished ovarian reserve, endometriosis, and infertility caused by radiation or chemotherapy. PCOS is often associated with lipodystrophy and insulin resistance. Disorders relating to male infertility include without limitation oligospermia and spermatogenesis caused by medications.

Somatic mutations in mitochondrial DNA increase significantly with age, which can result in defective mitochondria. Moreover, respiratory chain activity diminishes with age. Non-limiting examples of aging-related disorders are described below.

In some embodiments, one or more NR/NAR derivatives are used to treat a mitochondria-related disease or condition selected from lipodystrophy (including congenital/genetic and acquired, partial and generalized, and severe), metabolic syndrome, obesity, types 1 and 2 diabetes, NAFLD, NASH, ALD, ASH, autoimmune hepatitis, cholestatic liver disease, hemochromatosis, alpha-1 antitrypsin deficiency, other hereditary inborn errors of metabolism, and renal ischemia and IRI.

Diseases and conditions characterized by acute NAD⁺ depletion due to DNA damage include without limitation exposure to radiation (e.g., UV and ionizing radiation such as X-ray), radiation or chemotherapy-induced disorders (e.g., dermatitis, myositis, myocarditis, colitis, prostatitis, hepatitis, pneumonitis, neuropathies and bone marrow failure), burn injuries (including first-degree burns, second-degree burns and third-degree burns), chemical exposure with manifestation of exfoliative dermatitis, exposure to chemical warfare agents, Stevens-Johnson syndrome, acute respiratory distress syndrome, inhalational lung injury due to smoke or chemical toxins, trauma-related crush injuries (including those with bone fractures), peripheral nerve injuries, spinal cord injuries, and contusion to internal organs (such as the heart, lung, liver and kidney). Such diseases and conditions can generate a large amount of ROS such as superoxide, peroxides and hydroxyl radical, which cause DNA damage and hence cell damage or cell death. In other words, DNA damage induced by, e.g., radiation, chemotherapy or oxidative stress can cause acute NAD⁺ depletion that results in systemic toxicity and systemic disorders (e.g., dermatitis, pneumonitis, bone marrow failure and neuropathies), as well as local toxicity and local disorders. Exemplary chemical warfare agents include blister agents (e.g., vesicants, nitrogen mustards, sulfur mustards, arsenicals and urticants [e.g., phosgene]), blood agents (e.g., cyanide), pulmonary agents (e.g., phosgene), and nerve agents (e.g., G-series agents [e.g., sarin and soman]. GV-series agents and V-series agents).

Reduced NAD⁺ levels are associated with aging, which leads to aging-related metabolic dysfunction and disorders (e.g., inflammatory disorders). For example, the expression and activity of CD38, which rapidly degrades NAD⁺ and its precursor NMN, increase during the aging process. Furthermore, oxidative stress increases with aging. Therefore, the NR and NAR derivatives described herein are useful for treating aging-related disorders and conditions. Furthermore, the NR and NAR derivatives described herein can extend the lifespan of cells by, e.g., slowing or delaying the aging/senescence of cells, promoting the survival of cells, preventing apoptosis of cells, extending the proliferative capacity of cells, increasing cellular resistance to stress (e.g., oxidative stress), mimicking the effects of calorie restriction or promoting wound healing, or any combination thereof. In addition, NAD⁺ repletion improves stem cell function. Aging-related disorders and conditions include, but are not limited to, aging/senescence, hypertension, eye disorders (e.g., AMD, cataracts and keratoconjunctivitis sicca [dry eye syndrome]), hearing loss, bone disorders (e.g., osteoporosis), muscle disorders (e.g., muscle atrophy and sarcopenia), neurodegenerative disorders (e.g., dementias [e.g., Alzheimer's disease] and Parkinson's disease), metabolic disorders (e.g., metabolic decline, metabolic syndrome, diabetes [including T1D and T2D], and obesity), cardiovascular disorders (e.g., arteriosclerosis), inflammatory disorders (e.g., chronic inflammation, arthritis and COPD), fibrotic disorders (e.g., IPF), DNA-repair syndromes (e.g., Cockayne syndrome), and tumors and cancers. Because of their cytoprotective and antioxidant properties, for example, the NR and NAR derivatives can be used to prevent or mitigate hearing loss, including noise-induced hearing loss, trauma-induced hearing loss and progressive hearing loss syndromes.

By enhancing cell viability, providing cytoprotection or/and increasing cell lifespan, the NR and NAR derivatives of the disclosure can be used to treat disorders characterized by cell degeneration or death. For example, retinal disorders characterized by cell degeneration or death include, but are not limited to, AMD, retinitis pigmentosa, cone-rod dystrophy/degeneration, diabetic retinopathy, Leber's congenital amaurosis, and vision loss.

The cytoprotective NR and NAR derivatives can be used to treat other disorders and conditions characterized by cell degeneration or/and cell death, including without limitation neuronal disorders (e.g., Alzheimer's disease, Creutzfeld-Jakob disease, Parkinson's disease, ALS and multiple sclerosis), degeneration of the brain (e.g., cerebellar degeneration and traumatic brain injury [TBI]), muscle disorders (e.g., muscular dystrophies such as Duchenne MD, facioscapulohumeral MD and myotonic dystrophy), ischemic disorders (e.g., myocardial ischemia/infarction and cerebral ischemia [stroke]/infarction), atherosclerosis, myelodysplastic syndromes (e.g., aplastic anemia), hepatitis (e.g., alcoholic hepatitis, fulminant hepatitis, hepatitis A, hepatitis B, hepatitis C, hepatitis D and hepatitis E), joint disorders (e.g., osteoarthritis), skin atrophy, lichen planus, skin damage caused by UV light, graft rejections, alopecia, AIDS, and cell damage or/and cell death caused by trauma (e.g., to the brain or the spinal cord), surgery, medications, chemicals, biological and chemical toxins, and radiation (e.g., ionizing radiation such as X-ray). To prevent cell damage or/and cell death that may result from, e.g., a medical intervention such as surgery or radiation therapy, one or more NR/NAR derivatives can be administered to the subject prior to or/and shortly after the intervention.

In part because of their ability to reduce inflammation and oxidative stress, to protect cells from the effects of DNA damage and to enhance cell viability and lifespan, the NR and NAR derivatives described herein are useful for treating skin disorders and conditions. The skin disorders and conditions can be associated with or caused by, e.g., natural aging, inflammation, oxidative stress or sun damage. Such skin disorders and conditions include without limitation skin wrinkles, dermatitis/eczema (e.g., atopic dermatitis, contact dermatitis [allergic and irritant], exfoliative dermatitis and seborrheic dermatitis), psoriasis (e.g., plaque psoriasis), skin damage caused by sunlight or other light sources (e.g., sunburn, actinic keratosis and xeroderma pigmentosum), keratinization disorders, erythemas (e.g., erythema multiforme and erythema nodosum), dermatomyositis, discoid lupus erythematosus, pemphigoid (e.g., bullous pemphigoid), pemphigus (e.g., pemphigus vulgaris), epidermolysis bullosa, burns (e.g., first-degree burns, second-degree burns and third-degree burns, and thermal burns, radiation burns, chemical burns and electrical burns), wounds, skin cancers, and acne.

Diseases/disorders and conditions, including mitochondrial diseases and mitochondria-related diseases and conditions, that are associated with (e.g., are caused by or result in) secondary mitochondrial dysfunction (SMD) include without limitation neurodegenerative disorders {e.g., dementias (e.g., Alzheimer's disease), motor neuron disorders (e.g., Parkinson's disease, amyotrophic lateral sclerosis, spinal muscular atrophy [SMA] and hereditary spastic paraplegia [e.g., type 7]), and ataxia (e.g., Friedreich's ataxia)}, neuronal activation disorders {e.g., neuropathies (e.g., Charcot-Marie-Tooth disease [e.g., types 2A and 2K] and drug-induced peripheral neuropathies)}, muscle disorders (e.g., myopathies [e.g., Bethlem myopathy, inflammatory myopathies and statin-induced myopathy] and muscular dystrophies [e.g., limb-girdle MD]), neuromuscular disorders {e.g., dystonias (e.g., torsion dystonia [e.g., type 6])}, neurodevelopmental disorders (e.g., autism spectrum disorder and Rett syndrome), metabolic disorders (e.g., diabetes [e.g., types 1 and 2], insulin resistance, fatty acid/beta oxidation disorders [e.g., MTP deficiency], organic acidemias/acidurias [e.g., methylmalonic acidemia and propionic acidemia], urea cycle disorders [e.g., argininosuccinic aciduria], and purine/pyrimidine synthesis disorders [e.g., Lesch-Nyhan syndrome]), cardiovascular disorders (heart diseases and atherosclerosis), liver disorders (e.g., Wilson's disease), renal disorders (e.g., acute kidney injury and chronic kidney disease), disorders of the immune system (including the innate immune system) {e.g., autoimmune disorders (e.g., multiple sclerosis, systemic lupus erythematosus [lupus] and autoimmune skin disorders [e.g., lupus and pemphigus vulgaris])}, fibrotic disorders (e.g., pulmonary fibrosis [e.g., idiopathic and connective tissue disease-related lung fibrosis] and renal fibrosis [e.g., renal tubulointerstitial fibrosis]), proliferative disorders {e.g., tumors (e.g., tuberous sclerosis complex) and cancers (e.g., of the adrenal gland [e.g., pheochromocytoma], neuroendocrine system [e.g., paraganglioma], and hematopoietic and lymphoid tissues [e.g., acute lymphoblastic leukemia])}, chromosomal disorders (e.g., Down syndrome (trisomy 21), 8921.11 deletion syndrome, and contiguous gene syndromes (e.g., DiGeorge syndrome [22q11.2 deletion syndrome], 22q13 duplication and deletion syndromes, and 15q11q13 duplication syndrome)), aging-related disorders (e.g., neurodegenerative disorders [e.g., Alzheimer's and Parkinson's diseases] and muscle disorders), and SMD induced by environmental factors (e.g., psychological stress, radiation, infections, and mitotoxic agents [e.g., alcohol] and drugs [e.g., acetaminophen, doxorubicin, propofol, risperidone and statins]). There may be overlap in the categorization of disorders. As an example, certain neurodegenerative disorders, neuronal activation disorders, muscle disorders and neuromuscular disorders may be included in one or more, or all, of those categories. For instance, SMA may be regarded as a neurodegenerative disorder, a muscle disorder and a neuromuscular disorder.

Partly because of their cytoprotective properties, the NR and NAR derivatives disclosed herein can promote donor graft preservation in organ transplantation. Therefore, the NR and NAR derivatives can be applied to cells, tissue or organ employed in transplantation and cell therapies, such as solid-tissue grafts, organ transplants, cell suspensions, stem cells and bone marrow cells. The cells, tissue or organ may be an autograft, an alograft, a syngraft or a xenograft. The cells, tissue or organ can be treated with one or more NR/NAR derivatives prior to, concurrently with or/and post administration/implantation of the cells, tissue or organ into a recipient. The cells, tissue or organ can be treated with one or more NR/NAR derivatives prior to removal of the cells, tissue or organ from the donor, ex vivo after removal of the cells, tissue or organ from the donor, or post administration/implantation into the recipient. For example, the donor or/and the recipient can be treated systemically with one or more NR/NAR derivatives, or can have a subset of cells, tissue or organ treated locally with one or more NR/NAR derivatives. In certain embodiments, the cells, tissue or organ (or the donor or/and the recipient) are treated with an additional therapeutic agent that prolongs graft survival, such as an immunosuppressant, a cytokine or an angiogenic factor, or any combination thereof.

As an example, one or more NR/NAR derivatives alone or with one or more other therapeutic agents (e.g, a PARP inhibitor) can be administered to the donor or/and the recipient to promote liver regeneration for various clinical scenarios. Such clinical scenarios include prevention of liver decompensation in the donor or/and the recipient following liver segment/mass resection, prevention of liver failure in a recipient of a split liver or living donor transplantation where a sub-optimal liver mass is transplanted, and prevention of liver-related morbidity in the donor of a living donor transplantation. Liver regeneration is important in patients with acute liver failure, where about 70-90% of liver cells often die due to acute injury.

As another example, since enhancement of NAD⁺ levels promotes differentiation of transplanted cells, the use of one or more NR/NAR derivatives can improve engraftment of a bone marrow transplant, which can minimize cytopenia (including neutropenia, lymphopenia, anemia and thrombocytopenia), the need for growth factors and complications of infection. As an additional example, the use of one or more NR/NAR derivatives can prevent graft versus host disease (GVHD) in an allogeneic transplant.

In some embodiments, one or more NR/NAR derivatives are used in culture medium as a component of an ex vivo therapy, such as a chimeric antigen receptor (CAR) T-cell therapy. A CAR-T cell therapy can be autologous or allogeneic. In certain embodiments, the ex vivo therapy utilizes hematopoietic stem cells (HSC), embryonic stem cells (ESC) or pluripotent stem cells (PSC). One or more NR/NAR derivatives can be used to improve the yield of pancreatic endocrine cells during the final stages of in vitro ESC and PSC differentiation into pancreatic islet-like, insulin-secreting cells.

In further embodiments, the NR and NAR derivatives are used to enhance mitochondrial or cellular function or/and cellular energy production in oocytes, postnatal female germline stem cells or/and pre-implantation embryos prior to or/and following in vitro fertilization, or following exposure of ovaries, oocytes, postnatal female germline cells or/and preimplantation embryos in vivo. In some embodiments, one or more NR/NAR derivatives are used with a solution selected from cell culture medium, oocyte retrieval solution, oocyte washing solution, oocyte in vitro maturation medium, ovarian follicle in vitro maturation medium, oocyte in vitro fertilization medium, vitrification solution and cryopreservation solution in assisted reproduction techniques such as in vitro fertilization. The disclosure encompasses compositions comprising an isolated oocyte, oogonial stem cell (OSC) or OSC progeny, and one or more NR/NAR derivatives.

The therapeutically effective amount and the frequency of administration of, and the length of treatment with, an NR or NAR derivative to treat a disease/disorder or condition disclosed herein may depend on various factors, including the nature and severity of the disease/disorder or condition, the potency of the compound, the route of administration, the age, body weight, general health, gender and diet of the subject, and the response of the subject to the treatment, and can be determined by the treating physician. In some embodiments, the therapeutically effective amount of an NR or NAR derivative to treat a disease/disorder or condition disclosed herein, or to bring about a biological effect (e.g., increase NAD⁺ level, enhance mitochondrial or cellular function, improve metabolic health or cell viability, or provide cytoprotection), is about 1-1000 mg, 1-100 mg, 100-500 mg or 500-1000 mg (e.g., per day or per dose), or as deemed appropriate by the treating physician, which can be administered in a single dose or in divided/multiple doses. In further embodiments, the therapeutically effective amount of an NR or NAR derivative is about 1-50 mg, 50-100 mg, 100-200 mg, 200-300 mg, 300-400 mg, 400-500 mg, 500-600 mg, 600-700 mg, 700-800 mg, 800-900 mg or 900-1000 mg (e.g., per day or per dose). In additional embodiments, the therapeutically effective amount of an NR or NAR derivative is about 50 mg, 100 mg, 150 mg, 200 mg, 250 mg, 300 mg, 400 mg, 500 mg, 600 mg, 700 mg, 800 mg, 900 mg or 1000 mg (e.g., per day or per dose). The therapeutically effective amount of an NR or NAR derivative can also be higher than 1.0 g, such as about 1.0-1.5 g, 1.5-2.0 g, 2.0-2.5 g or 2.5-3.0 g (e.g., per day or per dose).

In some embodiments, the therapeutically effective amount of an NR or NAR derivative is about 100-500 mg, 100-200 mg, 200-300 mg, 300-400 mg or 400-500 mg per day, which can be administered in a single dose (e.g., N mg once daily) or in divided/multiple doses (e.g., N/2 mg twice daily). In further embodiments, the therapeutically effective amount of an NR or NAR derivative is about 100 mg, 150 mg, 200 mg, 250 mg, 300 mg, 400 mg or 500 mg per day. In certain embodiments, the therapeutically effective amount of an NR or NAR derivative is about 200-300 mg per day, or about 200 mg, 250 mg or 300 mg per day.

The therapeutically effective dose of an NR or NAR derivative can be administered one, two or more (e.g., three or four) times a day, once every two days, once every three days, twice a week or once a week, or as deemed appropriate by the treating physician. In certain embodiments, the therapeutically effective dose of an NR or NAR derivative is administered once or twice daily. As an illustrative example, if the therapeutically effective dose of an NR or NAR derivative is about 300 mg per day, 300 mg of the compound can be taken once daily, or 150 mg of the compound can be taken twice daily.

Where a more rapid establishment of a therapeutic level of an NR or NAR derivative is desired, such as in the treatment of an ischemia-reperfusion injury, the compound can be administered under a dosing schedule in which a loading dose is administered, followed by (i) one or more additional loading doses and then one or more therapeutically effective maintenance doses, or (ii) one or more therapeutically effective maintenance doses without an additional loading dose, as deemed appropriate by the treating physician. In such a case, a loading dose of a drug is larger (e.g., about 1.5, 2, 3, 4 or 5 times larger) than a subsequent maintenance dose and is designed to establish a therapeutic level of the drug more quickly. The one or more therapeutically effective maintenance doses can be any therapeutically effective amount/dose described herein. In certain embodiments, the loading dose is about three times larger than the maintenance dose. In some embodiments, a loading dose of an NR or NAR derivative is administered on day 1 and a maintenance dose is administered on day 2 and thereafter for the duration of therapy. In other embodiments, a first loading dose of an NR or NAR derivative is administered on day 1, a second loading dose is administered on day 2, and a maintenance dose is administered on day 3 and thereafter for the duration of therapy. In certain embodiments, the first loading dose is about three times larger than the maintenance dose, and the second loading dose is about two times larger than the maintenance dose.

The length of treatment with an NR or NAR derivative can be based on, e.g., the nature and severity of the disease/disorder or condition and the response of the subject to the treatment. In certain embodiments, a therapeutically effective amount of an NR or NAR derivative is administered over a period of about 1, 2, 3, 4, 5 or 6 days, or about 1, 2, 3, 4, 5 or 6 weeks, to treat an acute disease/disorder or condition. Acute disorders and conditions include without limitation damage and injury to tissues and organs (e.g., the brain, spinal cord, kidney and liver) and ischemic disorders (e.g., myocardial ischemia/infarction and cerebral ischemia/infarction). In other embodiments, a therapeutically effective amount of an NR or NAR derivative is administered over a period of at least about 6 weeks, 8 weeks (2 months), 3 months, 6 months, 1 year, 2 years, 3 years, 4 years, 5 years, 10 years or longer to treat a chronic disease/disorder or condition. It is understood that the delineation between acute and chronic may vary based on, e.g., the particular disease/disorder or condition.

An NR or NAR derivative can also be taken pro re nata (as needed) until clinical manifestations of the condition disappear or clinical targets are achieved. For example, an NR or NAR derivative can be taken until attainment of a target blood glucose level, blood pressure, blood levels of lipids, body weight or body mass index, or any combination thereof. If clinical manifestations of the condition re-appear or the clinical targets are not maintained, administration of the NR or NAR derivative can resume. Under an alternative pro re nata treatment and also at the treating physician's discretion, the dose of the NR or NAR derivative or/and its dosing frequency can be reduced upon improvement of clinical outcome(s) and then can be increased (e.g., to the previously effective dose or/and dosing frequency) if the patient's clinical status subsequently worsens.

An NR or NAR derivative can be administered via any suitable route. Potential routes of administration of an NR or NAR derivative include without limitation oral, parenteral (including intradermal, subcutaneous, intravascular, intravenous, intra-arterial, intramuscular, intraperitoneal, intracavitary, intramedullary, intrathecal and topical), and topical (including dermal/epicutaneous, transdermal, mucosal, transmucosal, intranasal [e.g., by nasal spray or drop], pulmonary [e.g., by oral or nasal inhalation], ocular [e.g., by eye drop], buccal, sublingual, rectal [e.g., by suppository] and vaginal [e.g., by suppository]). In some embodiments, an NR or NAR derivative is administered orally {e.g., as a tablet or capsule, optionally with an enteric coating (e.g., Opadryo Enteric [94 Series])}. In other embodiments, an NR or NAR derivative is administered parenterally (e.g., intravenously, subcutaneously, intramuscularly, intrathecally or topically [e.g., sublingually]).

The mode of administration can depend on, e.g., the particular disease/disorder or condition being treated. As an example, for treatment of an ocular or retinal disorder, an NR or NAR derivative can be administered, e.g., by eye drop. As another example, for treatment of a skin disorder or condition, a topical composition containing an NR or NAR derivative can be applied to the affected area(s) of the skin. As an additional example, for treatment of an airway disorder, an NR or NAR derivative can be administered by oral inhalation.

An NR or NAR derivative can be administered at any time convenient to the patient, such as in the morning or/and at nighttime (e.g., bedtime). Moreover, an NR or NAR derivative can be taken substantially with food (e.g., with a meal or within about 1 hour or 30 minutes before or after a meal) or substantially without food (e.g., at least about 1 or 2 hours before or after a meal).

The disclosure provides a method of treating a disease/disorder or condition described herein, or bringing about a biological effect described herein, comprising administering to a subject in need of treatment a therapeutically effective amount of one or more NR/NAR derivatives or a pharmaceutical composition comprising the same. The disclosure further provides one or more NR/NAR derivatives, or a composition comprising one or more NR/NAR derivatives, for use as a medicament. In addition, the disclosure provides for the use of one or more NR/NAR derivatives in the preparation of a medicament. The medicament containing the one or more NR/NAR derivatives can be used to treat any disease/disorder or condition described herein or to bring about any biological effect described herein. In certain embodiments, the one or more NR/NAR derivatives are or include a compound of Formula II or IV. In further embodiments, the one or more NR/NAR derivatives are or include a compound of Formula I and a compound of Formula II, or a compound of Formula III and a compound of Formula IV. The one or more NR/NAR derivatives can optionally be used with one or more additional therapeutic agents (e.g., a PARP inhibitor or/and a mitochondrial uncoupler).

The description and all of the embodiments relating to therapeutic use of the NR and NAR derivatives disclosed herein, including without limitation the diseases/disorders and conditions that can be treated, the biological effects that can be achieved, the therapeutically effective amount, loading dose/maintenance dose, the frequency and route of administration, the length of treatment and combination therapies, also apply to therapeutic use of other NR and NAR derivatives (e.g., NRTA, NRHTA, NARTA and NARHTA), and to therapeutic use of NR, NRH, NAR and NARH, alone or in combination with one or more other therapeutic agents described herein (e.g., a PARP inhibitor or/and a mitochondrial uncoupler).

Combination Therapies with Other Therapeutic Agents

One or more NR/NAR derivatives disclosed herein can be used alone or in combination with one or more additional therapeutic agents to treat a disease/disorder or condition disclosed herein, or to bring about a biological effect disclosed herein. The additional therapeutic agent(s) can be administered prior to, concurrently with or subsequent to administration of the NR/NAR derivative(s). Furthermore, the additional therapeutic agent(s) and the NR/NAR derivative(s) can be administered in the same pharmaceutical composition or in separate compositions.

Other types of therapeutic agents that can be used in combination with the NR and NAR derivatives of the disclosure include without limitation sirtuin-activating agents, AMPK-activating agents, CD38 inhibitors, PARP inhibitors, mitochondrial uncouplers, stimulators of cellular oxygen consumption, NMDA receptor antagonists, acetylcholinesterase inhibitors, antidiabetics, anti-obesity agents, antiplatelet agents, anticoagulants, antihypertensive agents, antioxidants, anti-inflammatory agents, analgesics, anesthetics, anticancer agents, antivirals, antibiotics, antifungals, natural compounds, vitamins and vaccines. The additional therapeutic agents can also include, e.g., farnesoid X receptor agonists and sunblocks.

Combination therapies with PARP inhibitors or/and mitochondrial uncouplers are described in detail in separate sections below. The disclosure in this section can also apply to combination therapies with PARP inhibitors or/and mitochondrial uncouplers.

Sirtuin-activating agents include agents that increase the activity, level (e.g., expression) or signaling of a sirtuin such as SIRT1 or SIRT3. SIRT1 and SIRT3's beneficial properties are described above. Sirtuin-activating agents mimic calorie restriction, enhance mitochondrial and cellular function, enhance cell viability, increase cell lifespan, increase mitochondrial biogenesis, protect against fatty liver and muscle wasting, and have anti-inflammatory, antidiabetic, cardioprotective and anti-aging effects, among other therapeutic effects. SIRT1-activating agents include without limitation lamin A, methylene blue, resveratrol, SRT-1460, SRT-1720, SRT-2104, SRT-2183, and analogs, derivatives, fragments and salts thereof. In addition to resveratrol, other polyphenols that activate sirtuins such as SIRT1 include, but are not limited to, butein, fisetin, isoliquiritigenin, piceatannol, quercetin, and analogs, derivatives and salts thereof. Metformin increases the activity of sirtuins such as SIRT1 by increasing NAD⁺ levels via activation of the NAD⁺ salvage pathway enzyme nicotinamide phosphoribosyltransferase (NAMPT) and by increasing the NAD⁺/NADH ratio. Other sirtuin-activating agents include amino acids with a branched side chain and metabolites thereof, including without limitation leucine and its metabolites such as hydroxymethylbutyrate and keto-isocaproic acid/isocaproate. Such amino acids increase the levels and stimulate the signaling of sirtuins such as SIRT1 and SIRT3.

AMPK-activating agents include agents that increase the activity, level (e.g., expression) or signaling of 5′-AMP-activated protein kinase (AMPK). AMPK plays an important role in cellular energy homeostasis, largely through stimulation of glucose and fatty acid uptake and oxidation when cellular energy is low. Activation of AMPK stimulates lipolysis, hepatic and skeletal muscle fatty acid oxidation, ketogenesis and glucose uptake, inhibits cholesterol and triglyceride synthesis and lipogenesis (including adipocyte lipogenesis), and modulates insulin secretion by pancreatic β-cells. Activation of AMPK also stimulates autophagy, mitochondrial biogenesis and antioxidant defenses, and improves cell function and health. AMPK-activating agents include without limitation sirtuin-activating agents (e.g., resveratrol, quercetin, metformin, and amino acids with a branched side chain and metabolites thereof), biguanides (e.g., buformin, metformin and phenformin), thiazolidinedione PPAR-7 agonists (infra, such as pioglitazone and rosigitazone), cannabinoids, 5-aminoimidazole-4-carboxamide-1-β-D-riboside, berberine, curcumine, 2.4-dinitrophenol (DNP), epigallocatechin-3-gallate, α-lipoic acid, A-769662. MK-8722, O-304. PF-793, PT-1, adiponectin, ghrelin, leptin, interleukin-6 (IL-6), and analogs, derivatives, fragments and salts thereof.

NAD⁺ and its precursor NMN are rapidly degraded by the extracellular glycohydrolase CD38. CD38 expression and activity increase during the aging process, which reduces NAD⁺ levels and leads to aging-related metabolic dysfunction and disorders (e.g., inflammatory disorders). Inhibition of CD38 increases NAD⁺ levels and thereby improves mitochondrial and cellular function and increases the activity of sirtuins such as SIRT1 and SIRT3. CD38 inhibitors include, but are not limited to, flavonoids (e.g., apigenin and quercetin), thiazoloquin(az)olin(on)es disclosed in C. Haffner et al., J. Med. Chem., 58:3548-3571 (2015) (e.g., compounds 76a, 76c, 77a, 77c, 77d, 78a, 78c, 78d, 78e, 79a, 79c and 79d), and analogs, derivatives and salts thereof.

Cellular oxygen consumption is a reliable indicator of mitochondrial activity since mitochondrial activity is responsible for nearly all oxygen use by cells. Mitochondria play critical roles in various cellular processes including energy production and biosynthesis. Agents that increase mitochondrial activity can be used, e.g., to treat mitochondrial diseases (e.g., Leigh syndrome and LHON), mitochondria-related diseases and conditions (e.g., metabolic disorders and neurodegenerative disorders [e.g., Alzheimer's disease, Parkinson's disease, ALS, Friedreich's ataxia and FXTAS]), to aid recovery from injury (e.g., TBI) or illness, and to delay aging. Stimulators of cellular oxygen consumption increase mitochondrial activity through increased mitochondrial function or/and number. Stimulators of cellular oxygen consumption include without limitation mitochondrial uncouplers, acarbose, chlormadinone (e.g., chlormadinone acetate), desoxymetasone, dichlorophene, enilconazole, flumazenil, quinidine (e.g., quinidine gluconate), succinylsulfathiazole, toltrazuril, and analogs, derivatives and salts thereof.

In some embodiments, one or more NR/NAR derivatives described herein are used in combination with an N-methyl-D-aspartate receptor (NMDAR) antagonist to treat a disorder characterized by neurodegeneration or neurotoxicity, such as a dementia (e.g., Alzheimer's disease) or a motor neuron disorder (e.g., Parkinson's disease). In certain embodiments, the NMDAR antagonist is an uncompetitive antagonist (or channel blocker) that has a moderate affinity (e.g., a Kᵢ or IC₅₀ from about 200 nM to about 10 μM) for the dizocilpine (MK-801)/phencyclidine-binding site at or near the Mg²-binding site in the opened ion channel of activated NMDAR, which allows the antagonist to inhibit NMDAR-mediated excitotoxicity while preserving physiological NMDAR activity. Such NMDAR uncompetitive antagonists include without limitation alaproclate, amantadine, atomoxetine, budipine, delucemine, dextrallorphan, dextromethorphan, dextrorphan, dexanabinol, eliprodil, ketamine, lanicemine, minocycline, memantine, nitromemantine, NEFA (a tricyclic small molecule), neramexane, orphenadrine, procyclidine, ARL/FPL 12495/12495AA (des-glycine metabolite of remacemide), and analogs, derivatives and salts thereof. In some embodiments, the NMDAR antagonist is memantine, nitromemantine, amantadine, lanicemine, neramexane, dextrallorphan, dextromethorphan, dextrorphan (metabolite of dextromethorphan) or procyclidine, or a salt thereof. In certain embodiments, the NMDAR antagonist is memantine, nitromemantine, dextrallorphan, dextromethorphan or dextrorphan, or a salt thereof.

In further embodiments, one or more NR/NAR derivatives disclosed herein are used in combination with an acetylcholinesterase inhibitor (AChEI) to treat a cognitive disorder (e.g., a dementia such as Alzheimer's disease, Lewy body dementia or Parkinson-associated dementia) or a neuromuscular disorder (e.g., myasthenia gravis). Reversible AChEIs include, but are not limited to, neostigmine, physostigmine, pyridostigmine, rivastigmine, ambenonium, demecarium, donepezil, edrophonium, ladostigil, and analogs, derivatives and salts thereof.

Other therapeutic agents that can be used in conjunction with one or more NR/NAR derivatives to treat Parkinson's disease include without limitation levodopa, dopamine agonists (e.g., apomorphine, bromocriptine, cabergoline, lisuride, pergolide, piribedil, pramipexole, ropinirole and rotigotine), catechol-O-methyltransferase (COMT) inhibitors (e.g., entacapone, opicapone and tolcapone), monoamine oxidase B (MAO-B) inhibitors (e.g., ladostigil, safinamide, selegiline and rasagiline), peripheral aromatic L-amino acid decarboxylase inhibitors (e.g., carbidopa), and analogs, derivatives and salts thereof.

In additional embodiments, one or more NR/NAR derivatives disclosed herein are used in combination with one or more antidiabetic agents to treat hyperglycemia, insulin resistance or diabetes (e.g., T1D or T2D), or a disorder associated therewith (e.g., NAFLD or NASH). In certain embodiments, the one or more antidiabetic agents are or include a biguanide (e.g., metformin), a thiazolidinedione (e.g., pioglitazone or rosiglitazone), a GLP-1 agonist (e.g., dulaglutide or semaglutide) or a SGLT2 inhibitor (e.g., empagliflozin or tofogliflozin), or any combination thereof.

Antidiabetic agents include without limitation:

AMP-activated protein kinase (AMPK) agonists, including biguanides (e.g., buformin, metformin and phenformin) and allosteric AMPK activators (e.g., MK-8722 and PF-793);

peroxisome proliferator-activated receptor gamma (PPAR-γ) agonists, including thiazolidinediones (e.g., balaglitazone, ciglitazone, darglitazone, englitazone, lobeglitazone, netoglitazone, pioglitazone, deuterated (R)-pioglitazone [e.g., DRX-065], rivoglitazone, rosiglitazone and troglitazone), saroglitazar (dual PPAR-α/γ agonist) and IVA-337 (triple PPAR-α/δ/γ agonist);

glucagon-like peptide-1 (GLP-1) receptor agonists, including exendin-4, albiglutide, dulaglutide, exenatide, liraglutide, lixisenatide, semaglutide, taspoglutide, AC-3174, CNT0736, CNT03649, HM11260C (LAPS-Exendin), NN9926 (OG9S7GT), TT401 and ZYOGi;

dual GLP-1 receptor (GLP-1R)/glucagon receptor (GCGR) agonists, including longer-acting oxyntomodulin analogs {e.g., lipid-conjugated OXM analogs (e.g., DualAG disclosed in A. Pocai et al., Diabetes, 58:2258-2266 [2009]), PEGylated OXM analogs, cross-linked OXM analogs disclosed in A. Muppidi et al., ACS Chem. Biol., 11:324-328 (2016) and OX-SR disclosed in R. Scott et al., Peptides, 104:70-77 (2018)}, HM12525A, JNJ-54728518, LY2944876 (TT-401), MED10382, MK-8521, MOD-6031, NN9277, SAR425899, SP-1373 and ZP2929;

dual GLP-1R/gastric inhibitory peptide receptor (GIPR) agonists, including Cpd86, LY3298176, NN9709 (MAR709), SAR438335, ZP-DI-70 and ZP-I-98;

triple GLP-1R/GIPR/GCGR agonists, including HM15211 and MAR423;

dipeptidyl peptidase 4 (DPP-4) inhibitors, including alogliptin, anagliptin, dutogliptin, evogliptin, gemigliptin, gosogliptin, linagliptin, omarigliptin, saxagliptin, septagliptin, sitagliptin, des-fluoro-sitagliptin, teneligliptin, trelagliptin and vildagliptin; glucokinase activators, including piragliatin, ARRY-403, HMS-5552, TMG-123 and TTP-399;

inhibitors of α-glucosidases, including acarbose, miglitol and voglibose;

ketohexokinase (KHK) inhibitors, including PF-06835919;

sodium-glucose transport protein 2 (SGLT2) inhibitors, including canagliflozin (also inhibits SGLT1), dapagliflozin, empagliflozin, ertugliflozin, ipragliflozin, remogliflozin etabonate, sotagliflozin (also inhibits SGLT1) and tofogliflozin;

blockers of ATP-dependent K+(K_{ATP}) channels on pancreatic beta cells, including meglitinides (e.g., mitiglinide, nateglinide and repaglinide) and sulfonylureas {including first generation (e.g., acetohexamide, carbutamide, chlorpropamide, glycyclamide [tolhexamide], metahexamide, tolazamide and tolbutamide) and second generation (e.g., glibenclamide [glyburide], glibornuride, gliclazide, glimepiride, glipizide, gliquidone, glisoxepide and glyclopyramide)};

insulin and analogs thereof, including fast-acting insulin (e.g., insulin aspart, insulin glulisine and insulin lispro), intermediate-acting insulin (e.g., NPH insulin), and long-acting insulin (e.g., insulin degludec, insulin detemir and insulin glargine):

amylin and analogs thereof, including pramlintide; and

analogs, derivatives and salts thereof.

In further embodiments, one or more NR/NAR derivatives described herein are used in combination with one or more anti-obesity agents to treat obesity or hyperlipidemia or a disorder associated therewith, such as a metabolic disorder (e.g., T2D, metabolic syndrome or NAFLD) or a cardiovascular disorder (e.g., atherosclerosis or CAD). Obesity also promotes inflammatory processes. In certain embodiments, the one or more anti-obesity agents are or include a lipase inhibitor (e.g., orlistat) or/and an antihyperlipidemic agent (e.g., a statin such as atorvastatin, or/and a fibrate such as fenofibrate).

Anti-obesity agents include, but are not limited to:

appetite suppressants (anorectics), including amphetamine, dexamphetamine, amfepramone, clobenzorex, mazindol, phentermine (with or without topiramate) and lorcaserin;

pro-satiety agents, including ciliary neurotrophic factor (e.g., axokine) and longer-acting analogs of amylin, calcitonin, cholecystokinin (CCK), glucagon (GCG), GLP-1, gastric inhibitory peptide (GIP, also called glucose-dependent insulinotropic polypeptide), leptin, oxyntomodulin (OXM), pancreatic polypeptide (PP), peptide YY (PYY) and neuropeptide Y (NPY);

lipase inhibitors, including caulerpenyne, cetilistat, ebelactone A and B, esterastin, lipstatin, orlistat, percyquinin, panclicin A-E, valilactone and vibralactone;

melanocortin 4 (MC₄) receptor agonists, including selective MC₄R agonists (e.g., AZD-2820, LY-2112688, MK-0493, PF-00446687, PG-931, PL-6983, Ro 27-3225 and THIQ [drug name]) and non-selective MC₄R agonists (e.g., afamelanotide, bremelanotide, melanotan II, modimelanotide and setmelanotide);

agents that increase energy expenditure or/and fat burning, including longer-acting glucagon analogs, glucagon receptor agonists (e.g., NN9030) and dual GLP-1 receptor/glucagon receptor agonists (supra); triiodothyronine (T₃) and thyroid hormone receptor-beta (THR-β) agonists (e.g., MB07344, MB07811. MGL-3196, MGL-3745, VK0214 and VK2809); fibroblast growth factor 21 (FGF21) and analogs and derivatives thereof (e.g., BMS-986036 [PEGylated FGF21] and BMS-986171); and mitochondrial uncouplers (infra);

antihyperlipidemic agents;

other agents that reduce body weight or/and fat mass, including dual GLP-R/GIPR agonists (supra) and triple GLP-1R/GIPR/GCGR agonists (supra); and

analogs, derivatives and salts thereof.

Antihyperlipidemic agents include without limitation:

HMG-CoA reductase inhibitors, including statins {e.g., atorvastatin, cerivastatin, fluvastatin, mevastatin, monacolins (e.g., monacolin K [lovastatin]), pitavastatin, pravastatin, rosuvastatin and simvastatin} and flavanones (e.g., naringenin);

squalene synthase inhibitors, including lapaquistat, zaragozic acid and RPR-107393;

fatty acid synthase inhibitors, including TVB-2640;

acetyl-CoA carboxylase (ACC) inhibitors, including anthocyanins, avenaciolides, chloroacetylated biotin, cyclodim, diclofop, firsocostat (GS-0976, NDI-010976 or ND-630), gemcabene, haloxyfop, soraphens (e.g., soraphen Ala), 5-(tetradecyloxy)-2-furancarboxylic acid (TOFA), CP-640186, DRM-01, PF-05175157, PF-05221304, QLT-091382; 7-(4-propyloxy-phenylethynyl)-3,3-dimethyl-3,4 dihydro-2H-benzo[b][1,4]dioxepine; N-ethyl-N′-(3-{[4-(3,3-dimethyl-1-oxo-2-oxa-7-azaspiro[4.5]dec-7-yl)piperidin-1-yl]-carbonyl}-1-benzothien-2-yl)urea; 5-(3-acetamidobut-1-ynyl)-2-(4-propyloxyphenoxy)thiazole; and 1-(3-{[4-(3,3-dimethyl-1-oxo-2-oxa-7-azaspiro[4.5]dec-7-yl)piperidin-1-yl]-carbonyl}-5-(pyridin-2-yl)-2-thienyl)-3-ethylurea;

ATP citrate lyase (ACL) inhibitors, including bempedoic acid (ETC-1002), 2-furoic acid, (−)-hydroxycitric acid, BMS-303141, MEDICA-16 and SB-204990;

PPAR-α agonists, including fibrates (e.g., bezafibrate, ciprofibrate, clinofibrate, clofibric acid, clofibrate, aluminum clofibrate [alfibrate], clofibride, etofibrate, fenofibric acid, fenofibrate, gemfibrozil, ronifibrate and simfibrate), isoflavones (e.g., daidzein and genistein), and perfluoroalkanoic acids (e.g., perfluorooctanoic acid and perfluorononanoic acid);

PPAR-δ agonists, including elafibranor (dual PPAR-α/δ agonist), lanifibranor (triple PPAR-α/δ/γ agonist), GFT505 (dual PPAR-α/δ agonist), GW0742, GW501516 (dual PPAR-β/δ agonist), sodelglitazar (GW677954), seladelpar (MBX-8025), and isoflavones (e.g., daidzein and genistein);

PPAR-γ agonists, including thiazolidinediones (supra), saroglitazar (dual PPAR-α/γ agonist), IVA-337 (triple PPAR-α/δ/γ agonist), 4-oxo-2-thioxothiazolines (e.g., rhodanine), berberine, honokiol, perfluorononanoic acid, cyclopentenone prostaglandins (e.g., cyclopentenone 15-deoxy-Δ-prostaglandin J₂ [15d-PGJ₂]), and isoflavones (e.g., daidzein and genistein);

liver X receptor (LXR) agonists, including endogenous ligands (e.g., oxysterols such as 22(R)-hydroxycholesterol, 24(S)-hydroxycholesterol, 27-hydroxycholesterol and cholestenoic acid) and synthetic agonists (e.g., acetyl-podocarpic dimer, hypocholamide, N,N-dimethyl-3p-hydroxy-cholenamide [DMHCA], BMS-852927, GW3965 and T0901317);

retinoid X receptor (RXR) agonists, including endogenous ligands (e.g., 9-cis-retinoic acid) and synthetic agonists (e.g., bexarotene, AGN 191659, AGN 191701, AGN 192849, BMS649, LG100268, LG100754 and LGD346);

G protein-coupled bile acid receptor 1 (TGR5) agonists, including RDX-009, INT-777 and INT-767 (dual TGR5/FXR agonist);

triiodothyronine and thyroid hormone receptor-beta agonists (supra);

ketohexokinase inhibitors (supra);

inhibitors of acyl-CoA cholesterol acyltransferase (ACAT, also called sterol O-acyltransferase [SOAT], including ACAT1 [SOAT1] and ACAT2 [SOAT2]), including avasimibe, pactimibe, pellitorine, terpendole C and flavanones (e.g., naringenin);

inhibitors of stearoyl-CoA desaturase-1 (SCD-1, also called stearoyl-CoA delta-9 desaturase) activity or expression, including aramchol, CAY-10566, CVT-11127, SAR-224, SAR-707, XEN-103; 3-(2-hydroxyethoxy)-4-methoxy-N-[5-(3-trifluoromethylbenzyl)thiazol-2-yl]benzamide and 4-ethylamino-3-(2-hydroxyethoxy)-N-[5-(3-trifluoromethylbenzyl)thiazol-2-yl]benzamide; 1′-{6-[5-(pyridin-3-ylmethyl)-1,3,4-oxadiazol-2-yl]pyridazin-3-yl1-5-(trifluoromethyl)-3,4-dihydrospiro[chromene-2,4′-piperidine]; 5-fluoro-1′-16-[5-(pyridin-3-ylmethyl)-1,3,4-oxadiazol-2-yl]pyridazin-3-yl}-3,4-dihydrospiro[chromene-2,4′-piperidine]; 6-[5-(cyclopropylmethyl)-4,5-dihydro-1′H, 3H-spiro[1,5-benzoxazepine-2,4′-piperidin]-1′-yl]-N-(2-hydroxy-2-pyridin-3-ylethyl)pyridazine-3-carboxamide; 6-[4-(2-methylbenzoyl)piperidin-1-yl]pyridazine-3-carboxylic acid (2-hydroxy-2-pyridin-3-ylethyl)amide; 4-(2-chlorophenoxy)-N-[3-(methyl carbamoyl)phenyl]piperidine-1-carboxamide; the cis-9, trans-11 isomer and the trans-10, cis-12 isomer of conjugated linoleic acid, substituted heteroaromatic compounds disclosed in WO 2009/129625 A1, anti-sense polynucleotides and peptide-nucleic acids (PNAs) that target mRNA for SCD-1, and SCD-1-targeting siRNAs;

cholesterylester transfer protein (CETP) inhibitors, including anacetrapib, dalcetrapib, evacetrapib, torcetrapib and AMG 899 (TA-8995);

inhibitors of microsomal triglyceride transfer protein (MTTP) activity or expression, including implitapide, lomitapide, dirlotapide, mitratapide, CP-346086, JTT-130, SLx-4090, anti-sense polynucleotides and PNAs that target mRNA for MTTP, MTTP-targeting microRNAs (e.g., miRNA-30c), and MTTP-targeting siRNAs;

GLP-1 receptor agonists (supra), glucagon receptor agonists (supra) and dual GLP-1 receptor/glucagon receptor agonists (supra);

inhibitors of pro-protein convertase subtilisin/kexin type 9 (PCSK9) activity or expression, including berberine (reduces PCSK9 level), annexin A2 (inhibits PCSK9 activity), anti-PCSK9 antibodies (e.g., alirocumab, bococizumab, evolocumab, LGT-209, LY3015014 and RG7652), peptides that mimic the epidermal growth factor-A (EGF-A) domain of the LDL receptor which binds to PCSK9, PCSK9-binding adnectins (e.g., BMS-962476), anti-sense polynucleotides and PNAs that target mRNA for PCSK9, and PCSK9-targeting siRNAs (e.g., inclisiran [ALN-PCS] and ALN-PCS02);

FGF21 and analogs and derivatives thereof (supra);

apolipoprotein mimetic peptides, including apoA-I mimetics (e.g., 2F, 3F, 3F-1, 3F-2, 3F-14, 4F, 4F-P-4F, 4F-IHS-4F, 4F2, 5F, 6F, 7F, 18F, 5A, 5A-C1, 5A-CHI, 5A-CH2, 5A-H1, 18A, 37 pA [18A-P-18A], ELK [name], ELK-A, ELK-1F, ELK-1K1A1E, ELK-1LK, ELK-1W, ELK-2A, ELK-2A2K2E, ELK-2E2K, ELK-2F, ELK-3E3EK, ELK-3E3K3A, ELK-3E3LK, ELK-PA, ELK-P2A, ELKA [name], ELKA-CH2, ATI-5261, CS-6253, ETC-642, FAMP [name], FREL [name] and KRES [name]) and apoE mimetics (e.g., Ac-hE18A-NH₂ [AEM-28], Ac-[R]hE18A-NH₂, AEM-28-14, EpK, hEp, mR18L, COG-112, COG-133 and COG-1410);

omega-3 fatty acids, including docosahexaenoic acid (DHA), docosapentaenoic acid (DPA), eicosapentaenoic acid (EPA), α-linolenic acid (ALA), fish oils (which contain, e.g., DHA and EPA), and esters (e.g., glyceryl and ethyl esters) thereof; and

analogs, derivatives and salts thereof.

In other embodiments, one or more NR/NAR derivatives of the disclosure are used in combination with an antiplatelet agent or/and an anticoagulant to treat a thrombotic or hemostatic disorder, such as a cardiovascular disorder (e.g., myocardial ischemia/infarction) or a cerebrovascular disorder (e.g., ischemic stroke). In certain embodiments, the antiplatelet agent is or includes a COX-1 inhibitor (e.g., aspirin) or/and a P2Y₁₂ inhibitor (e.g., clopidogrel), and the anticoagulant is or includes a direct factor Xa inhibitor (e.g., apixaban or rivaroxaban) or/and a direct thrombin inhibitor (e.g., dabigatran).

Antiplatelet agents include without limitation:

cyclooxygenase (e.g., COX-1) inhibitors, including baspirin, naproxen, triflusal and 2-hydroxy-4-trifluoromethylbenzoic acid (the main metabolite of triflusal);

thromboxane (e.g., A₂) synthase inhibitors, including isbogrel, ozagrel, picotamide, ridogrel, samixogrel, terbogrel and EV-077;

thromboxane (e.g., A₂) receptor antagonists, including dipyridamole, ifetroban, isbogrel, picotamide, ramatroban, ridogrel, samixogrel, terbogrel, terutroban, EV-077 and TRA-418;

adenosine diphosphate (ADP) receptor/P2Y₁₂ inhibitors, including cangrelor, clopidogrel, prasugrel, ticagrelor and ticlopidine;

adenosine reuptake inhibitors, including cilostazol and dipyridamole;

glycoprotein IIb/IIIa inhibitors, including abciximab, eptifibatide, tirofiban, TRA-418, and prostacyclin and analogs thereof;

phosphodiesterase (e.g., PDE3 or/and PDE5) inhibitors, including cilostazol and dipyridamole;

protease-activated receptor 1 (PAR1) antagonists, including vorapaxar;

prostacyclin and analogs thereof, including ataprost, beraprost (e.g., esuberaprost), 5,6,7-trinor-4,8-inter-m-phenylene-9-fluoro-PGI₂, carbacyclin, isocarbacyclin, clinprost (isocarbacyclin methyl ester), ciprostene, eptaloprost, cicaprost (metabolite of eptaloprost), iloprost, pimilprost, SM-10906 (des-methyl pimilprost), naxaprostene, taprostene, treprostinil, CS-570, OP-2507 and TY-11223; and

analogs, derivatives and salts thereof.

Anticoagulants include, but are not limited to:

vitamin K antagonists, including 4-hydroxycoumarins (e.g., acenocoumarol, brodifacoum, coumatetralyl, dicoumarol, phenprocoumon, tioclomarol and warfarin) and 1,3-indandiones (e.g., clorindione, diphenadione, fluindione and phenindione);

indirect factor Xa inhibitors, including heparin (unfractionated), low molecular weight (MW) heparin (e.g., Fraxiparine), low MW heparin derivatives (e.g., bemiparin, certoparin, dalteparin, enoxaparin, nadroparin, parnaparin, reviparin and tinzaparin), heparin analogs (e.g., fondaparinux and idraparinux), and heparinoids (e.g., danaparoid, sulodexide and dermatan sulfate);

direct factor Xa inhibitors, including apixaban, betrixaban, darexaban, edoxaban, eribaxaban, letaxaban, otamixaban, razaxaban, rivaroxaban, LY-517717 and YM-466;

direct thrombin (factor IIa) inhibitors (DTIs), including univalent DTIs (e.g., argatroban, dabigatran, inogatran, melagatran and ximelagatran) and bivalent DTIs (e.g., hirudin and hirudin analogs [e.g., bivalirudin, desirudin and lepirudin]); and

analogs, derivatives and salts thereof.

In additional embodiments, one or more NR/NAR derivatives disclosed herein are used in combination with one or more antihypertensive agents. Hypertension is a clinical feature of or is a major risk factor for a wide range of disorders. Hypertension-associated disorders include without limitation cardiovascular disorders (e.g., cardiomyopathy, heart failure, atherosclerosis, arteriosclerosis, coronary artery diseases [e.g., myocardial ischemia/infarction], and peripheral vascular diseases [e.g., peripheral artery disease]), cerebrovascular disorders (e.g., stroke and cerebral infarction), metabolic disorders (e.g., metabolic syndrome and T2D), kidney disorders (e.g., diabetic nephropathy, glomerulonephritis, renal ischemia, nephrotic syndrome, and kidney failure [e.g., acute kidney injury and chronic kidney disease]), liver failure (e.g., cirrhosis), and eye disorders (e.g., retinopathy, damage to blood vessels in the eye, and vision loss).

Antihypertensive agents include without limitation:

antagonists of the renin-angiotensin-aldosterone system (RAAS), including renin inhibitors (e.g., aliskiren), angiotensin-converting enzyme (ACE) inhibitors (e.g., benazepril, captopril, enalapril, fosinopril, lisinopril, moexipril, perindopril, quinapril, ramipril and trandolapril), angiotensin II receptor type 1 (AT₁) antagonists (e.g., azilsartan, candesartan, eprosartan, fimasartan, irbesartan, losartan, olmesartan medoxomil, olmesartan, telmisartan and valsartan), and aldosterone receptor antagonists (e.g., eplerenone and spironolactone);

diuretics, including loop diuretics (e.g., bumetanide, ethacrynic acid, furosemide and torsemide), thiazide diuretics (e.g., bendroflumethiazide, chlorothiazide, hydrochlorothiazide, epitizide, methyclothiazide and polythiazide), thiazide-like diuretics (e.g., chlorthalidone, indapamide and metolazone), cicletanine (an early distal tubular diuretic), potassium-sparing diuretics (e.g., amiloride, eplerenone, spironolactone and triamterene), and theobromine;

calcium channel blockers, including dihydropyridines (e.g., amlodipine, levamlodipine, cilnidipine, clevidipine, felodipine, isradipine, lercanidipine, nicardipine, nifedipine, nimodipine, nisoldipine and nitrendipine) and non-dihydropyridines (e.g., diltiazem and verapamil);

α₂-adrenoreceptor agonists, including clonidine, guanabenz, guanfacine, methyldopa and moxonidine;

α₁-adrenoreceptor antagonists (alpha blockers), including doxazosin, indoramin, nicergoline, phenoxybenzamine, phentolamine, prazosin, terazosin and tolazoline;

β-adrenoreceptor (β₁ or/and β₂) antagonists (beta blockers), including atenolol, betaxolol, bisoprolol, carteolol, carvedilol, labetalol, metoprolol, nadolol, nebivolol, oxprenolol, penbutolol, pindolol, propranolol and timolol;

mixed alpha/beta (e.g., α₁/β₁) blockers, including bucindolol, carvedilol and labetalol;

endothelin receptor antagonists, including selective ET_{A} receptor antagonists (e.g., ambrisentan, atrasentan, edonentan, sitaxentan, zibotentan and BQ-123) and dual ET_{A}/ET_{B} antagonists (e.g., bosentan, macitentan and tezosentan);

other vasodilators, including hydralazine, minoxidil, theobromine, sodium nitroprusside, organic nitrates (e.g., isosorbide mononitrate, isosorbide dinitrate and nitroglycerin, which are converted to nitric oxide in the body), endothelial nitric oxide synthase (eNOS) stimulators (e.g., cicletanine), activators of soluble guanylate cyclase (e.g., cinaciguat and riociguat), phosphodiesterase type 5 (PDE5) inhibitors (e.g., avanafil, benzamidenafil, dasantafil, dynafil, lodenafil, mirodenafil, sildenafil, tadalafil, udenafil, vardenafil, dipyridamole, papaverine, propentofylline, zaprinast and T-1032), prostaglandin E₁ (alprostadil) and analogs thereof (e.g., limaprost amd misoprostol), prostacyclin and analogs thereof (supra), non-prostanoid prostacyclin receptor agonists (e.g., 1-phthalazinol, ralinepag, selexipag, ACT-333679 [MRE-269, active metabolite of selexipag], and TRA-418), phospholipase C (PLC) inhibitors, protein kinase C (PKC) inhibitors (e.g., BIM-1, BIM-2, BIM-3. BIM-8, chelerythrine, cicletanine, gossypol, miyabenol C, myricitrin, ruboxistaurin and verbascoside), and Rho-associated protein kinase (ROCK) inhibitors (e.g., fasudil, ripasudil and Y-27632);

minerals, including magnesium and magnesium sulfate; and analogs, derivatives and salts thereof.

In certain embodiments, the one or more antihypertensive agents are or include a thiazide or thiazide-like diuretic (e.g., hydrochlorothiazide or chlorthalidone), a calcium channel blocker (e.g., amlodipine or nifedipine), an ACE inhibitor (e.g., benazepril, captopril or perindopril) or an angiotensin II receptor antagonist (e.g., olmesartan medoxomil, olmesartan, telmisartan or valsartan), or any combination thereof.

Oxidative stress is associated with a broad range of disorders. Therefore, in some embodiments one or more NR/NAR derivatives described herein are used in combination with one or more antioxidants to treat a disorder whose pathogenesis or pathophysiology involves oxidative stress or/and oxidative damage/injury. Such oxidative disorders include without limitation neurodegenerative disorders (e.g., Alzheimer's, Huntington's and Parkinson's diseases, ALS and multiple sclerosis), metabolic disorders (e.g., types 1 and 2 diabetes and metabolic syndrome), cardiovascular disorders (e.g., atherosclerosis, heart failure, myocardial ischemia/infarction and IRI), cerebrovascular disorders (e.g., stroke and IRI), kidney disorders (e.g., diabetic nephropathy), liver disorders (e.g., cirrhosis), and eye disorders (e.g., AMD). Furthermore, oxidants (e.g., ROS) and oxidized molecules (e.g., oxidized lipids) can be highly inflammatory.

Antioxidants include without limitation:

vitamins and analogs thereof, including vitamin A, vitamin B₃ (e.g., niacin [nicotinic acid] and nicotinamide), vitamin C (ascorbic acid), vitamin E (including tocopherols [e.g., α-tocopherol] and tocotrienols), and vitamin E analogs (e.g., trolox [water-soluble]);

carotenoids, including carotenes (e.g., β-carotene), xanthophylls (e.g., lutein, zeaxanthin and meso-zeaxanthin), and carotenoids in saffron (e.g., crocetin);

sulfur-containing antioxidants, including glutathione (GSH), N-acetyl-L-cysteine (NAC), bucillamine, S-nitroso-N-acetyl-L-cysteine (SNAC), S-allyl-L-cysteine (SAC), S-adenosyl-L-methionine (SAM), α-lipoic acid and taurine;

scavengers of ROS and radicals, including carnosine, N-acetylcarnosine, curcuminoids (e.g., curcumin, demethoxycurcumin and tetrahydrocurcumin), cysteamine, ebselen, glutathione, hydroxycinnamic acids and derivatives (e.g., esters and amides) thereof (e.g., caffeic acid, rosmarinic acid and tranilast), melatonin and metabolites thereof, nitrones (e.g., disufenton sodium [NXY-059]), nitroxides (e.g., XJB-5-131), polyphenols (e.g., flavonoids [e.g., apigenin, genistein, luteolin, naringenin and quercetin]), superoxide dismutase mimetics (infra), tirilazad, vitamin C, vitamin E and analogs thereof (e.g., α-tocopherol and trolox), and xanthine derivatives (e.g., pentoxifylline);

inhibitors of enzymes that produce ROS, including NADPH oxidase (NOX) inhibitors (e.g., apocynin, decursin and decursinol angelate [both inhibit NOX-1, -2 and -4 activity and expression], diphenylene iodonium, and GKT-831 [formerly GKT-137831, a dual NOX1/4 inhibitor]), NADH:ubiquinone oxidoreductase (complex I) inhibitors (e.g., metformin and rotenone), and myeloperoxidase inhibitors (e.g., azide, 4-aminobenzoic acid hydrazide and PF-06667272, and apoE mimetics such as AEM-28 and AEM-28-14);

substances that mimic or increase the activity or production of antioxidant enzymes, including superoxide dismutase (SOD) {e.g., SOD mimetics such as manganese (III)- and zinc (III)-porphyrin complexes (e.g., MnTBAP, MnTMPyP and ZnTBAP), manganese (II) penta-azamacrocyclic complexes (e.g., M40401 and M40403), manganese (III)-salen complexes (e.g., those disclosed in U.S. Pat. No. 7,122,537) and OT-551 (a cyclopropyl ester prodrug of tempol hydroxylamine), and resveratrol and apoA-I mimetics such as 4F (both increase expression)), catalase (e.g., catalase mimetics such as manganese (III)-salen complexes [e.g., those disclosed in U.S. Pat. No. 7,122,537], and zinc [increases activity]), glutathione peroxidase (GPx) (e.g., apomorphine and zinc [both increase activity], and beta-catenin, etoposide and resveratrol [all three increase expression]), glutathione reductase (e.g., 4-tert-butylcatechol and redox cofactors such as flavin adenine dinucleotide [FAD] and NADPH [all three enhance activity]), glutathione S-transferase (GST) (e.g., phenylalkyl isothiocyanate-cysteine conjugates e.g., S-[N-benzyl(thiocarbamoyl)]-L-cysteine}, phenobarbital, rosemary extract and carnosol [all enhance activity]), thioredoxin (Trx) (e.g., geranylgeranylacetone, prostaglandin E₁ and sulforaphane [all increase expression]), NADPH-quinone oxidoreductase 1 (NQO1) {e.g., flavones [e.g., 0-naphthoflavone (5,6-benzoflavone)] and triterpenoids [e.g., oleanolic acid analogs such as TP-151 (CDDO), TP-155 (CDDO methyl ester), TP-190, TP-218, TP-222, TP-223 (CDDO carboxamide), TP-224 (CDDO monomethylamide), TP-225, TP-226 (CDDO dimethylamide), TP-230, TP-235 (CDDO imidazolide), TP-241, CDDO monoethylamide, CDDO mono(trifluoroethyl)amide, and (+)-TBE-B], all of which increase expression by activating Nrf2}, heme oxygenase 1 (HO-1) {e.g., curcuminoids (e.g., curcumin), triterpenoids (e.g., oleanolic acid analogs [supra, such as TP-225]), and apoA-I mimetics (supra, such as 4F), all of which increase expression}, and paraoxonase 1 (PON-1) (e.g., apoE mimetics [supra, such as AEM-28 and AEM-28-14] and apoA-I mimetics [supra, such as 4F], both types increasing activity);

activators of transcription factors that upregulate expression of antioxidant enzymes, including activators of nuclear factor (erythroid-derived 2)-like 2 (NFE2L2 or Nrf2) {e.g., bardoxolone methyl, OT-551, fumarates (e.g., dimethyl and monomethyl fumarate), dithiolethiones (e.g., oltipraz), flavones (e.g., β-naphthoflavone), isoflavones (e.g., genistein), sulforaphane, trichostatin A, triterpenoids (e.g., oleanolic acid analogs [supra, such as TP-225]), and melatonin (increases Nrf2 expression)};

mitochondrial and mitochondria-targeted antioxidants, including ubiquinone (coenzyme Q, such as CoQ₁₀), ubiquinol (a reduced and more bioavailable form of ubiquinone, such as ubiquinol-10), ubiquinone/ubiquinol analogs (e.g., idebenone and mitoquinone) and derivatives, MitoE and MitoQ;

other kinds of antioxidants, including anthocyanins, benzenediol abietane diterpenes (e.g., carnosic acid), cyclopentenone prostaglandins (e.g., 15d-PGJ₂), flavonoids {e.g., flavonoids in Ginkgo biloba (e.g., myricetin and quercetin [increases levels of GSH, SOD, catalase, GPx and GST]), prenylflavonoids (e.g., isoxanthohumol), flavones (e.g., apigenin), isoflavones (e.g., genistein), flavanones (e.g., naringenin) and flavanols (e.g., catechin and epigallocatechin-3-gallate)}, omega-3 fatty acids and esters thereof (supra), phenylethanoids (e.g., tyrosol and hydroxytyrosol), retinoids (e.g., all-trans retinol [vitamin A]), stilbenoids (e.g., resveratrol), uric acid, apoA-I mimetics (e.g., 4F), apoE mimetics (e.g., AEM-28 and AEM-28-14), and minerals (e.g., selenium and zinc [e.g., zinc monocysteine]); and

analogs, derivatives and salts thereof.

In certain embodiments, the one or more antioxidants are or include a vitamin or an analog thereof (e.g., vitamin E or an analog thereof such as α-tocopherol or trolox), an ROS or radical scavenger (e.g., melatonin or/and glutathione), or a mitochondrial antioxidant/“vitamin” (e.g., ubiquinone-10 or ubiquinol-10) or an analog thereof, or any combination thereof. In other embodiments, the antioxidant or/and the natural compound are selected from resveratrol, pterostilbene, ellagic acid, urolithin A, quercetin, coenzyme Q (e.g., CoQ₁₀), glutathione, N-acetyl-L-cysteine, α-lipoic acid, melatonin, creatine, S-adenosyl methionine, leucine, pyruvic acid/pyruvate and combinations thereof.

In some embodiments, one or more NR/NAR derivatives are used in conjunction with one or more B vitamins selected from thiamine (B₁), riboflavin (B₂), niacin (B₃), pantothenic acid (B₅), pyridoxine (B), biotin (B₇), folic acid (B₉) and cobalamin (B₁₂). In certain embodiments, one or more NR/NAR derivatives are used in conjunction with vitamin B₁, B₂, B₃ or B₆, or any combination thereof.

In additional embodiments, one or more NR/NAR derivatives disclosed herein are used in combination with one or more anti-inflammatory agents to treat an inflammatory disorder. Inflammation contributes to the pathogenesis or pathophysiology of a wide range of disorders. Furthermore, inflammation is a major stimulant of fibrosis. In certain embodiments, the one or more anti-inflammatory agents are or include an NSAID or/and an inhibitor of a pro-inflammatory cytokine or a receptor therefor or the production thereof (e.g., TNF-α, IL-4, IL-6 or IL-23, or any combination thereof).

Anti-inflammatory agents include without limitation:

non-steroidal anti-inflammatory drugs (NSAIDs), including those listed below;

immunomodulators, including imides (e.g., thalidomide, lenalidomide, pomalidomide and apremilast) and xanthine derivatives (e.g., lisofylline, pentoxifylline and propentofylline);

immunosuppressants, including interferon-beta (IFN-β), glucocorticoids (infra), antimetabolites (e.g., hydroxyurea [hydroxycarbamide], antifolates [e.g., methotrexate], and purine analogs [e.g., azathioprine, mercaptopurine and thioguanine]), pyrimidine synthesis inhibitors (e.g., leflunomide and teriflunomide), calcineurin inhibitors (e.g., ciclosporin [cyclosporine A], pimecrolimus and tacrolimus), inosine-5′-monophosphate dehydrogenase (IMPDH) inhibitors (e.g., mycophenolic acid and derivatives thereof [e.g., mycophenolate sodium and mycophenolate mofetil]), mechanistic/mammalian target of rapamycin (mTOR) inhibitors (e.g., rapamycin [sirolimus], deforolimus [ridaforolimus], everolimus, temsirolimus, umirolimus [biolimus A9], zotarolimus and RTP-801), modulators of sphingosine-1-phosphate receptors (e.g., SiPR1) (e.g., fingolimod), and serine C-palmitoyltransferase inhibitors (e.g., myriocin);

anti-inflammatory cytokines and compounds that increase their production, including IL-10 and analogs and derivatives thereof (e.g., PEG-ilodecakin) and compounds that increase IL-10 production {e.g., S-adenosyl-L-methionine, melatonin, metformin, rotenone, curcuminoids (e.g., curcumin), prostacyclin and analogs thereof (supra), triterpenoids (e.g., oleanolic acid analogs [supra, such as TP-225]), and apoA-I mimetics (supra, such as 4F)};

inhibitors of pro-inflammatory cytokines or receptors therefor, including inhibitors of (e.g., antibodies or fragments thereof targeting) tumor necrosis factor-alpha (TNF-α) (e.g., adalimumab, certolizumab pegol, golimumab, infliximab, etanercept, bupropion, curcumin, catechins and ART-621) or the receptor therefor (TNFR1), inhibitors of thymic stromal lymphopoietin (e.g., anti-TSLP antibodies and fragments thereof [e.g., tezepelumab and M702] and immunoconjugates comprising the extracellular domain of TSLPR) or the receptor therefor (TSLPR), inhibitors of (e.g., antibodies or fragments thereof targeting) pro-inflammatory interferons (e.g., interferon-alpha [IFN-α]) or receptors therefor, inhibitors of (e.g., antibodies or fragments thereof targeting) pro-inflammatory interleukins or receptors therefor {e.g., IL-1 (e.g., IL-1α and IL-1β [e.g., canakinumab and rilonacept]) or IL-1R (e.g., anakinra and isunakinra [EBI-005]), IL-2 or IL-2R (e.g., basiliximab and daclizumab), IL-4 or IL-4R (e.g., dupilumab), IL-5 (e.g., mepolizumab and reslizumab) or IL-5R, IL-6 (e.g., clazakizumab, elsilimomab, olokizumab, siltuximab and sirukumab) or IL-6R (e.g., sarilumab and tocilizumab), IL-8 or IL-8R, IL-12 (e.g., briakinumab and ustekinumab) or IL-12R, IL-13 or IL-13R, IL-15 or IL-15R, IL-17 (e.g., ixekizumab and secukinumab) or IL-17R (e.g., brodalumab), IL-18 (e.g., GSK1070806) or IL-18R, IL-20 (e.g., the antibody 7E) or IL-20R, IL-22 (e.g., fezakinumab) or IL-22R, IL-23 (e.g., briakinumab, guselkumab, risankizumab, tildrakizumab [SCH-900222], ustekinumab and BI-655066) or IL-23R, IL-31 (e.g., anti-IL-31 antibodies disclosed in U.S. Pat. No. 9,822,177) or IL-31R (e.g., anti-IL-31 receptor A antibodies such as nemolizumab), IL-33 or IL-33R, and IL-36 or IL-36R}, and inhibitors of monocyte chemoattractant protein 1 (MCP-1) {e.g., bindarit, anti-MCP1 antibodies (e.g., 5D3-F7 and 10F7), MCP1-binding peptides (e.g., HSWRHFHTLGGG), and MCP1-binding RNA aptamers (e.g., ADR22 and mNOX-E36 [a spiegelmer])} or receptors therefor (e.g., CCR2 antagonists such as spiropiperidines [e.g., RS-29634, RS-102895 and RS-504393]);

inhibitors of the production of pro-inflammatory cytokines or receptors therefor, including inhibitors of the production of TNF-α {e.g., N-acetyl-L-cysteine, S-adenosyl-L-methionine, L-carnitine, hydroxychloroquine, melatonin, parthenolide, pirfenidone, sulfasalazine, mesalazine (5-aminosalicylic acid), taurine, flavonoids (e.g., epigallocatechin-3-gallate [EGCG], naringenin and quercetin), omega-3 fatty acids and esters thereof, glucocorticoids, immunomodulatory imides and xanthine derivatives, PDE4 inhibitors, serine protease inhibitors (e.g., gabexate and nafamostat), prostacyclin and analogs thereof, SOCS1 mimetics (infra), myxoma virus M013 protein, Yersinia YopM protein, apoA-I mimetics (e.g., 4F), and apoE mimetics (e.g., AEM-28 and hEp)}, IFN-α (e.g., alefacept), IL-1 (e.g., IL-1α and IL-1β) (e.g., chloroquine, hydroxychloroquine, nafamostat, pirfenidone, sulfasalazine, mesalazine, prostacyclin and analogs thercof, glucocorticoids, TNF-α inhibitors, PAR1 antagonists [e.g., vorapaxar], M013 protein, YopM protein and apoA-I mimetics [e.g., 4F]), IL-1β (e.g. melatonin, metformin, rotenone, flavonoids [e.g., EGCG and naringenin], annexin A1 mimetics, and caspase-1 inhibitors [e.g., belnacasan, pralnacasan and parthenolide]), IL-2 (e.g., glucocorticoids, calcineurin inhibitors and PDE4 inhibitors), IL-4 (e.g., glucocorticoids and serine protease inhibitors [e.g., gabexate and nafamostat]), IL-5 (e.g., glucocorticoids), IL-6 (e.g., nafamostat, parthenolide, prostacyclin and analogs thereof, tranilast, L-camitine, taurine, flavonoids [e.g., EGCG, naringenin and quercetin], omega-3 fatty acids and esters thereof, glucocorticoids, immunomodulatory imides, TNF-α inhibitors, M013 protein and apoE mimetics [e.g., AEM-28 and hEp]), IL-8 (e.g., alefacept and glucocorticoids), IL-12 (e.g., apilimod, PDE4 inhibitors and YopM protein), IL-15 (e.g., YopM protein), IL-17 (e.g., protein kinase C inhibitors such as sotrastaurin), IL-18 (e.g., MO13 protein, YopM protein and caspase-1 inhibitors), IL-23 (e.g., apilimod, alefacept and PDE4 inhibitors), and MCP-1 (e.g., EGCG, melatonin and tranilast);

inhibitors of pro-inflammatory transcription factors or their activation or expression, including inhibitors of NF-κB or its activation or expression {e.g., aliskiren, melatonin, minocycline and parthenolide (both inhibit NF-κB nuclear translocation), nafamostat, niclosamide, (−)-DHMEQ, IT-603, IT-901, PBS-1086, flavonoids (e.g., EGCG and quercetin), hydroxycinnamic acids and esters thereof (e.g., ethyl caffeate), lipoxins (e.g., 15-epi-LXA4 and LXB4), omega-3 fatty acids and esters thereof, stilbenoids (e.g., resveratrol), statins (e.g., rosuvastatin), triterpenoids (e.g., oleanolic acid analogs such as TP-225), TNF-α inhibitors, apoE mimetics (e.g., AEM-28), M013 protein, penetratin, and activators of sirtuin 1 (SIRT1, which inhibits NF-κB) (e.g., flavones [e.g., luteolin], phenylethanoids [e.g., tyrosol, which induces SIRT1 expression], stilbenoids [e.g., resveratrol, which increases SIRT1 activity and expression] and lamin A)}, and inhibitors of STAT (signal transducer and activator of transcription) proteins or their activation or expression {e.g., Janus kinase 1 (JAK1) inhibitors (e.g., itacitinib, upadacitinib, GLPG0634 and GSK2586184), JAK2 inhibitors (e.g., lestaurtinib, pacritinib, CYT387, TG101348, SOCS1 mimetics and SOCS3 mimetics), JAK3 inhibitors (e.g., ASP-015K, R348 and VX-509), dual JAK1/JAK2 inhibitors (e.g., baricitinib and ruxolitinib), dual JAK1/JAK3 inhibitors (e.g., tofacitinib), suppressor of cytokine signaling (SOCS) mimetic peptides (e.g., SOCS1 mimetics [e.g., SOCS1-KIR, NcwSOCS1-KIR, PS-5 and Tkip] and SOCS3 mimetics), niclosamide, hydroxycinnamic acids and esters thereof (e.g., rosmarinic acid), and lipoxins (e.g., 15-epi-LXA4 and LXB4)};

inhibitors of pro-inflammatory prostaglandins (e.g., prostaglandin E₂ [PGE₂]) or receptors therefor (e.g., EP₃) or the production thereof, including cyclooxygenase inhibitors (e.g., NSAIDs [including non-selective COX-1/COX-2 inhibitors such as aspirin and selective COX-2 inhibitors such as coxibs], glucocorticoids [which inhibit COX activity and expression], omega-3 fatty acids and esters thereof, curcuminoids [e.g., curcumin], stilbenoids [e.g., resveratrol, which inhibits COX-1 and -2 activity and expression], and vitamin E and analogs thereof [e.g., α-tocopherol and trolox]), cyclopentenone prostaglandins (e.g., prostaglandin J₂ [PGJ₂], Δ12-PGJ₂ and 15-deoxy-Δ12,14-PGJ₂), hydroxycinnamic acids and esters thereof (e.g., ethyl caffeate, which suppresses COX-2 expression), and triterpenoids (e.g., oleanolic acid analogs such as TP-225, which suppress COX-2 expression);

inhibitors of leukotrienes or receptors therefor or the production thereof, including cysteinyl leukotriene receptor 1 (cysLTR1) antagonists (e.g., cinalukast, gemilukast [dual cysLTR1/cysLTR2 antagonist], iralukast, montelukast, pranlukast, tomelukast, verlukast, zafirlukast, CP-195494, CP-199330, ICI-198615, MK-571 and lipoxins [e.g., LXA4 and 15-epi-LXA4]), cysLTR2 antagonists (e.g., HAMI-3379), 5-lipoxygenase (5-LOX) inhibitors (e.g., baicalein, caffeic acid, curcumin, hyperforin. γ-linolenic acid [GLA], meclofenamic acid, meclofenamate sodium, minocycline, tipelukast [MN-001], zileuton, MK-886, and omega-3 fatty acids and esters thereof), and immunomodulatory xanthine derivatives;

inhibitors of phospholipase A2 (e.g., secreted and cytosolic PLA2), including glucocorticoids, arachidonyl trifluoromethyl ketone, bromoenol lactone, chloroquine, cytidine 5-diphosphoamines, darapladib, quinacrine, vitamin E, RO-061606, ZPL-521, lipocortins (annexins, such as annexin A1), and annexin mimetic peptides (e.g., annexin A1 mimetics [e.g., Ac2-26 and CGEN-855A]);

suppressors of C-reactive protein (CRP) activity or level, including statins (e.g., rosuvastatin), thiazolidinediones (supra), DPP-4 inhibitors (supra), stilbenoids (e.g., resveratrol), epigallocatechin-3-gallate and CRP-i2;

mast cell stabilizers, including cromoglicic acid (cromolyn), ketotifen, methylxanthines, nedocromil, nicotinamide, olopatadine, omalizumab, pemirolast, quercetin and zinc sulfate;

phosphodiesterase inhibitors, including PDE4 inhibitors (e.g., apremilast, cilomilast, ibudilast, piclamilast, roflumilast, crisaborole, diazepam, luteolin, mesembrenone, rolipram, AN2728 and E6005) and dual PDE3/4 inhibitors (e.g., tipelukast);

specialized pro-resolving mediators (SPMs), including metabolites of polyunsaturated fatty acids (PUFAs) such as lipoxins (e.g., LXA4, 15-epi-LXA4, LXB4 and 15-epi-LXB4), resolvins (e.g., resolvins derived from 5Z,8Z,1Z,14Z,17Z-eicosapentaenoic acid [EPA], resolvins derived from 4Z,7Z,10Z,13Z,16Z,19Z-docosahexaenoic acid [DHA], and resolvins derived from 7Z,10Z,13Z,16Z,19Z-docosahexaenoic acid [n-3 DPA]), protectins/neuroprotectins (e.g., DHA-derived protectins/neuroprotectins and n-3 DPA-derived protectins/neuroprotectins), maresins (e.g., DHA-derived maresins and n-3 DPA-derived maresins), n-3 DPA metabolites, n-6 DPA (4Z,7Z,10Z,13Z,16Z-docosapentaenoic acid) metabolites, oxo-DHA metabolites, oxo-DPA metabolites, docosahexaenoyl ethanolamide metabolites, cyclopentenone prostaglandins (e.g., A12-PGJ₂ and 15-deoxy-Δ12,14-PGJ₂), and cyclopentenone isoprostanes (e.g., 5,6-epoxyisoprostane A₂ and 5,6-epoxyisoprostane E₂);

other kinds of anti-inflammatory agents, including pirfenidone, nintedanib, vitamin A, omega-3 fatty acids and esters thereof, apoA-I mimetics (e.g., 4F), apoE mimetics (e.g., AEM-28 and AEM-28-14), and antioxidants (e.g., sulfur-containing antioxidants); and

analogs, derivatives, fragments and salts thereof.

Non-steroidal anti-inflammatory drugs (NSAIDs) include without limitation:

acetic acid derivatives, such as aceclofenac, bromfenac, diclofenac, etodolac, indomethacin, ketorolac, nabumetone, sulindac, sulindac sulfide, sulindac sulfone and tolmetin;

anthranilic acid derivatives (fenamates), such as flufenamic acid, meclofenamic acid, mefenamic acid and tolfenamic acid;

enolic acid derivatives (oxicams), such as droxicam, isoxicam, lornoxicam, meloxicam, piroxicam and tenoxicam;

propionic acid derivatives, such as fenoprofen, flurbiprofen, ibuprofen, dexibuprofen, ketoprofen, dexketoprofen, loxoprofen, naproxen and oxaprozin;

salicylates, such as diflunisal, salicylic acid, acetylsalicylic acid (aspirin), choline magnesium trisalicylate, salsalate and mesalazine;

COX-2-selective inhibitors, such as apricoxib, celecoxib, etoricoxib, firocoxib, fluorocoxibs (e.g., fluorocoxibs A-C), lumiracoxib, mavacoxib, parecoxib, rofecoxib, tilmacoxib (JTE-522), valdecoxib, 4-O-methylhonokiol, niflumic acid, DuP-697, CG100649, GW406381, NS-398, SC-236, SC-58125, benzothieno[3,2-d]pyrimidin-4-one sulfonamide thio-derivatives, and COX-2 inhibitors derived from Tribulus terrestris;

other kinds of NSAIDs, such as monoterpenoids (e.g., eucalyptol and phenols [e.g., carvacrol]), anilinopyridinecarboxylic acids (e.g., clonixin), sulfonanilides (e.g., nimesulide), and dual inhibitors of lipooxygenase (e.g., 5-LOX) and cyclooxygenase (e.g., COX-2) {e.g., chebulagic acid, licofelone, 2-(3,4,5-trimethoxyphenyl)-4-(N-methylindol-3-yl)thiophene, and di-tert-butylphenol-based compounds (e.g., DTPBHZ, DTPINH, DTPNHZ and DTPSAL)}; and

analogs, derivatives and salts thereof.

The glucocorticoid class of corticosteroids has anti-inflammatory and immunosuppressive properties. Glucocorticoids include without limitation hydrocortisone types (e.g., cortisone and derivatives thereof [e.g., cortisone acetate], hydrocortisone and derivatives thereof [e.g., hydrocortisone acetate, hydrocortisone-17-aceponate, hydrocortisone-17-buteprate, hydrocortisone-17-butyrate and hydrocortisone-17-valerate], prednisolone, methylprednisolone and derivatives thereof [e.g., methylprednisolone aceponate], prednisone, and tixocortol and derivatives thereof [e.g., tixocortol pivalate]), betamethasone types (e.g., betamethasone and derivatives thereof [e.g., betamethasone dipropionate, betamethasone sodium phosphate and betamethasone valerate], dexamethasone and derivatives thereof [e.g., dexamethasone sodium phosphate], and fluocortolone and derivatives thereof [e.g., fluocortolone caproate and fluocortolone pivalate]), halogenated steroids (e.g., alclometasone and derivatives thereof [e.g., alclometasone dipropionate], beclometasone and derivatives thereof [e.g., beclometasone dipropionate], clobetasol and derivatives thereof [e.g., clobetasol-17-propionate], clobetasone and derivatives thereof [e.g., clobetasone-17-butyrate], desoximetasone and derivatives thereof [e.g., desoximetasone acetate], diflorasone and derivatives thereof [e.g., diflorasone diacetate], diflucortolone and derivatives thereof [e.g., diflucortolone valerate], fluprednidene and derivatives thereof [e.g., fluprednidene acetate], fluticasone and derivatives thereof [e.g., fluticasone propionate], halobetasol [ulobetasol] and derivatives thereof [e.g., halobetasol proprionate], halometasone and derivatives thereof [e.g., halometasone acetate], and mometasone and derivatives thereof [e.g., mometasone furoate]), acetonides and related substances (e.g., amcinonide, budesonide, ciclesonide, desonide, fluocinonide, fluocinolone acetonide, flurandrenolide [flurandrenolone or fludroxycortide], halcinonide, triamcinolone acetonide and triamcinolone alcohol), carbonates (e.g., prednicarbate), and analogs, derivatives and salts thereof.

In additional embodiments, one or more NR/NAR derivatives of the disclosure are used in conjunction with one or more antifibrotic agents to treat a fibrotic disorder. In some embodiments, the one or more antifibrotic agents are or include an anti-inflammatory agent or/and an antioxidant (e.g., vitamin E or an analog thereof [e.g., α-tocopherol or trolox], a sulfur-containing antioxidant or an ROS or radical scavenger [e.g., melatonin], or any combination thereof). In certain embodiments, the one or more antifibrotic agents are or include pirfenidone (which among its various antifibrotic and anti-inflammatory properties described herein also reduces fibroblast proliferation) or/and nintedanib (which blocks signaling of fibroblast growth factor receptors [FGFRs], platelet-derived growth factor receptors [PDGFRs] and vascular endothelial growth factor receptors [VEGFRs] involved in fibroblast proliferation, migration and transformation).

In further embodiments, the one or more antifibrotic agents are or include one or more agents that have anti-hyperglycemic or/and insulin-sensitizing activity for treatment of a fibrotic disorder in which hyperglycemia, diabetes or insulin resistance contributes to development of fibrosis. Examples of such a disorder include diabetic nephropathy, which is characterized by renal fibrosis, and NASH and cirrhosis, both of which are characterized by hepatic fibrosis. Use of anti-hyperglycemic or/and insulin-sensitizing agent(s) can curtail or prevent, e.g., renal inflammation and fibrosis or hepatic inflammation and fibrosis. In certain embodiments, the one or more antifibrotic agents are or include a PPAR-γ agonist (e.g., a thiazolidinedione [supra], such as pioglitazone or rosiglitazone). PPARγ-activating thiazolidinediones have both anti-hyperglycemic and insulin-sensitizing properties.

Antifibrotic agents include without limitation:

inhibitors of collagen accumulation, including protein kinase C (PKC) inhibitors (supra, inhibit collagen production), 5-lipoxygenase inhibitors (e.g., tipelukast, which reduces collagen I, LOXL2 and TIMP-1 production), colchicine and its metabolite colchiceine (both inhibit collagen synthesis and deposition), dilinoleoyl-phosphatidylcholine (inhibits collagen production induced by transforming growth factor-beta1 [TGF-β1]), luteolin (reduces fibrosis in part by increasing expression of matrix metalloproteinase 9 [MMP-9] and metallothionein, which degrade the extracellular matrix [ECM]), malotilate (reduces procollagen I α2 [Col1a2] expression), melatonin (inhibits expression of procollagens I and III), S-nitroso-N-acetyl-L-cysteine (reduces collagen I amount in part by activating MMP-13 and suppressing tissue inhibitor of metalloproteinases 2 [TIMP-2]), oxymatrine {reduces procollagen I α1 (Col1a1) (and α-smooth muscle actin [α-SMA]) expression}, pioglitazone (reduces collagen I [and α-SMA] production), pirfenidone (reduces production of procollagens I and II and inhibits TGF-β-stimulated collagen production), quercetin (reduces Col1a1 and procollagen III α1 [Col3a1] expression), resveratrol (reduces collagen I [and α-SMA] production), RGD mimetics and analogs (infra, reduce collagen I accumulation in part by increasing secretion of collagenases), safironil (reduces collagen I [and α-SMA] production), statins (e.g., atorvastatin, lovastatin and simvastatin [all three reduce collagen production]), tranilast (inhibits procollagen expression and fibroblast proliferation), valproic acid (reduces collagen deposition), inhibitors of collagen cross-linking {e.g., β-penicillamine and lysyl oxidase-like 2 (LOXL2, which promotes collagen cross-linking) inhibitors (e.g., β-aminopropionitrile and anti-LOXL2 antibodies [e.g., simtuzumab and AB-0023])}, procollagen-proline dioxygenase (or prolyl 4-hydroxylase, which forms more stable hydroxylated collagen) inhibitors (e.g., malotilate, HOE-077, S-0885 and S-4682), and procollagen glucosyltransferase (or galactosylhydroxylysine glucosyltransferase, which is important for collagen fibril formation) inhibitors (e.g., malotilate);

inhibitors of pro-fibrotic growth factors (e.g., transforming growth factor-beta [including TGF-β1], connective tissue growth factor [CTGF] and platelet-derived growth factor [including PDGF-B, PDGF-C and PDGF-D]) or their production, activation or signaling, including TGF-β inhibitors {e.g., anti-TGF-β antibodies (e.g., fresolimumab [GC1008] and CAT-192) and soluble TGF-β receptors (e.g., sTGFβR1, sTGFβR2 and sTGFβR3)}, TGFβR antagonists {e.g., TGFβR1 (ALK5) antagonists (e.g., galunisertib [LY-2157299], EW-7197, GW-788388, LY-2109761, SB-431542, SB-525334, SKI-2162, SM-16, and inhibitory Smads [e.g., Smad6 and Smad7])}, anti-CTGF antibodies (e.g., FG-3019), PDGF inhibitors (e.g., squalamine, PP1, anti-PDGF aptamers [e.g., E10030], anti-PDGF antibodies [e.g., those targeting PDGF-B, PDGF-C and PDGF-D], and soluble PDGF receptors [e.g., sPDGFRα and sPDGFRβ]), PDGFR (e.g., PDGFRα or/and PDGFRs) antagonists (e.g., anti-PDGFR antibodies [e.g., REGN2176-3]), bone morphogenic protein-7 (BMP-7) (directly antagonizes TGF-β1 signaling and Smad3 activation, and promotes mesenchymal-to-epithelial transition), decorin (inhibits TGF-β1 activity and collagen fiber formation), N-acetyl-L-cysteine (inhibits TGF-β expression and activation by monomerization of the biologically active TGF-β dimer), S-nitroso-N-acetyl-L-cysteine (suppresses TGF-β1), L-carnitine (reduces PDGF-B expression), epigallocatechin-3-gallate (suppresses activation of Smad2 and Smad3 [and Akt]), galectin-7 (binds to and inhibits phosphorylated Smad2 and Smad3), Leu-Ser-Lys-Leu (inhibits TGF-β1 activation), α-lipoic acid (inhibits TGF-β signaling via inhibition of Smad3 and AP-1), luteolin (inhibits TGF-β and PDGF signaling), melatonin (inhibits TGF-β and CTGF expression and Smad3 activation), naringenin (suppresses Smad3 expression and activation), niacin (reduces TGF-β expression), pirfenidone (reduces TGF-β production), quercetin (reduces expression of TGF-β1, CTGF, PDGF-B and Smad3), resveratrol (suppresses TGF-β expression), simvastatin (reduces TGF-β1 [and α-SMA]expression), taurine (reduces TGF-β1 [and α-SMA] expression), tranilast (inhibits TGF-β1 expression), vitamin E and analogs thereof (e.g., α-tocopherol and trolox, both of which suppress TGF-β expression), and αᵥβ₆ integrin (which activates TGF-β1) inhibitors (e.g., anti-αᵥβ₆ antibodies such as STX-100);

receptor tyrosine kinase (TK) inhibitors, including epidermal growth factor receptor (EGFR) TK inhibitors (e.g., afatinib, brigatinib, erlotinib, gefitinib, icotinib, lapatinib, osimertinib and isoflavones [e.g., genistein]), PDGFR TK inhibitors (e.g., crenolanib, imatinib and AG-1295), dual FGFR/VEGFR TK inhibitors (e.g., brivanib and brivanib alaninate), dual PDGFR/VEGFR TK inhibitors (e.g., axitinib, sorafenib, sunitinib, vatalanib and X-82), and triple FGFR/PDGFR/VEGFR TK inhibitors (e.g., nintedanib and pazopanib);

anti-EGFR antibodies, such as cetuximab, matuzumab, nimotuzumab, panitumumab and zalutumumab;

anti-inflammatory agents, including those listed above, such as anti-inflammatory cytokines (e.g., IL-10), inhibitors of pro-inflammatory cytokines or their receptors or their production (e.g., TNF-α [e.g., an anti-TNF-α antibody such as infliximab or an immunomodulator such as pentoxifylline], IL-1β, IL-6 and MCP-1), colchicine, curcuminoids (e.g., curcumin), malotilate, nintedanib, pirfenidone and tranilast;

antioxidants, including those listed above, such as vitamins and analogs thereof (e.g., vitamin E and analogs thereof such as α-tocopherol and trolox), sulfur-containing antioxidants (e.g., glutathione, NAC, SNAC, SAC [also suppresses α-SMA expression] and SAM), ROS and radical scavengers (e.g., melatonin and glutathione), Nrf2 activators {e.g., fumarates (e.g., dimethyl and monomethyl fumarate), trichostatin A, and triterpenoids (e.g., oleanolic acid analogs [supra, such as TP-225])}, and omega-3 fatty acids and esters thereof (e.g., Lovaza fish oil);

antagonists of the renin-angiotensin-aldosterone system (RAAS), including those listed above, such as renin inhibitors (e.g., aliskiren [reduces hepatic steatosis, oxidative stress, inflammation and fibrosis]), ACE inhibitors (e.g., captopril [inhibits fibroblast proliferation and reduces fibrotic lung response] and perindopril [inhibits liver fibrosis]), and angiotensin II receptor type 1 (AT₁) antagonists (e.g., candesartan [inhibits liver fibrosis], irbesartan and losartan) (activation of AT₁ by angiotensin II activates PLC, leading to increased cytosolic Ca²⁺ concentration and hence PKC stimulation, also activates tyrosine kinases and promotes ECM formation);

inhibitors of the accumulation or effects of advanced glycation end-products (AGEs, which inter alia increase arterial stiffness and stimulate mesangial matrix expansion), including inhibitors of AGE formation (e.g., aminoguanidine, aspirin, benfotiamine, carnosine, α-lipoic acid, metformin, pentoxifylline, pimagedine, pioglitazone, pyridoxamine, taurine and vitamin C), cleavers of AGE crosslinks (e.g., aminoguanidine, N-phenacylthiazolium bromide, rosmarinic acid, alagebrium [ALT-711], ALT-462, ALT-486 and ALT-946), and inhibitors of AGE effects (e.g., natural phenols such as curcumin and resveratrol);

other kinds of antifibrotic agents, including RGD mimetics and analogs (inhibit adhesion of fibroblasts and immune cells to ECM glycoproteins) (e.g., NS-11, SF-6,5 and GRGDS), galectin-3 (which is critical for liver fibrosis) inhibitors (e.g., GM-CT-01 and GR-MD-02), marinobufagenin inhibitors (e.g., resibufogenin, spironolactone and canrenone), trichostatin A (inhibits TGFβ1-induced epithelial-to-mesenchymal transition), and PPAR-γ agonists (e.g., thiazolidinediones [supra], saroglitazar and IVA-337); and

analogs, derivatives, fragments and salts thereof.

Non-alcoholic fatty liver disease (NAFLD), the most common liver disorder in developed countries, is characterized by fatty liver that occurs when fat, in particular free fatty acids and triglycerides, accumulates in liver cells (hepatic steatosis) due to causes other than excessive alcohol consumption, such as nutrient overload, high caloric intake and metabolic dysfunction (e.g., hyperlipidemia and impaired glucose control). A liver can remain fatty without disturbing liver function, but a fatty liver can progress to become non-alcoholic steatohepatitis (NASH), a condition in which steatosis is accompanied by inflammation, hepatocyte ballooning and cell injury with or without fibrosis of the liver. Fibrosis is the strongest predictor of mortality from NASH. NASH is the most extreme form of NAFLD. NASH is a progressive disease, with about 20% of patients developing cirrhosis of the liver and about 10% dying from a liver disease, such as cirrhosis or a liver cancer (e.g., hepatocellular carcinoma).

NAFLD, including NASH, is associated with obesity, metabolic syndrome and insulin resistance. For instance, insulin resistance contributes to progression of fatty liver to hepatic inflammation and fibrosis and thus NASH. Furthermore, obesity drives and exacerbates NASH, and weight loss can alleviate NASH.

In some embodiments, one or more NR/NAR derivatives described herein are used in combination with one or more additional therapeutic agents to treat NAFLD, such as NASH. In some embodiments, the one or more additional therapeutic agents are selected from antidiabetic agents, anti-obesity agents, anti-inflammatory agents, antifibrotic agents, antioxidants, and combinations thereof.

Therapeutic agents that can be used to treat NAFLD (e.g., NASH) include without limitation:

PPAR agonists, including PPAR-δ agonists (e.g., MBX-8025, elafibranor [dual PPAR-α/δ agonist], lanifibranor [triple PPAR-α/δ/γ agonist] and GW501516 [dual PPAR-β/δ agonist]), PPAR-γ agonists (e.g., thiazolidinediones such as pioglitazone, deuterated (R)-pioglitazone [e.g., DRX-065] and rosiglitazone, and saroglitazar [dual PPAR-α/γ agonist]), and triple PPAR-α/δ/γ agonists (e.g., IVA-337)-PPAR-δ and -γ agonism increases insulin sensitivity, PPAR-α agonism reduces liver steatosis and PPAR-δ agonism inhibits activation of macrophages and Kupffer cells;

GLP-1R agonists (e.g., exenatide, liraglutide, semaglutide and AC-3174), dual GLP-1R/GCGR agonists (e.g., MEDI0382 and SP-1373), dual GLP-1R/GIPR agonists and dual GLP-1R/FGF21 agonists (e.g., YH-25724)—such agonists reduce liver steatosis, inflammation and fibrosis;

farnesoid X receptor (FXR) agonists, such as obeticholic acid, tropifexor (UN-452) AGN-242266, AKN-083, EDP-305, EYP-001, GNF-5120, GS-9674, INT-767, INT-2228, UN-452, LMB-763, M450, M480, M780, M790, Px-102, Px-103, PX20606 and TERN-101-FXR agonists reduce liver gluconeogenesis, lipogenesis, steatosis, inflammation and fibrosis;

G protein-coupled bile acid receptor 1 (TGR5) agonists, such as RDX-009, INT-777 and INT-767 (dual TGR5/FXR agonist)-TGR5 agonists reduce insulin resistance and liver steatosis, inflammation and fibrosis;

thyroid hormone receptor-beta agonists, such as MGL-3196. MGL-3745 and VK2809-THR-β agonists reduce liver steatosis;

fibroblast growth factor 19 (FGF19) and analogs and derivatives thereof, such as NGM-282-FGF19 analogs reduce liver gluconeogenesis and steatosis;

fibroblast growth factor 21 (FGF21) and analogs and derivatives thereof, such as BMS-986036, BMS-986171 and PF-05231023-FGF21 analogs reduce liver steatosis, cell injury and fibrosis;

HMG-CoA reductase inhibitors, including statins (e.g., atorvastatin, pitavastatin and rosuvastatin)—statins reduce steatohepatitis and fibrosis;

ACC inhibitors, such as NDI-010976 (liver-targeted) and firsocostat (GS-0976)-ACC inhibitors reduce de novo lipogenesis and liver steatosis and fibrosis;

SCD-1 inhibitors, such as aramchol—SCD-1 inhibitors reduce liver steatosis and increase insulin sensitivity;

ATP citrate lyase inhibitors, such as bempedoic acid—ACL inhibitors reduce liver steatosis;

ketohexokinase inhibitors, such as PF-06835919-KHK inhibitors reduce liver lipogenesis and inflammation;

SGLT2 inhibitors, such as canagliflozin, dapagliflozin, empagliflozin, ipragliflozin and luseogliflozin—SGLT2 inhibitors reduce body weight, liver ALT level and fibrosis;

vascular adhesion protein-1 (VAP-1) inhibitors, such as N-[4-(2-{4-[(2-amino-1H-imidazol-4-yl)methyl]phenyl}ethyl)thiazol-2-yl]acetamide hydrochloride, 2-bromoethylamine, semicarbazide, ASP8232, BI-1467335 (PXS-4728A), PXS-4681A, PRX-167700 and TERN-201-VAP-1 inhibitors increase insulin sensitivity and reduce liver inflammation and fibrosis;

antagonists of CCR2 or/and CCR5 and inhibitors of chemokines binding to CCR2 or/and CCR5, such as cenicriviroc (CCR2/CCR5 antagonist) and propagermanium (CCR2 antagonist)—antagonists of CCR2 (binds to CCL2 [MCP1]) and CCR5 (binds to CCL5 [RANTES]) inhibit activation and migration of inflammatory cells (e.g., macrophages) to the liver and reduce liver fibrosis;

antagonists of CCR3 and inhibitors of chemokines (e.g., CCL5 and CCL11 [eotaxin-1]) binding to CCR3, such as bertilimumab [CCL11 inhibitor]—such antagonists and inhibitors inhibit activation and migration of inflammatory cells (e.g., eosinophils) to the liver;

apoptosis inhibitors, including apoptosis signal-regulating kinase 1 (ASK1) inhibitors (e.g., selonsertib) and caspase inhibitors (e.g., emricasan [pan-caspase inhibitor])—apoptosis inhibitors reduce liver steatosis, inflammation and fibrosis;

TGF-β inhibitors (e.g., fresolimumab) and TGF-βR antagonists (e.g., galunisertib)—they reduce liver fibrosis;

lysyl oxidase-like 2 (LOXL2) inhibitors, such as simtuzumab—LOXL2 is a key matrix enzyme in collagen formation and is highly expressed in the liver;

galectin-3 inhibitors, such as GR-MD-02 and TD139-galectin-3 is critical for development of liver fibrosis;

inhibitors of lysophosphatidic acid (LPA) or receptors therefor (e.g., LPAR1) or the production thereof, such as LPAR1 antagonists (e.g., AR-479, BMS-986020, BMT-053011, ITMN-10534, KI-16198 and UD-009), autotaxin inhibitors (e.g., AM-063, GLPG-1690, HA-130, ONO-8430506, PAT-048, PAT-505, PF-8380, S-32826 and X-165) and anti-autotaxin DNA aptamers (e.g., RBO11 and RB014)—such inhibitors and antagonists inhibit myofibroblast proliferation and hence liver fibrosis;

antioxidants, including vitamin E (e.g., α-tocopherol) and scavengers of ROS and free radicals (e.g., cysteamine, glutathione, melatonin and pentoxifylline [also anti-inflammatory via inhibition of TNF-α and phosphodiesterases])—vitamin E reduces liver steatosis, hepatocyte ballooning and lobular inflammation; and

analogs, derivatives and salts thereof.

In some embodiments, the one or more additional therapeutic agents for treatment of NAFLD (e.g., NASH) are or include a PPAR agonist (e.g., a PPAR-δ agonist such as elafibranor or/and a PPAR-γ agonist such as pioglitazone), a HMG-CoA reductase inhibitor (e.g., a statin such as rosuvastatin), an FXR agonist (e.g., obeticholic acid) or an antioxidant (e.g., vitamin E), or any combination thereof. In certain embodiments, the one or more additional therapeutic agents for treatment of NAFLD (e.g., NASH) are or include vitamin E or/and pioglitazone.

In other embodiments, one or more NR/NAR derivatives of the disclosure are used in combination with one or more anticancer agents to treat a tumor (benign or malignant) or a cancer. For brevity, the term “anticancer agents” as used herein encompasses antitumor agents.

In some embodiments, the one or more anticancer agents are or include radiation therapy, chemotherapy or cancer immunotherapy, or any combination or all thereof.

In some embodiments, the chemotherapeutic agent is or includes a PARP inhibitor, a TGF-β inhibitor or a cytotoxic agent, or any combination or all thereof. Examples of PARP inhibitors are described above. In certain embodiments, the PARP inhibitor is olaparib.

Transforming growth factor-beta (TGF-β) is a cytokine that promotes the growth of pre-cancer and cancer cells, angiogenesis and invasion of cancer cells. TGF-β also converts effector T-cells, which normally attack cancer cells with an inflammatory (immune) reaction, into regulatory T-cells that suppress the immune reaction. An increase in TGF-β expression often correlates with the malignancy of many cancers. Therefore, inhibitors of TGF-β or the production, activation or signaling thereof can be used to treat tumors and cancers. Since TGF-β (including TGF-β1) is also a major driver of collagen production and fibrosis, inhibitors of TGF-β or the production, activation or signaling thereof are listed among antifibrotic agents above.

Anticancer cytotoxic agents include without limitation:

alkylating agents, including aziridines (e.g., diaziquone, mytomycin and thiotepa), nitrogen mustards (e.g., mannomustine, mustine [mechlorethamine or chlormethine], aniline mustard, bendamustine, benzoic acid mustard, chlorambucil, C6-galactose mustard, melphalan, ossichlorin [nitromin], prednimustine, uramustine, nitrogen mustard carbamates [e.g., estramustine], and oxazaphosphorines [e.g., cyclophosphamide, ifosfamide, mafosfamide, and trofosfamide]), nitrosoureas (e.g., carmustine, fotemustine, lomustine, nimustine, N-nitroso-N-methylurea, ranimustine, semustine and streptozotocin), platinum-containing compounds (e.g., cisplatin, carboplatin and oxaliplatin), alkylsulfonates (e.g., busulfan, mannosulfan and treosulfan), hydrazines (e.g., dacarbazine and procarbazine), imidazotetrazines (e.g., mitozolomide and temozolomide), and triazines (e.g., hexamethylmelamine [altretamine]);

cytotoxic antibiotics, including anthracyclines (e.g., aclarubicin, daunorubicin, doxorubicin, epirubicin, idarubicin, mitoxantrone, pirarubicin and valrubicin), actinomycins (e.g., actinomycin D), bleomycins (e.g., bleomycins A₂ and B₂), mitomycins (e.g., mitomycin C), and plicamycins;

antimetabolites, including antifolates (e.g., aminopterin, methotrexate, pemetrexed and pralatrexate), deoxynucleoside analogs (e.g., 5-azacytidine [azacitidine], 5-aza-2′-deoxycytidine [decitabine], cladribine, clofarabine, cytarabine, decitabine, fludarabine, gemcitabine, nelarabine and pentostatin), fluoropyrimidines (e.g., 5-fluorouracil, capecitabine, 5-fluoro-5′-deoxyuridine [doxifluridine] and trifluridine), and thiopurines (e.g., thioguanine, azathioprine and mercaptopurine);

antimicrotubule agents, including dolastatins (e.g., dolastatin 15), epothilones (e.g., epothilones A-F), halichondrins (e.g., halichondrin B) and analogs thereof (e.g., eribulin), maytansine, maytansinoids (e.g., ansamitocin, emtansine, mertansine, ravtansine and soravtansine), taxanes (e.g., paclitaxel, docetaxel and cabazitaxel), vinca alkaloids (e.g., vinblastine, vincristine, vindesine, vinflunine and vinorelbine), colchicine, nocodazole, podophyllotoxin and rhizoxin;

histone deacetylase inhibitors, including trichostatins (e.g., trichostatin A), romidepsin, panobinostat and vorinostat;

kinase inhibitors, including bortezomib, erlotinib, gefitinib, imatinib, vemurafenib, vismodegib, curcumin, cyclocreatine, deguelin, fostriecin, hispidin, staurosporine and derivatives thereof (e.g., midostaurin), and tyrphostins (e.g., tyrphostins AG 34 and AG 879);

topoisomerase I inhibitors, including camptothecin, irinotecan and topotecan;

topoisomerase II-targeting agents, including topoisomerase II poisons (e.g., etoposide, tafluposide, teniposide, doxorubicin and mitoxantrone) and topoisomerase II inhibitors (e.g., novobiocin, merbarone and aclarubicin);

DNA or RNA synthesis inhibitors, including 3-amino-1,2,4-benzotriazine 1,4-dioxide, cytosine β-D-arabinofuranoside, 5,6-dichlorobenzimidazole 1-β-D-ribofuranoside, ganciclovir and hydroxyurea;

protein synthesis inhibitors, including homoharringtonine;

cell growth and differentiation regulators, including retinoids (e.g., all-trans retinol [vitamin A], 11-cis retinol, all-trans retinal [vitamin A aldehyde], 11-cis retinal, all-trans retinoic acid [tretinoin], 9-cis-retinoic acid [alitretinoin], 11-cis retinoic acid, 13-cis-retinoic acid [isotretinoin], all-trans retinyl esters, etretinate, acitretin, adapalene, bexarotene and tazarotene);

cell proliferation inhibitors, including mTOR inhibitors (e.g., everolimus, novolimus, ridaforolimus, sirolimus [rapamycin], temsirolimus, umirolimus [biolimus A9] and zotarolimus), apigenin, cholecalciferol (vitamin D₃) and sex hormone-binding globulin;

apoptosis inducers, including 17-allylamino-17-demethoxygeldanamycin, melatonin, mevinolin, psoralen, thapsigargin, troglitazone, inhibitors of histone deacetylases (e.g., romidepsin), and RXR agonists (supra, such as retinoids [e.g., bexarotene]); and

analogs, derivatives and salts thereof.

Cancer immunotherapeutic agents include agents that block immune checkpoints and agents that stimulate the immune system. In certain embodiments, the cancer immunotherapeutic agent is or includes an anti-PD-1 antibody or an anti-PD-L1 antibody, or/and an anti-CTLA-4 antibody.

Anticancer agents that block immune checkpoints include without limitation:

inhibitors of programmed cell death 1 (PD-1) receptor or ligands thereof (e.g., PD-L1 and PD-L2), including anti-PD-1 antibodies (e.g., cemiplimab, nivolumab, pembrolizumab, pidilizumab and MEDI-0680 [AMP-514]), anti-PD-1 fusion proteins (e.g., AMP-224 [containing an antibody F_{c} domain and PD-L2]), anti-PD-L1 antibodies (e.g., avelumab, atezolizumab, durvalumab, and BMS-936559 [MDX-1105]), and small-molecule inhibitors of PD-L1 (e.g., BMS-1001 and BMS-1166);

inhibitors of cytotoxic T lymphocyte-associated protein 4 (CTLA-4) receptor or ligands thereof, including anti-CTLA-4 antibodies (e.g., ipilimumab and tremelimumab);

inhibitors of killer cell immunoglobulin-like receptors (KIRs) or ligands thereof, including anti-KIR antibodies (e.g., lirilumab);

inhibitors of lymphocyte activation gene 3 (LAG-3) receptor or ligands thereof, including anti-LAG-3 antibodies (e.g., BMS-986016 and GSK2831781);

inhibitors of T-cell immunoglobulin and mucin domain-containing 3 (TIM-3, also called hepatitis A virus cellular receptor 2 [HAVCR2]), including anti-TIM3 antibodies (e.g., LY3321367. MBG453 and TSR-022);

inhibitors of indoleamine 2,3-dioxygenase (IDO or IDO1), including indoximod (1-methyl-D-tryptophan), navoximod, α-methyl-tryptophan, β-carboline (9H-pyrido[3,4-b]indole or norharmane), epacadostat (INCB024360), BMS-986205, NLG-919, and COX-2 inhibitors (e.g., coxibs [supra], which downregulate the expression of IDO); and

analogs, derivatives, fragments and salts thereof.

Anticancer agents that stimulate the immune system include, but are not limited to:

agonists of tumor necrosis factor receptor superfamily member 4 (TNFRSF4, OX40 or CD134), including OX40-targeting antibodies (e.g., MEDI-6469 and 9B12) and ligands for OX40 (e.g., OX40L);

agonists of TNFRSF member 5 (TNFRSF5 or CD40), including CD40-targeting antibodies (e.g., dacetuzumab and CP-870,893) and ligands for CD40 (e.g., CD40L [CD154]);

agonists of TNFRSF member 9 (TNFRSF9, 4-1BB or CD137), including 4-1BB-targeting antibodies (e.g., urelumab and PF-05082566) and ligands for 4-1BB (e.g., 4-1BBL);

agonists of TNFRSF member 18 (TNFRSF8, glucocorticoid-induced TNFR-related protein [GITR] or CD357), including GITR-targeting antibodies (e.g., DTA-1 and TRX518) and ligands for GITR (e.g., GITRL);

agonists of toll-like receptors (TLRs), including ligands for TLR9 (e.g., unmethylated CpG oligodeoxynucleotides [CpG ODNs], such as agatolimod);

cytokines and hormones that stimulate immune cells, including IL-6 and epinephrine (stimulator of, e.g., natural killer cells); and

analogs, derivatives, fragments and salts thereof.

Angiogenesis is important for the transition of a benign tumor to a malignant tumor (i.e., a cancer), and for metastasis of a cancer. Thus, anticancer agents include angiogenesis inhibitors. Angiogenesis inhibitors include without limitation inhibitors of vascular endothelial growth factors (VEGFs) {e.g., squalamine, ACU-6151, decorin, anti-VEGF antibodies and fragments thereof (e.g., bevacizumab, ranibizumab, brolucizumab, ENV1305, ESBA903 and ESBA1008), anti-VEGF immunoconjugates (e.g., KSI-301), anti-VEGF aptamers (e.g., pegaptanib), anti-VEGF designed ankyrin repeat proteins (DARPins) (e.g., abicipar pegol), soluble VEGFRs (e.g., sVEGFR1), and soluble fusion proteins containing one or more extracellular domains of one or more VEGFRs (e.g., VEGFR1, VEGFR2 and VEGFR3) (e.g., aflibercept, conbercept and OPT-302)}, inhibitors of receptors for VEGFs (e.g., VEGFR1 and VEGFR2) (e.g., acrizanib [LHA-510], axitinib, fruquintinib, pazopanib, regorafenib, sorafenib, sunitinib, tivozanib, isoxanthohumol, pristimerin, KPI-285, PAN-90806, PF-337210, PP1, TG100572, TG100801 [a TG100572 prodrug], X-82, D-(LPR), decorin, and anti-VEGFR antibodies and fragments thereof [e.g., ramucirumab]), inhibitors of platelet-derived growth factors (PDGFs) {e.g., squalamine, PP1, decorin, anti-PDGF aptamers (e.g., E10030 and pegpleranib), anti-PDGF antibodies and fragments thereof (e.g., rinucumab), and soluble PDGFRs} or receptors therefor (PDGFRs) (e.g., axitinib, imatinib, nilotinib, pazopanib, sorafenib, sunitinib, X-82, and anti-PDGFR antibodies and fragments thereof [e.g., REGN2176-3]), inhibitors of fibroblast growth factors (FGFs) (e.g., squalamine, decorin, anti-FGF antibodies and fragments thereof, anti-FGF aptamers and soluble FGFRs) or receptors therefor (FGFRs) (e.g., erdafitinib, pazopanib and anti-FGFR antibodies and fragments thereof), inhibitors of angiopoictins (e.g., decorin, anti-angiopoietin antibodies and fragments thereof such as nesvacumab and REGN910-3, and soluble angiopoietin receptors) or receptors therefor (e.g. antibodies and fragments thereof against angiopoietin receptors), bispecific anti-VEGF/anti-angiopoietin antibodies and fragments thereof (e.g., anti-VEGF/anti-angiopoietin-2 antibodies such as ABP-201 and RG7716), inhibitors of integrins (e.g., ALG-1001, JSM-6427, SF0166, and anti-integrin antibodies and fragments thereof), tissue factor (TF) inhibitors (e.g., anti-TF antibodies and fragments thereof and fusion proteins thereof [e.g., ICON-1]), kallikrein inhibitors (e.g., avoralstat, ecallantide, BCX7353, KVD001, and anti-kallikrein antibodies and fragments thereof [e.g., DX-2930]), serine/arginine-protein kinase 1 (SRPK1) inhibitors (e.g., SPHINX31), Src kinase inhibitors (e.g., SKI-606, TG100572 and TG100801), anecortave (anecortave acetate), angiostatin (e.g., angiostatin K1-3), αᵥβ₃ inhibitors (e.g., etaracizumab), apoA-I mimetics (e.g., L-4F and L-5F), apoE mimetics (e.g., apoEdp), azurin(50-77) (p28), berberine, bleomycins, borrelidin, carboxyamidotriazole, cartilage-derived angiogenesis inhibitors (e.g., chondromodulin I and troponin I), castanospermine, CM101, corticosteroids (including glucocorticoids), cyclopropene fatty acids (e.g., sterculic acid), α-difluoromethylomithine, endostatin, everolimus, fumagillin, genistein, heparin, interferon-α, interleukin-12, interleukin-18, itraconazole, KV11, linomide, 2-methoxyestradiol, pigment epithelium-derived factor (PEDF), platelet factor-4, PPAR-α agonists (e.g., fibrates), PPAR-γ agonists (e.g., thiazolidinediones), prolactin, rapamycin (sirolimus), sphingosine-1-phosphate inhibitors (e.g., sonepcizumab), squalene, staurosporine, angiostatic steroids (e.g., tetrahydrocortisol) plus heparin, stilbenoids, suramin, SU5416, tasquinimod, tecogalan, tetrathiomolybdate, thalidomide and derivatives thereof (e.g., lenalidomide and pomalidomide), thiabendazole, thrombospondins (e.g., thrombospondin 1), TNP-470, tranilast, triterpenoids (e.g., oleanolic acid analogs such as TP-225), (+)-TBE-B, tumstatin and fusion proteins thereof (e.g., OCU200), vasostatin, vasostatin 48, Withaferin A, and analogs, derivatives, fragments and salts thereof.

Other kinds of anticancer agents include, but are not limited to:

inhibitors of class IA phosphoinositide 3-kinase p110α (PI3K-α), including alpelisib, buparlisib (pan-PI3K), copanlisib (PI3K-α/8), pictilisib (pan-PI3K), taselisib, voxtalisib (pan-PI3K), GNE-477, INK-1117, PWT-33597, SF-1126 (pan-PI3K) and ZSTK-474;

drug-efflux pump inhibitors, including β-glycoprotein inhibitors (e.g., mifepristone and verapamil);

cell adhesion inhibitors, such as cimetidine;

Golgi apparatus disruptors, such as brefeldins (e.g., brefeldin A);

ionizing radiation, such as X-ray;

radiopharmaceuticals, such as ¹³¹I-iodide, ¹³¹I-MIBG (m-iodobenzylguanidine), ²²³Ra-dichloride, ¹⁵³Sm-EDTMP (ethylenediaminotetramethylenephosphoric acid), and ⁸⁹Sr-chloride;

sensitizers of cancer cells to radiation, including PARP inhibitors (infra), berberine and indomethacin;

enhancers of cell survival after treatment with cytotoxic drugs or radiation, such as pifithrin-α;

vaccines, including those that stimulate the immune system to recognize proteins produced by tumor/cancer cells and thereby to attack tumor/cancer cells; and

analogs, derivatives and salts thereof.

Many tumors and cancers are characterized by mutation of PI3K-α resulting in a more active kinase. PI3K-α overactivity contributes significantly to cellular transformation and the development of cancer, including being a key driver of the proliferation and metastatic potential of many solid tumors. Accordingly, in some embodiments one or more NR/NAR derivatives of the disclosure are used in combination with a PI3K-α inhibitor to treat a tumor or cancer. In certain embodiments, the tumor or cancer is a solid tumor or cancer (e.g., of the breast [e.g., HR-positive/HER2-negative breast cancer or triple-negative breast cancer], endometrium or urothelium, or a hyperinsulinemia-associated or obesity-associated solid tumor or cancer [infra] such as colorectal cancer) or a lymphoma (e.g., a non-Hodgkin lymphoma, a B-cell lymphoma, chronic lymphocytic leukemia [CLL] or follicular lymphoma). In certain embodiments, the PI3K-α inhibitor is a selective PI3K-α inhibitor, such as alpelisib.

In some embodiments, one or more NR/NAR derivatives described herein are used in combination with one or more additional therapeutic agents to treat a primary mitochondrial disease (PMD) or a disorder or condition associated with secondary mitochondrial dysfunction (SMD). When mitochondrial dysfunction occurs, the ETC complexes can produce a high level of oxidative stress, which in turn can cause ETC dysfunction. Therefore, in some embodiments, the one or more additional therapeutic agents are or include antioxidants or/and vitamins. Certain vitamins are antioxidants, or cofactors of mitochondrial enzymes or precursors thereof. In certain embodiments, the one or more additional therapeutic agents are or include ubiquinone (coenzyme Q, such as CoQ₁₀) or ubiquinol (a reduced and more biovailable form of ubiquinone, such as ubiquinol-10), or an analog (e.g., idebenone or mitoquinone) or derivative thereof.

Therapeutic agents that can be used to treat a PMD or a disorder or condition associated with SMD include without limitation:

vitamins and analogs thereof, including vitamin B (thiamine), vitamin B₂ (riboflavin), vitamin B₃ (e.g., niacin [nicotinic acid] and nicotinamide), vitamin B₅ (pantothenic acid), vitamin B₆ (pyridoxine), vitamin B₇/B₈ (biotin), vitamin B₉ (folate), vitamin B₁₂ (cobalamin or methylcobalamin), vitamin C (ascorbic acid), vitamin E (including tocopherols [e.g., α-tocopherol] and tocotrienols), and vitamin E analogs (e.g., trolox [water-soluble]);

antioxidants, including glutathione and precursors thereof (e.g., N-acetyl-L-cysteine);

mitochondrial antioxidants and “vitamins”, including ubiquinone (e.g., CoQ₁₀), ubiquinol (e.g., ubiquinol-10), and ubiquinone/ubiquinol analogs (e.g., idebenone and mitoquinone);

other agents supporting mitochondrial processes, including L-carnitine and derivatives thereof (e.g., acetyl-L-carnitine and propionyl-L-carnitine), creatine (e.g., creatine monohydrate), and α-lipoic acid;

minerals, including zinc; and

analogs, derivatives and salts thereof.

An NR or NAR derivative can enhance the immune response to an acute or chronic viral, bacterial or fungal infection when used in conjunction with an antiviral, antibacterial or antifungal agent. In certain embodiments, the antibiotic is ethionamide and optionally SMARt-420 for treatment of, e.g., tuberculosis. Ethionamide has antibiotic properties against mycobacteria such as M. tuberculosis. SMARt-420 reverses resistance of, e.g., M. tuberculosis to ethionamide and increases the bacteria's sensitivity to ethionamide.

An NR or NAR derivative can also enhance and direct the adaptative immune response to a vaccine antigen, thereby improving the effectiveness of the vaccine. An NR or NAR derivative can be utilized as a component of a vaccine adjuvant. In certain embodiments, an NR or NAR derivative is administered in combination with a vaccine to a subject in order to enhance the effectiveness of the vaccine.

The optional additional therapeutic agent(s) independently can be administered in any suitable mode, including without limitation oral, parenteral (including intramuscular, intradermal, subcutaneous, intravascular, intravenous, intra-arterial, intraperitoneal, intracavitary, intramedullary, intrathecal and topical), and topical (including dermal/epicutaneous, transdermal, mucosal, transmucosal, intranasal [e.g., by nasal spray or drop], pulmonary [e.g., by oral or nasal inhalation], ocular [e.g., by eye drop], buccal, sublingual, rectal [e.g., by suppository] and vaginal [e.g., by suppository]). In certain embodiments, an additional therapeutic agent is administered orally. In other embodiments, an additional therapeutic agent is administered parenterally (e.g., intravenously, subcutaneously, intramuscularly, intrathecally or topically [e.g., sublingually]).

The optional additional therapeutic agent(s) independently can be administered in any suitable frequency, including without limitation daily (one, two or more times per day), once every two or three days, twice weekly or once weekly, or on a pro re nata (as-needed) basis, which can be determined by the treating physician. The dosing frequency can depend on, e.g., the mode of administration chosen. The length of treatment with the optional additional therapeutic agent(s) can be determined by the treating physician and can independently be, e.g., at least about 1 day, 2 days, 3 days, 1 week, 2 weeks, 3 weeks, 4 weeks (1 month), 6 weeks, 2 months, 3 months, 6 months, 1 year, 2 years, 3 years, 4 years, 5 years or longer.

The therapeutically effective amount of, the frequency and route of administration of, and the length of treatment with, an optional additional therapeutic agent can be based in part on recommendations for that therapeutic agent and can be determined by the treating physician.

In some embodiments, an NR or NAR derivative and an additional therapeutic agent are administered in separate pharmaceutical compositions. In other embodiments, an NR or NAR derivative and an additional therapeutic agent are administered in the same pharmaceutical composition, such as in a fixed-dose combination dosage form. In some embodiments, the fixed-dose combination dosage form is formulated for controlled-release, slow-release or sustained-release of the NR or NAR derivative or/and the additional therapeutic agent. In certain embodiments, the fixed-dose combination dosage form is formulated for oral administration, such as once or twice daily and such as in the form of a tablet, capsule or pill. In other embodiments, the fixed-dose combination dosage form is formulated for parenteral administration, such as intravenously, subcutaneously, intramuscularly, intrathecally or topically (e.g., sublingually).

Combination Therapies with PARP Inhibitors

When activated by DNA damage, poly(ADP-ribose) polymerase (PARP) recruits other proteins that repair single-stranded DNA breaks (“nicks”). PARP activity is necessary for repair of DNA nicks. PARP expression and activity are upregulated under diverse conditions that lead to DNA damage and ultimately cell injury or cell death, including hypoxia. However, PARP is a major consumer of NAD⁺ in the cell, and markedly increased PARP activity can deplete NAD⁺ and cause profound mitochondrial and cellular dysfunction. Therefore, PARP inhibition can increase NAD⁺ level (e.g., in mitochondria, the cytosol or/and the nucleus, such as total cellular NAD⁺ level) and thereby can enhance mitochondrial function (e.g., oxidative metabolism) and biogenesis and cellular function (e.g., increase the activity of sirtuins such as SIRT1 and SIRT3).

PARP inhibitors are currently approved as antitumor/anticancer agents. DNA damage occurs countless times during each cell cycle, and failure to repair damaged DNA leads to the death of tumor/cancer cells. Some PARP inhibitors mainly block PARP enzyme activity and do not trap PARP on DNA, while other PARP inhibitors both block PARP enzyme activity and act as PARP poison. In the latter case, PARP bound to a PARP inhibitor becomes trapped at the site of a DNA nick, and such a trapped PARP-DNA complex (PARP poison) is more toxic to cells than the unrepaired single-strand DNA breaks that accumulate in the absence of PARP activity because it blocks DNA replication. PARP inhibitors include without limitation niraparib, olaparib, pamiparib (BGB290), rucaparib, talazoparib, veliparib, 4-amino-1,8-naphthalimide, CEP9722, E7016, PJ34, and analogs, derivatives and salts thereof.

The inventors have surprisingly discovered that the combination of nicotinamide riboside plus olaparib at a dose much lower than its chemotherapeutic dose synergistically increases NAD⁺ level (e.g., in mitochondria, the cytosol or/and the nucleus, such as total cellular NAD⁺ level) and provides cytoprotection (reduces cytotoxicity) under DNA damage-inducing conditions (see Example 6 below). Without intending to be bound by theory, low-level PARP inhibition by a PARP inhibitor (e.g., olaparib) at a low dose can reduce the rate of NAD⁺ consumption by PARP, increase NAD⁺ level and hence enhance mitochondrial and cellular function and provide cytoprotection. Moreover, low-level PARP inhibition can avoid the trapping of PARP at the site of a DNA nick, thereby allowing the cellular DNA-repair machinery to repair damaged DNA.

In some embodiments, one or more nicotinyl riboside compounds in combination with a PARP inhibitor increase NAD⁺ level (e.g., total cellular NAD⁺ level, such as that in target cells) by at least about 20%, 30%, 50%, 100% (2-fold), 150%, 200% (3-fold), 4-fold, 5-fold, 6-fold, 7-fold, 8-fold, 9-fold or 10-fold in vitro, ex vivo or in vivo. In certain embodiments, one or more nicotinyl riboside compounds in combination with a PARP inhibitor increase NAD⁺ level (e.g., total cellular NAD⁺ level, such as that in target cells) by at least about 50%, 100% (2-fold), 3-fold or 5-fold in vitro, ex vivo or in vivo.

In further embodiments, one or more nicotinyl riboside compounds in combination with a PARP inhibitor increase the number of viable cells (e.g., target cells) by at least about 10%, 20%, 30%, 50%, 100% (2-fold), 150%, 200% (3-fold), 4-fold or 5-fold in vitro, ex vivo or in vivo. In certain embodiments, one or more nicotinyl riboside compounds in combination with a PARP inhibitor increase the number of viable cells (e.g., target cells) by at least about 20%, 50%, 100% or 200% in vitro, ex vivo or in vivo.

In some embodiments, one or more nicotinyl riboside compounds are used in combination with a PARP inhibitor at a dose significantly lower than its recommended dose as an antitumor/anticancer agent to treat a non-tumor/non-cancer disease/disorder or condition disclosed herein, or to bring about a biological effect disclosed herein (e.g., increase NAD⁺ level or/and provide cytoprotection). The PARP inhibitor can inhibit one or more members of the PARP family, such as PARP-1 or/and PARP-2. In certain embodiments, the PARP inhibitor is a selective or non-selective inhibitor of PARP-1. The non-tumor/non-cancer disease or condition can be, e.g., any mitochondrial disease, mitochondria-related disease or condition, or disease or condition characterized by acute NAD⁺ depletion due to DNA damage described herein. In certain embodiments, the disease or condition is a metabolic disorder (e.g., obesity or type 2 diabetes). One or more other therapeutic agents described herein (e.g., a mitochondrial uncoupler) can optionally be used in combination with one or more nicotinyl riboside compounds and a PARP inhibitor. In some embodiments, the one or more nicotinyl riboside compounds are or comprise one or more of NR, NRH, NAR and NARH, or/and one or more NR/NAR derivatives (such as one or more NR/NAR derivatives disclosed herein). In certain embodiments, the one or more nicotinyl riboside compounds are or comprise NR or/and NRH. In other embodiments, the one or more nicotinyl riboside compounds are or comprise nicotinamide riboside triacetate (NRTA, i.e., NR having an acetate group at each of the C-2, C-3 and C-5 positions of riboside), the reduced form of NRTA (NRHTA), nicotinic acid riboside triacetate (NARTA), or the reduced form of NARTA (NARHTA), or any combination thereof. The use of one or more nicotinyl riboside compounds in combination with a PARP inhibitor (e.g., olaparib) at a significantly sub-chemotherapeutic dose can synergistically increase NAD⁺ level (e.g., in mitochondria, the cytosol or/and the nucleus, such as total cellular NAD⁺ level) or/and provide cytoprotection (e.g., reduce cell injury, damage or death), or can have a synergistic therapeutic effect.

A PARP inhibitor at a significantly sub-chemotherapeutic dose can be used in combination with one or more nicotinyl riboside compounds to treat any non-tumor/non-cancer disease/disorder or condition associated with DNA damage. The DNA damage can be due to any cause, such as radiation (e.g., UV or an ionizing radiation such as X-ray), a chemical, a chemotherapeutic agent, oxidative stress or hypoxia. The disease/disorder or condition can be acute or chronic, and can be associated with NAD⁺ depletion or/and cell injury, damage, degeneration or death. Such diseases/disorders and conditions include without limitation diseases and conditions characterized by acute NAD⁺ depletion due to DNA damage and described above. In certain embodiments, the disease/disorder or condition is an acute life-threatening cardiovascular (e.g., myocardial ischemia/infarction/IRI) or cerebrovascular (e.g., cerebral ischemia/infarction/IRI) disorder, or a neurodegenerative disorder.

In certain embodiments, a PARP inhibitor at a significantly sub-chemotherapeutic dose is used with one or more nicotinyl riboside compounds to treat a liver disorder or an inflammatory disorder, such as a disorder associated with systemic inflammatory response syndrome (SIRS). Liver disorders associated with SIRS include acute-on-chronic liver failure (ACLF) and alcoholic hepatitis. Alcoholic hepatitis is characterized by PARP activation and severe NAD⁺ depletion, and hence the utility of a PARP inhibitor and a nicotinyl riboside compound.

In some embodiments, the dose of a PARP inhibitor to treat a non-tumor/non-cancer disease/disorder or condition disclosed herein, or to bring about a biological effect disclosed herein, in combination with one or more nicotinyl riboside compounds is no more than about 10%, 5%, 1%, 0.5% or 0.1% of the recommended dose of the PARP inhibitor as an antitumor/anticancer agent. In certain embodiments, the dose of a PARP inhibitor for such a use is no more than about 1% of the recommended dose of the PARP inhibitor as an antitumor/anticancer agent. In some embodiments, the PARP inhibitor is olaparib, and the dose (e.g., per day or per dose) of olaparib to treat a non-tumor/non-cancer disease/disorder or condition disclosed herein, or to bring about a biological effect disclosed herein, in combination with one or more nicotinyl riboside compounds is no more than about 10 mg, 5 mg, 1 mg, 0.5 mg or 0.1 mg; or is from about 0.01 or 0.1 mg to about 10 mg, from about 0.01 or 0.1 mg to about 1 mg, or from about 1 mg to about 10 mg; or is about 0.01-0.1 mg, 0.1-0.5 mg, 0.5-1 mg, 1-5 mg or 5-10 mg; or is about 10 μg, 50 μg, 0.1 mg, 0.5 mg, 1 mg, 5 mg or 10 mg. In certain embodiments, the dose (e.g., per day or per dose) of olaparib for such a use is no more than about 1 mg.

A PARP inhibitor can be administered in any suitable frequency. In certain embodiments, the PARP inhibitor is administered once or twice daily.

The dose or therapeutically effective amount, the frequency of administration and the route of administration of a nicotinyl riboside compound used in conjunction with a low dose of a PARP inhibitor can be, e.g., any dose or therapeutically effective amount, any frequency of administration and any route of administration of the NR/NAR derivatives of the disclosure described herein. In some embodiments, the dose of a nicotinyl riboside compound (e.g., NR, NRH, NRTA, NRHTA or an NR/NAR derivative disclosed herein) is from about 1, 50 or 100 mg to about 500 or 1000 mg per day, which can be administered (e.g., orally) in a single dose (e.g., N mg once daily) or in divided/multiple doses (e.g., N/2 mg twice daily). In certain embodiments, the dose of a nicotinyl riboside compound is about 1-100 mg, 100-500 mg or 500-1000 mg per day, or about 50 mg, 100 mg, 150 mg, 200 mg, 250 mg, 300 mg, 400 mg, 500 mg, 600 mg, 700 mg, 800 mg, 900 mg or 1000 mg per day. The dose of a nicotinyl riboside compound can also be higher than 1.0 g per day, such as about 1.0-1.5 g, 1.5-2.0 g, 2.0-2.5 g or 2.5-3.0 g per day. In further embodiments, the dose of a nicotinyl riboside compound is about 1-50 mg, 50-100 mg, 100-200 mg, 200-300 mg, 300-400 mg or 400-500 mg per day. In certain embodiments, the dose of a nicotinyl riboside compound is from about 10, 50 or 100 mg to about 200 or 300 mg per day. In some embodiments, a lower dose of a nicotinyl riboside compound is used to treat a less severe non-tumor/non-cancer disease/disorder or condition, while a higher dose of a nicotinyl riboside compound is used to treat a more severe non-tumor/non-cancer disease/disorder or condition.

A nicotinyl riboside compound can be administered in any suitable frequency. In certain embodiments, a nicotinyl riboside compound is administered once or twice daily.

The length of treatment with a PARP inhibitor and one or more nicotinyl riboside compounds to treat a non-tumor/non-cancer disease/disorder or condition disclosed herein, or to bring about a biological effect disclosed herein, can be determined by the treating physician. For example, the length of treatment with the PARP inhibitor and the one or more nicotinyl riboside compounds can independently be at least about 1 day, 2 days, 3 days, 1 week, 2 weeks, 3 weeks, 4 weeks (1 month), 6 weeks, 2 months, 3 months, 6 months, 1 year, 2 years, 3 years, 4 years, 5 years or longer. The PARP inhibitor and the one or more nicotinyl riboside compounds can also be taken pro re nata (as needed).

The synergistic effects of a combination of one or more nicotinyl riboside compounds and a low dose of a PARP inhibitor, such as in elevating NAD⁺ level and enhancing cytoprotection, can be exploited prophylactically to prevent a non-tumor/non-cancer disease/disorder or condition, or potentially to prevent a tumor or cancer. As an example, one or more nicotinyl riboside compounds and a low dose of a PARP inhibitor can be given prior to a surgery to reduce morbidity caused by general anesthesia or hypoxia- or hypotension-induced cytotoxicity. For instance, one or more nicotinyl riboside compounds and a low dose of a PARP inhibitor can be given prior to a cardiac procedure (e.g., angioplasty or valvular surgery) to reduce morbidity and mortality due to hypotensive or bleeding episodes. As another example, one or more nicotinyl riboside compounds and a low dose of a PARP inhibitor can be applied to the skin to prevent sunlight-induced skin injury.

One or more nicotinyl riboside compounds and a PARP inhibitor can be administered to a subject via any suitable route. In certain embodiments, the one or more nicotinyl riboside compounds or/and the PARP inhibitor are administered orally. In other embodiments, the one or more nicotinyl riboside compounds or/and the PARP inhibitor are administered parenterally (e.g., intravenously, subcutaneously, intramuscularly, intrathecally or topically [e.g., sublingually]). The route of administration of the one or more nicotinyl riboside compounds and the PARP inhibitor can depend in part on the disorder or condition being treated. For example, the one or more nicotinyl riboside compounds or/and the PARP inhibitor can be administered dermally or transdermally to treat a skin disorder or condition.

One or more nicotinyl riboside compounds and a PARP inhibitor can be administered in the same pharmaceutical composition or in separate compositions. In some embodiments, the one or more nicotinyl riboside compounds (e.g., NR or/and NRH, NRTA or/and NRHTA, or one or more NR/NAR derivatives disclosed herein) and the PARP inhibitor (e.g., olaparib) are administered in a fixed-dose combination dosage form, where the dose of the PARP inhibitor is significantly lower than its recommended dose as an antitumor/anticancer agent. In some embodiments, the fixed-dose combination dosage form is a controlled-release, slow-release or sustained-release form. In certain embodiments, the fixed-dose combination dosage form is formulated for oral administration, such as once or twice daily and such as in the form of a tablet, capsule or pill. In other embodiments, the fixed-dose combination dosage form is formulated for parenteral administration, such as intravenously, subcutaneously, intramuscularly, intrathecally or topically (e.g., sublingually). In further embodiments, the one or more nicotinyl riboside compounds or/and the PARP inhibitor are administered as a complex with a dendrimer (e.g., a PAMAM or/and PEG dendrimer) or via a dendrimer-containing composition. The dendrimer can optionally have one or more moieties for targeting to specific organ(s), tissue(s), cell type(s) or organelle(s), such as one or more N-acetylgalactosamine moieties for targeting to the liver for treatment of, e.g., a liver or metabolic disorder.

In other embodiments, one or more nicotinyl riboside compounds or/and a PARP inhibitor are utilized in ex vivo therapy, including in any ex vivo therapy described herein. In yet other embodiments, one or more nicotinyl riboside compounds and a PARP inhibitor are employed to enhance DNA editing, such as in the use of a CRISPR, transcription activator-like effector nuclease (TALEN) or Arcus nuclease to promote non-homologous end joining (NHEJ) or homology-directed repair (HDR). Low-level PARP inhibition by a low dose of a PARP inhibitor permits repair of single-stranded DNA breaks.

Combination Therapies with Mitochondrial Uncouplers

Cellular respiration converts chemical energy derived from nutrients into ATP. The oxidation of nutrients in the mitochondrial matrix generates high-energy electron carriers nicotinamide adenine dinucleotide (NADH) and flavin adenine dinucleotide (FADH₂) via the Krebs (or tricarboxylic acid [TCA]) cycle. Mitochondrial ATP production is driven by the transfer of electrons from NADH and FADH₂ to O₂ through Complexes I-IV of the electron transport chain (ETC) in the mitochondrial inner membrane (MIM). Electron flow through the ETC releases energy, which allows components of Complexes 1, III and IV to pump protons (H+) from the mitochondrial matrix across the MIM into the mitochondrial intermembrane space against their concentration gradient. The resulting proton and electrical gradients, known as the proton motive force, provides energy for ATP synthesis. Protons flow down their concentration gradient through the MIM ATP synthase into the mitochondrial matrix to drive the synthesis of ATP by phosphorylation of ADP. The whole process is called oxidative phosphorylation. Any proton re-entry into the mitochondrial matrix that bypasses ATP synthase leads to uncoupling of oxidative phosphorylation.

Uncoupling proteins (UCPs) are regulated proton channels in the MIM that facilitate proton transfer into the mitochondrial matrix independent of ATP synthase, thereby uncoupling mitochondrial respiration from ATP synthesis. Activation of UCPs by reactive species and fatty acids causes induced proton leak. The energy lost in dissipation of the proton gradient via UCPs generates heat (thermogenesis) rather than ATP. UCPs play a role in normal physiology, including maintenance of body temperature in mammals (e.g., non-shivering heat generation in cold exposure or hibernation), regulation of mitochondrial respiration and ATP synthesis, reduction of mitochondrial production of reactive oxygen species (ROS), and release of calcium ions from mitochondria in neurons.

Like UCPs, protonophoric mitochondrial uncouplers (or “uncouplers” for short) transport protons across the MIM into the mitochondrial matrix while bypassing ATP synthase. Protonophoric uncouplers typically are aromatic weak acids (e.g., pKₐ of about 4-10) that are lipophilic and capable of distributing the negative charge over a number of atoms via α-orbitals that delocalize a proton's positive charge when the proton complexes to the compound. Without intending to be bound by theory, the anionic form of the uncoupler associates with a proton in the acidic pH environment of the mitochondrial intermembrane space, and the resulting neutral form of the uncoupler passively diffuses across the lipid bilayer of the MIM. Once inside the basic pH environment of the mitochondrial matrix, the neutral form of the uncoupler dissociates into H⁺ and the anionic form of the uncoupler, which returns to the intermembrane space by electrostatic attraction to protons there to continue the catalytic uncoupler cycling mechanism. Delocalization of the negative charge renders the anionic form of the uncoupler more hydrophobic and hence facilitates its crossing of the MIM to the intermembrane space. The theoretical maximum number of cycles for a weak acid uncoupler is about 1000/sec based on Brownian motion.

Uncouplers can also induce proton leak into the mitochondrial matrix by a mechanism other than weak acid/anion cycling. For example, fatty acids (e.g., long-chain fatty acids) and alkylsulfonates (e.g., undecanosulfonate) activate UCP. Other agents that increase the expression or/and activity of UCPs include other small molecules (e.g., retinoic acids and analogs thereof, catecholamines [e.g., epinephrine and norepinephrine], flavonoids [e.g., quercetin], and certain antidiabetics [e.g., metformin, des-fluoro-sitagliptin and thiazolidinediones]) and certain macromolecules (e.g., leptin and thyroid hormones). In addition, certain uncouplers interact with the MIM adenine nucleotide translocase (infra).

Protonophoric uncouplers uncouple the ETC from ATP synthesis via a decrease in the pH difference between the intermembrane space and the mitochondrial matrix and in the MIM membrane potential, thereby reducing ATP production in the mitochondrial matrix. Uncouplers increase TCA cycle flux, cellular oxygen consumption, mitochondrial respiration, ETC activity and electron-transfer speed, heat generation, autophagy (including mitophagy to remove damaged or dysfunctional mitochondria) and mitochondrial biogenesis, and reduce intramitochondrial Ca²⁺ concentration, mitochondrial swelling, and mitochondrial production and accumulation of ROS. Reduction of the mitochondrial membrane potential by uncoupling increases cytosolic Ca²⁺ level by closing voltage-dependent uniporters involved in Ca²⁺ influx to the mitochondrial matrix and increasing release of Ca²⁺ ions from mitochondria, which activates adenylyl cyclase and hence the formation of the second messenger cyclic AMP (cAMP), cAMP induces the expression of a variety of genes, including those for neurotrophins such as brain-derived neurotrophic factor (BDNF). BDNF enhances NAD⁺ level, protects neurons against excitotoxicity, and promotes neuronal growth, repair of damaged neurons, synaptic plasticity and cognition. Furthermore, increased cAMP concentration in vascular smooth muscle cells has vasodilating and hence antihypertensive effects. Mild uncoupling mimics beneficial effects of calorie restriction, including activation of AMPK and cAMP response element-binding (CREB) protein; increased expression of BDNF, sirtuins 1 and 3, peroxisome proliferator-activated receptor gamma coactivator 1-alpha (PGC-1α), and glucose transporter 1 (GLUT1); increase in mitochondrial biogenesis, autophagy, mitochondrial bioenergetics and cellular resiliency; and reduction of cellular stress, metabolic stress, oxidative stress, mTORC1 signaling and apoptosis.

In addition, mild mitochondrial uncoupling increases NADH oxidation to NAD⁺ in the ETC, and increases glucose uptake and mitochondrial biogenesis by energy-intensive cells (e.g., neurons and muscle, heart, liver, kidney and immune cells), which maintain sufficient energy/ATP production (e.g., by an increased number of mitochondria) to support the function (e.g., metabolic function) and survival of energy-intensive cells. NADH and ATP can inhibit certain TCA cycle enzymes as checks on the TCA cycle, and NADH can promote ROS production by mitochondrial-matrix flavoproteins. By reducing NADH and ATP levels in the mitochondrial matrix, mild uncoupling enhances TCA cycle flux in the matrix and reduces mitochondrial ROS production. Uncoupling also reduces ROS production and levels by reducing mitochondrial membrane potential, which results in neutralization of ROS and less electron leakage.

Mitochondrial uncouplers include without limitation benzoic acid, phenol, 2-methylphenol, 3-methylphenol, 4-methylphenol, all the dimethylphenol regioisomers (e.g., 2,4-dimethylphenol and 2,6-dimethylphenol), all the trimethylphenol regioisomers (e.g., 2,4,6-trimethylphenol), 2-isopropylphenol, 3-isopropylphenol, 4-isopropylphenol, all the di-isopropylphenol regioisomers (e.g., 2,4-di-isopropylphenol and 2,6-di-isopropylphenol), all the tri-isopropylphenol regioisomers (e.g., 2,4,6-tri-isopropylphenol), 2-tert-butylphenol, 3-tert-butylphenol, 4-tert-butylphenol, all the di-tert-butylphenol regioisomers (e.g., 2,4-di-tert-butylphenol and 2,6-di-tert-butylphenol), all the tri-tert-butylphenol regioisomers (e.g., 2,4,6-tri-tert-butylphenol), 2-methoxyphenol, 3-methoxyphenol, 4-methoxyphenol, all the dimethoxyphenol regioisomers (e.g., 2,4-dimethoxyphenol and 2,6-dimethoxyphenol), all the trimethoxyphenol regioisomers (e.g., 2,4,6-trimethoxyphenol), all tert-butyl-methoxyphenol regioisomers (e.g., butylated hydroxyanisole [BHA], which comprises 2-tert-butyl-4-methoxyphenol or/and 3-tert-butyl-4-methoxyphenol), all di-tert-butyl-methylphenol regioisomers (e.g., 2,6-di-tert-butyl-4-methylphenol, which is commonly known as butylated hydroxytoluene [BHT]), 2-chlorophenol, 3-chlorophenol, 4-chlorophenol, all the dichlorophenol regioisomers (e.g., 2,4-dichlorophenol and 2,6-dichlorophenol), all the trichlorophenol regioisomers (e.g., 2,4,6-trichlorophenol), all the tetrachlorophenol regioisomers, pentachlorophenol, 2-fluorophenol, 3-fluorophenol, 4-fluorophenol, all the difluorophenol regioisomers (e.g., 2,4-difluorophenol and 2,6-difluorophenol), all the trifluorophenol regioisomers (e.g., 2,4,6-trifluorophenol), all the tetrafluorophenol regioisomers, pentafluorophenol, 2-cyanophenol, 3-cyanophenol, 4-cyanophenol, all the dicyanophenol regioisomers (e.g., 2,4-dicyanophenol and 2,6-dicyanophenol), all the tricyanophenol regioisomers (e.g., 2,4,6-tricyanophenol), 2-nitrophenol, 2-nitroanisole, 3-nitrophenol, 3-nitroanisole, 4-nitrophenol, 4-nitroanisole, 2,3-dinitrophenol (2,3-DNP), 2,3-dinitroanisole, 2,4-dinitrophenol (2,4-DNP, or commonly known as DNP), 2,4-dinitroanisole (2,4-DNP methyl ether, or commonly known as DNP methyl ether, a liver-targeted DNP prodrug), MP201 (a DNP prodrug), 2,5-dinitrophenol (2,5-DNP), 2,5-dinitroanisole, 2,6-dinitrophenol (2,6-DNP), 2,6-dinitroanisole, 3,4-dinitrophenol (3,4-DNP), 3,4-dinitroanisole, 3,5-dinitrophenol (3,5-DNP), 3,5-dinitroanisole, all the trinitrophenol regioisomers (e.g., 2,4,6-trinitrophenol [picric acid]), all the dinitrocresol regioisomers (e.g., 2-methyl-3,5-dinitrophenol, which is commonly known as dinitro-ortho-cresol), naturally occurring phenols {including simple phenols (e.g., catechol, resorcinol and hydroquinone), alkylresorcinols (e.g., adipostatin A [cardol], bilobol, hexylresorcinol, olivetol and DB-2073), monoterpenoid phenols (e.g., carvacrol and thymol), diterpenoid phenols (e.g., carnosol), dioxophenols (e.g., sesamol), phenolic aldehydes (e.g., vanillin and isovanillin), phenolic acids (e.g., salicylic acids, vanillic acid and gallic acid), hydroxylated phenylacetic acids (e.g., 4-hydroxyphenylacetic acid and homogentisic acid), hydroxylated phenylethanoids (e.g., tyrosol, hydroxytyrosol and oleocanthal), hydroxylated phenylpropenes (e.g., eugenol), hydroxylated phenylpropanones (e.g., gingerol and raspberry ketone), hydroxycinnamic acids (e.g., caffeic acid, chicoric acid, ortho-coumaric acid, meta-coumaric acid, para-coumaric acid, ferulic acid, rosmarinic acid and sinapinic acid), curcuminoids (e.g., curcumin, demethoxycurcumin, bisdemethoxycurcumin), hydroxylated coumarins (e.g., aesculetin, scopoletin and umbelliferone), hydroxylated isocoumarins (e.g., hydrangenol, phyllodulcin and thunberginols A, C, D, E and G), hydroxylated chromones (e.g., eugenin), hydroxylated naphthoquinones (e.g., alkannin, juglone, nigrosporin B and plumbagin), xanthonoids (e.g., mangostin and norathyriol), stilbenoids (e.g., piceatannol, pinosylvin, pterostilbene resveratrol and stilbestrol), dihydrostilbenoids (e.g., combretastatin, combretastatin B-1, dihydro-resveratrol and isonotholaenic acid), hydroxylated anthraquinones (e.g., aloe emodin), hydroxylated flavones (e.g., acacetin, apigenin, chrysin, diosmetin and luteolin), hydroxylated flavonols (e.g., fisetin, galangin, isorhamnetin, kaempferol, myricetin, pachypodol, quercetin and rhamnazin), hydroxylated flavanones (e.g, eriodictyol, homoeriodictyol, hesperetin, naringenin and silibinin), hydroxylated flavanonols (e.g., astilbin, aromadendrin [dihydrokaempferol] and taxifolin [dihydroquercetin]), hydroxylated flavan-3-ols [e.g., (+)-catechin, (−)-epicatechin, (+)-gallocatechin, (−)-epigallocatechin, (−)-epicatechin gallate and (−)-epigallocatechin gallate], hydroxylated aurones (e.g., aureusidin and leptosidin), hydroxylated isoflavones (e.g., alpinumisoflavone, biochanin A, daidzein, formononetin, genistein and glycitein), hydroxylated isoflavanes (e.g., laxiflorane and lonchocarpane), hydroxylated isoflavenes (e.g., glabrene, haginin D and 2-methoxyjudaicin), biflavonoids (e.g., amentoflavone, morelloflavone and ochnaflavone), anthocyanidins (e.g, aurantinidin, capensinidin, cyanidin, delphinidin, europinidin, hirsutidin, malvidin, pelargonidin, peonidin, petunidin, pulchellidin and rosinidin), hydroxylated coumestans (e.g., coumestrol, 4′-methoxycoumestrol, plicadin, repensol, trifoliol and wedelolactone), lignans (e.g., enterodiol, enterolactone, lariciresinol, secoisolariciresinol, matairesinol, hydroxymatairesinol, pinoresinol and syringaresinol), pterocarpans (e.g., glyceollins I and III, glycinol, glycyrrhizol A, medicarpin and phaseolin), other polyphenols (e.g., ellagic acid), capsaicin and cannabinoids}, retinoids (e.g., acitretin, adapalene, bexarotene and tazarotenic acid), lipophilic aromatic NSAIDs with a weak-acid group or convertible to such compounds {including acetic acid derivatives (e.g., diclofenac, aceclofenac, etodolac, ketorolac, indomethacin, 6-methoxy-2-naphthylacetic acid [6-MNA], nabumetone, sulindac and tolmetin), propionic acid derivatives (e.g., fenbufen, fenoprofen, flurbiprofen, ibuprofen, dexibuprofen, ketoprofen, dexketoprofen, loxoprofen, naproxen and oxaprozin), enolic acid derivatives (oxicams) (e.g., isoxicam, meloxicam, piroxicam, droxicam, tenoxicam and lornoxicam [chlortenoxicam]), anthranilic acid derivatives (fenamates) (e.g, flufenamic acid, meclofenamic acid, mefenamic acid and tolfenamic acid), salicylates (e.g., salicylic acid, acetylsalicylic acid [aspirin], methyl salicylate, diflunisal and salsalate), sulfonanilides (e.g., nimesulide), 3,5-pyrazolidinediones (e.g., azapropazone and phenylbutazone), selective COX-2 inhibitors (coxibs) (e.g., celecoxib, lumiracoxib, rofecoxib, valdecoxib, parecoxib and SC-236) and others (e.g., clonixin and licofelone)}, lipophilic aromatic antidiabetic agents with a weak-acid group or convertible to such compounds {including AMPK agonists (e.g., biguanides such as phenformin), PPAR-γ agonists (e.g., saroglitazar and thiazolidinediones such as balaglitazone, ciglitazone, darglitazone, englitazone, lobeglitazone, netoglitazone, pioglitazone, rivoglitazone, rosiglitazone and troglitazone), SGLT2 inhibitors (e.g., remogliflozin etabonate, phloretin and phlorizin), and KArP channel blockers (e.g., meglitinides [e.g., mitiglinide, nateglinide and repaglinide], frt-generation sulfonylureas [e.g., acetohexamide, carbutamide, chlorpropamide, glycyclamide (tolhexamide), metahexamide, tolazamide and tolbutamide], and second-generation sulfonylureas [e.g., glibenclamide (glyburide), glibornuride, gliclazide, glimepiride, glipizide, gliquidone, glisoxepide and glyclopyramide])}, anilides (e.g., bupivacaine, NNC-0112-0000-2604 and salicylanilides [e.g., niclosamide and S13]), N-phenylcarbamates. O-organyl-N-phenylthiocarbamates, N-phenylureas (e.g., SR4), N-phenylthioureas, sulfonanilides (e.g., endosidin 9, nimesulide and those disclosed in WO 2019/226490 A1), 2-acyl/aroyl-indan-1,3-diones [e.g., 2-heptanoyl-indan-1,3-dione and 2-(3′,4′-dichlorobenzoyl)-indan-1,3-dione], 2-aryl-1,3-indandiones (e.g., 2-β-chlorophenyl-indan-1,3-dione), benzimidazoles (e.g., TTFB), cyanotriazoles (e.g., OPC-163493, which preferentially localizes in the liver and kidneys), dithiocarbazates (e.g., S-alkyl, N-acyl/aroyldithiocarbazates [e.g., PDTC-9, NDTC-9 and IDTC-9]), N-phenylanthranilic acids (e.g., fenamate NSAIDs), phenylhydrazones (e.g., CCCP and FCCP), 3,5-pyrazolidinediones (e.g., p,p′-dichlorophenylbutazone), ellipticine, niclosamide (e.g., niclosamide ethanolamine), DK-520 (n-octanoyl ester of niclosamide), niclosamide analogs having —Cl or —CF₃ in place of the —NO₂ group, nitazoxanide (NTZ), tizoxanide (des-acetyl NTZ, an active metabolite of NTZ), oxyclozanide, usnic acid (e.g., (+)-usnic acid),

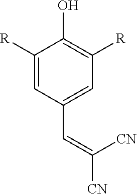

(both R═H, Me, Et, n-Pr, iso-Pr, n-Bu, iso-Bu and sec-Bu),

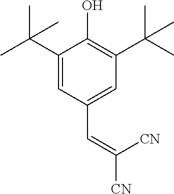 [SMILES: CC(C)(C)c1cc(C=C(C#N)C#N)cc(C(C)(C)C)c1O]

(tyrphostin A9, AG17 or SF-6847),

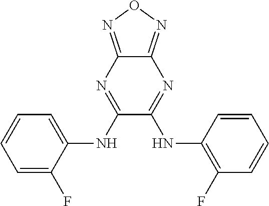 [SMILES: Fc1ccccc1Nc1nc2nonc2nc1Nc1ccccc1F]

(BAM15, which selectively depolarizes mitochondrial membrane and primarily distributes to the liver),

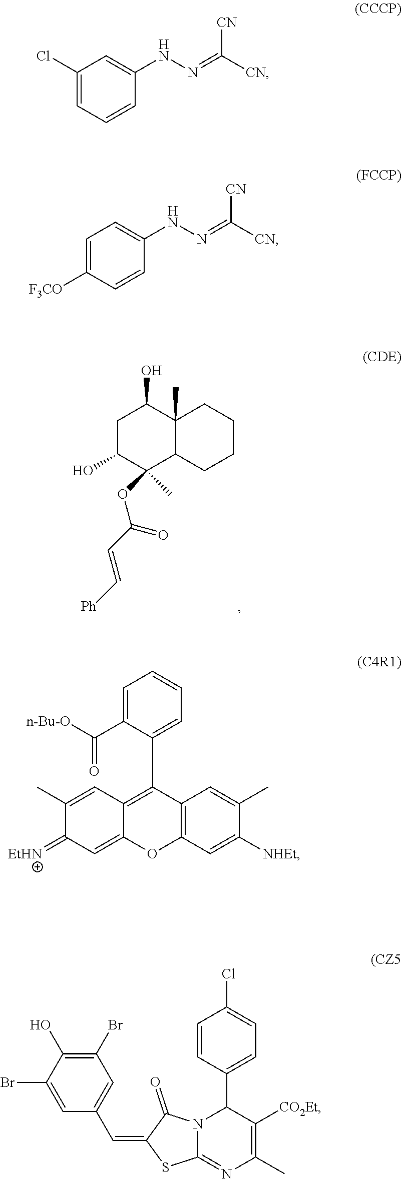

which preferentially uncouples in white adipose tissue),

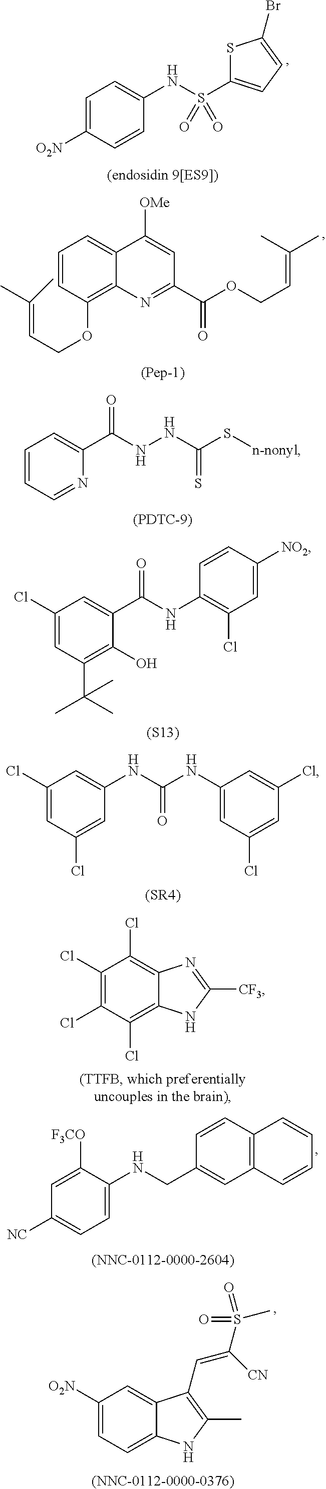

and analogs, derivatives, prodrugs (including the corresponding uncouplers having a methyl ether at an aromatic hydroxyl [e.g., phenolic] group), metabolites, salts, targeted forms (including the corresponding uncouplers having a methyl ether at an aromatic hydroxyl [e.g., phenolic] group for targeting to the liver), and controlled-release, slow-release and sustained-release forms (including liposomes, cholestosomes and lipid, polymeric or dendrimeric nanoparticles encapsulating the uncouplers) thereof.

Prodrugs generally are designed to serve one or more purposes. Such purposes can be, for example: 1) to increase the bioavailability of the parent drug in view of the intended route of administration; 2) to increase the absorption or penetration of the parent drug passively or actively through a biological barrier (e.g., the intestinal or cutaneous epithelium, a mucous membrane, the blood-brain or blood-retina barrier, or the cell membrane); 3) to increase the exposure to or the area under the curve (AUC) of the parent drug; 4) to increase the half-life or residency/elimination time of the parent drug; 5) to increase the aqueous solubility of the parent drug; 6) to deliver the parent drug in a more controlled, slow or sustained manner; 7) to lower the Cₘₐₓ (peak plasma concentration) or/and to delay the tₘₐₓ (time to reach Cₘₐₓ) of the parent drug; and 8) to target the parent drug to specific cell type(s), tissue(s) or organ(s).

In some embodiments, prodrugs of uncouplers having at least one aromatic hydroxyl (e.g., phenolic) group are esters or acetal esters at one or more, or all, aromatic hydroxyl (e.g., phenolic) group(s). In some embodiments, each ester or acetal ester promoiety independently has the formula —C(═O)R or —CHR¹OC(═O)R^{j}, respectively, wherein R¹ is hydrogen or methyl and R is alkyl, cycloalkyl, heterocyclyl, aryl or heteroaryl, each of which can optionally be substituted. In certain embodiments, R is: 1) linear or branched C₁-C₇ alkyl (e.g., methyl, ethyl, n-propyl, isopropyl, n-butyl, isobutyl, sec-butyl, tert-butyl, n-pentyl, n-hexyl or n-heptyl), which can optionally be substituted with —OH, —O—(C₁-C₄ alkyl), —OC(═O)—(C₁-C₄ alkyl), a monoethylene glycol or polyethylene glycol group ending in —OH or —OMe (abbreviated as M/P-EG-OH or M/P-EG-OMe), —NH₂, —NH(C₁-C₄ alkyl), —N(C₁-C₄ alkyl)₂, or —NHC(═O)—(C₁-C₄ alkyl); 2) C₃-C₁₀ cycloalkyl (e.g., cyclopropyl, cyclobutyl, cyclopentyl, cyclohexyl, cycloheptyl, cyclo-octyl, norbornyl or adamantyl); 3) 3-6-membered heterocyclyl (e.g., oxiranyl, oxetanyl, tetrahydrofuranyl, tetrahydropyranyl, aziridinyl, azetidinyl, pyrrolidinyl, piperidinyl, morpholinyl or piperazinyl); or 4) C₆-C₁₀ aryl (e.g., phenyl or naphthyl) or 5-10-membered heteroaryl (e.g., pyrrolyl, pyridyl or quinolyl), either of which can optionally be substituted with one or more substituents selected from —F, —Cl, —Br, linear or branched C₁-C₄ alkyl, —CF₃, —OH, —O-(linear or branched C₁-C₄ alkyl), —O(CH₂)₂₋₄Z, —OC(═O)—(C₁-C₄ alkyl), M/P-EG-OH, M/P-EG-OMe, —NH₂, —NH(C₁-C₄ alkyl), and —N(C₁-C₄ alkyl)₂, wherein Z is —OH, —O-(linear or branched C₁-C₄ alkyl), —NH₂, —NH(C₁-C₄ alkyl), —N(C₁-C₄ alkyl)₂, or —N-heterocyclyl (e.g., —N-aziridine, —N-azetidine, —N-pyrrolidine, —N-piperidine, —N-morpholine, —N-piperazine or —N-piperazine-N-Me). In certain embodiments, R is phenyl ortho-, meta- or para-substituted with —O(CH₂)₂Z wherein Z is N-pyrrolidine, —N-piperidine, —N-morpholine, —N-piperazine or —N-piperazine-N-Me, such as in 2,4-DNP-OC(═O)-phenyl-para-[-O(CH₂)₂Z] wherein Z is N-pyrrolidine, —N-piperidine, —N-morpholine, —N-piperazine or —N-piperazine-N-Me. In other embodiments, R is —(CH₂)ₘC(═O)NR²R³ wherein m is 1, 2, 3 or 4 and 1) R² and R³ independently are hydrogen, C₁-C₆ alkyl or C₃-C₆ cycloalkyl, either of which can optionally be substituted, or R² and R³ and the nitrogen atom to which they are attached form an optionally substituted 3-6 membered heterocyclic ring; or 2) R² is hydrogen or methyl and R³ is —(CH₂), CH₂X wherein n is 0, 1, 2, 3, 4 or 5 and X is —OH, —O—(C₁-C₄ alkyl), —OC(═O)—(C₁-C₄ alkyl), M/P-EG-OH, M/P-EG-OMe, —NH₂, —NH(C₁-C₄ alkyl), —N(C₁-C₄ alkyl)₂, —N-heterocyclyl (e.g., —N-aziridine, —N-azetidine, —N-pyrrolidine, —N-piperidine, —N-morpholine, —N-piperazine or —N-piperazine-N-Me), —NHC(═O)—(C₁-C₄ alkyl), C₃-C₆ cycloalkyl, C₆-C₁₀ aryl (e.g., phenyl or naphthyl) or 5-10-membered heteroaryl (e.g., pyrrolyl, pyridyl or quinolyl), any of which can optionally be substituted. In certain embodiments, m is 1 or/and n is 0 or 1. In certain embodiments, R² is hydrogen where R³ is —(CH₂)CH₂X. To control the release and widen the therapeutic/safety window of an uncoupler with a lower therapeutic/safety index (e.g., 2,4-DNP, which is commonly known as DNP), the ester or acetal ester promoiety can have greater steric hindrance adjacent to the ester bond (e.g., R is tert-butyl, norbornyl, adamantyl, 2,6-dimethylphenyl, 2,6-di-isopropylphenyl or 2,6-di-tert-butylphenyl) or/and R¹ can be methyl, which would slow down chemical or enzymatic hydrolysis of the ester bond in the body and thereby lower the Cₘₐₓ of the uncoupler.

In other embodiments, ester prodrugs of uncouplers having at least one aromatic hydroxyl (e.g., phenolic) group have either of the following structures:

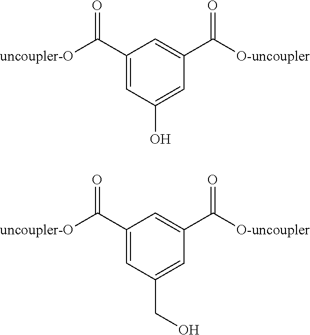

The phenolic or benzylic hydroxyl group of the central phenyl ring can optionally be derivatized, e.g., as an alkyl (e.g., methyl) ether.

In further embodiments, prodrugs of uncouplers having at least one aromatic hydroxyl (e.g., phenolic) group are amino acid esters or amino acid acetal esters at one or more, or all, aromatic hydroxyl (e.g., phenolic) group(s), or form an ester bond or an acetal ester bond with a dipeptide or tripeptide at one or more, or all, such group(s). An amino acid, dipeptide or tripeptide acetal ester promoiety has the general formula —CHR¹OC(═O)R^{j}, wherein R¹ is hydrogen or methyl and —C(═O)R is an amino acid, dipeptide or tripeptide moiety. Prodrugs having an amino acid, dipeptide or tripeptide moiety can have increased oral bioavailability and residency time via active transport by peptide transporters such as PepT1, which facilitates intestinal absorption and renal reabsorption. An amino acid promoiety, or an amino acid moiety of an amino acid acetal ester promoiety, can be any natural amino acid, any natural but non-proteinogenic amino acid (e.g., ornithine, citrulline, homoarginine or β-alanine), or any unnatural amino acid (e.g., —C(═O)CH₂—N-piperazine), and can be an α-amino acid, a β-amino acid (e.g., β-alanine) or so on. In some embodiments, the amino acid is selected from glycine, alanine, valine, leucine, isoleucine, methionine, proline, tryptophan, phenylalanine, tyrosine, serine, threonine, cysteine, asparagine, glutamine, aspartic acid, glutamic acid, lysine, arginine and histidine. In certain embodiments, the amino acid is glycine, alanine, β-alanine or valine. The amino acid can be the L-isomer, the D-isomer or a D/L (e.g., racemic) mixture. In certain embodiments, the amino acid is the L-isomer. In other embodiments, the amino acid is the D-isomer. To control the release and improve the safety of an uncoupler with a lower safety index (e.g., DNP), the promoiety can contain a D-amino acid, which would slow down the prodrug's transport through peptide transporters and slow down enzymatic hydrolysis of the amino acid ester bond in enterocytes and the blood and thereby lower the Cₘₐₓ of the uncoupler. An amino acid with a sterically bulkier group at the alpha position (e.g., valine or isoleucine) would also slow down chemical or enzymatic hydrolysis of the amino acid ester bond. Each amino acid of a promoiety containing a dipeptide or tripeptide independently can be any amino acid described in this paragraph. In certain embodiments, each amino acid of a promoiety containing a dipeptide or tripeptide independently is glycine, alanine or valine.

In still further embodiments, prodrugs of uncouplers having at least one aromatic hydroxyl (e.g., phenolic) group are phosphate esters or phosphate acetal esters at one or more, or all, aromatic hydroxyl (e.g., phenolic) group(s). Phosphate esters or phosphate acetal esters can be targeted to mitochondria via phosphate transporters. In some embodiments, each phosphate ester or phosphate acetal ester promoiety independently has the formula —P(═O)(OR¹)(OR²) or —CHR³OP(═O)(OR¹)(OR²), respectively, wherein R³ is hydrogen or methyl, and R¹ and R² independently are metal cation (M⁺), hydrogen, alkyl, cycloalkyl or aryl, each of which can optionally be substituted, or R¹ and R² together with the oxygen atoms to which they are attached and the phosphorous atom form a heterocyclic ring. In certain embodiments, R¹ and R² independently are: 1) Na⁺ or K⁺; 2) hydrogen; 3) linear or branched C₁-C₆ alkyl (e.g., methyl, ethyl, n-propyl, isopropyl, n-butyl, isobutyl, sec-butyl, tert-butyl, n-pentyl or n-hexyl), which can optionally be substituted with M/P-EG-OMe, —O—(C₁-C₄ alkyl) or phenyl (e.g., R¹ or/and R² are benzyl); 4) C₃-C₆ cycloalkyl (e.g., cyclopropyl, cyclobutyl, cyclopentyl or cyclohexyl); or 5) C₆-C₁₀ aryl (e.g., phenyl or naphthyl), which can optionally be substituted with one or more substituents selected from —F, —Cl, —Br, linear or branched C₁-C₄ alkyl, —CF₃, —OH, —O-(linear or branched C₁-C₄ alkyl), —O(CH₂)₂₋₄Z, M/P-EG-OH, M/P-EG-OMe, —NH₂, —NH(C₁-C₄ alkyl), and —N(C₁-C₄ alkyl)₂, wherein Z is —OH, —O-(linear or branched C₁-C₄ alkyl), —NH₂, —NH(C₁-C₄ alkyl), —N(C₁-C₄ alkyl)₂, or —N-heterocyclyl (e.g., —N-aziridine, —N-azetidine, —N-pyrrolidine, —N-piperidine, —N-morpholine, —N-piperazine or —N-piperazine-N-Me). In other embodiments, R and R² together with the oxygen atoms to which they are attached and the phosphorous atom form a 5- or 6-membered ring (e.g., a 1,3,2-dioxaphospholanyl or 1,3,2-dioxaphosphinanyl ring). To control the release and improve the safety of an uncoupler with a lower safety index (e.g., DNP), the phosphate ester or phosphate acetal ester promoiety can have greater steric hindrance near the —P(═O)—O-uncoupler or —P(═O)—OCHR³—O-uncoupler phosphate ester bond (e.g., R¹ and R² are isopropyl, tert-butyl, 2,6-dimethylphenyl, 2,6-di-isopropylphenyl or 2,6-di-tert-butylphenyl) or/and R³ can be methyl, which would slow down chemical or enzymatic hydrolysis of that phosphate ester bond in the body and thereby lower the Cₘₐₓ of the uncoupler.

In other embodiments, prodrugs of uncouplers having at least one aromatic hydroxyl (e.g., phenolic) group are carbonates at one or more, or all, aromatic hydroxyl (e.g., phenolic) group(s). In some embodiments, each carbonate promoiety independently has the formula —C(═O)OR, wherein R is alkyl, cycloalkyl or aryl, each of which can optionally be substituted. In certain embodiments, R is: 1) linear or branched C₁-C₆ alkyl (e.g., methyl, ethyl, n-propyl, isopropyl, n-butyl, isobutyl, sec-butyl, tert-butyl, n-pentyl or n-hexyl), which can optionally be substituted with —OH, —O—(C₁-C₄ alkyl), M/P-EG-OH, M/P-EG-OMe, —NH₂, —NH(C₁-C₄ alkyl), —N(C₁-C₄ alkyl)₂, or —NHC(═O)—(C₁-C₄ alkyl); 2) C₃-C₁₀ cycloalkyl (e.g., cyclopropyl, cyclobutyl, cyclopentyl, cyclohexyl, cycloheptyl, cyclo-octyl, norbornyl or adamantyl); or 3) C₆-C₁₀ aryl (e.g., phenyl or naphthyl), which can optionally be substituted with one or more substituents selected from —F, —Cl, —Br, linear or branched C₁-C₄ alkyl, —CF₃, —OH, —O-(linear or branched C₁-C₄ alkyl), —O(CH₂)₂₋₄Z, M/P-EG-OH, M/P-EG-OMe, —NH₂, —NH(C₁-C₄ alkyl), and —N(C₁-C₄ alkyl)₂, wherein Z is —OH, —O-(linear or branched C₁-C₄ alkyl), —NH₂, —NH(C₁-C₄ alkyl), —N(C₁-C₄ alkyl)₂, or —N-heterocyclyl (e.g., —N-aziridine, —N-azetidine, —N-pyrrolidine, —N-piperidine, —N-morpholine, —N-piperazine or —N-piperazine-N-Me). In general, carbonates are more stable to chemical or enzymatic hydrolysis than esters. Where R is —(CH₂)₂X or —(CH₂)₃X and X is —OH or —NH₂, the free hydroxyl or amino group could potentially undergo relatively slow self-cleavage of the carbonate bond under basic physiological conditions (e.g., about pH 7.4) to release the parent uncoupler and a cyclic carbonate or carbamate. To control the release and improve the safety of an uncoupler with a lower safety index (e.g., DNP), the carbonate promoiety can have greater steric hindrance adjacent to the carbonate bond (e.g., R is isopropyl, tert-butyl, norbornyl, adamantyl, 2,6-dimethylphenyl, 2,6-di-isopropylphenyl or 2,6-di-tert-butylphenyl), which would slow down chemical or enzymatic hydrolysis of the carbonate bond in the body and thereby lower the Cₘₐₓ of the uncoupler.

In still other embodiments, prodrugs of uncouplers having at least one aromatic hydroxyl (e.g., phenolic) group are carbamates at one or more, or all, aromatic hydroxyl (e.g., phenolic) group(s). In some embodiments, each carbamate promoiety independently has the formula —C(═O)NR¹R², wherein 1) R¹ and R² independently are hydrogen, alkyl or cycloalkyl, either of which can optionally be substituted; 2) R¹ is hydrogen or methyl and R² is alkyl or cycloalkyl, either of which can optionally be substituted; or 3) R¹ and R² together with the nitrogen atom to which they are attached form a heterocyclyl group that can optionally be substituted. In certain embodiments, R² is: 1) linear or branched C₁-C₆ alkyl (e.g., methyl, ethyl, n-propyl, isopropyl, n-butyl, isobutyl, sec-butyl, tert-butyl, n-pentyl or n-hexyl), which can optionally be substituted with —OH, —O—(C₁-C₄ alkyl), M/P-EG-OH, M/P-EG-OMe, —NH₂, —NH(C₁-C₄ alkyl), —N(C₁-C₄ alkyl)₂, or —NHC(═O)—(C₁-C₄ alkyl); or 2) C₃-C₁₀ cycloalkyl (e.g., cyclopropyl, cyclobutyl, cyclopentyl, cyclohexyl, cycloheptyl, cyclo-octyl, norbornyl or adamantyl). In other embodiments, R¹ and R² together with the nitrogen atom to which they are attached form a 3-8-membered heterocyclyl group {e.g., —NR¹R² is —N-aziridine, —N-azetidine, —N-pyrrolidine, —N-piperidine, —N-morpholine, —N-piperazine, —N-piperazine-N-[C₁-C₄ alkyl or —C(═O)—(C₁-C₄ alkyl)], —N-azepane or —N-azocane}. In general, carbamates are more stable to chemical or enzymatic hydrolysis than carbonates. For example, carbamate prodrugs of DNP (e.g., —NR¹R² is —N-piperidine, —N-morpholine, —N-piperazine or —N-piperazine-N-Me) can be slowly hydrolyzed to DNP in the blood to provide slow release and a lower Cₘₐₓ of DNP. Where R² is —(CH₂)₂X or —(CH₂)₃X and X is —OH or —NH₂, the free hydroxyl or amino group could potentially undergo slow self-cleavage of the carbamate bond under basic physiological conditions (e.g., about pH 7.4) to release the parent uncoupler and a cyclic carbamate or urea.

In additional embodiments, prodrugs of uncouplers having at least one aromatic hydroxyl (e.g., phenolic) group are ethers at one or more, or all, aromatic hydroxyl (e.g., phenolic) group(s). The cytochrome P450 system in the liver can oxidize an ether to a hemiacetal or hemiketal that degrades to the parent drug and an aldehyde or ketone, or to an ester that is hydrolyzed to the parent drug and a carboxylic acid. Oxidative metabolism of an ether prodrug by cytochrome P450 in the liver can provide controlled or slow release of the parent drug and hence lower the Cₘₐₓ and improve the safety of the drug. Moreover, use of an ether prodrug can promote targeting of the parent drug to the liver for treatment of, e.g., a liver or metabolic disorder. In some embodiments, the carbon atom of the ether promoiety that is attached to (alpha to) the ether oxygen atom has at least two hydrogen atoms. In some embodiments, the ether promoiety is a linear C₁-C₆ alkyl (e.g., methyl, ethyl, n-propyl, n-butyl, n-pentyl or n-hexyl) that can optionally be substituted, such as with a group that increases aqueous solubility (e.g., —OH or —NH₂) or a phenyl group that can optionally be substituted. The phenyl group can optionally be substituted with, e.g., one or more substituents selected from —F, —Cl, —Br, linear or branched C₁-C₄ alkyl, —CF₃, —OH, —O-(linear or branched C₁-C₄ alkyl), —O(CH₂)₂₋₄Z, M/P-EG-OH, M/P-EG-OMe, —NH₂, —NH(C₁-C₄ alkyl), and —N(C₁-C₄ alkyl)₂, wherein Z is —OH, —O-(linear or branched C₁-C₄ alkyl), —NH₂, —NH(C₁-C₄ alkyl), —N(C₁-C₄ alkyl)₂, or —N-heterocyclyl (e.g., —N-aziridine, —N-azetidine, —N-pyrrolidine, —N-piperidine, —N-morpholine, —N-piperazine or —N-piperazine-N-Me). In certain embodiments, the ether promoiety is methyl. In other embodiments, the ether promoiety is benzyl whose phenyl group can optionally be substituted. In some embodiments, the ether prodrug has bipartite structure A or tripartite structure B below, whose oxidative metabolism by cytochrome P450 yields two or three molecules of the uncoupler, respectively. The benzylic hydroxyl group in structure A increases aqueous solubility of the ether prodrug, but it can be derivatized as another group (e.g., —O-alkyl such as —OMe) if desired. Other types of prodrugs (e.g., esters, amino acid esters, phosphate esters, carbonates and carbamates) corresponding to bipartite structure A and tripartite structure B can also be made and utilized.

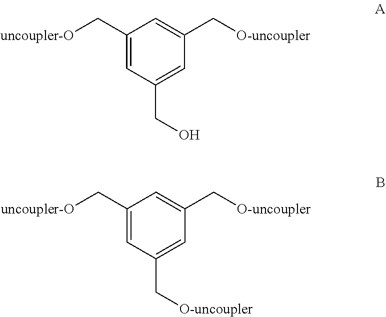

In certain embodiments, prodrugs of uncouplers having at least one aromatic hydroxyl (e.g., phenolic) group are prodrugs of BHA, BHT, 2-nitrophenol, 3-nitrophenol, 4-nitrophenol, 2,3-DNP, 2,4-DNP (commonly known as DNP), 2,5-DNP, 2,6-DNP, 3,4-DNP, 3,5-DNP, niclosamide, tizoxanide, oxyclozanide, (+)-usnic acid and NNC-0112-0000-2604.

As mitochondria (viz., the mitochondrial matrix) are the only organelle with a basic pH environment, lipophilic cations preferentially target mitochondria. Mitochondria-targeted uncouplers include without limitation

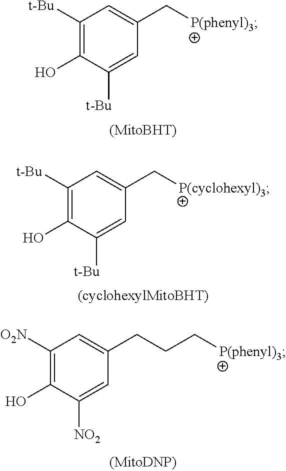

MitoQₙ series

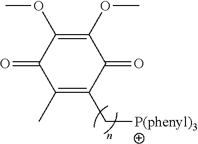

wherein n is 1, 2, 3, 4, 5, 6, 7, 8, 9, 10, 11, 12, 13, 14, 15 or 16; alkyTPP series H₃C(CH₂)ₘP⁺ (phenyl)₃ wherein m is 0, 1, 2, 3, 4, 5, 6, 7, 8, 9, 10, 11, 12, 13, 14 or 15; cationic cyanine dyes; and analogs, derivatives, prodrugs, metabolites, salts (e.g., phosphate salts), and controlled-release, slow-release and sustained-release forms (including liposomes, cholestosomes and lipid, polymeric or dendrimeric nanoparticles encapsulating the uncouplers) thereof. In certain embodiments, the counterion of a cationic uncoupler is phosphate. Phosphate can promote transport of the cationic uncoupler into mitochondria via phosphate permeases/transporters.

Mitochondrial uncoupling can also be induced by metal cations such as Ag⁺ and Cd²⁺ and metal cation complexes such as copper(II) complexes of 1,10-phenanthroline-derived ligands. Without intending to be bound by theory, metal cations may induce uncoupling by a non-protonophoric mechanism whereby electrophoretic transfer of the metal cations into the negatively charged environment of the mitochondrial matrix dissipates the electrochemical gradient across the MIM and hence the proton motive force.

In some embodiments, the uncoupler is one providing mild uncoupling. Mild uncoupling can be achieved by, e.g., use of a particular uncoupler, use of a prodrug of an uncoupler (which can lower the peak plasma concentration of the uncoupler, for example), targeting of an uncoupler to specific tissue(s) or organ(s) (e.g., white adipose tissue or/and the liver to achieve desired metabolic effects of uncoupling and to minimize side effects), administration of a low dose of an uncoupler (e.g., as a bolus or via a controlled-, slow- or sustained-release formulation), or use of an uncoupler in combination with one or more nicotinyl riboside compounds, or any combination thereof. In certain embodiments, an uncoupler alone, or in combination with one or more nicotinyl riboside compounds (e.g., one or more of NR, NRH, NRTA, NRHTA and NR/NAR derivatives disclosed herein), reduces ATP production or level (e.g., mitochondrial ATP production or total cellular ATP level, such as that in target cells) by no more than about 10% or 5% in vitro, ex vivo or in vivo, or by no more than about 10%, 8%, 6% or 4% in vitro, ex vivo or in vivo, which can depend on, e.g., the uncoupler's potency or/and concentration. In some embodiments, an uncoupler alone, or in combination with one or more nicotinyl riboside compounds, reduces ATP production or level (e.g., mitochondrial ATP production or total cellular ATP level, such as that in target cells) by about 1 or 2% to about 10%, or by about 1 or 2% to about 5% or by about 5-10%, in vitro, ex vivo or in vivo. Mild uncoupling, whether by use of an uncoupler alone or in combination with one or more nicotinyl riboside compounds, can avoid ATP depletion or excessive reduction of cellular ATP level in part by stimulating mitochondrial biogenesis and hence ATP production by an increased number of mitochondria.

Modest or moderate reduction of ATP production or level by mild uncoupling has beneficial effects. For example, reduced ATP level enhances the TCA cycle flux to compensate for reduced ATP level. As another example, reduced ATP level activates AMPK, which is a sensor of reduced ATP level. AMPK activation promotes a metabolic shift from glycolysis to fatty acid/beta oxidation for ATP generation and activation of sirtuins (e.g., sirtuin-1) through AMPK-sirtuin cross-talk.

Use of a nicotinyl riboside compound restores NAD⁺ consumed by the uncoupling-induced TCA cycle and preserves NAD⁺ level to sustain TCA cycle flux and ETC activity, thereby avoiding significant reduction of mitochondrial membrane potential and allowing sustained mild uncoupling. Preservation or increase of NAD⁺ level (e.g., total cellular NAD⁺ level) also preserves or increases the activity of NAD⁺-dependent enzymes (e.g., sirtuins and PARPs) and thereby maintains or enhances cellular health and function. In some embodiments, one or more nicotinyl riboside compounds (e.g., one or more of NR, NRH, NRTA, NRHTA and NR/NAR derivatives disclosed herein) in combination with an uncoupler (e.g., one providing mild uncoupling) increase NAD⁺ level (e.g., total cellular NAD⁺ level, such as that in target cells) by at least about 10%, 20%, 30%, 50%, 100%, 150% or 200% in vitro, ex vivo or in vivo.

In certain embodiments, one or more nicotinyl riboside compounds in combination with an uncoupler increase NAD⁺ level (e.g., total cellular NAD⁺ level, such as that in target cells) by at least about 20%, 50% or 100% in vitro, ex vivo or in vivo.

Use of a nicotinyl riboside compound also increases the NAD⁺/NADH ratio. The NAD⁺/NADH ratio regulates important cellular pathways (e.g., the TCA cycle) and covalent modification (e.g., phosphorylation or succinylation) of enzymes, and low NAD⁺/NADH ratios are associated with, e.g., metabolic disorders (e.g., diabetes and NASH). Increased NAD⁺/NADH ratio promotes a favorable redox state for sustained or enhanced TCA cycle flux and improves cellular health and function. In some embodiments, one or more nicotinyl riboside compounds (e.g., one or more of NR, NRH, NRTA, NRHTA and NR/NAR derivatives disclosed herein) in combination with an uncoupler (e.g., one providing mild uncoupling) increase NAD⁺/NADH ratio (e.g., cellular NAD⁺/NADH ratio or blood/plasma/serum NAD⁺/NADH ratio) by at least about 10%, 20%, 30%, 50%, 100%, 150% or 200% in vitro, ex vivo or in vivo. In certain embodiments, one or more nicotinyl riboside compounds in combination with an uncoupler increase NAD⁺/NADH ratio (e.g., cellular NAD⁺/NADH ratio or blood/plasma/serum NAD⁺/NADH ratio) by at least about 20%, 50% or 100% in vitro, ex vivo or in vivo.

Increasing the NAD⁺/NADH ratio is equivalent to reducing the NADH/NAD⁺ ratio. The redox status of the cell plays an important role in maintaining cellular integrity and homeostatic functions. For example, elevated NADH/NAD⁺ ratios are associated with reductive stress and reductive stress in the liver is associated with metabolic disorders such as insulin resistance, and decreased reductive stress improves insulin homeostasis. Alpha-hydroxybutyrate (AHB) is a marker of reductive stress, and elevated plasma/serum AHB level is associated with impaired glucose tolerance and insulin resistance. Elevated plasma/serum AHB level is also associated with increased lactate production, mitochondrial dysfunction, and reduced TCA cycle flux. In some embodiments, one or more nicotinyl riboside compounds (e.g., one or more of NR, NRH, NRTA, NRHTA and NR/NAR derivatives disclosed herein) in combination with an uncoupler (e.g., one providing mild uncoupling) reduce NADH/NAD⁺ ratio (e.g., cellular NADH/NAD⁺ ratio or blood/plasma/serum NADH/NAD⁺ ratio) by at least about 10%, 20%, 30%, 50%, 100%, 150% or 200% in vitro, ex vivo or in vivo. In certain embodiments, one or more nicotinyl riboside compounds in combination with an uncoupler reduce NADH/NAD⁺ ratio (e.g., cellular NADH/NAD⁺ ratio or blood/plasma/serum NADH/NAD⁺ ratio) by at least about 20%, 50% or 100% in vitro, ex vivo or in vivo. In further embodiments, one or more nicotinyl riboside compounds (e.g., one or more of NR, NRH, NRTA, NRHTA and NR/NAR derivatives disclosed herein) in combination with an uncoupler (e.g., one providing mild uncoupling) reduce AHB level (e.g., blood/plasma/serum AHB level) by at least about 10%, 20%, 30%, 50%, 100%, 150% or 200% in vitro, ex vivo or in vivo. In certain embodiments, one or more nicotinyl riboside compounds in combination with an uncoupler reduce AHB level (e.g., blood/plasma/serum AHB level) by at least about 20%, 50% or 100% in vitro, ex vivo or in vivo.

Use of an uncoupler providing mild uncoupling in combination with one or more nicotinyl riboside compounds can provide mild or moderate reduction in ATP level while preserving or enhancing NAD⁺ level (e.g., in mitochondria, the cytosol or/and the nucleus, such as total cellular NAD⁺ level) and the NAD⁺/NADH ratio, which maintains or improves cellular and mitochondrial function and health/viability. Sustained mild uncoupling through the use of an uncoupler with one or more nicotinyl riboside compounds provides beneficial effects of calorie restriction described herein, including activation of AMPK and sirtuins, increased fuel (e.g., fatty acids and glucose) oxidation due to enhanced TCA cycle flux under a favorable redox state, and reduced oxidative stress. Improved cellular function includes improved metabolic function.

In certain embodiments, the uncoupler providing mild uncoupling is nitazoxanide (NTZ), tizoxanide, niclosamide (e.g., niclosamide ethanolamine or DK-520) or oxyclozanide, or an analog, a derivative, a prodrug, a metabolite, a salt, a targeted form, or a controlled-, slow- or sustained-release form thereof. NTZ is approved for treatment of various helminthic, protozoal and viral infections. NTZ has high oral bioavailability and can reach the CNS. Unlike medium-strength uncouplers (e.g., DNP) and strong uncouplers (e.g., FCCP), the uncoupler NTZ has a good safety profile—4 g of oral NTZ does not cause any significant adverse effect in healthy adults. Like NTZ, niclosamide is approved as an antihelminthic and can have a sufficiently wide therapeutic window. Analogs and prodrugs of niclosamide, and prodrugs of analogs of niclosamide, that provide similar uncoupling activity as niclosamide include without limitation Compound Nos. 3,9-12,16-19 and 31-36 (Compound No. 32 is DK-520) disclosed in R. Mook et al., Bioorg. Med. Chem., 23:5829-5838 (2015). Another antihelminthic that acts at least in part by uncoupling of oxidative phosphorylation in the target parasites is oxyclozanide.

In further embodiments, the uncoupler providing mild uncoupling is an uncoupler with a wide dynamic range. The dynamic range is the ratio of the concentration causing maximum uncoupling to the concentration providing the minimum measurable uncoupling. An uncoupler with a wide dynamic range causes uncoupling that increases slightly, or very slightly, as its concentration rises. In certain embodiments, the dynamic range is at least about 10⁶ in vitro or in vivo. Uncouplers providing mild uncoupling can achieve a wide dynamic range via, e.g., a high-affinity interaction with the mitochondrial adenine nucleotide translocase (ANT) which causes limited but appreciable uncoupling at very low uncoupler concentrations, along with more conventional uncoupling at much higher uncoupler concentrations. The ANT is a MIM protein that imports ADP and exports ATP during oxidative phosphorylation. The ANT causes basal proton leak, and uncoupling at the ANT is not by the conventional weak acid/anion cycling mechanism since it is also caused by substituted triphenylphosphonium compounds (e.g., MitoQₙ series and alkylTPP series) that have no anionic form and cannot protonate/de-protonate. Other uncouplers that may act at the ANT include cationic cyanine dyes and copper(II) complexes of 1,10-phenanthroline-derived ligands. Without intending to be bound by theory, uncoupling at the ANT may be due to hydrophobic binding of the uncoupler to the ANT, which allosterically induces proton leak via the ANT into the mitochondrial matrix. An alternative, non-protonophoric mechanism of uncoupling by hydrophobic cations is electrophoretic translocation of the hydrophobic cations into the negatively charged environment of the mitochondrial matrix, which dissipates the mitochondrial membrane potential and hence the proton motive force. Covalent attachment of an uncoupler to a mitochondrially targeted hydrophobic cation such as a triphenylphosphonium moiety sensitizes the uncoupler to the mitochondrial membrane potential and increases the effect. An uncoupler with a wide dynamic range can have a sufficiently high therapeutic index (or a sufficiently wide therapeutic/safety window).

Uncouplers providing mild uncoupling and having a wide dynamic range (e.g., at least about 106 in vitro or in vivo) include, but are not limited to, benzoic acid, BHA, BHT, NNC-0112-0000-2604, NNC-0112-0000-0376, MitoBHT, cyclohexylMitoBHT, MitoDNP, MitoQio and decylTPP. Such uncouplers can have a wide dynamic range in, e.g., metabolically active tissues and organs such as the liver and tissues and organs involved in energy metabolism relevant to body weight such as skeletal muscles. Accumulation of mitochondria-targeted uncouplers in mitochondria is driven in part by the mitochondrial membrane potential. Uncoupling by mitochondria-targeted uncouplers can be self-limiting because their uncoupling decreases the membrane potential, which reduces their accumulation in mitochondria and hence attenuates uncoupling, and thereby results in a wide dynamic range. In certain embodiments, the uncoupler is BHT or MitoBHT, or an analog, a derivative, a prodrug, a metabolite, a salt, a targeted form, or a controlled-, slow- or sustained-release form thereof.

Self-limiting mitochondrial uncoupling can also be promoted by use of a more weakly acidic uncoupler, such as an uncoupler with a pKₐ of about 7-10 or 7-8. A more weakly acidic uncoupler can also have reduced off-target effects on non-mitochondrial organelles that have a lower pH (e.g., reduced depolarization of the plasma membrane), which suppresses ionization of the weak-acid group of the uncoupler and hence cycling of the protonated and de-protonated forms of the uncoupler.

Cyanotriazole uncouplers can also have a sufficiently wide therapeutic window. Cyanotriazole uncouplers include without limitation those disclosed in US 2016/0229816 by S. Sato et al., including Example Nos. 1-150, 220, 275, 276, 298, 423, 504, 600, 607, 610, 613, 617, 620, 623, 627, 639, 640, 644-646, 649, 657, 659, 663, 718, 790, 807, 931, 934, 944, 989, 1004, 1017, 1018, 1248, 1505, 1573, 1672, 1676, 1806, 1808 and 1810-1831, and tautomers and pharmaceutically acceptable salts thereof. In some embodiments, the cyanotriazole uncoupler is selected from Example Nos. 11, 14,15,21-23, 29, 41, 47, 49, 50-52, 55, 60, 70, 75, 77-79, 90, 92, 98, 100, 108, 120, 122, 137, 146, 147, 220, 275, 276, 298, 423, 504, 600, 607, 610, 613, 617, 620, 623, 627, 639, 640, 644-646, 649, 657, 659, 663, 718, 790 807, 931, 934, 944, 989, 1004, 1017, 1018, 1248, 1505, 1573, 1672, 1676, 1806, 1808 and 1810-1831 in US 2016/0229816, and tautomers and pharmaceutically acceptable salts thereof. In narrower embodiments, the cyanotriazole uncoupler is selected from Example Nos. 22, 23, 41, 50, 52, 55, 60, 70, 90, 92, 100, 108, 120, 137, 607, 613, 617, 620, 623, 644, 645, 649, 657, 659, 663, 718, 944, 989, 1017, 1505 and 1573 in US 2016/0229816, and tautomers and pharmaceutically acceptable salts thereof. In even narrower embodiments, the cyanotriazole uncoupler is selected from Example Nos. 23, 41, 60, 70, 92, 137, 613, 620, 644, 659, 663, 718 and 1573 in US 2016/0229816, and tautomers and pharmaceutically acceptable salts thereof. In certain embodiments, the cyanotriazole uncoupler is Example No. 60 or 92 in US 2016/0229816, or a tautomer or pharmaceutically acceptable salt thereof. Example No. 92 is OPC-163493 and preferentially localizes in the liver and kidneys. The structure of some cyanotriazole uncouplers disclosed in US 2016/0229816, with only one tautomer shown for simplicity, is shown in Table 3.

TABLE 3
Representative cyanotriazole uncouplers disclosed in US 2016/0229816

| Ex. No. | |
|---|---|
| 23 | 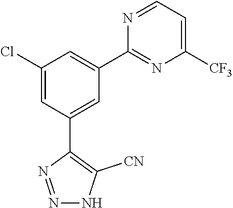 [SMILES: N#Cc1[nH]nnc1-c1cc(Cl)cc(-c2nccc(C(F)(F)F)n2)c1] |
| 41 | 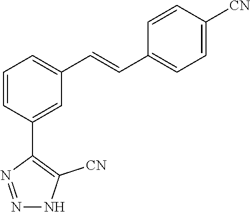 [SMILES: N#Cc1ccc(/C=C/c2cccc(-c3nn[nH]c3C#N)c2)cc1] |
| 613 | 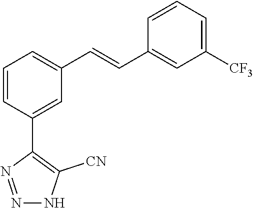 [SMILES: N#Cc1[nH]nnc1-c1cccc(/C=C/c2cccc(C(F)(F)F)c2)c1] |
| 620 | 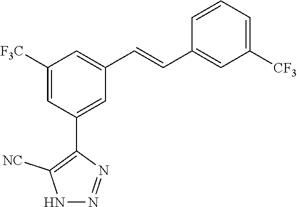 [SMILES: N#Cc1[nH]nnc1-c1cc(/C=C/c2cccc(C(F)(F)F)c2)cc(C(F)(F)F)c1] |
| 60 | 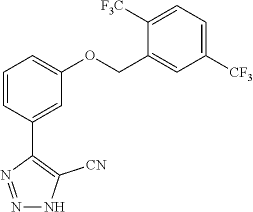 [SMILES: N#Cc1[nH]nnc1-c1cccc(OCc2cc(C(F)(F)F)ccc2C(F)(F)F)c1] |
| 70 | 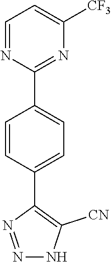 [SMILES: N#Cc1[nH]nnc1-c1ccc(-c2nccc(C(F)(F)F)n2)cc1] |
| 92 | 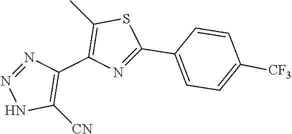 [SMILES: Cc1sc(-c2ccc(C(F)(F)F)cc2)nc1-c1nn[nH]c1C#N] |
| 137 | 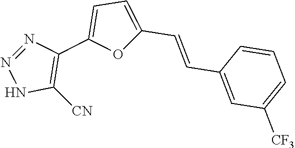 [SMILES: N#Cc1[nH]nnc1-c1ccc(/C=C/c2cccc(C(F)(F)F)c2)o1] |
| 644 | 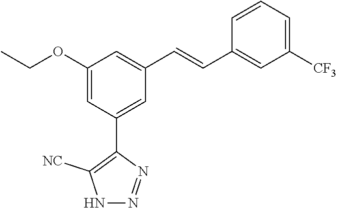 [SMILES: CCOc1cc(/C=C/c2cccc(C(F)(F)F)c2)cc(-c2nn[nH]c2C#N)c1] |
| 659 | 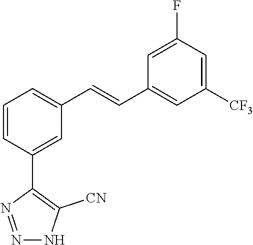 [SMILES: N#Cc1[nH]nnc1-c1cccc(/C=C/c2cc(F)cc(C(F)(F)F)c2)c1] |
| 663 | 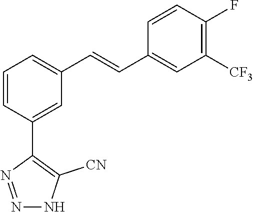 [SMILES: N#Cc1[nH]nnc1-c1cccc(/C=C/c2ccc(F)c(C(F)(F)F)c2)c1] |
| 718 | 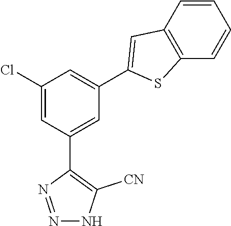 [SMILES: N#Cc1[nH]nnc1-c1cc(Cl)cc(-c2cc3ccccc3s2)c1] |
| 1573 | 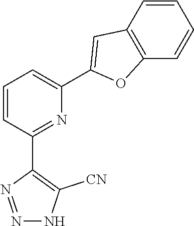 [SMILES: N#Cc1[nH]nnc1-c1cccc(-c2cc3ccccc3o2)n1] |

Sulfonanilide uncouplers disclosed in WO2019/226490 include Example Nos. 1 through 248, and pharmaceutically acceptable salts thereof. In some embodiments, the sulfonanilide uncoupler is selected from

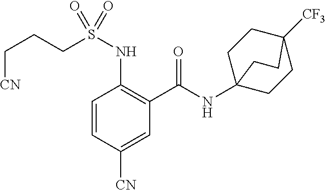 [SMILES: N#CCCCS(=O)(=O)Nc1ccc(C#N)cc1C(=O)NC12CCC(C(F)(F)F)(CC1)CC2]

and Example Nos. 105, 134, 140, 157, 162, 165, 173, 183, 185, 193, 204, 229, 230, 239, 240 and 245 in WO2019/226490, and pharmaceutically acceptable salts thereof. In further embodiments, the sulfonanilide uncoupler is Example No. 134, 157 or 230 in WO2019/226490. In certain embodiments, the sulfonanilide uncoupler is Example No. 134 in WO2019/226490, which is N-(4-cyanobicyclo[2.2.2]octan-1-yl)-4-fluoro-2-[(3,3,3-trifluoropropyl)sulfonamido]benzamide and preferentially distributes to the liver. The structure of some sulfonanilide uncouplers disclosed in WO2019/226490 is shown in Table 4.

TABLE 4
Representative sulfonanilide uncouplers disclosed in WO 2019/226490

| Ex. No. | |
|---|---|
| 140 | 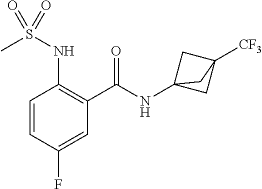 [SMILES: CS(=O)(=O)Nc1ccc(F)cc1C(=O)NC12CC(C(F)(F)F)(C1)C2] |
| 157 | 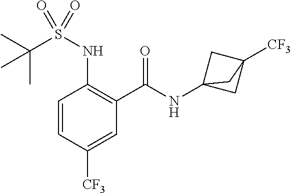 [SMILES: CC(C)(C)S(=O)(=O)Nc1ccc(C(F)(F)F)cc1C(=O)NC12CC(C(F)(F)F)(C1)C2] |
| 162 | 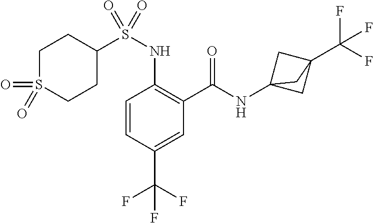 [SMILES: O=C(NC12CC(C(F)(F)F)(C1)C2)c1cc(C(F)(F)F)ccc1NS(=O)(=O)C1CCS(=O)(=O)CC1] |
| 105 | 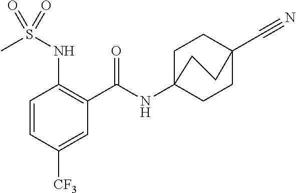 [SMILES: CS(=O)(=O)Nc1ccc(C(F)(F)F)cc1C(=O)NC12CCC(C#N)(CC1)CC2] |
| 134 | 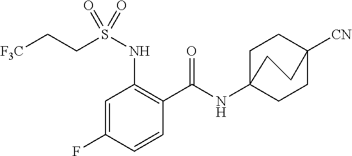 [SMILES: N#CC12CCC(NC(=O)c3ccc(F)cc3NS(=O)(=O)CCC(F)(F)F)(CC1)CC2] |
| 173 | 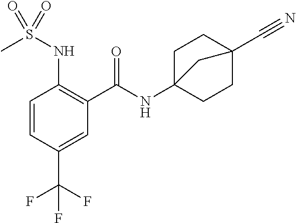 [SMILES: CS(=O)(=O)Nc1ccc(C(F)(F)F)cc1C(=O)NC12CCC(C#N)(CC1)C2] |
| 165 | 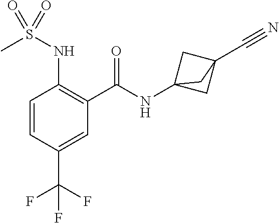 [SMILES: CS(=O)(=O)Nc1ccc(C(F)(F)F)cc1C(=O)NC12CC(C#N)(C1)C2] |
| 193 | 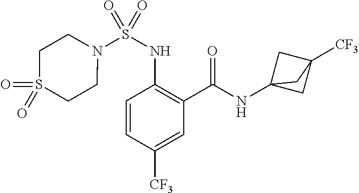 [SMILES: O=C(NC12CC(C(F)(F)F)(C1)C2)c1cc(C(F)(F)F)ccc1NS(=O)(=O)N1CCS(=O)(=O)CC1] |
| 245 | 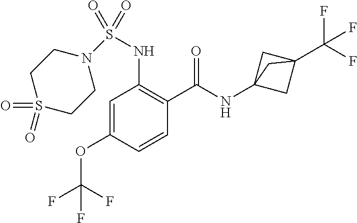 [SMILES: O=C(NC12CC(C(F)(F)F)(C1)C2)c1ccc(OC(F)(F)F)cc1NS(=O)(=O)N1CCS(=O)(=O)CC1] |
| 229 | 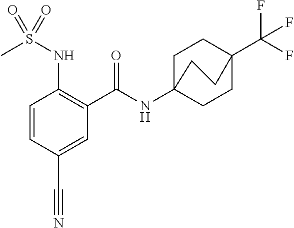 [SMILES: CS(=O)(=O)Nc1ccc(C#N)cc1C(=O)NC12CCC(C(F)(F)F)(CC1)CC2] |
| 239 | 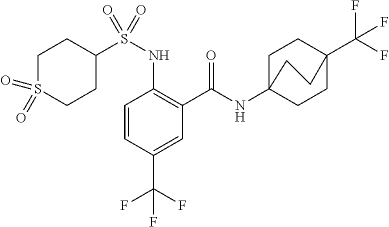 [SMILES: O=C(NC12CCC(C(F)(F)F)(CC1)CC2)c1cc(C(F)(F)F)ccc1NS(=O)(=O)C1CCS(=O)(=O)CC1] |
| 183 | 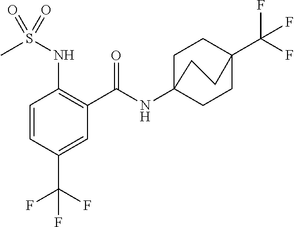 [SMILES: CS(=O)(=O)Nc1ccc(C(F)(F)F)cc1C(=O)NC12CCC(C(F)(F)F)(CC1)CC2] |
| 185 | 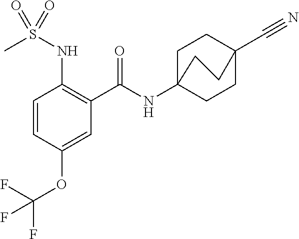 [SMILES: CS(=O)(=O)Nc1ccc(OC(F)(F)F)cc1C(=O)NC12CCC(C#N)(CC1)CC2] |
| 204 | 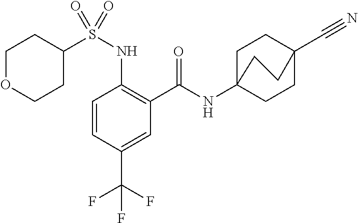 [SMILES: N#CC12CCC(NC(=O)c3cc(C(F)(F)F)ccc3NS(=O)(=O)C3CCOCC3)(CC1)CC2] |
| 230 | 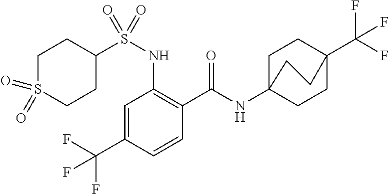 [SMILES: O=C(NC12CCC(C(F)(F)F)(CC1)CC2)c1ccc(C(F)(F)F)cc1NS(=O)(=O)C1CCS(=O)(=O)CC1] |
| 240 | 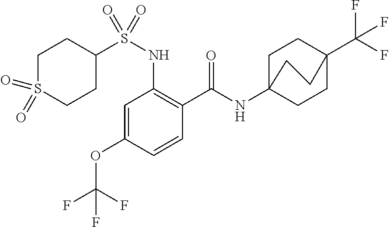 [SMILES: O=C(NC12CCC(C(F)(F)F)(CC1)CC2)c1ccc(OC(F)(F)F)cc1NS(=O)(=O)C1CCS(=O)(=O)CC1] |

Other uncouplers that can have a sufficiently wide therapeutic window include without limitation phenol, phenols having one or more alkyl groups, phenols having one or more electron-withdrawing atoms or groups, phenols having one or more alkyl groups and one or more electron-withdrawing atoms or groups, naturally occurring phenols, retinoids, NSAIDs, antidiabetic agents, ellipticine, usnic acid (e.g., (+)-usnic acid), BAM15, C4R1, and salts thereof.

In some embodiments, the uncoupler is provided by way of a controlled-, slow- or sustained-release composition. Such a composition can be administered orally or parenterally (e.g., intravenously, subcutaneously, intramuscularly, intrathecally or topically). Such a composition can lower the peak plasma concentration (Cₘₐₓ) of the uncoupler and increase its area under the curve (AUC) plasma concentration over an extended period of time, thereby improving its safety and efficacy. Release of a low but effective amount of even an uncoupler with an otherwise narrow therapeutic window (e.g., DNP) over a prolonged period of time can significantly widen its therapeutic window and bring about mild uncoupling. In certain embodiments, such a composition releases the uncoupler over at least about 12 hr, 24 hr, 48 hr or 72 hr. In some embodiments, such a composition has a controlled-, slow- or sustained-release coating (e.g., polymeric coating) which degrades over time or/and through which (e.g., through pores created by the polymer in the coating) the uncoupler diffuses over time. In certain embodiments, such a composition comprises an uncoupler (e.g., NTZ, niclosamide, OPC-163493, DNP, BAM15 or Example No. 134 in WO 2019/226490) coated with a controlled-, slow- or sustained-release polymeric coating, such as a coating comprising a hydrophilic polymer and optionally a hydrophobic polymer. In further embodiments, such a composition is in the form of a pellet, particle, bead or sphere containing an uncoupler (e.g., NTZ, niclosamide, OPC-163493, DNP, BAM15 or Example No. 134 in WO 2019/226490) and having a controlled-, slow- or sustained-release polymeric coating, such as a coating comprising a hydrophilic polymer and optionally a hydrophobic polymer. In certain embodiments, such a composition is an oral solid dosage form (e.g., a tablet, capsule or pill) comprising an uncoupler and having a controlled-, slow- or sustained-release polymeric coating. In other embodiments, such a composition is an oral solid dosage form (e.g., a tablet, capsule or pill) comprising a plurality of uncoupler-containing pellets, particles, beads or spheres coated with a controlled-, slow- or sustained-release polymeric coating, wherein the dosage form can optionally have an enteric coating (e.g., Opadry® Enteric [94 Series]). Such compositions, including compositions and oral solid dosage forms comprising an uncoupler and having a controlled-, slow- or sustained-release polymeric coating, pellets/particles/beads/spheres containing an uncoupler and having such a coating, and oral solid dosage forms containing such pellets/particles/beads/spheres, can comprise one or more excipients such as a filler or inert diluent (e.g., lactose, mannitol or microcrystalline cellulose [MCC]) or/and a binding agent (e.g., MCC, hydroxypropyl methyl cellulose [HPMC] or starch). In some embodiments, the controlled-, slow- or sustained-release coating of such compositions comprises one or more hydrophilic polymers selected from hydroxypropyl cellulose (HPC), HPMC, methyl cellulose (MC), ethyl cellulose (EC), and ethyl methyl cellulose (EMC). In certain embodiments, such a coating comprises HPC and EC. In some embodiments, the polymeric coating further comprises a plasticizer (e.g., dibutyl sebacate [DBS]) or/and a lubricant (e.g., magnesium stearate or talc). In certain embodiments, the controlled-, slow- or sustained-release composition comprises or is a pellet, particle, bead or sphere containing an uncoupler (e.g., DNP in a concentration of, e.g., about 1-5% w/w or about 2% or 3% w/w), mannitol (e.g., about 60-70% w/w or about 66% w/w), MCC (e.g., about 25-40% w/w or about 32% w/w) and HPMC (e.g., about 0.1-0.5% w/w or about 0.2% w/w), and having a coating comprising HPC and EC and optionally a plasticizer (e.g., DBS) or/and a lubricant (e.g., talc). The concentration of the uncoupler (e.g., DNP) can also be higher in such a pellet, particle, bead or sphere, such as about 5-10%, 10-15%, 15-20%, 20-25% or 25-30% w/w, or about 12% w/w for example.

In some embodiments, two or more mitochondrial uncouplers are used, optionally in combination with one or more other therapeutic agents described herein (e.g., one or more nicotinyl riboside compounds). One of the uncouplers can enhance the activity of the other uncoupler(s). For example, dodecylTPP can enhance the activity of an uncoupler with an otherwise low therapeutic index such as DNP or FCCP, thereby reducing the effective dose of the other uncoupler and increasing its therapeutic index. In some embodiments, the two or more uncouplers are or comprise a cationic mitochondria-targeted uncoupler and a weak-acid uncoupler (e.g., pKₐ of about 4-10). Interaction of a cationic mitochondria-targeted uncoupler with the anionic form of a weak-acid uncoupler can localize the weak-acid uncoupler to mitochondria and minimize off-target effects (e.g., depolarization of the plasma membrane). In further embodiments, at least one of the two or more mitochondrial uncouplers is an uncoupler providing mild uncoupling.

In view of the beneficial effects of uncoupling, mitochondrial uncouplers can be utilized to treat the diseases/disorders and conditions described herein, including mitochondrial diseases and mitochondria-related diseases and conditions. In some embodiments, the mitochondrial disease is a primary mitochondrial disease. In other embodiments, the mitochondrial disease or the mitochondria-related disease or condition is associated with (e.g., is caused by or results in) secondary mitochondrial dysfunction. To treat a disease or condition described herein, an uncoupler can be used alone or in combination with one or more additional therapeutic agents, such as one or more nicotinyl riboside compounds or/and one or more other therapeutic agents described herein.

In some embodiments, one or more nicotinyl riboside compounds are used in combination with a mitochondrial uncoupler. The use of one or more nicotinyl riboside compounds enhances NAD⁺ levels and the NAD⁺/NADH ratio and hence has beneficial effects associated therewith. Enhanced NAD⁺ levels also permit increased TCA cycle flux and ETC activity and thus sustained uncoupling. In addition, the use of one or more nicotinyl riboside compounds with an uncoupler can significantly increase the therapeutic index of that uncoupler, provide safe and effective, sustained uncoupling, and allow for chronic combination therapy, as described below. Use of one or more nicotinyl riboside compounds in combination with an uncoupler can significantly enhance the safety or/and the efficacy of the uncoupler, or can have synergistic effect(s).

In some embodiments, the one or more nicotinyl riboside compounds are or comprise one or more of NR, NRH, NAR and NARH, or/and one or more NR/NAR derivatives (such as one or more NR/NAR derivatives disclosed herein). In certain embodiments, the one or more nicotinyl riboside compounds are or comprise NR or/and NRH. In other embodiments, the one or more nicotinyl riboside compounds are or comprise nicotinamide riboside triacetate (NRTA, i.e., NR having an acetate group at each of the C-2, C-3 and C-5 positions of riboside), the reduced form of NRTA (NRHTA), nicotinic acid riboside triacetate (NARTA), or the reduced form of NARTA (NARHTA), or any combination thereof.

The use of an uncoupler with a nicotinyl riboside compound improves cellular, mitochondrial and metabolic function. Accordingly, in some embodiments a mitochondrial uncoupler (e.g., one providing mild uncoupling such as NTZ, niclosamide, OPC-163493, controlled-release DNP, BAM15 or Example No. 134 in WO 2019/226490) is used in combination with one or more nicotinyl riboside compounds (e.g., NR or/and NRH, NRTA or/and NRHTA, or one or more NR/NAR derivatives disclosed herein) to treat a metabolic disorder. In certain embodiments, the metabolic disorder is a disorder associated with abnormal or ectopic lipid accumulation or storage (e.g., a lipid storage droplet disorder such as CGI-58 deficiency [Chanarin-Dorfman syndrome], MTP deficiency or ApoB deficiency), lypodystrophy (e.g., congenital or acquired lipodystrophy, partial or generalized lipodystrophy, or severe lipodystrophy, such as HIV-associated lipodystrophy or ART-induced lipodystrophy), obesity, metabolic syndrome, hypercholesterolemia (e.g., familial hypercholesterolemia), insulin resistance, diabetes (e.g., T2D), a liver disorder(e.g., NAFLD, NASH, ALD, ASH or hepatotoxicity), or a lysosomal storage disease (e.g., a lipid storage disorder such as LAL deficiency [including Wolman disease and CESD], Gaucher disease or Niemann-Pick disease). Other metabolic disorders are described elsewhere herein. One or more other therapeutic agents described herein (e.g., an anti-obesity agent, an antihyperlipidemic agent, an antidiabetic agent or an antihypertensive agent, or any combination thereof) can optionally be used in combination with one or more nicotinyl riboside compounds and an uncoupler to treat a metabolic disorder.

For example, an uncoupler providing mild uncoupling can be used with one or more nicotinyl riboside compounds, optionally in conjunction with one or more other therapeutic agents, to treat familial hypercholesterolemia (e.g., homozygous or heterozygous FH). FH can cause severe steatosis and cardiovascular diseases (e.g., atherosclerosis and CAD). The one or more other therapeutic agents can comprise, e.g., an antihyperlipidemic agent, such as a statin (e.g., atorvastatin) or/and an MTTP inhibitor (e.g., lomitapide).

As another example, an uncoupler providing mild uncoupling can be used with one or more nicotinyl riboside compounds, optionally in conjunction with one or more additional therapeutic agents, to treat hepatotoxicity or a complication thereof such as liver failure (e.g., acute liver failure [ALF] or acute-on-chronic liver failure (ACLF]). Hepatotoxicity in general is chemical-induced liver damage and includes drug-induced liver injury (DILI). DILI can cause acute and chronic liver disease, and is responsible for about 50% of ALF cases. ALF is characterized by catastrophic mitochondrial failure and ROS generation leading to massive cell death (often about 70-90% of liver cells die). Chemicals (including medications) that can cause hepatotoxicity (including DILI) include acetaminophen, NSAIDs, glucocorticoids, hydrazine-containing drugs (e.g., isoniazid and iproniazid), antibiotics (e.g., amoxicillin, amoxicillin/clavulanic acid and anti-tuberculosis drugs such as isoniazid, pyrazinamide and rifampicin), natural products (e.g., amanita mushrooms and green tea extract), alternative remedies (including herbal supplements and Chinese herbal remedies), and industrial toxins (e.g., arsenic, carbon tetrachloride and vinyl chloride). Acetaminophen followed by anti-tuberculosis drugs are the most common causes of ALF. Patterns of liver injury caused by chemicals (including medications) include zonal necrosis, hepatitis, cholestasis, steatosis, granulomas, vascular lesions and neoplasms. An uncoupler can also be used with one or more nicotinyl riboside compounds to prevent hepatotoxicity (including DILI) in patients who are scheduled to take a medication (e.g., an anti-tuberculosis drug, an NSAID or a glucocorticoid) for an active disease (e.g., tuberculosis or an inflammatory disorder) or for a disease (e.g., tuberculosis or an inflammatory disorder) that is diagnosed (e.g., a positive tuberculosis test) but not yet active. Mitochondrial unfolded protein response and reduced TCA cycle flux are associated with hepatotoxicity (including DILI). Combination therapy with an uncoupler and one or more nicotinyl riboside compounds enhances TCA cycle flux and mitochondrial function, reduces oxidative stress, inflammation and cell death, and promotes liver regeneration. In some embodiments, the one or more additional therapeutic agents for treatment of hepatotoxicity (e.g., DILI) or liver failure (e.g., ALF) are or comprise an antioxidant, an anti-inflammatory agent or a PARP inhibitor, or any combination thereof. In certain embodiments, the one or more additional therapeutic agents for treatment of acetaminophen-induced liver injury or liver failure (e.g., ALF) are or comprise N-acetylcysteine, which can treat acetaminophen overdose and function as an antioxidant.

In some embodiments, an uncoupler (e.g., one providing mild uncoupling) that preferentially distributes or is targeted to the liver is utilized for treatment of a liver or metabolic disorder, or a disorder that can be ameliorated by action in the liver. Certain uncouplers such as BAM15, Example No. 134 in WO 2019/226490 and OPC-163493 preferentially distribute to the liver. Liver targeting can be achieved by use of an uncoupler prodrug that is converted to the uncoupler by cytochrome P450 enzymes in the liver, such as an ether prodrug (e.g., DNP methyl ether). Liver targeting can also be achieved by encapsulation of an uncoupler in liposomes, micelles, cholestosomes or lipid, polymeric or dendrimeric nano-/microparticles bearing a plurality of an N-acetylgalactosamine moiety. Moreover, uncouplers that are substrates of transmembrane organic anion-transporting polypeptides preferentially expressed in the liver (e.g., OATP1B1 and OATP1B3) are preferentially taken up by hepatocytes.

In some embodiments, a mitochondrial uncoupler (e.g., one providing mild uncoupling such as NTZ, niclosamide, OPC-163493, controlled-release DNP, BAM15 or Example No. 134 in WO 2019/226490) is used in combination with one or more nicotinyl riboside compounds (e.g., NR or/and NRH, NRTA or/and NRHTA, or one or more NR/NAR derivatives disclosed herein) to treat obesity or an obesity-associated condition. Obesity-associated conditions include without limitation hypertension, hyperlipidemia, hypercholesterolemia, hypertriglyceridemia, hyperglycemia (fasting and postprandial), impaired glucose tolerance or glucose intolerance, prediabetes, diabetes (e.g., T2D), insulin resistance, hyperinsulinemia, metabolic syndrome, polycystic ovary syndrome, hyperphagia-associated disorders (e.g., Alstrom syndrome. Bardet-Biedl syndrome and Prader-Willi syndrome), cardiovascular diseases {including heart failure (e.g., congestive heart failure), ischemia (e.g., myocardial and cerebral ischemia), artery constriction (e.g., atherosclerosis), coronary artery diseases (e.g., angina, acute coronary syndrome, ischemic cardiomyopathy and myocardial infarction), cerebrovascular diseases (e.g., stroke), peripheral vascular diseases (e.g., peripheral artery disease [including intermittent claudication and critical limb ischemia]), thrombotic/embolic disorders (e.g., deep vein thrombosis and pulmonary embolism), and ischemia-reperfusion injury (IRI)}, lipid storage disorders (including lipid storage droplet disorders), liver disorders (e.g., non-alcoholic fatty liver disease [NAFLD], non-alcoholic steatohepatitis [NASH], alcoholic liver disease [ALD] and alcoholic steatohepatitis [ASH]), kidney disorders (e.g., chronic kidney disease [CKD] and diabetic nephropathy/kidney disease), urological disorders (e.g., urinary incontinence), rheumatic disorders (e.g., osteoarthritis, gout, low back pain and knee joint pain), neurological disorders (e.g., dementia such as Alzheimer's disease), respiratory disorders (e.g., obstructive sleep apnea, obesity hypoventilation syndrome and asthma), male and female infertility, and tumors and cancers (e.g., breast [e.g., postmenopausal], colon, rectal, colorectal, endometrial, esophageal [e.g., adenocarcinoma], gallbladder, kidney [e.g., renal cell], liver [e.g., hepatocellular carcinoma], ovarian, pancreatic, stomach [e.g., gastric cardia], thyroid and uterine [e.g., corpus uteri] tumors and cancers, and adenocarcinomas, melanoma, meningioma, soft tissue sarcomas [e.g., liposarcoma], leukemias and multiple myeloma). Obesity is also characterized by chronic low-grade inflammation and increased oxidative stress, where ROS can cause oxidative damage. Disorders associated with oxidative stress are described below.

Body weight can be reduced, and obesity can be treated, by increasing energy expenditure. Use of an uncoupler increases cellular energy expenditure by weakening the coupling between fuel oxidation and ATP synthesis and causing loss of calories as heat. To maintain the proton motive force weakened by uncoupling, to compensate for lack of efficiency in ATP synthesis and to support the energy need of the cell, mitochondria increase the oxidation of fuel such as fatty acids or/and glucose, TCA cycle flux and ETC activity in response to uncoupling. Increased fatty acid/beta oxidation and TCA cycle flux reduce de novo lipogenesis, synthesis of pro-inflammatory lipids (e.g., cicosanoids and docosanoids), and accumulation of lipids. In addition, uncoupling increases pyruvate influx to mitochondria, which promotes the complete oxidation of glucose. Other anti-obesity properties of uncouplers include inhibition of acetyl-CoA carboxylase and HMG-CoA reductase (such as via activation of AMPK).

Accordingly, in some embodiments a mitochondrial uncoupler (e.g., one providing mild uncoupling such as NTZ, niclosamide, OPC-163493, controlled-release DNP, BAM15 or Example No. 134 in WO 2019/226490) is used in combination with one or more nicotinyl riboside compounds (e.g., NR or/and NRH, NRTA or/and NRHTA, or one or more NR/NAR derivatives disclosed herein) to increase energy expenditure (e.g., whole-body energy expenditure or basal metabolic rate [BMR]) in a subject. In certain embodiments, use of the uncoupler with the one or more nicotinyl riboside compounds increases energy expenditure (e.g., whole-body energy expenditure or BMR) by at least about 2%, 5%, 10%, 15% or 20%, or by about 2-5%, 5-10%, 10-15% or 15-20%.

In some embodiments, energy expenditure is measured by cellular oxygen consumption. In certain embodiments, cellular oxygen consumption is measured in a Seahorse assay. In some embodiments, use of an uncoupler with one or more nicotinyl riboside compounds increases cellular oxygen consumption rate (OCR, such as in a Seahorse assay) by at least about 5%, 10%, 15%, 20%, 25% or 30%. Low OCRs are associated with T-cell senescence, inflammation and organ dysfunction, all of which can be at least partially reversed with NAD⁺ repletion.

Citrate synthase converts acetyl-CoA to citrate in the first step of the TCA cycle. Citrate synthase activity or flux correlates with OCR, mitochondrial oxidation rate and TCA cycle flux. In some embodiments, use of an uncoupler with one or more nicotinyl riboside compounds increases citrate synthase flux (V_{CS}, as measured by, e.g., positional isotopomer nuclear magnetic resonance tracer analysis [PINTA]) by at least about 10%, 20%, 30%, 50%, 100% (2-fold), 3-fold, 4-fold or 5-fold. In certain embodiments, use of an uncoupler with one or more nicotinyl riboside compounds increases V_{CS} (as measured by, e.g., PINTA) by at least about 30%, 50% or 100%.

A goal of safe and effective uncoupling is to increase energy expenditure by increasing TCA cycle flux without causing excessive reduction of intracellular ATP level and excessive production of body heat. The best-studied uncoupler, DNP, was effective in causing weight loss and was approved for the treatment of obesity in the US in the 1930s. However, DNP was withdrawn in 1938 as an anti-obesity drug due to its narrow therapeutic window, with an about 3-10-fold dose greater than the minimum effective dose for reducing body weight causing excessive systemic uncoupling, which led to fatally high body temperature due to the heat generated by uncoupling. Deaths resulting from the use of DNP as a weight-loss drug spurred the passing of the US Food, Drug and Cosmetic Act in 1938.

Use of a prodrug, a targeted form, or a controlled-, slow- or sustained-release composition of DNP can significantly improve safety by providing a substantially lower Cₘₐₓ of DNP. For example, DNP methyl ether is a liver-targeted prodrug that is preferentially metabolized by the cytochrome P450 system in the liver to DNP in a controlled release-like fashion, which can significantly increase the therapeutic index and reduce adverse effects due to systemic mitochondrial uncoupling. Another DNP prodrug, MP201 (which has a carbon-chain linker attached to the hydroxyl group), has significantly slower absorption, an about 20-fold lower Cₘₐₓ, an about 10-fold greater AUC and an about 3-fold longer residency/elimination time than DNP when taken orally, and thus has a significantly higher therapeutic index than DNP and can exert mild uncoupling. Furthermore, a DNP prodrug {e.g., one in which the hydroxyl group forms a carbamate bond with a promoiety, such as in 2,4-DNP-OC(═O)—N-morpholine, 2,4-DNP-OC(═O)—N-piperidine, 2,4-DNP-OC(═O)—N-piperazine or 2,4-DNP-OC(═O)—N-piperazine-N-Me} which is slowly hydrolyzed to DNP in the blood can provide slow release of DNP for mild systemic uncoupling. In addition, a controlled-, slow- or sustained-release formulation of DNP can provide a low but therapeutic dose of DNP over an extended period (e.g., days) and mild systemic uncoupling. A controlled-, slow- or sustained-release composition of DNP can be in the form of, e.g., liposomes, cholestosomes or lipid, polymeric or dendrimeric nanoparticles encapsulating DNP, or as described above, and can be administered, e.g., orally or parenterally (e.g., by intravenous, subcutaneous, intramuscular or intrathecal injection or by oral inhalation). Controlled-, slow- or sustained-release compositions of DNP are also described in US 2016/0199310, including in Example 1. A controlled-, slow- or sustained-release composition of DNP can be designed to form a depot at the site of administration (e.g., subcutaneous or intramuscular injection). A controlled-, slow- or sustained-release composition can also deliver a prodrug or targeted form of DNP, or the composition itself can be targeted to specific cell type(s), tissue(s) or organ(s), such as DNP being encapsulated in liposomes, cholestosomes or lipid, polymeric or dendrimeric nanoparticles bearing a plurality of a targeting moiety (e.g., a N-acetylgalactosamine moiety for targeting to the liver or an RGD-containing moiety for targeting to tumor/cancer cells with upregulated cell-membrane integrins). The concepts in this paragraph also apply to other uncouplers in general.

The use of one or more nicotinyl riboside compounds (e.g., NR or/and NRH, NRTA or/and NRHTA, or one or more NR/NAR derivatives disclosed herein) in combination with a mitochondrial uncoupler can significantly increase the therapeutic index of that uncoupler (see, e.g., Example 8), including an uncoupler of medium to high strength and an uncoupler used at high dose, provide safe and effective, sustained uncoupling, and allow for chronic combination therapy. Combination therapy with one or more nicotinyl riboside compounds can lower the concentration of the uncoupler at which the uncoupler has therapeutic effect, or/and can increase the concentration of the uncoupler at which the uncoupler has toxic effect. In certain embodiments, the use of one or more nicotinyl riboside compounds with an uncoupler increases the therapeutic index of the uncoupler by at least about 50%, 100% (2-fold), 3-fold, 4-fold, 5-fold, 10-fold, 20-fold, 50-fold or 100-fold. In some embodiments, the use of one or more nicotinyl riboside compounds with an uncoupler does not cause a significant increase in body temperature (e.g., at least about 1° C., 2° C., 3° C. or higher) for a significant length of time (e.g., at least about 30 min, 1 hr, 2 hr, 4 hr, 8 hr, 12 hr, 24 hr or longer), or significant systemic, hepatic or renal toxicity (e.g., a significant increase in blood/plasma/serum levels of one or more liver enzymes such as alanine transaminase [ALT] and aspartate transaminase [AST], or/and those of urea nitrogen or creatinine). Increase in the therapeutic index of an uncoupler can be determined, e.g., in animals or humans by determining whether co-administration of a nicotinyl riboside compound with an uncoupler reduces the lowest dose of the uncoupler effective for bringing about a certain therapeutic effect (e.g., reduction of the blood/plasma/serum level of a lipid [e.g., triglycerides or cholesterol], glucose or insulin, improvement in the glucose or insulin tolerance test, or reduction of liver steatosis, inflammation or fibrosis) compared to the lowest effective dose of the uncoupler given alone, and whether the dose of an uncoupler given alone and resulting in a significant adverse event or toxicity (e.g., a significant increase in body temperature, heart rate or the blood/plasma/serum level of a liver enzyme) is increased when the uncoupler is co-administered with a nicotinyl riboside compound.

It should be noted that the effective dose of uncouplers to treat a condition or disorder not related to body weight or obesity may be significantly lower than that for reducing body weight or treating obesity, and thus uncouplers may have a significantly wider therapeutic window for treatment of a condition or disorder that does not involve reduction of body weight or obesity. For example, because the effective dose of DNP (which readily crosses the intact blood-brain barrier) for neuroprotection (e.g., for treating a neurodegenerative disease [e.g., Alzheimer's, Huntington's or Parkinson's disease, multiple sclerosis or optic neuritis], epilepsy, traumatic brain injury, traumatic sciatic nerve damage or stroke) can be significantly lower than its effective dose for reducing body weight or treating obesity and significantly below its minimum toxic dose, DNP may have a sufficiently wide therapeutic window as a neuroprotector. Moreover, DNP may have hormesis-like effects when used for treatment of a condition or disorder that does not involve reduction of body weight or obesity, such as greater neuroprotective effects at lower doses and less efficacy at higher doses, which may allow DNP to be safely and effectively used for treatment of a condition or disorder not related to body weight or obesity. It should further be noted that even a modest weight loss (e.g., of about 8 pounds) can significantly improve metabolic endpoints (e.g, reduction or reversal of hepatic steatosis, hepatic insulin resistance and hyperglycemia in patients with T2D), so that the use of a low dose of an uncoupler with a relatively narrow therapeutic window (e.g., DNP) may be safe and effective for treatment of a condition or disorder related to body weight or obesity. Furthermore, chronic use (including over most of a person's adult life) of a low or very low dose of an uncoupler (including one with a relatively narrow therapeutic window such as DNP) which increases energy expenditure by a small degree but does not result in appreciable weight loss may still have therapeutic (e.g., metabolic and neurological) benefits (e.g., less oxidative stress and damage, lower blood glucose and lipid levels, lower lipid content in the liver and skeletal muscles, increased hepatic and peripheral insulin sensitivity, higher BDNF levels, enhanced neuroprotection and cognition, and longer lifespan), and may be beneficial as a wellness or anti-aging regimen. As described herein, the safety or/and the efficacy of an uncoupler as a therapeutic or wellness agent, whether or not related to body weight reduction or obesity, can be significantly enhanced when used in combination with one or more nicotinyl riboside compounds, including in chronic therapy.

Uncouplers providing mild uncoupling (e.g., NTZ, niclosamide, OPC-163493, controlled-release DNP, BAM15, Example No. 134 in WO 2019/226490 and uncouplers with a wide dynamic range), whose safety or/and efficacy can be significantly enhanced when used in combination with one or more nicotinyl riboside compounds, can stimulate the burning of calories and fat, and thereby reduce body weight and treat obesity or an obesity-associated condition. Uncouplers providing mild uncoupling can increase energy expenditure and reduce lipid content or fat accumulation in metabolically relevant tissues and organs such as skeletal muscles and the liver, as well as increase whole-body energy expenditure and reduce whole-body fat mass, without reducing lean mass or food intake. Mild uncoupling can also reduce fat accumulation in tissues and organs where excessive fat mass may be deleterious, such as the heart, pancreas and kidneys. An uncoupler can be targeted to the liver for treatment of, e.g., a liver or metabolic disorder by use of, e.g., BAM15 or Example No. 134 in WO 2019/226490 (both preferentially distribute to the liver), an uncoupler prodrug (e.g., an uncoupler having a methyl ether at an aromatic hydroxyl [e.g., phenolic] group, such as DNP methyl ether), or an uncoupler associated with a liver-targeting moiety (e.g., an uncoupler encapsulated in nanoparticles made of a lipid, polymer or dendrimer conjugated to a plurality of N-acetylgalactosamine moieties or in GalNAc-conjugated liposomes or cholestosomes). If desired, an uncoupler can be distributed to skeletal muscles or the entire body from the blood circulation, where the risk of an adverse event or toxicity (e.g., hyperthermia) due to excessive systemic uncoupling can be significantly reduced if the uncoupler is used in combination with one or more nicotinyl riboside compounds.

One or more other therapeutic agents described herein (e.g., an anti-obesity or antihyperlipidemic agent) can optionally be used in combination with one or more nicotinyl riboside compounds and an uncoupler to treat an obesity-associated condition. In certain embodiments, the obesity-associated condition is NAFLD, NASH, ALD or ASH, and the one or more other therapeutic agents are selected from antidiabetic agents, anti-obesity agents, anti-inflammatory agents, antifibrotic agents, antioxidants, and combinations thereof, as described above.

In some embodiments, a mitochondrial uncoupler (e.g., one providing mild uncoupling such as NTZ, niclosamide, OPC-163493, controlled-release DNP, BAM15 or Example No. 134 in WO 2019/226490) is used in combination with one or more nicotinyl riboside compounds (e.g., NR or/and NRH, NRTA or/and NRHTA, or one or more NR/NAR derivatives disclosed herein), optionally in conjunction with one or more other therapeutic agents (e.g., those described herein), to treat a disorder associated with abnormal or ectopic lipid accumulation or storage. Abnormal or ectopic lipid accumulation or storage can result in, e.g., lipotoxicity (and hence cellular dysfunction and death), metabolic complications (e.g., insulin resistance), and organ (e.g., liver or heart) damage and failure. Disorders associated with abnormal or ectopic lipid accumulation or storage include without limitation lipodystrophy (including congenital and acquired lipodystrophy, partial and generalized lipodystrophy, and severe lipodystrophy, such as HIV-associated lipodystrophy), steatosis (e.g., hepatic [including fatty liver diseases such as NAFLD, NASH, ALD and ASH], renal, cardiac and muscular steatosis), lipid storage disorders (e.g., LAL deficiency [including Wolman disease and CESD], Gaucher disease, Niemann-Pick disease and lipid storage droplet disorders such as CGI-58 deficiency [Chanarin-Dorfman syndrome], MTP deficiency and ApoB deficiency), fatty acid/beta oxidation disorders (e.g., MTP deficiency), dyslipoproteinemia-associated disorders (e.g., familial dysbetalipoproteinemia), insulin resistance-associated disorders (e.g., T2D), and others disclosed herein. The use of an uncoupler with one or more nicotinyl riboside compounds can reduce abnormal or ectopic lipid content by increasing lipid utilization and oxidation and enhancing mitochondrial efficiency and TCA cycle flux. In certain embodiments, the one or more other therapeutic agents are or comprise one or more anti-obesity or antihyperlipidemic agents, such as one or more agents that reduce lipid synthesis (e.g., a statin, an ACC inhibitor or/and an LXR agonist) or lipid uptake, or promote lipolysis (e.g., an GHRH analog such as tesamorelin), fatty acid/beta oxidation, or production of certain lipoproteins (e.g., apolipoprotein A1), or any combination thereof.

Lipodystrophy is characterized by fat accumulation in non-adipose tissues or organs and is associated with metabolic disorders that are also associated with obesity, including metabolic syndrome, hypertriglyceridemia, insulin resistance, diabetes (e.g., T2D), cardiovascular diseases (e.g., CAD), and NAFLD (e.g., NASH). Therefore, a mitochondrial uncoupler (e.g., one providing mild uncoupling such as NTZ, niclosamide, OPC-163493, controlled-release DNP, BAM15 or Example No. 134 in WO 2019/226490) and one or more nicotinyl riboside compounds (e.g., NR or/and NRH, NRTA or/and NRHTA, or one or more NR/NAR derivatives disclosed herein), optionally in conjunction with one or more other therapeutic agents (e.g., those described herein), can be used to treat lipodystrophy (including congenital and acquired, partial and generalized, and severe) or a disorder (e.g., a metabolic disorder) associated therewith. In certain embodiments, the lipodystrophy is congenital generalized lipodystrophy (CGL or Berardinelli-Seip syndrome), familial partial lipodystrophy (FPL or Köbberling-Dunnigan syndrome), acquired generalized lipodystrophy (AGL or Lawrence syndrome), acquired partial lipodystrophy (APL or Barraquer-Simons syndrome), or HIV-associated lipodystrophy (in the presence or absence of antiretroviral therapy). In certain embodiments, the one or more other therapeutic agents are or comprise a form of leptin (e.g., human recombinant leptin [e.g., metreleptin] or an intermediate-acting or long-acting analog of leptin) for lipodystrophy characterized by low leptin levels (e.g., generalized forms of lipodystrophy such as CGL and AGL, or severe lipodystrophy), a form of growth-hormone-releasing hormone (GHRH) (e.g., human recombinant GHRH or an intermediate-acting or long-acting analog of GHRH such as tesamorelin) for HIV-associated lipodystrophy (in the presence or absence of antiretroviral therapy), or/and one or more anti-obesity or antihyperlipidemic agents (e.g., one or more agents that reduce lipid synthesis, such as a statin, a fibrate or/and a thiazolidinedione).

In further embodiments, and uncoupler (e.g., one providing mild uncoupling) and one or more nicotinyl riboside compounds, optionally in conjunction with one or more other therapeutic agents described herein (e.g., an anti-obesity or antihyperlipidemic agent such as a melanocortin 4 receptor agonist [e.g., setmelanotide]), are used to treat hyperphagia (polyphagia) or a disorder associated therewith. Hyperphagia is an abnormally strong sensation of hunger or desire to eat that often leads to or is accompanied by overeating and does not subside after eating. Hyperphagia is often a result of hyperglycemia or hypoglycemia, and often leads to metabolic disorders such as insulin resistance and NASH. Hyperphagia is a symptom of many disorders, including diabetes (e.g., type 1), Alstrdöm syndrome, Bardet-Biedl syndrome, chromosome 22q13 and Xq26.3 duplication syndromes, congenital generalized lipodystrophy (e.g., types 1 and 2), familial renal glucosuria, frontotemporal dementia, hyperthyroidism (e.g., Graves' disease), hypotonia-cystinuria syndrome, Kleine-Levin syndrome, leptin/leptin receptor deficiency or dysfunction, Luscan-Lumish syndrome, macrosomia adiposa congenita, mental retardation (autosomal dominant 1), obesity, hyperphagia and developmental delay (OBHD), Pick's disease, Prader-Willi syndrome, pro-opiomelanocortin deficiency, and Schaaf-Yang syndrome.

In addition to their anti-obesity and fat-burning effects, uncouplers (e.g., those providing mild uncoupling such as NTZ, niclosamide, OPC-163493, controlled-release DNP, BAM15 and Example No. 134 in WO 2019/226490) can improve glucose tolerance, lower elevated fasting and postprandial blood glucose levels (e.g., by increasing hepatic glucose uptake and reducing hepatic gluconeogenesis), increase insulin sensitivity and reduce elevated blood insulin level. Reduction of ectopic fat in skeletal muscles and the liver, which are important insulin-sensitive tissues and organ, by uncoupling improves skeletal muscle and hepatic insulin sensitivity (e.g., by reducing diacylglycerol and triacylglycerol content in skeletal muscles and the liver, and reducing PKC-θ activity in skeletal muscles and PKC-ε activity in the liver), which consequently lowers elevated blood glucose and insulin levels and improves glucose tolerance. Therefore, uncouplers (e.g., those providing mild uncoupling) can be used to treat glucose intolerance, hyperinsulinemia and disorders characterized by high blood glucose level (e.g., T1D, T2D, severe insulin resistance syndrome [SIRS], metabolic syndrome and drug-induced hyperglycemia) or insulin resistance (e.g., T2D, SIRS, NAFLD, NASH and hepatitis C). One or more other therapeutic agents described herein (e.g., an antidiabetic agent) can optionally be used in combination with an uncoupler (e.g., one providing mild uncoupling) and one or more nicotinyl riboside compounds (e.g., NR or/and NRH, NRTA or/and NRHTA, or one or more NR/NAR derivatives disclosed herein) to treat such a condition or disorder. In certain embodiments, the uncoupler is an antidiabetic agent, which would exert antidiabetic effect(s) through other mechanism(s) of action as well as other beneficial effects through uncoupling.

Uncoupling reduces mitochondrial production and accumulation of reactive oxygen species (ROS). ROS can damage DNA and alter proteins, and thereby can cause cellular dysfunction, apoptosis and tissue degeneration. Uncouplers can also reduce oxidative stress by other means, such as by increasing the levels of antioxidants (e.g., increasing the ratio of glutathione to its disulfide form and the expression of antioxidant proteins via activation of nuclear factor erythroid 2-related factor 2 [Nrf2 or NFE2L2]). Accordingly, in some embodiments a mitochondrial uncoupler (e.g., one providing mild uncoupling such as NTZ, niclosamide, OPC-163493, controlled-release DNP, BAM15 or Example No. 134 in WO 2019/226490) is used in combination with one or more nicotinyl riboside compounds (e.g., NR or/and NRH, NRTA or/and NRHTA, or one or more NR/NAR derivatives disclosed herein) to treat a disorder characterized by oxidative stress. Disorders associated with oxidative stress, whether oxidative stress is a cause of the disorder or/and a result of the disorder that contributes to its pathological effects, include without limitation metabolic disorders (e.g., obesity, diabetes [e.g., T1D and T2D] and insulin resistance), cardiovascular disorders (e.g., narrowing of arteries such as atherosclerosis and ischemic injuries such as myocardial infarction and stroke), ischemia-reperfusion injuries (e.g., myocardial, cerebral and renal IRI s), inflammatory disorders (e.g., optic neuritis, keratoconjunctivitis sicca [dry eye syndrome], and cancers whose invasion or metastasis is promoted by ROS-induced pro-inflammatory cytokines), autoimmune disorders (e.g., multiple sclerosis, Guillain-Barré syndrome [GBS], myasthenia gravis, systemic lupus erythematosus [SLE], and Graves' disease), degenerative disorders such as neurodegenerative disorders (e.g., Alzheimer's, Huntington's and Parkinson's diseases, spinal muscular atrophy [SMA], ALS, multiple sclerosis, GBS, Friedreich's ataxia, Wolfram syndrome [DIDMOAD], Batten disease, optic neuritis, glaucoma, LHON and AMD) and musculodegenerative disorders (e.g., muscular dystrophy such as Duchenne MD and Becker MD), neuromuscular disorders (e.g., muscular dystrophy, SMA, epilepsy, Leigh syndrome, MELAS, MERRF, Pompe disease and myasthenia gravis), other muscle disorders (e.g., cachexia), neurodevelopmental disorders (e.g., autism spectrum disorder [ASD], Angelman syndrome, Rett syndrome and Fragile X syndrome), psychiatric disorders (e.g., ASD, depression, bipolar disorder, anxiety disorders and schizophrenia), mitochondrial diseases (e.g., Friedreich's ataxia, LHON, Leigh syndrome, MELAS and MERRF), lysosomal storage diseases (e.g., Pompe disease), disorders due to physical/body trauma (e.g., traumatic brain injury [TBI, including concussion], traumatic spinal cord injury [TSCI], traumatic sciatic nerve damage, trauma from a vehicle accident or a fall from a height, hearing impairment/loss due to blast noise, repetitive loud noise or a head trauma, severe burn, shock and sepsis), polycystic kidney disease, male infertility, and aging-related disorders (e.g., chronic inflammation, AMD, dry eye syndrome and hearing loss). Excessive ROS levels can induce apoptosis, which can occur in, e.g., Alzheimer's and Parkinson's diseases, TBI and TSCI. Uncouplers can provide cytoprotection and neuroprotection against increased ROS levels and oxidative damage resulting from mitochondrial dysfunction caused by ischemia, excitotoxicity or physical trauma. In some embodiments, an uncoupler is administered within about 2, 4, 6, 12 or 24 hours after the occurrence of an ischemia or body trauma. An uncoupler can also be taken prophylactically before the occurrence of a body trauma (e.g., a TBI or TSCI) where the likelihood of a body trauma is relatively high, such as in a military action or a sporting event (e.g., a boxing, American football or mixed martial arts match). Some uncouplers such as BHT, MitoBHT, MitoQₙ compounds, retinoids and many naturally occurring phenols also have other antioxidant (e.g., free radical-scavenging) properties. One or more other therapeutic agents described herein (e.g., an antioxidant) can optionally be used in combination with one or more nicotinyl riboside compounds and an uncoupler to treat a disorder associated with oxidative stress.

In some embodiments, a mitochondrial uncoupler (e.g., one providing mild uncoupling such as NTZ, niclosamide, OPC-163493, controlled-release DNP, BAM15 or Example No. 134 in WO 2019/226490) is used in combination with one or more nicotinyl riboside compounds (e.g., NR or/and NRH, NRTA or/and NRHTA, or one or more NR/NAR derivatives disclosed herein) to treat a neurological disorder. In certain embodiments, the neurological disorder is a neurodegenerative disorder (e.g., Alzheimer's, Huntington's or Parkinson's disease, ALS or multiple sclerosis), optic neuritis, Charcot-Marie-Tooth (CMT) disease, epilepsy, ischemic stroke or TBI. Other neurological disorders, including neurodegenerative disorders, are described elsewhere herein. Since uncoupling makes the mitochondrial matrix more acidic, uncoupling in neurons increases the release of calcium ions from mitochondria, which can promote neurotransmission, synaptic plasticity and gene transcription. For example, increased cytosolic Ca²⁺ level in neurons activates adenylyl cyclase and hence the production of the second messenger cyclic AMP, which is implicated in synaptic plasticity, memory formation, learning and cognition, such as via upregulated expression of BDNF. BDNF also protects neurons against excitotoxicity and promotes neuronal growth and repair of damaged neurons. Furthermore, uncoupling increases autophagy, which can break up abnormal protein aggregates such as α-synuclein aggregates in Parkinson's disease and tau aggregates in Alzheimer's disease. In addition, use of an uncoupler (e.g., one providing mild uncoupling) with one or more nicotinyl riboside compounds can preserve NAD⁺ level and energy production, which can protect neurons against excitotoxic or ischemic cell injury or death such as in Alzheimer's disease, epilepsy and stroke. One or more other therapeutic agents described herein (e.g., a neuroprotector or/and an antioxidant) can optionally be used in combination with one or more nicotinyl riboside compounds and an uncoupler to treat a neurological disorder.

Because BDNF is also a myokine outside of the brain and is important for muscle biology and strength, upregulated expression of BDNF via uncoupling is also useful for treating muscle disorders, including muscular dystrophy such as Duchenne muscular dystrophy (DMD) and Becker muscular dystrophy (BMD). DMD and BMD are caused by the loss of dystrophin, which is expressed only in muscles and the brain. The loss of dystrophin results in muscle loss and cognitive impairment. Elevated BDNF levels can increase muscle mass and strength (including those of heart and diaphragm muscles) and improve cognition in patients with neuromuscular disorders such as DMD and BMD.

Mitochondrial Ca²⁺ overload is associated with apoptosis as well as increased mitochondrial ROS production. Mitochondrial Ca²⁺ overload can induce the formation and opening of mitochondrial permeability transition pores (mPTP), which can result in the leaking of toxic mitochondrial molecules (e.g., ROS, apoptosis-inducing factor, caspase activators and cytochrome C) into the cytosol. Such molecules can induce apoptosis, which can occur in, e.g., neurodegenerative disorders (e.g., Alzheimer's, Huntington's and Parkinson's diseases, Friedreich's ataxia and Wolfram syndrome), neuromuscular disorders (e.g., DMD, epilepsy and Pompe disease), other muscle disorders (e.g., cachexia), lysosomal storage diseases (e.g., Pompe disease), and disorders due to body trauma (e.g., TBI). Uncoupling reduces the mitochondrial membrane potential and thereby closes voltage-dependent uniporters involved in Ca²⁺ influx to the mitochondrial matrix. Therefore, an uncoupler can be used to prevent mitochondrial Ca²⁺ overload and hence to exert cytoprotective (e.g., neuroprotective) effects in disorders associated therewith.

Furthermore, uncouplers (e.g., those providing mild uncoupling such as NTZ, niclosamide, OPC-163493, controlled-release DNP, BAM15 and Example No. 134 in WO 2019/226490) can reduce inflammation (including liver inflammation) and fibrosis (including liver fibrosis). Uncouplers can reduce mitochondrial ROS production and lipid accumulation in tissues and organs. Oxidized molecules, including oxidized lipids, can be highly pro-inflammatory, and inflammation is a strong driver of fibrosis. Uncouplers can also have anti-inflammatory and anti-fibrotic effects through other mechanisms, including inhibition of pro-inflammatory transcription factors (e.g., NF-κB and STAT3), reduced expression of pro-inflammatory cytokines (e.g., IL-1α, -1β, -4 and -6, and TNF-α), inhibition of the inositol-requiring enzyme-la (IRE-1α) branch of the unfolded protein response (IRE-1α signaling induces the expression of pro-inflammatory genes including NF-κB in response to endoplasmic reticulum [ER] stress), inhibition of fibrogenic epithelial-mesenchymal transition via inhibition of the Wnt/β-catenin pathway, inhibition of hepatic stellate cell activation (which leads to liver fibrosis), and reduced expression of apoptosis mediators (e.g., caspase-3) (apoptosis of hepatocytes contributes to liver inflammation and fibrosis). Therefore, uncouplers (e.g., those providing mild uncoupling) are useful for treating inflammatory disorders and fibrotic disorders, including liver disorders characterized by inflammation (e.g., autoimmune and viral hepatitis) or/and fibrosis (e.g., NASH, ASH, cirrhosis, PBC, PSC, hemochromatosis, Wilson's disease and liver schistosomiasis), kidney disorders characterized by inflammation or/and fibrosis (e.g., diabetic nephropathy), heart disorders characterized by inflammation or/and fibrosis (e.g., inflammatory [myocarditis] and non-inflammatory cardiomyopathy), pancreatic disorders characterized by inflammation or/and fibrosis (e.g., chronic pancreatitis), inflammatory gastrointestinal disorders (e.g., inflammatory bowel disease [e.g., Crohn's disease] and colitis [e.g., ulcerative colitis]), inflammatory skin disorders (e.g., psoriasis), and inflammatory aging-related disorders (e.g., chronic inflammation). Other inflammatory disorders and fibrotic disorders are described elsewhere herein. Autoimmune responses generally incite or are induced by an inflammatory reaction, so uncouplers (e.g., those providing mild uncoupling) are also useful for treating autoimmune disorders, including but not limited to rheumatoid arthritis, Sjögren syndrome, systemic scleroderma/sclerosis, SLE, multiple sclerosis, GBS, and skin disorders (e.g., pemphigus, pemphigoid and psoriasis). One or more other therapeutic agents described herein (e.g., an anti-inflammatory agent or/and an antifibrotic agent) can optionally be used in combination with one or more nicotinyl riboside compounds and an uncoupler to treat an inflammatory, autoimmune or fibrotic disorder. In certain embodiments, the uncoupler is an NSAID, which would exert anti-inflammatory effect(s) through other mechanism(s) of action as well as other beneficial effects through uncoupling.

As an example, an uncoupler can be used with a nicotinyl riboside compound, optionally in conjunction with one or more other therapeutic agents disclosed herein (e.g., an anti-inflammatory agent such as an inhibitor of TNF-α signaling), to treat TFAM deficiency or a disorder having similar clinical features (e.g., Kearns-Sayre syndrome). TFAM deficiency results in dysfunctional mitochondria (e.g., in T cells), premature aging (e.g., of T cells and the subject), aging-related features (e.g., chronic inflammation and metabolic, cardiovascular, cognitive and physical decline), and premature death (e.g., of T cells and the subject). Such a combination of therapeutic agents can increase TCA cycle flux and cellular oxygen consumption, enhance mitochondrial and cellular function, improve the fitness of cells and the subject, and prevent premature aging and death.

Moreover, uncouplers can suppress aberrant immune cell activation and aberrant inflammatory immune responses (e.g., as a result of a cytokine storm) by shifting energy metabolism from glycolysis to the TCA cycle. Such a shift results in quiescence of cells whose energy metabolism is predominantly glycolytic, including activated or hyperactivated immune cells (e.g., B cells, T cells, natural killer cells and macrophages), activated fibroblasts (involved in fibrosis), and tumor and cancer cells (infra). Therefore, an uncoupler, optionally in conjunction with a nicotinyl riboside compound or/and another therapeutic agent (an anti-inflammatory agent or immunosuppressant, or/and an antipathogenic agent [e.g., an antibiotic or antiviral]), can be used to treat a disorder associated with overactivation of the immune system (e.g., as a result of a cytokine storm). The immune system can become overactive in response to, e.g., a host agent (such as in an autoimmune disorder) or a foreign agent (e.g., a pathogen). In certain embodiments, the disorder is associated with a pathogenic (e.g., bacterial or viral) infection, such as one by a coronavirus (e.g., SARS-CoV-2 implicated in COVID-19). An uncoupler, optionally in conjunction with a nicotinyl riboside compound or/and another therapeutic agent (e.g., an anti-inflammatory agent), can also be used to treat systemic inflammatory response syndrome (SIRS). SIRS can have an infectious or non-infectious cause, can result from a cytokine storm, can be associated with a range of inflammatory disorders (e.g., alcoholic hepatitis), and can lead to shock and failure of one or more organs (e.g, acute kidney injury, acute-on-chronic liver failure [ACLF] or multiple organ dysfunction syndrome [MODS]).

In additional embodiments, a mitochondrial uncoupler (e.g., one providing mild uncoupling such as NTZ, niclosamide, OPC-163493, controlled-release DNP, BAM15 or Example No. 134 in WO 2019/226490) is used in combination with one or more nicotinyl riboside compounds (e.g., NR or/and NRH, NRTA or/and NRHTA, or one or more NR/NAR derivatives disclosed herein) to treat a tumor or cancer. Such a combination increases cellular NAD⁺ level and the NAD⁺/NADH ratio. Uncoupling or/and increasing cellular NAD⁺ level and the NAD⁺/NADH ratio suppress tumor/cancer cell growth, proliferation, invasion and metastasis by attenuating anaerobic respiration (viz., glycolysis) and anabolic pathways (e.g., the pentose phosphate pathway) in tumor/cancer cells and promoting pyruvate influx to mitochondria, the TCA cycle and aerobic (mitochondrial) respiration. Uncouplers can also have anti-tumor/cancer effects through other mechanisms, including reduced expression of c-Fos, c-Jun, c-Myc and β-catenin, and inhibition of aberrant Akt/PKB, MAPK/ERK, mTORC1, NF-κB, Notch, c-Src, STAT3 and Wnt/β-catenin signaling. Furthermore, uncouplers can enhance the immune response against tumor/cancer cells through reversal of T-cell exhaustion by, e.g., increasing TCA cycle flux. In addition, other properties of uncouplers can contribute to the inhibition of tumors and cancers. For example, the anti-obesity, insulin-sensitizing, anti-hyperinsulinemia, anti-hyperglycemia and anti-inflammatory properties of uncouplers can contribute to the inhibition of tumors and cancers associated with, e.g., obesity, insulin resistance, hyperinsulinemia, hyperglycemia or chronic inflammation. In certain embodiments, an uncoupler and one or more nicotinyl riboside compounds are used to treat a tumor or cancer of the adrenal gland (e.g., adrenocortical carcinoma), blood (e.g., leukemia), bone (e.g., an osteosarcoma), brain or spine (e.g., a glioma), head or neck, breast, colon, kidney (e.g., renal cell carcinoma), liver (e.g., hepatocellular carcinoma [HCC]), pancreas, lung (e.g., non-small cell lung cancer), endometrium, ovary or prostate, or an obesity-associated tumor or cancer (supra). One or more other therapeutic agents described herein (e.g., an anticancer agent) can optionally be used in combination with one or more nicotinyl riboside compounds and an uncoupler to treat a tumor or cancer.

Treatment of a tumor or cancer encompasses prevention of tumor/cancer recurrence (e.g., recurrence of liver cancer such as HCC after liver resection or radiofrequency ablation[RFA]) and occurrence of a secondary tumor/cancer (e.g., metastasis to the liver in a patient with breast or colorectal cancer). In certain embodiments, an uncoupler and one or more nicotinyl riboside compounds are used in combination with an anti-obesity or antihyperlipidemic agent (e.g., an ACC inhibitor such as firsocostat) to prevent recurrence of liver cancer (e.g., HCC) after liver resection or RFA. Furthermore, an uncoupler (e.g., one providing mild uncoupling), optionally in combination with one or more other therapeutic agents disclosed herein (e.g., one or more nicotinyl riboside compounds), can be used ex vivo to enhance the function of immune cells (e.g., T-cells) adapted (e.g., genetically modified) to treat a tumor or cancer, such as chimeric antigen receptor (CAR) T-cells and T-cell receptor (TCR)-engineered T-cells.

Many tumors and cancers are characterized by mutation of PI3K-α resulting in a more active kinase. PI3K-α activation contributes significantly to cellular transformation and the development of cancer, including being a key driver of the proliferation and metastatic potential of many solid tumors. Insulin signaling activates PI3K-α and hence the PI3K/Akt pathway, which promotes cell proliferation. Therefore, PI3K-α inhibitors can be used to treat tumors and cancers associated with an activated PI3K-α (e.g., due to an activating PI3K-α mutation or hyperinsulinemia). A common side effect of PI3K-α inhibitors is hyperglycemia due to inhibition of insulin signaling, which may lead to reduction of dose or discontinuation of the PI3K-α inhibitor. Uncouplers have anti-tumor/cancer effects in part by shifting the energy metabolism of tumor/cancer cells from glycolysis to the TCA cycle. Moreover, uncouplers increase whole-body insulin sensitivity and thus glucose uptake by, e.g., the liver and skeletal muscles, thereby reducing blood glucose and insulin levels. Reduction of blood glucose and insulin levels ameliorates the hyperglycemic side effect of PI3K-α inhibitors as well as diminishes PI3K-α activation. Accordingly, in some embodiments an uncoupler is used in combination with a PI3K-α inhibitor, optionally in conjunction with one or more other therapeutic agents described herein (e.g., a nicotinyl riboside compound or/and an antidiabetic agent such as an SGLT2 inhibitor) to treat a tumor or cancer associated with an activated PI3K-α (e.g., due to an activating PI3K-α mutation or hyperinsulinemia). In certain embodiments, the tumor or cancer is a solid tumor or cancer (e.g., of the breast [e.g., HR-positive/HER2-negative breast cancer or triple-negative breast cancer], endometrium or urothelium, or a hyperinsulinemia-associated or obesity-associated solid tumor or cancer [supra] such as colorectal cancer) or a lymphoma (e.g., a non-Hodgkin lymphoma, a B-cell lymphoma, chronic lymphocytic leukemia [CLL] or follicular lymphoma). In certain embodiments, the PI3K-α inhibitor is a selective PI3K-α inhibitor, such as alpelisib.

An uncoupler can also be used in combination with a PI3K-α inhibitor, optionally in conjunction with one or more other therapeutic agents described herein (e.g., a nicotinyl riboside compound or/and an anti-inflammatory agent), to treat an inflammatory disorder, such as an inflammatory respiratory disorder (e.g., asthma or COPD).

In further embodiments, an uncoupler (e.g., one providing mild uncoupling such as NTZ, niclosamide, OPC-163493, controlled-release DNP, BAM15 or Example No. 134 in WO 2019/226490), optionally in combination with one or more other therapeutic agents described herein (e.g., one or more nicotinyl riboside compounds such as NR or/and NRH, NRTA or/and NRHTA, or one or more NR/NAR derivatives disclosed herein), is utilized in the development of organoids and cell therapies derived from stem cells (e.g., embryonic stem cells [ESCs] or induced pluripotent stem cells [iPSCs]) or progenitor cells. Organoid types include without limitation cardiac organoids, gut organoids (including stomach/gastric organoids and intestinal organoids), kidney organoids, liver organoids, pancreatic organoids, hepato-biliary-pancreatic organoids, lung organoids, testicular organoids, cerebral organoids, retinal organoids, lingual organoids, epithelial organoids, thymic organoids, and thyroid organoids. Organoids can be used, e.g., as models for developmental biology, as models of human diseases, for development and testing of drugs, for individual tailoring of therapy (personalised medicine), or for organ transplant/replacement. By favoring the TCA cycle over glycolysis, uncoupling can reduce stemness and promote organoid development. As an example, increase in TCA cycle flux through the use of an uncoupler, optionally in combination with one or more nicotinyl riboside compounds, can result in terminal differentiation of pseudo-hepatocytes derived from stem cells (e.g., human ESCs or iPSCs) to true hepatocytes that are quiescent and metabolically active (including having functional cytochromes P450). The resulting hepatocytes can be used to develop hepatocyte cell therapies (e.g., for severe hepato-biliary disease), or hepatocytes or liver organoids that in turn can be used, e.g., as models of human liver disorders, in drug development/testing or personalised medicine for liver disorders, or for liver transplant/replacement, where the liver disorders can be, e.g., parenchymal liver disorders (e.g., hepatitis, liver fibrosis, NAFLD [e.g., NASH], ALD [e.g., ASH], chronic liver disease, and cirrhosis), those involving an enzyme deficiency (e.g., lysosomal acid lipase deficiency [including Wolman disease and CESD]), and those relating to a mitochondrial disease (e.g., MDDS). A human liver organoid can be utilized as a bio-artificial liver device for patients with, e.g., liver failure (e.g., acute or acute-on-chronic) or a severe metabolic disorder.

In some embodiments, the dose or therapeutically effective amount of the uncoupler (e.g., one providing mild uncoupling such as nitazoxanide, tizoxanide, niclosamide, OPC-163493, BHT, DNP, BAM15 or Example No. 134 in WO 2019/226490, or a prodrug, a targeted form, or a controlled-, slow- or sustained-release form thereof), optionally used in conjunction with one or more other therapeutic agents described herein (e.g., one or more nicotinyl riboside compounds), is from about 1, 50 or 100 mg to about 500 mg per day, which can be administered (e.g., orally) in a single dose (e.g., N mg once daily) or in divided/multiple doses (e.g., N/2 mg twice daily). In further embodiments, the dose of the uncoupler (e.g., one providing mild uncoupling) is about 1-100 mg, 100-200 mg, 200-300 mg, 300-400 mg or 400-500 mg per day. In still further embodiments, the dose of the uncoupler (e.g, one providing mild uncoupling) is from about 1 or 10 mg to about 100 mg, from about 1 or 10 mg to about 50 mg, or about 50-100 mg per day. In certain embodiments, the dose of the uncoupler (e.g., one providing mild uncoupling) is about 1-10 mg or 1-20 mg per day. In other embodiments, the dose of the uncoupler (e.g., one providing mild uncoupling) is about 1 μg-1 mg, 1-500 μg or 0.5-1 mg per day. In yet other embodiments, the dose of the uncoupler (e.g., one providing mild uncoupling) is about 1-100 μg, 100-200 μg, 200-300 μg, 300-400 μg or 400-500 μg per day. In some embodiments, the dose is higher for a weaker coupler and lower for a stronger uncoupler. The dose of an uncoupler can also be based on, e.g., the type of disorder being treated, the type of intended therapeutic regimen, the route of administration or the type of composition (e.g., a lower dose for a targeting composition [e.g., a dendrimer bearing one or more targeting moieties] or a sustained-release composition [e.g., a depot]), as described below.

In further embodiments, the concentration (e.g., in the blood/plasma/serum, such as the steady-state blood/plasma/serum concentration) of the uncoupler (e.g, one providing mild uncoupling such as nitazoxanide, tizoxanide, niclosamide, OPC-163493, BHT, DNP, BAM15 or Example No. 134 in WO 2019/226490, or a prodrug, a targeted form, or a controlled-, slow- or sustained-release form thereof) is from about 0.1, 0.5 or 1 μM to about 5, 10 or 15 μM, or from about 0.1, 0.5 or 1 μM to about 5 μM, about 5-10 μM or about 10-15 μM. The steady-state blood/plasma/serum concentration of the uncoupler can also be lower, such as from about 1 or 10 nM to about 100 nM, or about 1-50 nM or 50-100 nM. In certain embodiments, the concentration (e.g., in the blood/plasma/serum, such as the steady-state blood/plasma/serum concentration) of the uncoupler (e.g., one providing mild uncoupling) is from about 0.5 or 1 μM to about 5 μM, or about 5-10 μM.

The uncoupler (e.g., one providing mild uncoupling) can be administered in any suitable frequency. In certain embodiments, the uncoupler is administered once or twice daily. In other embodiments, a controlled-, slow- or sustained-release composition of the uncoupler, whether the active uncoupler or a prodrug thereof, is administered once daily (e.g., release of the uncoupler over about 24 hr) or twice daily (e.g, release of the uncoupler over about 12 hr), once every two days, twice weekly, once weekly, once every two weeks or once monthly. The uncoupler (e.g., one providing mild uncoupling) can be administered via any suitable route. In certain embodiments, the uncoupler is administered orally. In other embodiments, the uncoupler is administered parenterally (e.g., intravenously, subcutaneously, intramuscularly, intrathecally or topically [e.g., sublingually]).

The dose or therapeutically effective amount, the frequency of administration and the route of administration of a nicotinyl riboside compound used in conjunction with an uncoupler can be, e.g., any dose or therapeutically effective amount, any frequency of administration and any route of administration of the NR/NAR derivatives of the disclosure described herein. In some embodiments, the dose of a nicotinyl riboside compound (e.g., NR, NRH, NRTA, NRHTA or an NR/NAR derivative disclosed herein) is from about 1, 50 or 100 mg to about 500 or 1000 mg per day, which can be administered (e.g., orally) in a single dose (e.g., N mg once daily) or in divided/multiple doses (e.g., N/2 mg twice daily). In certain embodiments, the dose of a nicotinyl riboside compound is about 1-100 mg, 100-500 mg or 500-1000 mg per day, or about 50 mg, 100 mg, 150 mg, 200 mg, 250 mg, 300 mg, 400 mg, 500 mg, 600 mg, 700 mg, 800 mg, 900 mg or 1000 mg per day. The dose of a nicotinyl riboside compound can also be higher than 1.0 g per day, such as about 1.0-1.5 g, 1.5-2.0 g, 2.0-2.5 g or 2.5-3.0 g per day. In further embodiments, the dose of a nicotinyl riboside compound is about 1-50 mg, 50-100 mg, 100-200 mg, 200-300 mg, 300-400 mg or 400-500 mg per day. In certain embodiments, the dose of a nicotinyl riboside compound is from about 10, 50 or 100 mg to about 200 or 300 mg per day.

In some embodiments, the concentration (e.g., in the blood/plasma/serum, such as the steady-state blood/plasma/serum concentration) of a nicotinyl riboside compound (e.g., NR, NRH, NRTA, NRHTA or an NR/NAR derivative disclosed herein) is about 1-200 PM, 1-50 PM, 50-100 μM or 100-200 μM. The steady-state blood/plasma/serum concentration of a nicotinyl riboside compound can also be lower, such as about 10-100 nM, 0.1-0.5 μM or 0.5-1 μM. In certain embodiments, the one or more nicotinyl riboside compounds are or comprise NR, and the concentration (e.g., in the blood/plasma/serum, such as the steady-state blood/plasma/serum concentration) of NR is about 50-200 μM, 50-100 μM, 100-200 μM or 100 μM. In further embodiments, the one or more nicotinyl riboside compounds are or comprise NRH, and the concentration (e.g., in the blood/plasma/serum, such as the steady-state blood/plasma/serum concentration) of NRH is from about 1 or 5 μM to about 20 or 50 μM, or from about 1 or 5 μM to about 10 μM, or from about 10 μM to about 20 or 50 μM, or about 10 μM. In additional embodiments, the concentration of a nicotinyl riboside compound (e.g., NR, NRH, NRTA, NRHTA or an NR/NAR derivative disclosed herein) in a tissue or organ (e.g., a target tissue or organ such as white adipose tissue or the liver) is at least about 100, 200, 300, 400 or 500 nmol/g.

A nicotinyl riboside compound can be administered in any suitable frequency. In certain embodiments, a nicotinyl riboside compound is administered once or twice daily. Likewise, a nicotinyl riboside compound can be administered via any suitable route. In certain embodiments, a nicotinyl riboside compound is administered orally. In other embodiments, a nicotinyl riboside compound is administered parenterally (e.g., intravenously, subcutaneously, intramuscularly, intrathecally or topically [e.g., sublingually]).

In some embodiments, one or more nicotinyl riboside compounds (e.g., NR or/and NRH, NRTA or/and NRHTA, or one or more NR/NAR derivatives disclosed herein) are used in combination with a mitochondrial uncoupler (e.g., one providing mild uncoupling such as NTZ, niclosamide, OPC-163493, controlled-release DNP, BAM15 or Example No. 134 in WO 2019/226490) and a PARP inhibitor. NAD⁺ is required as a PARP substrate for generating ADP-ribose monomers for the synthesis of a polymeric ADP-ribose chain, which acts as a signal for other enzymes involved in the repair of single-strand DNA breaks. Overactivation of PARP may lead to depletion of NAD⁺ and ATP. Mild or modest PARP inhibition, such as through the use of a low or very low dose of a PARP inhibitor, can preserve cellular NAD⁺ and ATP levels and DNA repair, and thereby can maintain or improve cell function and viability.

Embodiments relating to combinations of one or more nicotinyl riboside compounds and a PARP inhibitor described elsewhere herein can also apply to combinations of one or more nicotinyl riboside compounds, an uncoupler (e.g., one providing mild uncoupling) and a PARP inhibitor. In some embodiments, the dose of a PARP inhibitor to treat a non-tumor/non-cancer disease/disorder or condition disclosed herein, or to bring about a biological effect disclosed herein, in combination with one or more nicotinyl riboside compounds and an uncoupler is no more than about 10%, 5%, 1%, 0.5% or 0.1% of the recommended dose of the PARP inhibitor as an antitumor/anticancer agent. In certain embodiments, the dose of a PARP inhibitor for such a use is no more than about 1% of the recommended dose of the PARP inhibitor as an antitumor/anticancer agent. In some embodiments, the PARP inhibitor is olaparib, and the dose (e.g., per day or per dose) of olaparib to treat a non-tumor/non-cancer disease/disorder or condition disclosed herein, or to bring about a biological effect disclosed herein, in combination with one or more nicotinyl riboside compounds and an uncoupler is no more than about 10 mg, 5 mg, 1 mg, 0.5 mg or 0.1 mg; or is from about 0.01 or 0.1 mg to about 10 mg, from about 0.01 or 0.1 mg to about 1 mg, or from about 1 mg to about 10 mg; or is about 0.01-0.1 mg, 0.1-0.5 mg, 0.5-1 mg, 1-5 mg or 5-10 mg; or is about 10 μg, 50 μg, 0.1 mg, 0.5 mg, 1 mg, 5 mg or 10 mg. In certain embodiments, the dose (e.g., per day or per dose) of olaparib for such a use is no more than about 1 mg. In some embodiments, the concentration (e.g., in the blood/plasma/serum, such as the steady-state blood/plasma/serum concentration) of olaparib is from about 1 nM to about 10, 20 or 50 nM, or about 1-10 nM, or from about 10 nM to about 20 or 50 nM, or about 5 nM. The steady-state blood/plasma/serum concentration of olaparib can also be lower, such as about 0.1-1 nM.

The PARP inhibitor can be administered in any suitable frequency. In certain embodiments, the PARP inhibitor is administered once or twice daily. Likewise, the PARP inhibitor can be administered via any suitable route. In certain embodiments, the PARP inhibitor is administered orally. In other embodiments, the PARP inhibitor is administered parenterally (e.g., intravenously, subcutaneously, intramuscularly, intrathecally or topically [e.g., sublingually]).

The length of treatment with one or more nicotinyl riboside compounds (e.g., NR or/and NRH, NRTA or/and NRHTA, or one or more NR/NAR derivatives disclosed herein) and a mitochondrial uncoupler (e.g., one providing mild uncoupling such as NTZ, niclosamide, OPC-163493, controlled-release DNP, BAM15 or Example No. 134 in WO 2019/226490), and optionally a PARP inhibitor (e.g., olaparib), to treat a disease/disorder or condition described herein, or to bring about a biological effect described herein, can be determined by the treating physician. For example, the length of treatment with the one or more nicotinyl riboside compounds and the uncoupler, and the optional PARP inhibitor, independently can be at least about 1 day, 2 days, 3 days, 1 week, 2 weeks, 3 weeks, 4 weeks (1 month), 6 weeks, 2 months, 3 months, 6 months, 1 year, 2 years, 3 years, 4 years, 5 years or longer.

In some embodiments, one or more nicotinyl riboside compounds or/and an uncoupler, or/and optionally a PARP inhibitor, independently are administered acutely (e.g., less than about 6 weeks, such as at least about 1 day, 2 days, 3 days, 1 week, 2 weeks, 3 weeks, 4 weeks or 5 weeks) to treat an acute disorder or condition, such as to prevent or minimize damage to threatened cells, tissue(s) or organ(s) as a result of an ischemia or body trauma. Even a short dosing of the one or more nicotinyl riboside compounds or/and the uncoupler, or/and the optional PARP inhibitor, may be sufficient depending on the nature and severity of the medical condition. For instance, one dose of an uncoupler may be sufficient to prevent or minimize damage from a mild head or brain injury (e.g., a mild concussion). In some embodiments, treatment with the one or more nicotinyl riboside compounds or/and the uncoupler, or/and the optional PARP inhibitor, independently begins within a short time of the occurrence of the ischemia or body trauma, such as within about 2, 4, 6, 12 or 24 hours after the occurrence of the ischemia or body trauma, or before engagement in an activity that has a high risk of a body trauma (e.g., a military action or a boxing, American football or mixed martial arts match).

In other embodiments, one or more nicotinyl riboside compounds or/and an uncoupler, or/and optionally a PARP inhibitor, independently are administered chronically (e.g., at least about 6 weeks, 2 months, 3 months, 6 months, 1 year, 2 years, 3 years, 4 years, 5 years or longer) to treat a chronic disease or condition, such as a chronic mitochondrial disease or a chronic mitochondria-related disease or condition. In some embodiments, the chronic mitochondria-related disease or condition is a metabolic disorder, an obesity-associated condition, an inflammatory disorder, an immune-related disorder, a fibrotic disorder, a disorder associated with oxidative stress (other than an ischemia, IRI or body trauma in certain embodiments), a neurological disorder, a proliferative disorder (e.g., a tumor or cancer), or an aging-related disorder. One or more nicotinyl riboside compounds or/and an uncoupler, or/and optionally a PARP inhibitor, can also be administered, such as in a low or very low dose, over a period of years (e.g., over at least about 5 or 10 years, or over most of a person's adult life) as a wellness or anti-aging regimen. In some embodiments, one or more nicotinyl riboside compounds or/and an uncoupler, or/and optionally a PARP inhibitor, are administered chronically to treat (e.g., to facilitate recovery from) an ischemia (e.g., a stroke), an IRI (e.g., a cerebral IRI) or a body trauma (e.g., a TBI). For example, chronic treatment with an uncoupler after a stroke, a cerebral IRI or a TBI can increase BDNF levels in the brain, which can promote neuronal growth, repair of damaged neurons, and formation of synapses.

One or more nicotinyl riboside compounds and an uncoupler, and optionally a PARP inhibitor, can also be taken pro re nata (as needed) until clinical manifestations of the condition disappear or clinical targets are achieved. For example, the one or more nicotinyl riboside compounds and the uncoupler, and the optional PARP inhibitor, can be taken until attainment of a target blood glucose level, blood pressure, blood levels of lipids, body weight, body mass index or percent body fat, or any combination thereof. If clinical manifestations of the condition re-appear or the clinical targets are not maintained, administration of the one or more nicotinyl riboside compounds and the uncoupler, and the optional PARP inhibitor, can resume. Under an alternative pro re nata treatment and also at the treating physician's discretion, the dose of the one or more nicotinyl riboside compounds and the uncoupler, and the optional PARP inhibitor, or/and their dosing frequency can be reduced upon improvement of clinical outcome(s) and then can be increased (e.g., to the previously effective dose or/and dosing frequency) if the patient's clinical status subsequently worsens.

One or more nicotinyl riboside compounds (e.g., NR or/and NRH, NRTA or/and NRHTA, or one or more NR/NAR derivatives disclosed herein) and a mitochondrial uncoupler (e.g., one providing mild uncoupling such as NTZ, niclosamide, OPC-163493, DNP methyl ether, controlled-release DNP, BAM15 or Example No. 134 in WO 2019/226490), and optionally a PARP inhibitor (e.g., olaparib), can be administered in the same pharmaceutical composition or in separate compositions. In some embodiments, one or more nicotinyl riboside compounds and an uncoupler, and optionally a PARP inhibitor, are administered in a fixed-dose combination dosage form. In some embodiments, the fixed-dose combination dosage form is a controlled-release, slow-release or sustained-release form. In certain embodiments, the fixed-dose combination dosage form is formulated for oral administration, such as once or twice daily and such as in the form of a tablet, capsule or pill. In other embodiments, the fixed-dose combination dosage form is formulated for parenteral administration, such as intravenously, subcutaneously, intramuscularly, intrathecally or topically (e.g., sublingually). In further embodiments, one or more nicotinyl riboside compounds or/and an uncoupler, or/and optionally a PARP inhibitor, are administered as a complex with a dendrimer (e.g., a PAMAM or/and PEG dendrimer) or via a dendrimer-containing composition. The dendrimer can optionally have one or more moieties for targeting to specific organ(s), tissue(s), cell type(s) or organelle(s), such as one or more N-acetylgalactosamine moieties for targeting to the liver for treatment of, e.g., a liver or metabolic disorder, or one or more RGD-containing moieties for targeting to tumor/cancer cells with upregulated cell-membrane integrins for treatment of a tumor or cancer.

### Representative Embodiments

The following embodiments of the disclosure are provided to illustrate the disclosure:

1. A compound of Formula I or II:

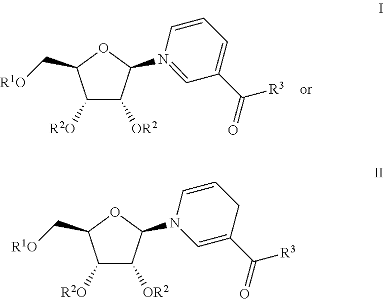

wherein:

R¹ is hydrogen,

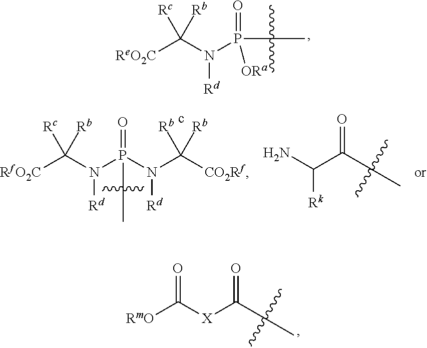

R^{a} is hydrogen, a counterion, linear or branched C₁-C₆ alkyl, C₃-C₆ cycloalkyl, phenyl, 1-naphthyl or 2-naphthyl, wherein the phenyl is optionally substituted with F, Cl, —NO₂, linear or branched C₁-C₄ alkyl, —CF₃ or —O-(linear or branched C₁-C₄ alkyl);
R^{b} and R^{c} at each occurrence independently are hydrogen, linear or branched C₁-C₅ alkyl, —CH₂-phenyl, —CH₂-3-indole or —CH₂-5-imidazole, wherein the alkyl is optionally substituted with —OH, —OR^{j}, —SH, —SR^{j}, —NH₂, —NHR^{j}, —N(R^{j})₂, —NHC(═O)R^{j}, —NHC(═NH)NH₂, —C(═O)NH₂, —CO₂H or —C(═O)OR^{j}, and the phenyl is optionally substituted with —OH or —OR^{j}, wherein R¹ at each occurrence independently is linear or branched C₁-C₄ alkyl;
R^{d} at each occurrence independently is hydrogen, methyl or linear or branched C₂-C₄ alkyl;
R^{e} and R^{f} at each occurrence independently are hydrogen, a counterion, linear or branched C₁-C₅ alkyl, C₃-C₆ cycloalkyl, —CH₂—(C₃-C₆ cycloalkyl), phenyl or —CH₂-phenyl, wherein the phenyl is optionally substituted with F, Cl, —NO₂, linear or branched C₁-C₄ alkyl, —CF₃ or —O-(linear or branched C₁-C₄ alkyl);
R^{k} is hydrogen, linear or branched C₁-C₆ alkyl, —CH₂-phenyl, —CH₂-3-indole or —CH₂-5-imidazole, wherein the alkyl is optionally substituted with —OH, —OR^{j}, —SH, —SR^{j}, —NH₂, —NHR^{j}, —N(R^{j})₂, —NHC(═O)R^{j}, —NHC(═NH)NH₂, —C(═O)NH₂, —CO₂H or —C(═O)OR^{j}, and the phenyl is optionally substituted with —OH or —OR^{j}, wherein R¹ at each occurrence independently is linear or branched C₁-C₄ alkyl;
R^{m} is hydrogen, a counterion, linear or branched C₁-C₆ alkyl, C₃-C₆ cycloalkyl, phenyl, —CH₂-phenyl or

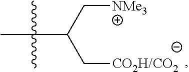

wherein the phenyl is optionally substituted with F, Cl, —NO₂, linear or branched C₁-C₄ alkyl, —CF₃ or —O-(linear or branched C₁-C₄ alkyl); and
X is cis or trans —HC═CH— or —(CH₂)— optionally substituted with —OH, —OR^{j} or —OC(═O)R^{j}, wherein R¹ is linear or branched C₁-C₄ alkyl and n is 1, 2, 3, 4, 5 or 6;

R² at each occurrence independently is hydrogen,

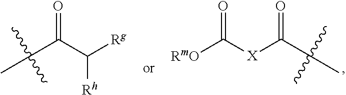

wherein:
R^{g} is hydrogen, linear or branched C₁-C₅ alkyl, —CH₂-phenyl, —CH₂-3-indole or —CH₂-5-imidazole, wherein the alkyl is optionally substituted with —OH, —OR^{j}, —SH, —SR^{j}, —NH₂, —NHR^{j}, —N(R^{j})₂, —NHC(═O)R^{j}, —NHC(═NH)NH₂, —C(═O)NH₂, —CO₂H or —C(═O)OR^{j}, and the phenyl is optionally substituted with —OH or —OR^{j}, wherein R¹ at each occurrence independently is linear or branched C₁-C₄ alkyl;
R^{h} is hydrogen, methyl or —NH₂;
or R^{g} and R^{h} together with the carbon atom to which they are connected form a C₃-C₆ cycloalkyl or phenyl ring, wherein the phenyl ring is optionally substituted with F, Cl, —NO₂, linear or branched C₁-C₄ alkyl, —CF₃ or —O-(linear or branched C₁-C₄ alkyl); and
R^{m} and X are as defined above; and

R³ is —NH₂, —NHRⁿ, —N(Rⁿ)₂, —OH, —OR^{o} or

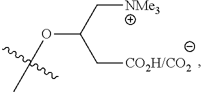

wherein:
Rⁿ at each occurrence independently is linear or branched C₁-C₆ alkyl or allyl, wherein the alkyl is optionally substituted with —OH or —O-(linear or branched C₁-C₃ alkyl), or both occurrences of Rⁿ and the nitrogen atom to which they are connected form a 3- to 6-membered heterocyclic ring; and
R^{o} is a counterion, linear or branched C₁-C₆ alkyl, C₃-C₆ cycloalkyl, phenyl or —CH₂-phenyl, wherein the phenyl is optionally substituted with F, Cl, —NO₂, linear or branched C₁-C₄ alkyl, —CF₃ or —O-(linear or branched C₁-C₄ alkyl);
or a pharmaceutically acceptable salt, solvate, hydrate, clathrate, polymorph or stereoisomer thereof;
with the proviso that:

1) R¹ and both occurrences of R² all are not hydrogen except when R³ is

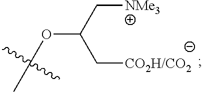

and

2) the compound of Formula I or II is not:

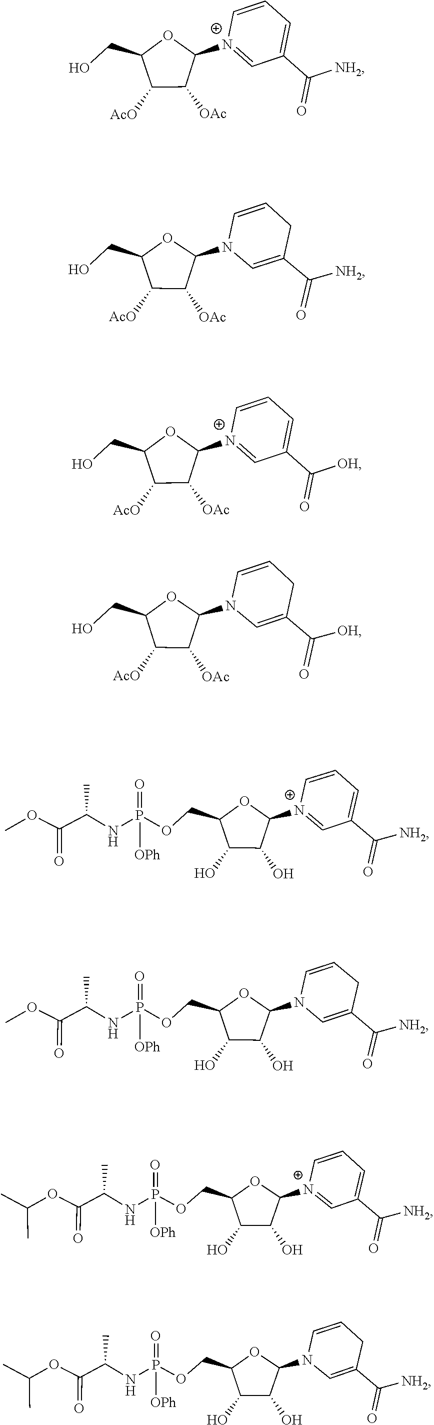

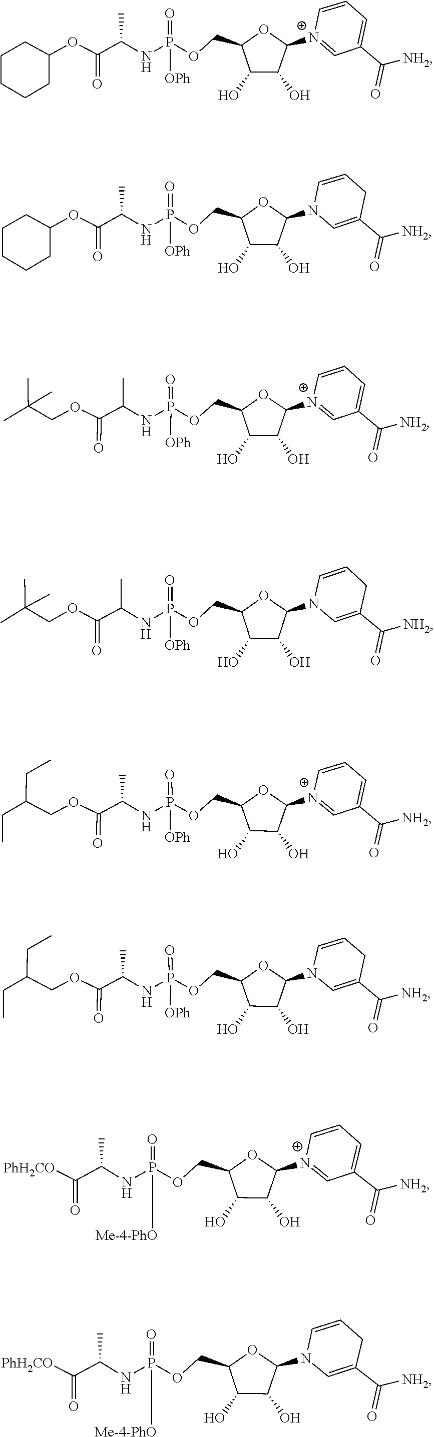

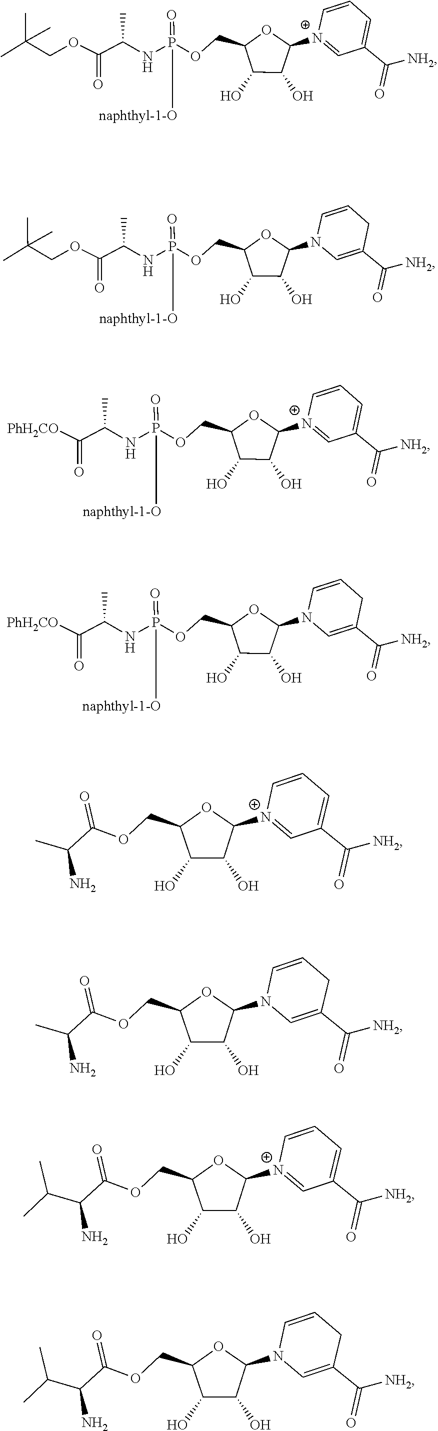

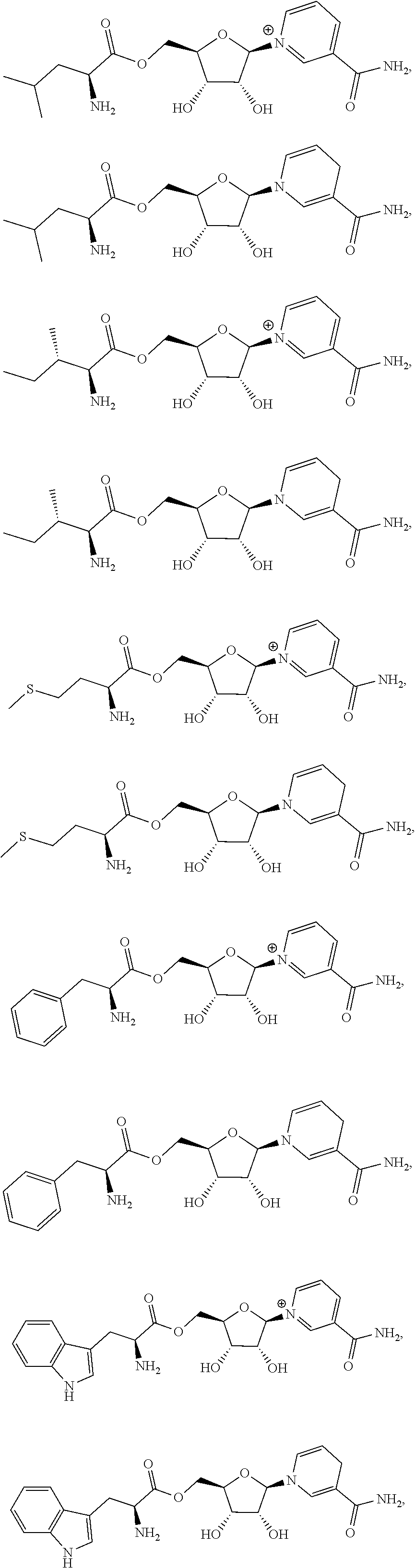

or a salt or stereoisomer thereof.
2. The compound of Formula I or II of embodiment 1, wherein when both occurrences of R² are acetyl:

1) R¹ is not hydrogen; or

2) R³ is not —NH₂ or —OH or a salt thereof; or

3) R¹ is not hydrogen and R³ is not —NH₂ or —OH or a salt thereof.

3. The compound of Formula I or II of embodiment 1, wherein when R¹ is

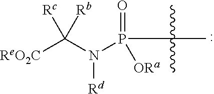

1) both occurrences of R² are not hydrogen; or

2) R³ is not —NH₂ or —OH or a salt thereof; or

3) both occurrences of R² are not hydrogen and R³ is not —NH₂ or —OH or a salt thereof.

4. The compound of Formula I or II of embodiment 1, wherein when R¹ is

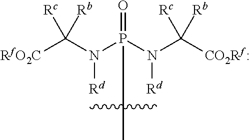

1) both occurrences of R² are not hydrogen; or

2) R³ is not —NH₂ or —OH or a salt thereof; or

3) both occurrences of R² are not hydrogen and R³ is not —NH₂ or —OH or a salt thereof.

5. The compound of Formula I or II of embodiment 1, wherein when R¹

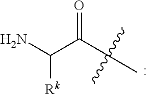

1) both occurrences of R² are not hydrogen; or

2) R³ is not —NH₂ or —OH or a salt thereof; or

3) both occurrences of R² are not hydrogen and R³ is not —NH₂ or —OH or a salt thereof.

6. The compound of Formula I or II of embodiment 1, wherein R¹ is hydrogen.
7. The compound of Formula I or II of embodiment 1 or 3, wherein R¹ is

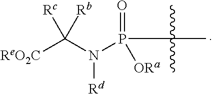

8. The compound of Formula I or II of embodiment 7, wherein R¹ is

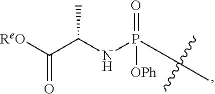

and R^{e} is linear or branched C₁-C₆ alkyl, such as methyl, ethyl or isopropyl.
9. The compound of Formula I or II of embodiment 1 or 4, wherein R¹ is

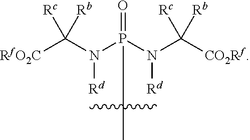

10. The compound of Formula I or II of embodiment 9, wherein R¹ is

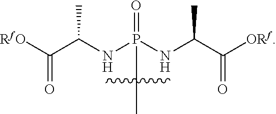

and both occurrences of R^{f} are linear or branched C₁-C₆ alkyl, such as methyl, ethyl or isopropyl.
11. The compound of Formula I or II of embodiment 1, wherein R¹ is

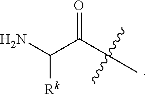

12. The compound of Formula I or II of embodiment 11, wherein R¹ is

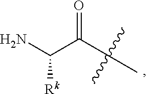

and R^{k} is linear or branched C₁-C₆ alkyl, such as methyl, ethyl or isopropyl.
13. The compound of Formula I or II of embodiment 1, wherein R¹, or/and R² at either occurrence or at both occurrences, is/are

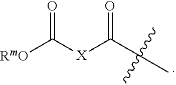

14. The compound of Formula I or II of embodiment 13, wherein:

X is trans —HC═CH—, —CH₂CH₂— or —CH(OH)CH₂—; and

R^{m} is hydrogen, a counterion, linear or branched C₁-C₆ alkyl (e.g., methyl, ethyl or isopropyl) or

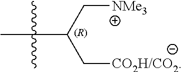

15. The compound of Formula I or II of embodiment 14, wherein R¹, or/and R² at either occurrence or at both occurrences, is/are selected from:

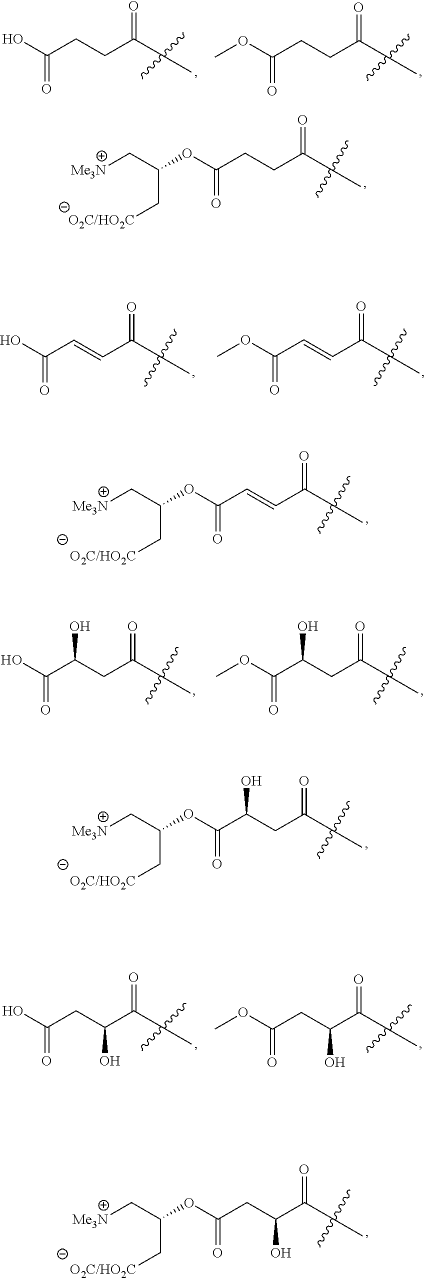

and salts thereof.
16. The compound of Formula I or II of any one of the preceding embodiments, wherein R² at each occurrence independently, or at both occurrences, is hydrogen, —C(═O)-(linear or branched C₁-C₆ alkyl),

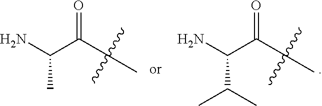

17. The compound of Formula I or II of embodiment 16, wherein R² at each occurrence independently, or at both occurrences, is hydrogen, acetyl or propanoyl.
18. The compound of Formula I or II of any one of the preceding embodiments, wherein R³ is —NH₂, —OH or a salt thereof, or

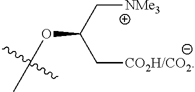

19. The compound of Formula I or II of embodiment 18, wherein R³ is

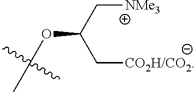

20. The compound of Formula I or II of embodiment 1 or 3, wherein:

1) R¹ is

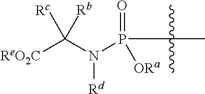

and both occurrences of R² are acetyl or propanoyl; or

2) R¹ is

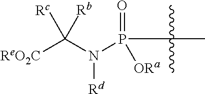

and R³ is —OH or a salt thereof; or

3) R¹ is

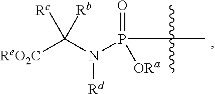

both occurrences of R² are acetyl or propanoyl, and R³ is —OH or a salt thereof.
21. The compound of Formula I or II of embodiment 20, wherein R¹ is

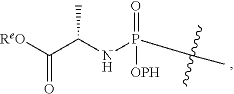

and R^{e} is linear or branched C₁-C₆ alkyl, such as methyl, ethyl or isopropyl.
22. The compound of Formula I or II of embodiment 1 or 3, wherein:

R¹ is

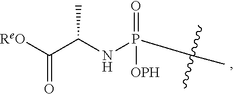

wherein R^{e} is linear or branched C₁-C₆ alkyl;

R² at both occurrences is —C(═O)-(linear or branched C₁-C₆ alkyl); and

R³ is —NH₂ or —OH or a salt thereof.

23. The compound of Formula I or II of embodiment 22, wherein:

R^{e} of the R¹ moiety is methyl, ethyl or isopropyl; and

R² at both occurrences is acetyl or propanoyl.

24. The compound of Formula I or II of embodiment 1 or 4, wherein:

R¹ is

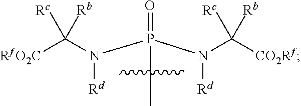

R² at each occurrence independently, or at both occurrences, is hydrogen, acetyl or propanoyl; and

R³ is —NH₂ or —OH or a salt thereof.

25. The compound of Formula I or II of embodiment 24, wherein for the R¹ moiety:

R^{b} and R^{c} at each occurrence independently are hydrogen or linear or branched C₁-C₅ alkyl, or each pair of R^{b} and R^{c} is hydrogen and linear or branched C₁-C₅ alkyl;

R^{d} at both occurrences is hydrogen; and

R^{f} at both occurrences is linear or branched C₁-C₆ alkyl.

26. The compound of Formula I or II of embodiment 25, wherein R¹ is

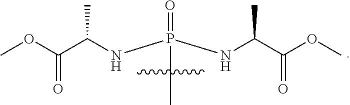

27. The compound of Formula I or II of embodiment 1 or 4, wherein:

R¹ is

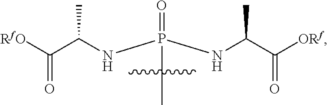

wherein R^{f} at both occurrences is linear or branched C₁-C₆ alkyl;

R² at each occurrence independently, or at both occurrences, is hydrogen or —C(═O)-(linear or branched C₁-C₆ alkyl); and

R³ is —NH₂ or —OH or a salt thereof.

28. The compound of Formula I or II of embodiment 27, wherein:

R^{f} of the R¹ moiety at both occurrences is methyl, ethyl or isopropyl; and

R² at each occurrence independently, or at both occurrences, is hydrogen, acetyl or propanoyl.

29. The compound of Formula I or II of embodiment 1, wherein:

R¹ is

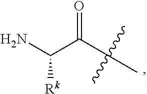

wherein R^{k} is linear or branched C₁-C₆ alkyl;

R² at each occurrence independently, or at both occurrences, is hydrogen or —C(═O)-(linear or branched C₁-C₆ alkyl); and

R³ is —NH₂ or —OH or a salt thereof.

30. The compound of Formula I or II of embodiment 29, wherein:

R^{k} of the R¹ moiety is methyl, ethyl or isopropyl; and

R² at each occurrence independently, or at both occurrences, is hydrogen, acetyl or propanoyl.

31. The compound of Formula I or II of embodiment 1, wherein:

R¹ is

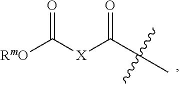

wherein:
X is cis or trans —HC═CH— or —(CH₂)— optionally substituted with —OH, —OR^{j} or —OC(═O)R^{j}, wherein R^{j} is linear or branched C₁-C₄ alkyl and n is 1, 2, 3, 4, 5 or 6; and
R^{m} is hydrogen, a counterion, linear or branched C₁-C₆ alkyl or

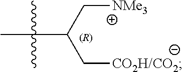

R² at each occurrence independently, or at both occurrences, is hydrogen or —C(═O)-(linear or branched C₁-C₆ alkyl); and

R³ is —NH₂ or —OH or a salt thereof.

32. The compound of Formula I or II of embodiment 31, wherein:

for the R¹ moiety, X is trans-HC—CH—, —CH₂CH₂— or —CH(OH)CH₂—, and R^{m} is hydrogen, a counterion, methyl, ethyl, isopropyl or

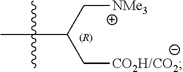

R² at each occurrence independently, or at both occurrences, is hydrogen, acetyl or propanoyl; and

R³ is-NH₂.

33. The compound of Formula I or II of embodiment 1, which is selected from:

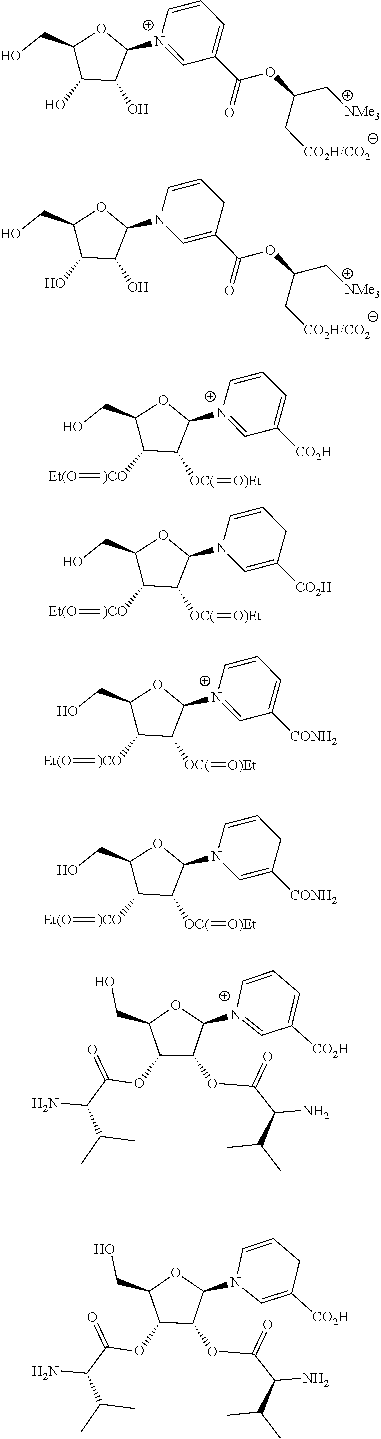

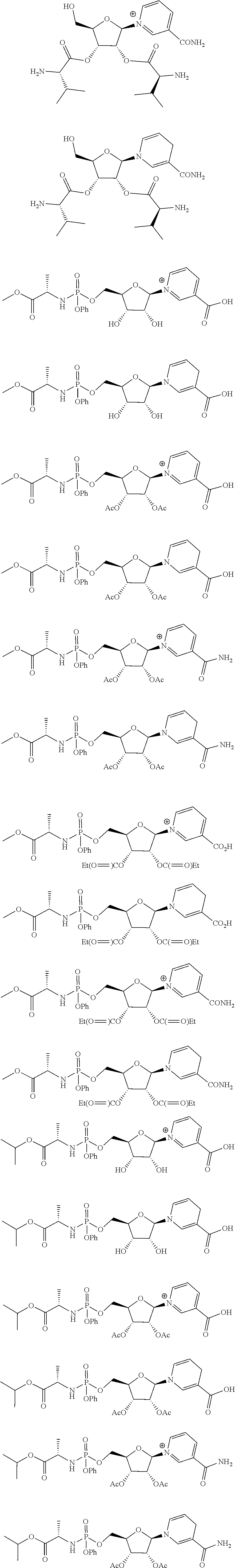

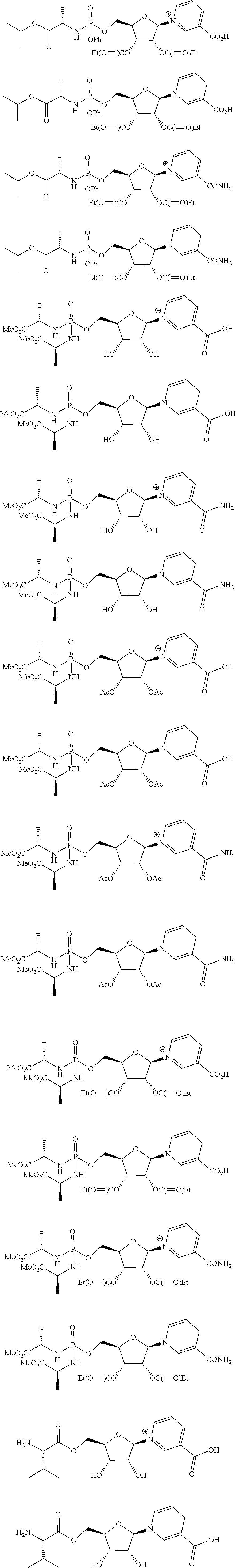

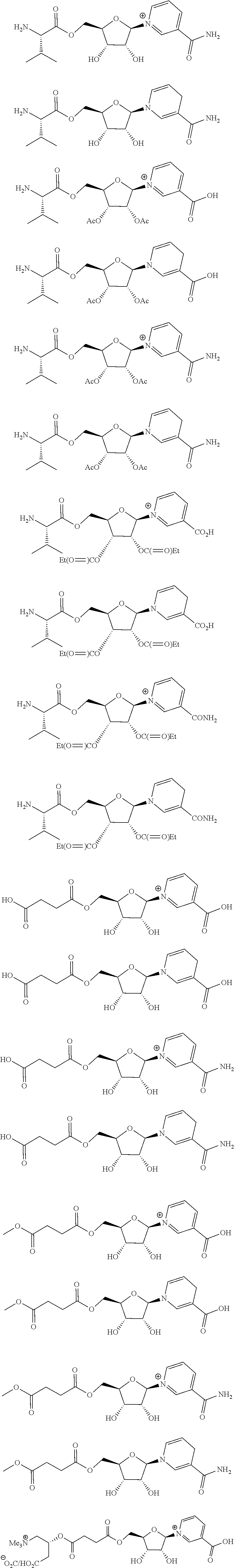

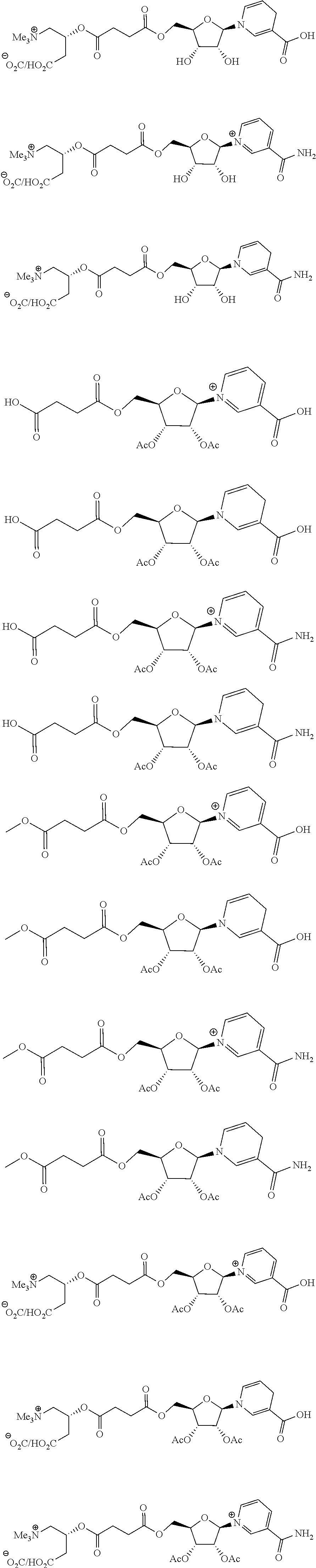

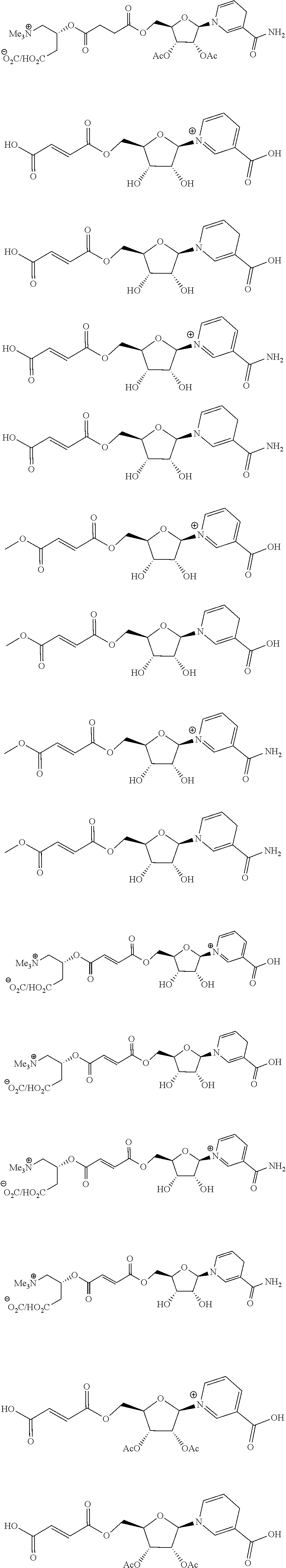

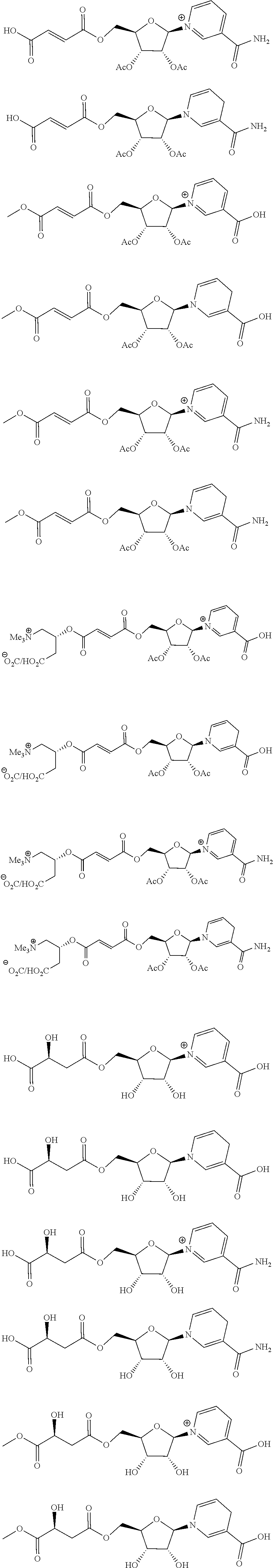

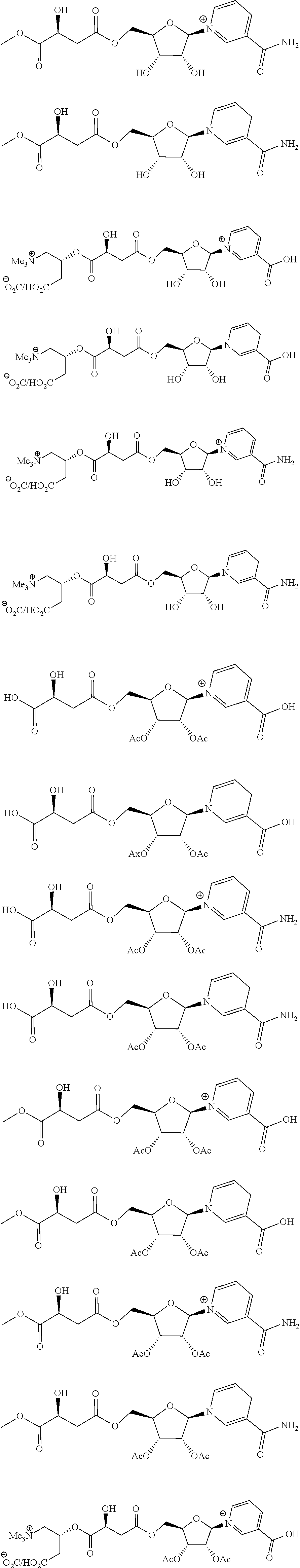

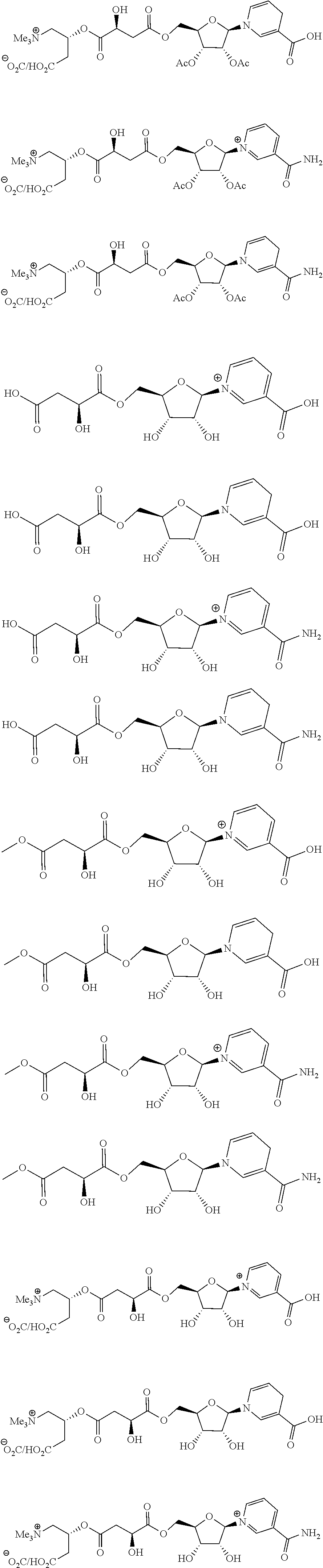

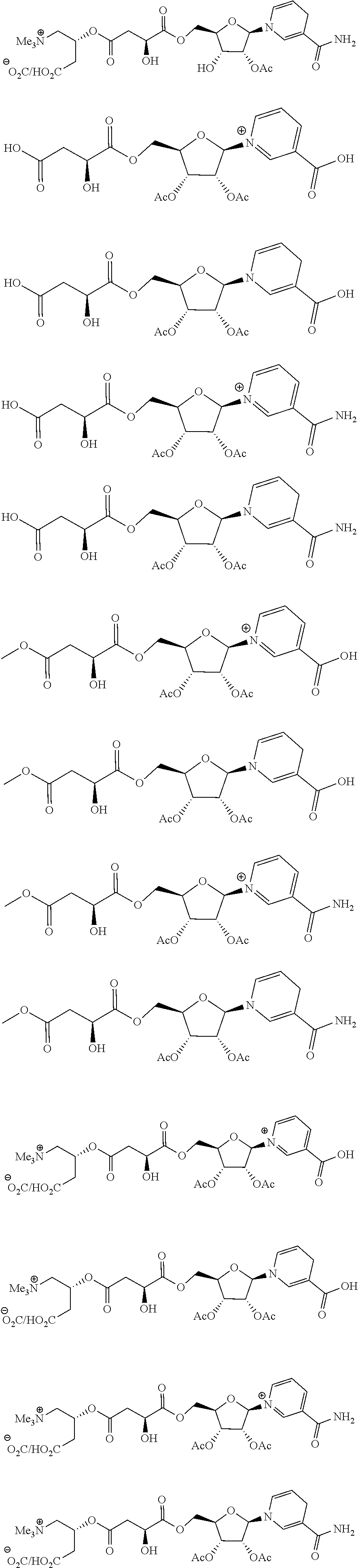

and pharmaceutically acceptable salts, solvates, hydrates, clathrates, polymorphs and stereoisomers thereof.
34. The compound of Formula I or II of any one of the preceding embodiments, which is a compound of Formula II.
35. A compound of Formula III or IV:

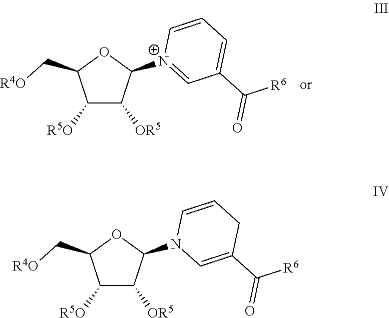

wherein:
R⁴ is hydrogen or —C(═O)R⁷, wherein R⁷ is linear or branched C₁-C₆ alkyl, C₃-C₆ cycloalkyl, or phenyl optionally substituted with F, Cl, —NO₂, linear or branched C₁-C₄ alkyl, —CF₃ or —O-(linear or branched C₁-C₄ alkyl);
R⁵ at each occurrence independently is hydrogen or —C(═O)R⁸, wherein R⁸ has the same definition as R⁷; and
R⁶ is

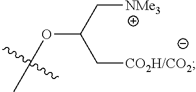

or a pharmaceutically acceptable salt, solvate, hydrate, clathrate, polymorph or stereoisomer thereof.
36. The compound of Formula III or IV of embodiment 35, wherein:

R⁴ is hydrogen or —C(═O)R⁷, wherein R⁷ is linear or branched C₁-C₆ alkyl; and

R⁵ at each occurrence independently, or at both occurrences, is hydrogen or —C(═O)R⁸, wherein R⁸ is linear or branched C₁-C₆ alkyl.

37. The compound of Formula III or IV of embodiment 36, wherein:

R⁴ is hydrogen, acetyl or propanoyl; and

R⁵ at each occurrence independently, or at both occurrences, is hydrogen, acetyl or propanoyl.

38. The compound of Formula III or IV of any one of embodiments 35 to 37, wherein R⁶ is

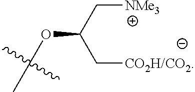

39. The compound of Formula III or IV of any one of embodiments 35 to 38, which is selected from:

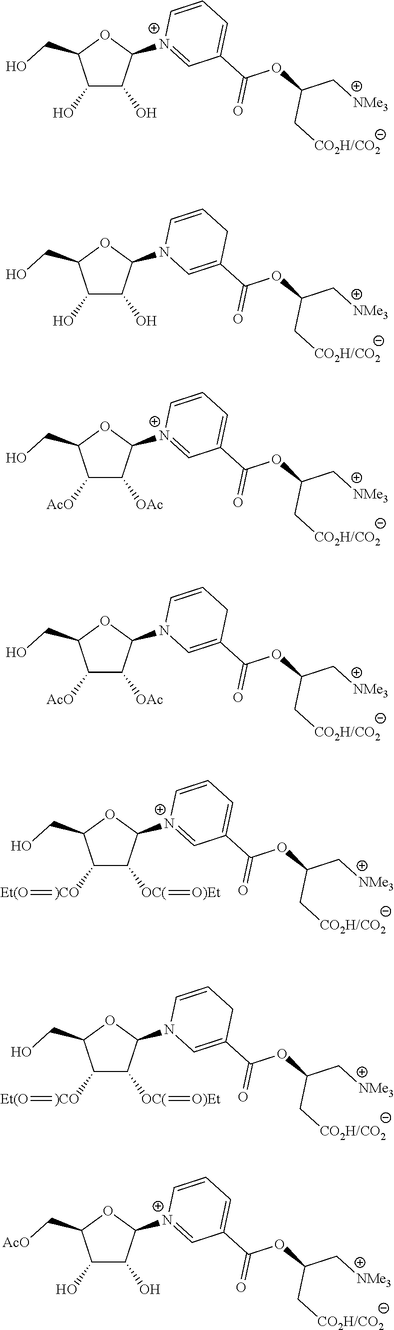

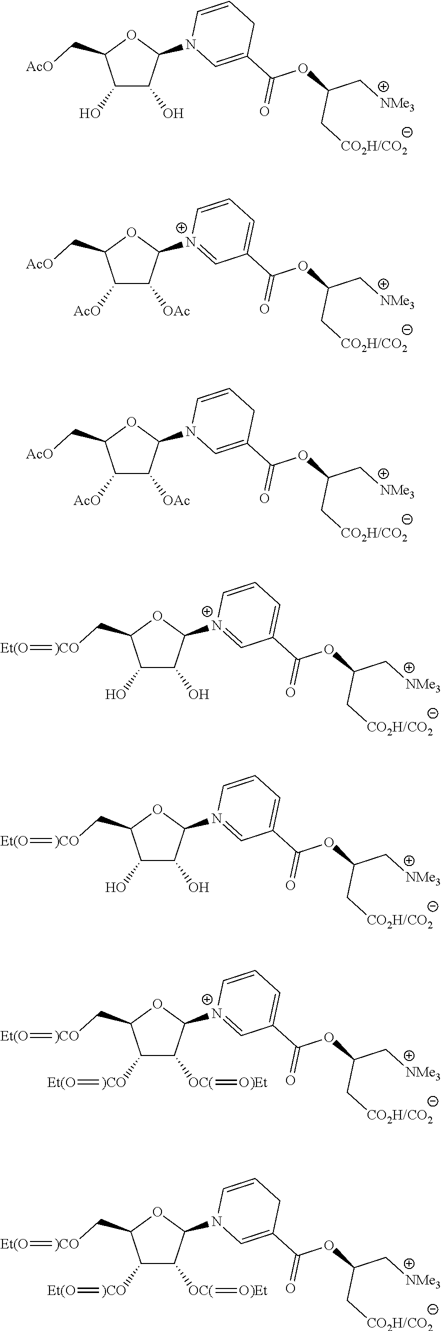

and pharmaceutically acceptable salts, solvates, hydrates, clathrates, polymorphs and stereoisomers thereof.
40. The compound of Formula II or IV of any one of embodiments 35 to 39, which is a compound of Formula IV.
41. The compound of Formula I, II, III or IV of any one of the preceding embodiments, which is a trifluoromethanesulfonate (triflate or ⁻OTf) salt, an acetate (⁻OAc) salt, a trifluoroacetate (⁻OTFA) salt, a formate salt or a chloride (Cl⁻) salt.
42. The compound of Formula I, II, III or IV of any one of the preceding embodiments, which has the beta-D-riboside configuration.
43. The compound of Formula I, II, III or IV of any one of the preceding embodiments, which is stereoisomerically pure (e.g., at least about 90%, 95%, 98% or 99% of the compound is the indicated stereoisomer).
44. The compound of Formula I, II, III or IV of any one of embodiments 1 to 41, which is a racemic mixture.
45. The compound of Formula I, II, III or IV of any one of embodiments 1 to 41, which has the D-riboside configuration and an approximately 1:1 ratio of beta-/alpha-anomers.
46. A pharmaceutical or cosmetic composition comprising one or more compounds of any one of the preceding embodiments or a pharmaceutically acceptable salt, solvate, hydrate, clathrate, polymorph or stereoisomer thereof, and one or more pharmaceutically acceptable excipients or carriers.
47. The pharmaceutical or cosmetic composition of embodiment 46, which comprises a compound of Formula II or IV.
48. The pharmaceutical or cosmetic composition of embodiment 46 or 47, which comprises a compound of Formula I and a compound of Formula II, or a compound of Formula III and a compound of Formula IV.
49. A method of treating a mitochondrial disease, a mitochondria-related disease or condition, or a disease or condition characterized by acute NAD⁺ depletion due to DNA damage, comprising administering to a subject in need of treatment a therapeutically effective amount of one or more compounds of any one of the preceding embodiments or a pharmaceutically acceptable salt, solvate, hydrate, clathrate, polymorph or stereoisomer thereof, or a pharmaceutical composition comprising the same.
50. The method of embodiment 49, wherein the mitochondrial disease is selected from mitochondrial myopathies; limb-girdle distribution weakness; mitochondrial transcription factor A (TFAM) deficiency; Kearns-Sayre syndrome (KSS); Pearson syndrome; Leigh syndrome; Barth syndrome; Friedreich's ataxia; ataxia neuropathy syndrome/spectrum (ANS, including mitochondrial recessive ataxia syndrome [MIRAS] and sensory ataxia neuropathy, dysarthria and ophthalmoplegia [SANDO]); neuropathy, ataxia and retinitis pigmentosa (NARP); mitochondrial DNA depletion syndrome (Alper's disease); mitochondrial neurogastrointestinal encephalopathy (MNGIE) syndrome; mitochondrial encephalopathy, lactic acidosis and stroke-like episodes (MELAS) syndrome; myoclonic epilepsy with ragged red fibers (MERRF); chronic progressive external ophthalmoplegia (CPEO); Leber's hereditary optic neuropathy (LHON); inherited forms of blindness and deafness (e.g., diabetes mellitus and deafness); acquired forms of reversible or permanent hearing loss (e.g., type 2 diabetes-associated hearing loss and hearing loss induced by ototoxic chemicals (e.g., heavy metals [e.g., lead], solvents [e.g., styrene and toluene] and asphyxiants [e.g., carbon monoxide]) and medications (e.g., loop diuretics [e.g., bumetanide and furosemide], NSAIDs [e.g., aspirin, celecoxib, diclofenac, ibuprofen and naproxen], PDE5 inhibitors, macrolide antibiotics, aminoglycosides [e.g., gentamicin], platinum-based chemotherapeutics [e.g., carboplatin and cisplatin], paracetamol and quinine)); and disorders (e.g., myopathy, neuropathy, lactic acidosis and lipodystrophy) resulting from mitochondrial toxicity due to, e.g., medications (e.g., antiviral drugs (including antiretroviral drugs [e.g., nucleoside analog reverse transcriptase inhibitors {NRTIs}] against HIV and nucleoside and nucleotide analogs against HBV. HCV and CMV)).
51. The method of embodiment 49 or 50, wherein the mitochondrial disease is a primary mitochondrial disease.
52. The method of embodiment 49, wherein the mitochondria-related disease or condition is a neurodegenerative disorder, a neuronal activation disorder, a muscle disorder, a metabolic disorder, a fatty acid/beta oxidation disorder, a disorder associated with abnormal or ectopic lipid accumulation or storage, a lysosomal storage disease (e.g., a lipid storage disorder), a disorder associated with oxidative stress, an inflammatory disorder, an immune-related disorder, a vascular disorder, a kidney disorder, a liver disorder, a proliferative disorder (e.g., a tumor or cancer), male or female infertility, or an aging-related disorder.
53. The method of embodiment 49 or 52, wherein the mitochondria-related disease or condition is selected from lipodystrophy (including congenital and acquired, partial and generalized, and severe), metabolic syndrome, obesity, types 1 and 2 diabetes, liver disorders (e.g., non-alcoholic fatty liver disease [NAFLD], non-alcoholic steatohepatitis [NASH], alcoholic liver disease [ALD], alcoholic steatohepatitis [ASH], autoimmune hepatitis and cholestatic liver disease), hemochromatosis and alpha-1 antitrypsin deficiency.
54. The method of embodiment 49, wherein the disease or condition characterized by acute NAD⁺ depletion due to DNA damage is selected from exposure to radiation (e.g., UV and ionizing radiation such as X-ray), radiation or chemotherapy-induced disorders (e.g., dermatitis, myositis, myocarditis, colitis, prostatitis, hepatitis, pneumonitis, neuropathies and bone marrow failure), burn injuries (including first-degree burns, second-degree burns and third-degree burns), chemical exposure with manifestation of exfoliative dermatitis, exposure to chemical warfare agents, Stevens-Johnson syndrome, acute respiratory distress syndrome, inhalational lung injury due to smoke or chemical toxins, trauma-related crush injuries (including those with bone fractures), peripheral nerve injuries, spinal cord injuries, and contusion to internal organs (such as the heart, lung, liver, and kidneys).
55. The method of any one of embodiments 49, 50 and 52 to 54, wherein the mitochondrial disease, the mitochondria-related disease or condition, or the disease or condition characterized by acute NAD⁺ depletion due to DNA damage is associated with (e.g., is caused by or results in) secondary mitochondrial dysfunction.
56. The method of any one of embodiments 49 to 55, wherein the one or more compounds are or comprise a compound of Formula II or IV.
57. The method of any one of embodiments 49 to 56, wherein the one or more compounds are or comprise a compound of Formula I and a compound of Formula II, or a compound of Formula III and a compound of Formula IV.
58. The method of any one of embodiments 49 to 57, wherein the one or more compounds or the pharmaceutical composition is/are administered orally, parenterally (e.g., intravenously, intradermally, subcutaneously, intramuscularly or intrathecally), or topically (e.g., transdermally, transmucosally, intranasally, pulmonarily [e.g., by oral inhalation], sublingually or rectally [e.g., by suppository]).
59. The method of any one of embodiments 49 to 57, wherein the one or more compounds or the pharmaceutical composition is/are used in culture medium for preparation of ex vivo therapy.
60. The method of embodiment 59, wherein the ex vivo therapy is a chimeric antigen receptor T-cell (CAR-T) therapy, a stem cell therapy, in vitro fertilization, organ transplantation or for attachment to a carrier molecule such as a dendrimer or an antibody conjugate.
61. The method of any one of embodiments 49 to 60, further comprising administering a therapeutically effective amount of at least one other therapeutic agent selected from sirtuin-activating agents, AMPK-activating agents, CD38 inhibitors, PARP inhibitors, mitochondrial uncouplers, stimulators of cellular oxygen consumption, NMDA receptor antagonists, acetylcholinesterase inhibitors, antidiabetics, anti-obesity agents, antiplatelet agents, anticoagulants, antihypertensive agents, antioxidants, anti-inflammatory agents, analgesics, anesthetics, anticancer agents, antivirals, antibiotics, antifungals, natural compounds, vitamins, vaccines, and combinations thereof.
62. The method of embodiment 61, wherein the at least one other therapeutic agent is or comprises a sirtuin-activating agent, a PARP inhibitor, an antioxidant, a natural compound or a vitamin, or any combination thereof.
63. The method of embodiment 61 or 62, wherein the sirtuin-activating agent is selected from polyphenols (e.g., butein, fisetin, isoliquiritigenin, piceatannol, quercetin and resveratrol), amino acids with a branched side chain (e.g., leucine), methylene blue, SRT-1460, SRT-1720, SRT-2104, SRT-2183, lamin A, and salts thereof.
64. The method of embodiment 61 or 62, wherein the PARP inhibitor is selected from niraparib, olaparib, pamiparib (BGB290), rucaparib, talazoparib, veliparib, 4-amino-1,8-naphthalimide, CEP9722, E7016, PJ34, and salts thereof.
65. The method of embodiment 64, wherein the PARP inhibitor (e.g., olaparib) is administered in a dose significantly lower than its recommended dose as an anticancer agent.
66. The method of embodiment 61 or 62, wherein the antioxidant or/and the natural compound is/are selected from resveratrol, pterostilbene, ellagic acid, urolithin A, quercetin, coenzyme Q (e.g., CoQ₁₀), glutathione, N-acetyl-L-cysteine, α-lipoic acid, melatonin, creatine, S-adenosyl methionine, leucine, pyruvic acid/pyruvate, salts thereof, and combinations thereof.
67. The method of embodiment 61 or 62, wherein the vitamin is a member of the vitamin B family selected from thiamine (B₁), riboflavin (B₂), niacin (B₃), pantothenic acid (B₅), pyridoxine (B₆), biotin (B₇), folic acid (B₉), cobalamin (B₁₂), and combinations thereof, such as B₁, B₂, B₃ or B₆ or any combination thereof.
68. The method of embodiment 61, wherein the anti-inflammatory agent is selected from NSAIDs, inhibitors of pro-inflammatory cytokines and receptors therefor and their production, and combinations thereof.
69. The method of embodiment 61, wherein the antidiabetic agent is selected from AMPK agonists (e.g., metformin), PPAR-γ agonists, GLP-1 agonists, SGLT2 inhibitors, and combinations thereof.
70. The method of embodiment 61, wherein the antibiotic comprises ethionamide and optionally SMARt-420.
71. The method of embodiment 61, wherein the anticancer agent comprises radiation therapy, chemotherapy or cancer immunotherapy, or any combination or all thereof.
72. The method of embodiment 71, wherein the chemotherapy comprises a PARP inhibitor (e.g., olaparib), a TGF-β inhibitor or a cytotoxic agent, or any combination or all thereof.
73. The method of embodiment 71, wherein the cancer immunotherapy comprises an anti-PD1 agent, an anti-PDL1 agent or an anti-CTLA4 agent, or any combination thereof.
74. One or more compounds of any one of embodiments 1 to 45 or a pharmaceutically acceptable salt, solvate, hydrate, clathrate, polymorph or stereoisomer thereof for use as a medicament.
75. A composition comprising one or more compounds of any one of embodiments 1 to 45 or a pharmaceutically acceptable salt, solvate, hydrate, clathrate, polymorph or stereoisomer thereof for use as a medicament.
76. Use of one or more compounds of any one of embodiments 1 to 45 or a pharmaceutically acceptable salt, solvate, hydrate, clathrate, polymorph or stereoisomer thereof in the preparation of a medicament.
77. The compound(s), the composition or the use of embodiment 74, 75 or 76, respectively, wherein the one or more compounds are or comprise a compound of Formula II or IV.
78. The compound(s), the composition or the use of embodiment 74, 75 or 76, respectively, wherein the one or more compounds are or comprise a compound of Formula I and a compound of Formula II, or a compound of Formula III and a compound of Formula IV.
79. The compound(s), the composition or the use of embodiment 74, 75 or 76, respectively, or embodiment 77 or 78, wherein the medicament is for use in treating a mitochondrial disease, a mitochondria-related disease or condition, or a disease or condition characterized by acute NAD⁺ depletion due to DNA damage.
80. The compound(s), the composition or the use of embodiment 79, which is in combination with the use of at least one other therapeutic agent.
81. A method of elevating nicotinamide adenine dinucleotide (NAD⁺) level or/and providing cytoprotection in at least one cell type, tissue or organ of a subject, comprising administering to the subject a therapeutically effective amount of, or contacting the at least one cell type, tissue or organ of the subject with, one or more compounds of any one of embodiments 1 to 45 or a pharmaceutically acceptable salt, solvate, hydrate, clathrate, polymorph or stereoisomer thereof, or a pharmaceutical composition comprising the same.
82. The method of embodiment 81, wherein the subject suffers from a disorder or condition characterized by NAD⁺ depletion or/and cell injury, damage or death.
83. The method of embodiment 82, wherein the NAD⁺ depletion or/and the cell injury, damage or death are associated with or result from DNA damage.
84. The method of any one of embodiments 81 to 83, wherein the one or more compounds elevate NAD⁺ level in the mitochondria, the cytoplasm or/and the nucleus of a cell (e.g., total cellular NAD⁺ level).
85. The method of any one of embodiments 81 to 84, wherein the providing cytoprotection comprises reducing cell injury, damage or death.
86. The method of any one of embodiments 81 to 85, wherein the one or more compounds are or comprise a compound of Formula II or IV.
87. The method of any one of embodiments 81 to 86, wherein the one or more compounds are or comprise a compound of Formula I and a compound of Formula II, or a compound of Formula III and a compound of Formula IV.
88. A method of increasing nicotinamide adenine dinucleotide (NAD⁺) level or/and providing cytoprotection in at least one cell type, tissue or organ of a subject, or treating a mitochondrial disease, a mitochondria-related disease or condition, or a disease or condition of a subject characterized by acute NAD⁺ depletion due to DNA damage, comprising administering to the subject a therapeutically effective amount of one or more nicotinyl riboside compounds and a therapeutically effective amount of a poly(ADP-ribose) polymerase (PARP) inhibitor, or contacting the at least one cell type, tissue or organ of the subject with one or more nicotinyl riboside compounds and a PARP inhibitor.
89. The method of embodiment 88, wherein the subject suffers from a disorder or condition characterized by NAD⁺ depletion or/and cell injury, damage or death.
90. The method of embodiment 89, wherein the NAD⁺ depletion or/and the cell injury, damage or death are associated with or result from DNA damage.
91. The method of any one of embodiments 88 to 90, wherein the mitochondrial disease is a primary mitochondrial disease.
92. The method of any one of embodiments 88 to 90, wherein the mitochondrial disease, the mitochondria-related disease or condition, or the disease or condition characterized by acute NAD⁺ depletion due to DNA damage is associated with (e.g., is caused by or results in) secondary mitochondrial dysfunction.
93. The method of any one of embodiments 88 to 92, wherein the increasing NAD⁺ level comprises increasing NAD⁺ level in the mitochondria, the cytoplasm or/and the nucleus of a cell (e.g., total cellular NAD⁺ level).
94. The method of embodiment 93, wherein the one or more nicotinyl riboside compounds and the PARP inhibitor increase NAD⁺ level (e.g., total cellular NAD⁺ level, such as that in target cells) by at least about 50%, 100% (2-fold), 3-fold or 5-fold ex vivo or in vivo.
95. The method of any one of embodiments 88 to 94, wherein the providing cytoprotection comprises reducing cell injury, damage or death.
96. The method of embodiment 95, wherein the one or more nicotinyl riboside compounds and the PARP inhibitor increase the number of viable cells (e.g., target cells) by at least about 20%, 50%, 100% or 200% ex vivo or in vivo.
97. The method of any one of embodiments 88 to 96, wherein the one or more nicotinyl riboside compounds are or comprise one or more of nicotinamide riboside (NR), reduced NR (NRH), nicotinic acid riboside (NAR), reduced NAR (NARH) and pharmaceutically acceptable salts and stereoisomers thereof, or/and one or more derivatives thereof (NR/NAR derivatives).
98. The method of embodiment 97, wherein the one or more nicotinyl riboside compounds are or comprise NR or/and NRH, or a pharmaceutically acceptable salt, solvate, hydrate, clathrate, polymorph or stereoisomer thereof.
99. The method of embodiment 97 or 98, wherein the one or more NR/NAR derivatives are or comprise nicotinamide riboside triacetate (NRTA) or/and reduced NRTA (NRHTA), or a pharmaceutically acceptable salt, solvate, hydrate, clathrate, polymorph or stereoisomer thereof.
100. The method of any one of embodiments 97 to 99, wherein the one or more NR/NAR derivatives are or comprise one or more compounds of any one of embodiments 1 to 45 or a pharmaceutically acceptable salt, solvate, hydrate, clathrate, polymorph or stereoisomer thereof.
101. The method of embodiment 100, wherein the one or more NR/NAR derivatives are or comprise a compound of Formula II or IV.
102. The method of embodiment 100 or 101, wherein the one or more NR/NAR derivatives are or comprise a compound of Formula I and a compound of Formula II, or a compound of Formula III and a compound of Formula IV.
103. The method of any one of embodiments 88 to 102, wherein the therapeutically effective amount of each of the one or more nicotinyl riboside compounds independently is from about 1, 50 or 100 mg to about 500 or 1000 mg per day, or is about 1-100 mg, 100-200 mg, 200-300 mg, 300-400 mg, 400-500 mg or 500-1000 mg per day.
104. The method of any one of embodiments 88 to 103, wherein the PARP inhibitor is selected from niraparib, olaparib, pamiparib (BGB290), rucaparib, talazoparib, veliparib, 4-amino-1,8-naphthalimide, CEP9722, E7016, PJ34, and pharmaceutically acceptable salts thereof.
105. The method of any one of embodiments 88 to 104, wherein the therapeutically effective amount of the PARP inhibitor is significantly lower than its recommended dose as an anticancer agent.
106. The method of embodiment 105, wherein the therapeutically effective amount of the PARP inhibitor is no more than about 10%, 5%, 1%, 0.5% or 0.1% (e.g., no more than about 1%) of its recommended dose as an anticancer agent.
107. The method of any one of embodiments 88 to 106, wherein the PARP inhibitor is olaparib, and the therapeutically effective amount (e.g., per day or per dose) of olaparib is no more than about 10 mg, 5 mg, 1 mg, 0.5 mg or 0.1 mg (e.g., no more than about 1 mg); or is from about 0.01 or 0.1 mg to about 10 mg, from about 0.01 or 0.1 mg to about 1 mg, or from about 1 mg to about 10 mg; or is about 0.01-0.1 mg, 0.1-0.5 mg, 0.5-1 mg, 1-5 mg or 5-10 mg; or is about 10 μg, 50 μg, 0.1 mg, 0.5 mg, 1 mg, 5 mg or 10 mg.
108. The method of any one of embodiments 88 to 107, wherein the one or more nicotinyl riboside compounds and the PARP inhibitor synergistically increase NAD⁺ level or/and provide cytoprotection (e.g., reduce cytotoxicity), or have a synergistic therapeutic effect.
109. A method of increasing nicotinamide adenine dinucleotide (NAD⁺) level or/and NAD⁺/NADH ratio in at least one cell type, tissue or organ of a subject, increasing energy expenditure in a subject, or treating a mitochondrial disease or a mitochondria-related disease or condition of a subject, comprising administering to the subject a therapeutically effective amount of one or more nicotinyl riboside compounds and a therapeutically effective amount of a mitochondrial uncoupler, or contacting the at least one cell type, tissue or organ of the subject with one or more nicotinyl riboside compounds and a mitochondrial uncoupler.
110. The method of embodiment 109, wherein the mitochondrial disease is a primary mitochondrial disease.
111. The method of embodiment 109, wherein the mitochondrial disease or the mitochondria-related disease or condition is associated with (e.g., is caused by or results in) secondary mitochondrial dysfunction.
112. The method of embodiment 109 or 111, wherein the mitochondria-related disease or condition is a metabolic disorder, an obesity-associated condition, a disorder associated with oxidative stress, a neurological disorder, an inflammatory disorder, an immune-related disorder, a fibrotic disorder, a proliferative disorder or an aging-related disorder.
113. The method of embodiment 112, wherein the metabolic disorder or the obesity-associated condition is a disorder associated with abnormal or ectopic lipid accumulation or storage (e.g., a lipid storage droplet disorder such as CGI-58 deficiency [Chanarin-Dorfman syndrome], MTP deficiency or ApoB deficiency), lipodystrophy (e.g., congenital or acquired lipodystrophy, partial or generalized lipodystrophy, or severe lipodystrophy, such as HIV-associated lipodystrophy or antiretroviral therapy [ART]-induced lipodystrophy), obesity, a hyperphagia-associated disorder (e.g., Alström syndrome, Bardet-Biedl syndrome or Prader-Willi syndrome), metabolic syndrome, hypercholesterolemia (e.g., familial hypercholesterolemia), insulin resistance, diabetes (e.g., type 2 diabetes [T2D]), a cardiovascular disease (e.g., cardiomyopathy, coronary artery disease [CAD], stroke or ischemia-reperfusion injury [IRI]), a liver disorder (e.g., non-alcoholic fatty liver disease [NAFLD], non-alcoholic steatohepatitis [NASH], alcoholic liver disease [ALD], alcoholic steatohepatitis [ASH] or hepatotoxicity), or a lysosomal storage disease (e.g., a lipid storage disorder such as lysosomal acid lipase [LAL] deficiency [e.g., Wolman disease or cholesteryl ester storage disease], Gaucher disease or Niemann-Pick disease).
114. The method of embodiment 112, wherein the disorder associated with oxidative stress is a metabolic disorder, a cardiovascular disorder, an ischemia-reperfusion injury, an inflammatory disorder, an autoimmune disorder, a degenerative disorder such as a neurodegenerative disorder or a musculodegenerative disorder, a neuromuscular disorder, other muscle disorder, a neurodevelopmental disorder, a psychiatric disorder, a mitochondrial disease, a lysosomal storage disease, a disorder due to physical/body trauma, polycystic kidney disease, male infertility or an aging-related disorder.
115. The method of embodiment 112, wherein the neurological disorder is a neurodegenerative disorder (e.g., Alzheimer's disease, Huntington's disease, Parkinson's disease, amyotrophic lateral sclerosis [ALS] or multiple sclerosis), optic neuritis, Charcot-Marie-Tooth (CMT) disease, epilepsy, ischemic stroke or traumatic brain injury (TBI).
116. The method of embodiment 112, wherein the inflammatory disorder or the fibrotic disorder is a liver disorder characterized by inflammation or/and fibrosis (e.g., NASH), a kidney disorder characterized by inflammation or/and fibrosis (e.g., diabetic nephropathy), a heart disorder characterized by inflammation or/and fibrosis (e.g., inflammatory [myocarditis] or non-inflammatory cardiomyopathy), or a pancreas disorder characterized by inflammation or/and fibrosis (e.g., chronic pancreatitis).
117. The method of embodiment 112, wherein the immune-related disorder is an autoimmune disorder or a disorder associated with overactivation of the immune system.
118. The method of embodiment 112, wherein the proliferative disorder is a tumor or cancer, such as an obesity-associated tumor or cancer (e.g. hepatocellular carcinoma) or a tumor or cancer associated with an activated class IA phosphoinositide 3-kinase p110α (PI3K-α) (e.g., a solid tumor or cancer).
119. The method of anyone of embodiments 109 to 118, wherein the one or more nicotinyl riboside compounds are or comprise one or more of nicotinamide riboside (NR), reduced NR (NRH), nicotinic acid riboside (NAR), reduced NAR (NARH) and pharmaceutically acceptable salts and stereoisomers thereof, or/and one or more derivatives thereof (NR/NAR derivatives).
120. The method of embodiment 119, wherein the one or more nicotinyl riboside compounds are or comprise one or more of NR, NRH, NAR, NARH, and pharmaceutically acceptable salts, solvates, hydrates, clathrates, polymorphs and stereoisomers thereof.
121. The method of embodiment 119 or 120, wherein the one or more NR/NAR derivatives are or comprise one or more of nicotinamide riboside triacetate (NRTA), reduced NRTA (NRHTA), nicotinic acid riboside triacetate (NARTA), reduced NARTA (NARHTA), and pharmaceutically acceptable salts, solvates, hydrates, clathrates, polymorphs and stereoisomers thereof.
122. The method of anyone of embodiments 119 to 121, wherein the one or more NR/NAR derivatives are or comprise one or more compounds of any one of embodiments 1 to 45 or a pharmaceutically acceptable salt, solvate, hydrate, clathrate, polymorph or stereoisomer thereof.
123. The method of embodiment 122, wherein the one or more NR/NAR derivatives are or comprise a compound of Formula II or IV.
124. The method of embodiment 122 or 123, wherein the one or more NR/NAR derivatives are or comprise a compound of Formula I and a compound of Formula II, or a compound of Formula III and a compound of Formula IV.
125. The method of any one of embodiments 109 to 124, wherein the therapeutically effective amount of each of the one or more nicotinyl riboside compounds independently is from about 1, 50 or 100 mg to about 500 or 1000 mg per day, or is about 1-100 mg, 100-200 mg, 200-300 mg, 300-400 mg, 400-500 mg or 500-1000 mg per day.
126. The method of any one of embodiments 109 to 125, wherein the uncoupler is selected from benzoic acid, phenol, 2-methylphenol, 3-methylphenol, 4-methylphenol, all the dimethylphenol regioisomers (e.g., 2,4-dimethylphenol and 2,6-dimethylphenol), all the trimethylphenol regioisomers (e.g., 2,4,6-trimethylphenol), 2-isopropylphenol, 3-isopropylphenol, 4-isopropylphenol, all the di-isopropylphenol regioisomers (e.g., 2,4-di-isopropylphenol and 2,6-di-isopropylphenol), all the tri-isopropylphenol regioisomers (e.g., 2,4,6-tri-isopropylphenol), 2-tert-butylphenol, 3-tert-butylphenol, 4-tert-butylphenol, all the di-tert-butylphenol regioisomers (e.g., 2,4-di-tert-butylphenol and 2,6-di-tert-butylphenol), all the tri-tert-butylphenol regioisomers (e.g., 2,4,6-tri-tert-butylphenol), 2-methoxyphenol, 3-methoxyphenol, 4-methoxyphenol, all the dimethoxyphenol regioisomers (e.g., 2,4-dimethoxyphenol and 2,6-dimethoxyphenol), all the trimethoxyphenol regioisomers (e.g., 2,4,6-trimethoxyphenol), all tert-butyl-methoxyphenol regioisomers (e.g., butylated hydroxyanisole [BHA], which comprises 2-tert-butyl-4-methoxyphenol or/and 3-tert-butyl-4-methoxyphenol), all di-tert-butyl-methylphenol regioisomers {e.g., 2,6-di-tert-butyl-4-methylphenol (commonly known as butylated hydroxytoluene [BHT])}, 2-chlorophenol, 3-chlorophenol, 4-chlorophenol, all the dichlorophenol regioisomers (e.g., 2,4-dichlorophenol and 2,6-dichlorophenol), all the trichlorophenol regioisomers (e.g., 2,4,6-trichlorophenol), all the tetrachlorophenol regioisomers, pentachlorophenol, 2-fluorophenol, 3-fluorophenol, 4-fluorophenol, all the difluorophenol regioisomers (e.g., 2,4-difluorophenol and 2,6-difluorophenol), all the trifluorophenol regioisomers (e.g., 2,4,6-trifluorophenol), all the tetrafluorophenol regioisomers, pentafluorophenol, 2-cyanophenol, 3-cyanophenol, 4-cyanophenol, all the dicyanophenol regioisomers (e.g., 2,4-dicyanophenol and 2,6-dicyanophenol), all the tricyanophenol regioisomers (e.g., 2,4,6-tricyanophenol), 2-nitrophenol, 2-nitroanisole, 3-nitrophenol, 3-nitroanisole, 4-nitrophenol, 4-nitroanisole, 2,3-dinitrophenol (2,3-DNP), 2,3-dinitroanisole, 2,4-dinitrophenol (2,4-DNP, or commonly known as DNP), 2,4-dinitroanisole (2,4-DNP methyl ether, or commonly known as DNP methyl ether), MP201 (a DNP prodrug), 2,5-dinitrophenol (2,5-DNP), 2,5-dinitroanisole, 2,6-dinitrophenol (2,6-DNP), 2,6-dinitroanisole, 3,4-dinitrophenol (3,4-DNP), 3,4-dinitroanisole, 3,5-dinitrophenol (3,5-DNP), 3,5-dinitroanisole, all the trinitrophenol regioisomers (e.g., 2,4,6-trinitrophenol [picric acid]), all the dinitrocresol regioisomers (e.g., 2-methyl-3,5-dinitrophenol [commonly known as dinitro-ortho-cresol]), naturally occurring phenols {including simple phenols (e.g., catechol, resorcinol and hydroquinone), alkylresorcinols (e.g., adipostatin A [cardol], bilobol, hexylresorcinol, olivetol and DB-2073), monoterpenoid phenols (e.g., carvacrol and thymol), diterpenoid phenols (e.g., carnosol), dioxophenols (e.g., sesamol), phenolic aldehydes (e.g., vanillin and isovanillin), phenolic acids (e.g., salicylic acids, vanillic acid and gallic acid), hydroxylated phenylacetic acids (e.g., 4-hydroxyphenylacetic acid and homogentisic acid), hydroxylated phenylethanoids (e.g., tyrosol, hydroxytyrosol and oleocanthal), hydroxylated phenylpropenes (e.g., eugenol), hydroxylated phenylpropanones (e.g., gingerol and raspberry ketone), hydroxycinnamic acids (e.g., caffeic acid, chicoric acid, ortho-coumaric acid, meta-coumaric acid, para-coumaric acid, ferulic acid, rosmarinic acid and sinapinic acid), curcuminoids (e.g., curcumin, demethoxycurcumin, bisdemethoxycurcumin), hydroxylated coumarins (e.g., aesculetin, scopoletin and umbelliferone), hydroxylated isocoumarins (e.g., hydrangenol, phyllodulcin and thunberginols A, C, D, E and G), hydroxylated chromones (e.g., eugenin), hydroxylated naphthoquinones (e.g., alkannin, juglone, nigrosporin B and plumbagin), xanthonoids (e.g., mangostin and norathyriol), stilbenoids (e.g., piceatannol, pinosylvin, pterostilbene resveratrol and stilbestrol), dihydrostilbenoids (e.g., combretastatin, combretastatin B-1, dihydro-resveratrol and isonotholaenic acid), hydroxylated anthraquinones (e.g., aloe emodin), hydroxylated flavones (e.g., acacetin, apigenin, chrysin, diosmetin and luteolin), hydroxylated flavonols (e.g., fisetin, galangin, isorhamnetin, kaempferol, myricetin, pachypodol, quercetin and rhamnazin), hydroxylated flavanones (e.g, eriodictyol, homoeriodictyol, hesperetin, naringenin and silibinin), hydroxylated flavanonols (e.g., astilbin, aromadendrin [dihydrokaempferol] and taxifolin [dihydroquercetin]), hydroxylated flavan-3-ols [e.g., (+)-catechin, (−)-epicatechin, (+)-gallocatechin, (−)-epigallocatechin, (−)-epicatechin gallate and (−)-epigallocatechin gallate], hydroxylated aurones (e.g., aureusidin and leptosidin), hydroxylated isoflavones (e.g., alpinumisoflavone, biochanin A, daidzein, formononetin, genistein and glycitein), hydroxylated isoflavanes (e.g., laxiflorane and lonchocarpane), hydroxylated isoflavenes (e.g., glabrene, haginin D and 2-methoxyjudaicin), biflavonoids (e.g., amentoflavone, morelloflavone and ochnaflavone), anthocyanidins (e.g, aurantinidin, capensinidin, cyanidin, delphinidin, europinidin, hirsutidin, malvidin, pelargonidin, peonidin, petunidin, pulchellidin and rosinidin), hydroxylated coumestans (e.g., coumestrol, 4′-methoxycoumestrol, plicadin, repensol, trifoliol and wedelolactone), lignans (e.g., enterodiol, enterolactone, lariciresinol, secoisolariciresinol, matairesinol, hydroxymatairesinol, pinoresinol and syringaresinol), pterocarpans (e.g., glyceollins I and III, glycinol, glycyrrhizol A, medicarpin and phaseolin), other polyphenols (e.g., ellagic acid), capsaicin and cannabinoids}, retinoids (e.g., acitretin, adapalene, bexarotene and tazarotenic acid), lipophilic aromatic NSAIDs with a weak-acid group or convertible to such compounds {including acetic acid derivatives (e.g., diclofenac, aceclofenac, etodolac, ketorolac, indomethacin, 6-methoxy-2-naphthylacetic acid [6-MNA], nabumetone, sulindac and tolmetin), propionic acid derivatives (e.g., fenbufen, fenoprofen, flurbiprofen, ibuprofen, dexibuprofen, ketoprofen, dexketoprofen, loxoprofen, naproxen and oxaprozin), enolic acid derivatives (oxicams) (e.g., isoxicam, meloxicam, piroxicam, droxicam, tenoxicam and lornoxicam [chlortenoxicam]), anthranilic acid derivatives (fenamates) (e.g, flufenamic acid, meclofenamic acid, mefenamic acid and tolfenamic acid), salicylates (e.g., salicylic acid, acetylsalicylic acid [aspirin], methyl salicylate, diflunisal and salsalate), sulfonanilides (e.g., nimesulide), 3,5-pyrazolidinediones (e.g., azapropazone and phenylbutazone), selective COX-2 inhibitors (coxibs) (e.g., celecoxib, lumiracoxib, rofecoxib, valdecoxib, parecoxib and SC-236) and others (e.g., clonixin and licofelone)}, lipophilic aromatic antidiabetic agents with a weak-acid group or convertible to such compounds {including AMPK agonists (e.g., biguanides such as phenformin), PPAR-γ agonists (e.g., saroglitazar and thiazolidinediones such as balaglitazone, ciglitazone, darglitazone, englitazone, lobeglitazone, netoglitazone, pioglitazone, rivoglitazone, rosiglitazone and troglitazone), SGLT2 inhibitors (e.g., remogliflozin etabonate, phloretin and phlorizin), and K_{AT}p channel blockers (e.g., meglitinides [e.g., mitiglinide, nateglinide and repaglinide], first-generation sulfonylureas [e.g., acetohexamide, carbutamide, chlorpropamide, glycyclamide (tolhexamide), metahexamide, tolazamide and tolbutamide], and second-generation sulfonylureas [e.g., glibenclamide (glyburide), glibornuride, gliclazide, glimepiride, glipizide, gliquidone, glisoxepide and glyclopyramide])}, anilides (e.g., bupivacaine. NNC-0112-0000-2604 and salicylanilides [e.g., niclosamide and S13]), N-phenylcarbamates, 0-organyl-N-phenylthiocarbamates, N-phenylureas (e.g., SR4). N-phenylthioureas, sulfonanilides (e.g., endosidin 9, nimesulide and those disclosed in WO 2019/226490 A1), 2-acyl/aroyl-indan-1,3-diones [e.g., 2-heptanoyl-indan-1,3-dione and 2-(3′,4′-dichlorobenzoyl)-indan-1,3-dione], 2-aryl-1,3-indandiones (e.g., 2-β-chlorophenyl-indan-1,3-dione), benzimidazoles (e.g., TTFB), cyanotriazoles (e.g., OPC-163493), dithiocarbazates (e.g., S-alkyl, N-acyl/aroyldithiocarbazates [e.g., PDTC-9, NDTC-9 and IDTC-9]), N-phenylanthranilic acids (e.g., fenamate NSAIDs), phenylhydrazones (e.g., CCCP and FCCP), 3,5-pyrazolidinediones (e.g., p,p′-dichlorophenylbutazone), ellipticine, niclosamide (e.g., niclosamide ethanolamine), DK-520 (n-octanoyl ester of niclosamide), niclosamide analogs having —Cl or —CF₃ in place of the —NO₂ group, nitazoxanide (NTZ), tizoxanide (des-acetyl NTZ), oxyclozanide, usnic acid (e.g., (+)-usnic acid), tyrphostin A9 (AG17 or SF-6847), tyrphostin A9 analogs having H, Me, Et, n-Pr, iso-Pr, n-Bu, iso-Bu and sec-Bu at both positions ortho to the phenolic —OH group, BAM15, CCCP, FCCP, CDE, C4R1, CZ5, endosidin 9 (ES9), Pcp-1, PDTC-9, S13, SR4, TTFB, NNC-0112-0000-2604, NNC-0112-0000-0376, MitoBHT, cyclohexylMitoBHT, MitoDNP, MitoQₙ series, alkylTPP series, cationic cyanine dyes, and analogs, derivatives, prodrugs (including ester prodrugs, amino acid ester prodrugs, phosphate ester prodrugs, carbonate prodrugs, carbamate prodrugs, and ether prodrugs such as the corresponding uncouplers having a methyl ether at an aromatic hydroxyl [e.g., phenolic] group), metabolites, salts, targeted forms (including ether prodrugs such as the corresponding uncouplers having a methyl ether at an aromatic hydroxyl [e.g., phenolic] group for targeting to the liver), and controlled-release, slow-release and sustained-release forms (including liposomes, cholestosomes and lipid, polymeric or dendrimeric nanoparticles encapsulating the uncouplers) thereof.
127. The method of any one of embodiments 109 to 126, wherein administration of the one or more nicotinyl riboside compounds in combination with the uncoupler provides mild, sustained mitochondrial uncoupling.
128. The method of embodiment 127, wherein the uncoupler providing mild uncoupling is selected from benzoic acid, phenol, the monoalkylphenols, the dialkylphenols, the trialkylphenols including BHT, BHA, the monochlorophenol regioisomers, the dichlorophenol regioisomers, trichlorophenol regioisomers, the monofluorophenol regioisomers, the difluorophenol regioisomers, trifluorophenol regioisomers, the monocyanophenol regioisomers, the dicyanophenol regioisomers, the mononitrophenol regioisomers, the mononitroanisole regioisomers, the dinitrophenol regioisomers including DNP, the dinitroanisole regioisomers including DNP methyl ether, MP201, the dinitrocresol regioisomers, naturally occurring phenols, retinoids, NSAIDs, antidiabetic agents, sulfonanilides (e.g., Example Nos. 105, 134, 140, 157, 162, 165, 173, 183, 185, 193, 204, 229, 230, 239, 240 and 245 in WO 2019/226490), cyanotriazoles (e.g., OPC-163493), niclosamide (e.g., niclosamide ethanolamine), DK-520 (n-octanoyl ester of niclosamide), niclosamide analogs having —Cl or —CF₃ in place of the —NO₂ group, nitazoxanide, tizoxanide, oxyclozanide, BAM15, NNC-0112-0000-2604, NNC-0112-0000-0376, MitoBHT, cyclohexylMitoBHT, MitoDNP, MitoQio, decylTPP, and analogs, derivatives, prodrugs (including carbamate prodrugs and ether prodrugs such as the corresponding uncouplers having a methyl ether at an aromatic hydroxyl [e.g., phenolic] group), metabolites, salts, targeted forms (including ether prodrugs such as the corresponding uncouplers having a methyl ether at an aromatic hydroxyl [e.g., phenolic] group for targeting to the liver), and controlled-release, slow-release and sustained-release forms (including liposomes, cholestosomes and lipid, polymeric or dendrimeric nanoparticles encapsulating the uncouplers) thereof.
129. The method of embodiment 127 or 128, wherein the uncoupler providing mild uncoupling is OPC-163493, niclosamide (e.g., niclosamide ethanolamine), DK-520, a niclosamide analog having —Cl or —CF₃ in place of the —NO₂ group, nitazoxanide, tizoxanide, oxyclozanide, BAM15, BHT, MitoBHT, a sulfonanilide disclosed in WO 2019/226490 (e.g., Example No. 105, 134, 140, 157, 162, 165, 173, 183, 185, 193, 204, 229, 230, 239, 240 or 245), DNP, a DNP carbamate prodrug [e.g., DNP-OC(═O)—N-morpholine, DNP-OC(═O)—N-piperidine, DNP-OC(═O)—N-piperazine or DNP-OC(═O)—N-piperazine-N-Me], a DNP ether prodrug (e.g., DNP methyl ether), other DNP prodrug (e.g., MP201), a controlled-, slow- or sustained-release form of DNP or another uncoupler (e.g., liposomes, cholestosomes or lipid, polymeric or dendrimeric nanoparticles encapsulating DNP or another uncoupler), or a pharmaceutically acceptable salt thereof.
130. The method of any one of embodiments 127 to 129, wherein the uncoupler providing mild uncoupling is nitazoxanide, tizoxanide, BAM15, DNP or N-(4-cyanobicyclo[2.2.2]octan-1-yl)-4-fluoro-2-[(3,3,3-trifluoropropyl)sulfonamido]benzamide, or an analog, derivative, prodrug, metabolite, salt, targeted form, or controlled-release, slow-release or sustained-release form thereof.
131. The method of embodiment 130, wherein the controlled-, slow- or sustained-release form of DNP is a pellet, particle, bead or sphere containing DNP and having a controlled-, slow- or sustained-release polymeric coating, or is a solid dosage form (e.g., a tablet, capsule or pill) comprising a plurality of such pellets, particles, beads or spheres.
132. The method of any one of embodiments 109 to 131, wherein the therapeutically effective amount of the uncoupler is from about 1, 50 or 100 mg to about 500 mg per day, or is about 1-100 mg, 1-50 mg, 50-100 mg, 100-200 mg, 200-300 mg, 300-400 mg or 400-500 mg per day.
133. The method of any one of embodiments 109 to 132, wherein the one or more nicotinyl riboside compounds or/and the uncoupler are encapsulated in liposomes, micelles, cholestosomes, or lipid, polymeric or dendrimeric nano-/microparticles or nano-/microspheres.
134. The method of any one of embodiments 109 to 133, wherein:

the one or more nicotinyl riboside compounds are NR, NRH, NRTA or NRHTA, or a pharmaceutically acceptable salt, solvate, hydrate, clathrate, polymorph or stereoisomer thereof; and

the uncoupler is nitazoxanide, tizoxanide. BAM15, DNP or N-(4-cyanobicyclo[2.2.2]octan-1-yl)-4-fluoro-2-[(3,3,3-trifluoropropyl)sulfonamido]benzamide, or a pharmaceutically acceptable salt, solvate, hydrate, clathrate, polymorph, stereoisomer, or controlled-release, slow-release or sustained-release form thereof.

135. The method of any one of embodiments 109 to 134, wherein the one or more nicotinyl riboside compounds or/and the uncoupler are administered orally.
136. The method of any one of embodiments 109 to 135, wherein the one or more nicotinyl riboside compounds or/and the uncoupler are administered parenterally, such as intravenously, subcutaneously, intramuscularly, intrathecally or topically (e.g., sublingually).
137. The method of any one of embodiments 109 to 136, wherein the one or more nicotinyl riboside compounds and the uncoupler increase NAD⁺ level (e.g., total cellular NAD⁺ level, such as that in target cells) by at least about 20%, 50% or 100% ex vivo or in vivo.
138. The method of any one of embodiments 109 to 137, wherein the one or more nicotinyl riboside compounds and the uncoupler increase NAD⁺/NADH ratio (e.g., cellular NAD⁺/NADH ratio or blood/plasma/serum NAD⁺/NADH ratio) by at least about 20%, 50% or 100% ex vivo or in vivo.
139. The method of any one of embodiments 109 to 138, wherein the one or more nicotinyl riboside compounds and the uncoupler reduce ATP production or level (e.g., mitochondrial ATP production or total cellular ATP level, such as that in target cells) ex vivo or in vivo by no more than about 10% or 5%, or by about 2-10%, 2-5% or 5-10%.
140. The method of any one of embodiments 109 to 139, wherein the one or more nicotinyl riboside compounds and the uncoupler increase energy expenditure (e.g., whole-body energy expenditure or basal metabolic rate [BMR]) by at least about 2%, 5%, 10%, 15% or 20%, or by about 2-5%, 5-10%, 10-15% or 15-20%.
141. The method of any one of embodiments 109 to 140, wherein administration of the one or more nicotinyl riboside compounds in combination with the uncoupler increases the safety or/and the efficacy of the uncoupler, or has synergistic effect(s).
142. The method of any one of embodiments 109 to 141, wherein:

the mitochondrial disease or the mitochondria-related disease or condition is a metabolic disorder or an obesity-associated condition;

the one or more nicotinyl riboside compounds are NR, NRH, NRTA or NRHTA, or a pharmaceutically acceptable salt, solvate, hydrate, clathrate, polymorph or stereoisomer thereof; and

the uncoupler is nitazoxanide, tizoxanide, BAM15, DNP or N-(4-cyanobicyclo[2.2.2]octan-1-yl)-4-fluoro-2-[(3,3,3-trifluoropropyl)sulfonamido]benzamide, or a pharmaceutically acceptable salt, solvate, hydrate, clathrate, polymorph, stereoisomer or controlled-release, slow-release or sustained-release form thereof.

143. The method of any one of embodiments 109 to 142, further comprising administering a therapeutically effective amount of one or more additional therapeutic agents.
144. The method of embodiment 143, wherein the one or more additional therapeutic agents are or comprise a poly(ADP-ribose) polymerase (PARP) inhibitor or/and a PI3K-α inhibitor (e.g., alpelisib).
145. The method of embodiment 144, wherein the PARP inhibitor is selected from niraparib, olaparib, pamiparib (BGB290), rucaparib, talazoparib, veliparib, 4-amino-1,8-naphthalimide, CEP9722, E7016, PJ34, and pharmaceutically acceptable salts thereof.
146. The method of embodiment 144 or 145, wherein the therapeutically effective amount of the PARP inhibitor is significantly lower than its recommended dose as an anticancer agent.
147. The method of embodiment 146, wherein the therapeutically effective amount of the PARP inhibitor is no more than about 10%, 5%, 1%, 0.5% or 0.1% (e.g., no more than about 1%) of its recommended dose as an anticancer agent.
148. The method of any one of embodiments 144 to 147, wherein the PARP inhibitor is olaparib, and the therapeutically effective amount (e.g., per day or per dose) of olaparib is no more than about 10 mg, 5 mg, 1 mg, 0.5 mg or 0.1 mg (e.g., no more than about 1 mg); or is from about 0.01 or 0.1 mg to about 10 mg, from about 0.01 or 0.1 mg to about 1 mg, or from about 1 mg to about 10 mg; or is about 0.01-0.1 mg, 0.1-0.5 mg, 0.5-1 mg, 1-5 mg or 5-10 mg; or is about 10 μg, 50 μg, 0.1 mg, 0.5 mg, 1 mg, 5 mg or 10 mg.
149. A method of reducing NADH/NAD⁺ ratio or/and plasma or serum α-hydroxybutyrate level in a subject with insulin resistance, type 2 diabetes, NASH, lipodystrophy, a lipid storage disorder, or a disorder associated with abnormal or ectopic lipid accumulation or storage, comprising administering to the subject a therapeutically effective amount of a nicotinyl riboside compound and a therapeutically effective amount of a mitochondrial uncoupler, and optionally a therapeutically effective amount of a PARP inhibitor.
150. A method of reducing plasma or serum lactate level in a subject with lactic acidosis due to alcoholic hepatitis or an acutely decompensated liver disease, comprising administering to the subject a therapeutically effective amount of a nicotinyl riboside compound and a therapeutically effective amount of a PARP inhibitor, and optionally a therapeutically effective amount of a mitochondrial uncoupler.
151. A method of treating a metabolic disorder such as insulin resistance, type 2 diabetes, NASH, lipodystrophy, a lipid storage disorder, or a disorder associated with abnormal or ectopic lipid accumulation or storage, comprising administering to a subject in need of treatment a therapeutically effective amount of a nicotinyl riboside compound and a therapeutically effective amount of a mitochondrial uncoupler, and optionally a therapeutically effective amount of a PARP inhibitor, wherein the treatment increases TCA cycle flux (e.g., V_{CS} as measured by PINTA), or/and reduces or improves:

1) blood, plasma or serum level of triglycerides, cholesterol, LDL, VLDL, lipoprotein(a) [Lp(a)] or pro-protein of apolipoprotein C3 (proC3); or

2) blood, plasma or serum level of glucose or insulin; or

3) blood, plasma or serum NADH/NAD⁺ ratio or α-hydroxybutyrate (AHB) level; or

4) blood, plasma or serum level of alanine transaminase (ALT), gamma-glutamyltransferase (GGT) or keratin 18 (CK18); or

5) a measure of liver fat, such as proton density fat fraction in magnetic resonance imaging (MRI-PDFF); or

6) a measure of liver stiffness, such as transient elastography or magnetic resonance elastography (MRE); or

7) a measure of liver fibrosis, such as enhanced liver fibrosis (ELF) score, FIB4 score or ALT platelet ratio (APRI) score; or

8) any combination thereof.

152. A pharmaceutical or cosmetic composition comprising one or more pharmaceutically acceptable excipients or carriers and:

1) one or more compounds of any one of embodiments 1 to 45; or

2) one or more nicotinyl riboside compounds and a PARP inhibitor; or

3) one or more nicotinyl riboside compounds and a mitochondrial uncoupler; or

4) one or more nicotinyl riboside compounds, a PARP inhibitor and a mitochondrial uncoupler.

153. The composition of embodiment 152, wherein the one or more nicotinyl riboside compounds are or comprise one or more of nicotinamide riboside (NR), reduced NR (NRH), nicotinic acid riboside (NAR), reduced NAR (NARH) and pharmaceutically acceptable salts and stereoisomers thereof, or/and one or more derivatives thereof (NR/NAR derivatives).
154. The composition of embodiment 152 or 153, wherein the one or more compounds, or the one or more nicotinyl riboside compounds, are or comprise:

1) a compound of Formula II or IV; or

2) a compound of Formula I and a compound of Formula II; or

3) a compound of Formula III and a compound of Formula IV.

155. The composition of any one of embodiments 152 to 154, wherein the one or more nicotinyl riboside compounds are or comprise one or more of NR, NRH, NAR, NARH, and pharmaceutically acceptable salts, solvates, hydrates, clathrates, polymorphs and stereoisomers thereof.
156. The composition of any one of embodiments 152 to 155, wherein the one or more nicotinyl riboside compounds are or comprise one or more of nicotinamide riboside triacetate (NRTA), reduced NRTA (NRHTA), nicotinic acid riboside triacetate (NARTA), reduced NARTA (NARHTA), and pharmaceutically acceptable salts, solvates, hydrates, clathrates, polymorphs and stereoisomers thereof.
157. The composition of any one of embodiments 152 to 156, wherein the amount of each of the one or more nicotinyl riboside compounds in the composition independently is about 1-100 mg, 100-500 mg or 500-1000 mg.
158. The composition of any one of embodiments 152 to 157, wherein the amount of the PARP inhibitor in the composition is significantly lower (e.g., at least about 10%, 5% or 1% lower) than its recommended dose as an anticancer agent.
159. The composition of any one of embodiments 152 to 158, wherein the PARP inhibitor is olaparib.
160. The composition of any one of embodiments 152 to 159, wherein the mitochondrial uncoupler is an uncoupler that provides mild uncoupling.
161. The composition of embodiment 160, wherein the uncoupler providing mild uncoupling is nitazoxanide, tizoxanide, BAM15, DNP or N-(4-cyanobicyclo[2.2.2]octan-1-yl)-4-fluoro-2-[(3,3,3-trifluoropropyl)sulfonamido]benzamide, or an analog, derivative, prodrug, metabolite, salt, targeted form, or controlled-release, slow-release or sustained-release form thereof.
162. The composition of embodiment 160 or 161, wherein the uncoupler providing mild uncoupling is OPC-163493, niclosamide (e.g., niclosamide ethanolamine), DK-520 (n-octanoyl ester of niclosamide), a niclosamide analog having —Cl or —CF₃ in place of the —NO₂ group, nitazoxanide, tizoxanide, oxyclozanide, BAM15, BHT, MitoBHT, a sulfonanilide disclosed in WO 2019/226490 (e.g., Example No. 105, 134, 140, 157, 162, 165, 173, 183, 185, 193, 204, 229, 230, 239, 240 or 245), DNP, a DNP carbamate prodrug [e.g., DNP-OC(═O)—N-morpholine, DNP-OC(═O)—N-piperidine, DNP-OC(═O)—N-piperazine or DNP-OC(═O)—N-piperazine-N-Me], a DNP ether prodrug (e.g., DNP methyl ether), other DNP prodrug (e.g., MP201), a controlled-, slow- or sustained-release form of DNP or another uncoupler (e.g., liposomes, cholestosomes or lipid, polymeric or dendrimeric nanoparticles encapsulating DNP or another uncoupler), or a pharmaceutically acceptable salt thereof.
163. The composition of any one of embodiments 152 to 162, wherein the amount of the uncoupler in the composition is about 1-100 mg, 100-300 mg or 300-500 mg.
164. The composition of any one of embodiments 152 to 163, wherein:

the one or more nicotinyl riboside compounds are NR, NRH, NRTA or NRHTA, or a pharmaceutically acceptable salt, solvate, hydrate, clathrate, polymorph or stereoisomer thereof; and

the uncoupler is nitazoxanide, tizoxanide, BAM15, DNP or N-(4-cyanobicyclo[2.2.2]octan-1-yl)-4-fluoro-2-[(3,3,3-trifluoropropyl)sulfonamido]benzamide, or a pharmaceutically acceptable salt, solvate, hydrate, clathrate, polymorph, stereoisomer or controlled-release, slow-release or sustained-release form thereof.

165. The composition of any one of embodiments 152 to 164, which is or comprises a liposome, micelle, cholestosome, or lipid, polymeric or dendrimeric nano-/microparticle or nano-/microsphere encapsulating the compounds/therapeutic agents.
166. The composition of any one of embodiments 152 to 165, which is a controlled-release, slow-release or sustained-release composition.
167. The composition of any one of embodiments 152 to 166, which is an oral dosage form, such as a tablet, capsule or pill.
168. The composition of any one of embodiments 152 to 166, which is formulated for parenteral (e.g., intravenous, subcutaneous, intramuscular, intrathecal or topical [e.g., sublingual]) administration.
169. The composition of any one of embodiments 152 to 168, which is for use in the treatment of a mitochondrial disease (e.g., a primary mitochondrial disease), a mitochondria-related disease or condition, a disease or condition associated with secondary mitochondrial dysfunction, a disease or condition characterized by acute NAD⁺ depletion due to DNA damage, or a skin disorder or condition.
170. The composition of embodiment 169, wherein the mitochondria-related disease or condition or the disease or condition associated with secondary mitochondrial dysfunction is a metabolic disorder, an obesity-associated condition, a disorder associated with oxidative stress, a neurological disorder, an inflammatory disorder, an immune-related disorder, a fibrotic disorder, a proliferative disorder (e.g., a tumor or cancer), or an aging-related disorder.
171. A kit comprising the pharmaceutical or cosmetic composition of any one of embodiments 152 to 168.
172. The kit of embodiment 171, further comprising instructions for using or administering the pharmaceutical or cosmetic composition to treat a mitochondrial disease (e.g., a primary mitochondrial disease), a mitochondria-related disease or condition, a disease or condition associated with secondary mitochondrial dysfunction, a disease or condition characterized by acute NAD⁺ depletion due to DNA damage, or a skin disorder or condition.
173. The kit of embodiment 172, wherein the mitochondria-related disease or condition or the disease or condition associated with secondary mitochondrial dysfunction is a metabolic disorder, an obesity-associated condition, a disorder associated with oxidative stress, a neurological disorder, an inflammatory disorder, an immune-related disorder, a fibrotic disorder, a proliferative disorder (e.g., a tumor or cancer), or an aging-related disorder.

### Synthesis of NR and NAR Derivatives

### Abbreviations:

ACN=acetonitrile
DCC=N,N′-dicyclohexylcarbodiimide
DMAP=4-dimethylaminopyridine

### DMF=N,N-dimethylformamide

DMP or 2.2-DMP=2.2-dimethoxypropane
HMDS=hexamethyldisilazide
MeOH=methanol
—OAc=acetate
p-TSA=para-toluenesulfonic acid
Py.=pyridine
tBuMgCl=tert-butylmagnesium chloride
TBSCl=tert-butyldimethylsilyl chloride
TEA=triethylamine
TFA=trifluoroacetic acid
THF=tetrahydrofuran
TLC=thin-layer chromatography
TMSOTf=trimethylsilyl trifluromethanesulfonate

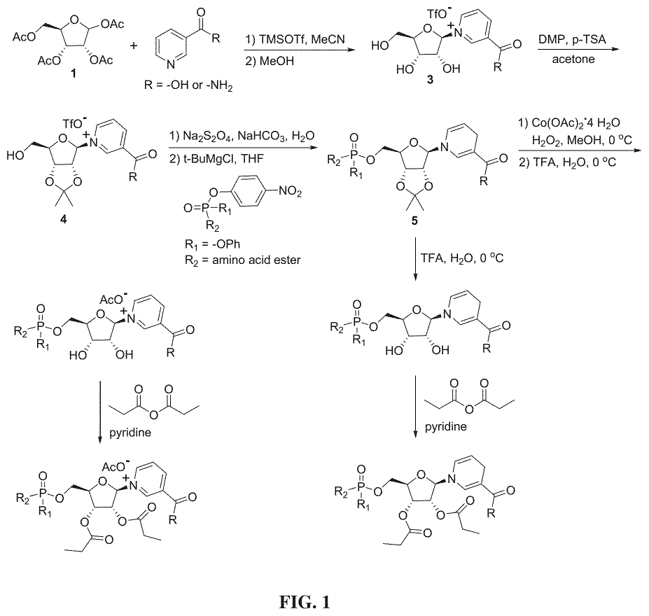

Compounds of Formulas I and II can be synthesized using the exemplary process shown in FIG. 1. The process in FIG. 1 can be adapted to prepare compounds of Formulas III and IV.

Compounds MP-05, MP-06, MP-07 and MP-08 are synthesized starting from peracetylated β-D-ribofuranose. Standard Vorbrüggen's conditions are employed to obtain common intermediates 3 and 4. Functional group manipulations of intermediate 4 using reported protocols for the respective target compounds result in the synthesis of MP-05, MP-06, MP-07 and MP-08.

Compounds MP-09 and MP-10 are synthesized from intermediate 3 with esterification of the required functional groups toward the end of the synthesis. Compounds MP-12 through MP-24 are synthesized from intermediate 4, with reduction of the nicotinamide ring followed by functional group modifications as shown in FIG. 1, and subsequent regeneration of the aromatic nicotinamide ring.

### Synthesis of MP-05 and MP-06:

FIG. 2 shows an exemplary process for synthesizing compounds MP-05 and MP-06. Their synthesis starts from commercially available peracetylated β-D-ribofuranose 1, the first step being the glycosylation of 1 with nicotinamide using Vorbrüggen's protocol. Selective protection of 5′-hydroxyl with TBSCl followed by bis-acylation using propanoyl chloride (or propionic anhydride) yields advanced intermediate 5. Deprotection of 5′-OTBS yields MP-05, and reduction of the nicotinamide ring with sodium dithionite generates MP-06.

### Synthesis of MP-07 and MP-08:

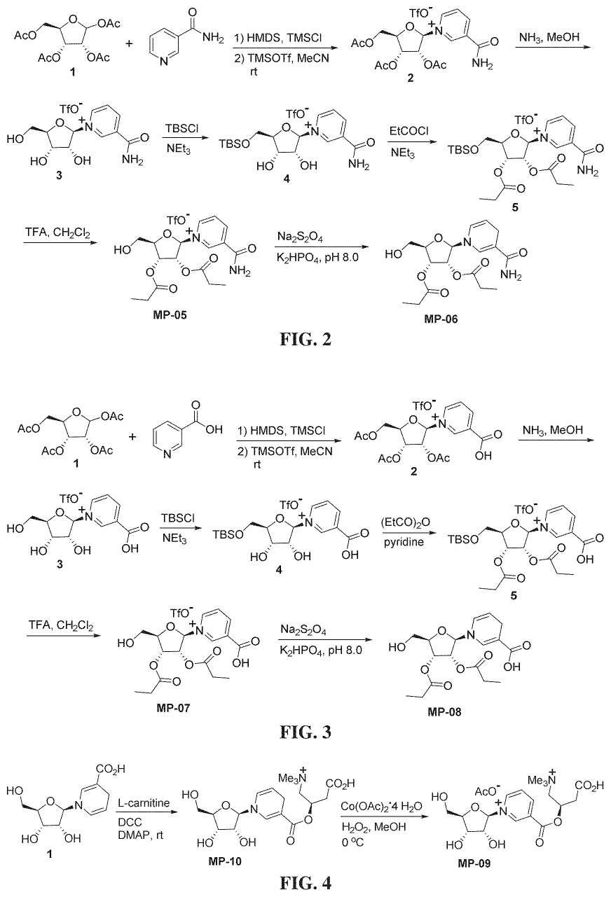

FIG. 3 shows an exemplary process for synthesizing NAR derivatives MP-07 and MP-08, which is similar to the process for synthesizing NR derivatives MP-05 and MP-06 in FIG. 2, except that nicotinic acid is used in lieu of nicotinamide in the Vorbrüggen glycosylation reaction.

### Synthesis of MP-09 and MP-10:

FIG. 4 shows an exemplary process for synthesizing compounds MP-09 and MP-10. NARH 1 in FIG. 4 is prepared by sodium dithionite reduction of intermediate 3 in FIG. 3. DCC-mediated coupling of NARH 1 with L-cartinine furnishes MP-10, whose oxidation by cobalt acetate yields MP-09. It is understood that both L-cartinine itself and the L-cartinine moiety of MP-09 and MP-10 can be a zwitterion.

### Synthesis of MP-14 and MP-16:

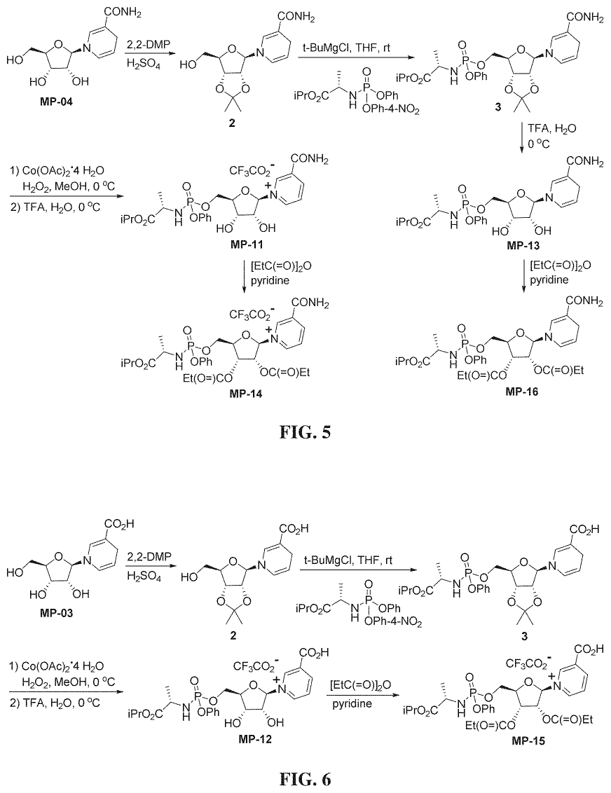

FIG. 5 shows an exemplary process for synthesizing compounds MP-14 and MP-16. NRH (MP-04) is prepared by sodium dithionite reduction of NR 3 in FIG. 2. After oxidation and deprotection of the dimethyl ketal group of 5′-phosphoramidate intermediate 3, bis-acylation of the resulting MP-11 generates MP-14. Similarly, deprotection of intermediate 3 without oxidation and bis-acylation of the resulting MP-13 generate MP-16.

Synthesis of MP-12 and MP-15 and their reduced forms:

FIG. 6 shows an exemplary process for synthesizing compounds MP-12 and MP-15. Oxidation and deprotection of the dimethyl ketal group of 5′-phosphoramidate intermediate 3 afford MP-12, whose bis-acylation produces MP-15. Deprotection of the dimethyl ketal group of intermediate 3 yields the reduced form of MP-12, and bis-propanoylation of the reduced form of MP-12 generates the reduced form of MP-15.

### Synthesis of MP-17, MP-20, MP-23 and MP-24:

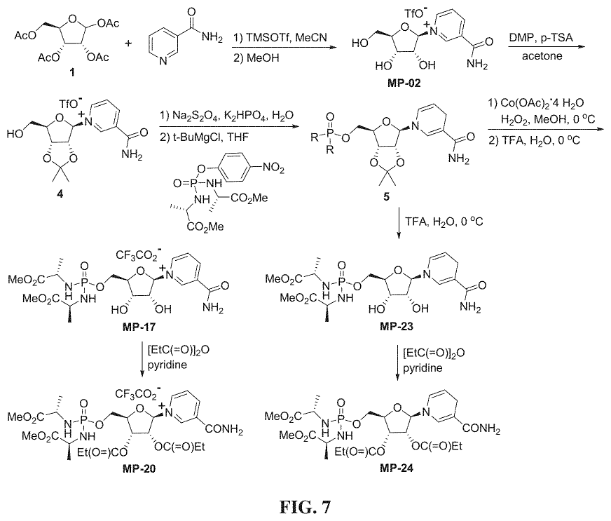

FIG. 7 shows an exemplary process for synthesizing MP-17, MP-20, MP-23 and MP-24. Reduction of intermediate 4 with sodium dithionite followed by coupling with the indicated phosphorodiamidate reagent affords common intermediate 5. Oxidation and deprotection of the dimethyl ketal group of intermediate 5 produce MP-17, whose bis-acylation yields MP-20. Similarly, deprotection of intermediate 5 without oxidation furnishes MP-23, whose bis-acylation yields MP-24.

### Synthesis of MP-18, MP-19, MP-21 and MP-22:

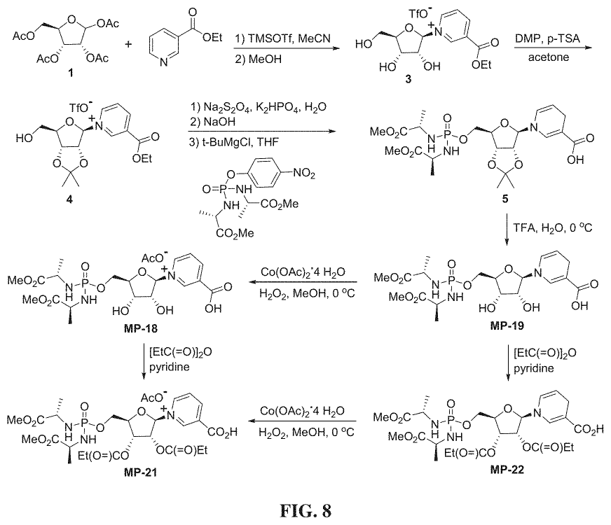

FIG. 8 shows an exemplary process for synthesizing MP-18, MP-19, MP-21 and MP-22. Intermediate 5 in FIG. 8 is prepared in a similar manner as intermediate 5 in FIG. 7 except that ethylnicotinate is used instead of nicotinamide in the Vorbrüggen glycosylation reaction. Deprotection of the dimethyl ketal group of intermediate 5 furnishes MP-19. Bis-acylation of MP-19 yields MP-22, while oxidation of MP-19 yields MP-18. MP-21 can be made by bis-acylation of MP-18 or oxidation of MP-22.

### EXAMPLES

The following examples are intended only to illustrate the disclosure. Other processes, assays, studies, protocols, procedures, methodologies, reagents and conditions may alternatively be used as appropriate.

### Example 1. Synthesis of 1-((2R,3R,4S,5R)-5-(((Bis(((S)-1-methoxy-1-oxopropan-2-yl)amino)phosphoryl)oxy)methyl)-3,4-dihydroxytetrahydrofuran-2-yl)-3-carboxamidepyridin-1-ium trifluoroacetate (MP-17) and 1-((2R,3R,4R,5R)-5-(((bis(((S)-1-methoxy-1-oxopropan-2-yl)amino)phosphoryl)oxy)methyl)-3,4-bis(propionyloxy)tetrahydrofuran-2-yl)-3-carboxamidepyridin-1-ium Trifluoroacetate (MP-20)

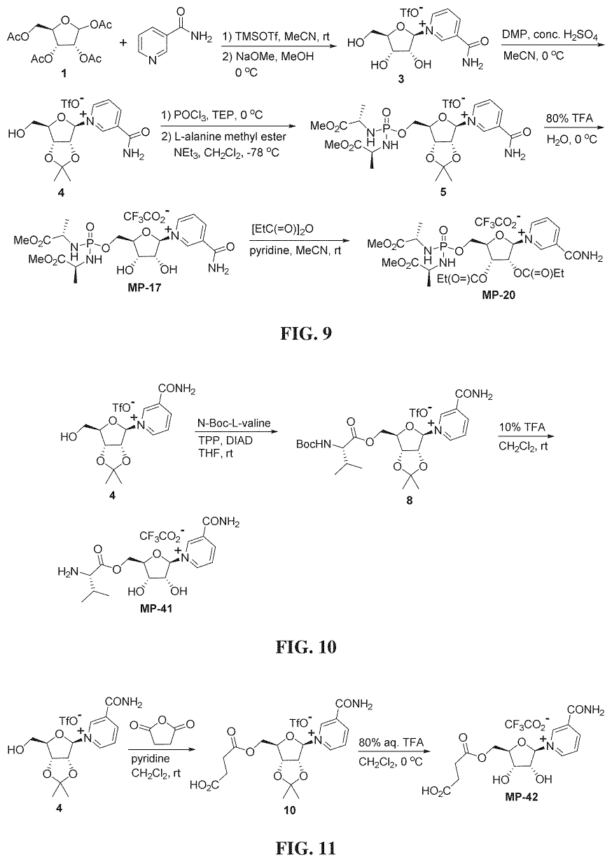

FIG. 9 shows the process for the synthesis of MP-17 and MP-20.

### 3-Carboxamide-1-(2,3,5-tri-O-acetyl-β-D-ribofuranosyl)pyridinium triflate (2)

To a well stirred solution of nicotinamide (115.2 g, 0.942 mol) in dry acetonitrile (1.5 L) was added trimethylsilyl trifluoromethanesulfonate (314 mL, 1.72 mol) in one portion. The nicotinamide was dissolved within 5 min. A solution of 1,2,3,5-tetra-1-acetyl-β-D-ribofuranose 1 (100 g, 0.314 mol) in dry acetonitrile (300 mL) was added all in one portion at room temperature under nitrogen atmosphere. The solution was stirred for 30 min at room temperature. The excess TMSOTf was quenched by the addition of 1.2 M NaHCO₃ solution (10 mL) followed by solid NaHCO₃ (85 g, 1.01 mol) in small portions. The suspension was stirred for 30 min at room temperature and the solids were filtered and washed with CH₂Cl₂ (500 mL). Combined filtrates were concentrated under reduced pressure to get a thick yellow residue. The residue was suspended in 2 L of CH₂Cl₂. The suspension was stirred at room temperature for 15 min, and then solids were filtered and washed with CH₂Cl₂ (2 L). The filtrate was concentrated under vacuum to obtain compound 2 (120 g crude of which 82% was product by LCMS) as a yellow syrup. ¹H NMR (400 MHz, DMSO-d₆): δ 9.46 (s, 1H), 9.24 (d, J=6.24 Hz, 1H), 9.06 (d, J=8.1 Hz, 1H), 8.67 (s, 1H), 8.40 (t, J=6.5 Hz, 1H), 8.25 (s, 1H), 6.65 (d, J=3.3 Hz, 1H), 5.61-5.62 (m, 1H), 5.44 (t, J=5.92 Hz, 1H), 4.70-4.71 (m, 1H), 4.45 (s, 2H), 2.15 (s, 3H) and 2.11 (s, 3H). LCMS (M⁺): 381.1.

### 3-Carboxamide-1-(β-D-ribofuranosyl)pyridinium Triflate (3)

To a well-stirred solution of crude compound 2 (120 g) in anhydrous MeOH (1.5 L) was added 1N NaOMe/MeOH (750 mL, 0.75 mol) dropwise over 10 min. The internal temperature was maintained below 5° C. and the reaction mixture was stirred at 0° C. for 0.5 hr, and the progress of the reaction was monitored by LCMS. Then 250 mL of 3 M HCl was added slowly, keeping the internal temperature below 5° C. Excess solvent was removed under reduced pressure below 20° C. to afford crude compound 3 (80 g, 78% pure by LCMS). ¹H NMR (400 MHz, D₂O): δ 9.49 (s, 1H), 9.16 (d, J=5.9 Hz, 1H), 8.88 (t, J=7.2 Hz, 1H), 8.57 (d, J=3.8 Hz, 1H), 6.15 (s, 1H), 4.37-4.38 (m, 2H), 4.25 (t, J=4.7 Hz, 1H), 3.95 (d, J=12.9 Hz, 1H) and 3.78-3.81 (m, 1H). LCMS (M⁺): 255.1.

### 3-Carboxamide-1-(2,3-O-isopropylidene-β-D-ribofuranosyl)pyridinium Triflate (4)

Into a 50 mL double-neck round-bottom flask containing concentrated sulfuric acid (160 mg, 1.62 mmol) was added dry acetonitrile (8 mL) at 0° C. under inert atmosphere. After stirring for 5 minutes, 2,2-dimethoxypropane (2.5 mL) and a solution of compound 3 (1 g, 2.475 mmol) in dry acetonitrile (2 mL) already cooled at 0° C. were added to the flask, and the resulting reaction mixture was stirred at 0° C. for 30 minutes. After completion of the reaction (monitored by TLC), excess acid was quenched with solid sodium carbonate (202 mg, 0.77 mmol) in the presence of water (0.2 mL) at 0° C. with stirring for 30 minutes, and then the reaction mixture was passed through a pad of Celite and the Celite bed was washed with acetonitrile (2×10 mL). Combined filtrates were concentrated under reduced pressure to afford a crude mass. Purification of the crude mass by silica-gel column chromatography with MeOH/CH₂Cl₂ (0-10%) yielded compound 4 (400 mg, 34%) as a waxy off-white solid. ¹H NMR (400 MHz, DMSO-d₆): δ 9.43 (s, 1H), 9.30 (d, J=6 Hz, 1H), 9.00 (t, J=7.9 Hz, 1H), 8.65 (s, 1H), 8.29 (t, J=7.8 Hz, 1H), 8.21 (s, 1H), 6.47 (s, 1H), 5.24-5.25 (m, 2H), 4.92 (d, J=5.7 Hz, 1H), 4.74 (s, 1H), 3.74-3.76 (m, 1H), 3.64-3.66 (m, 1H), 1.59 (s, 3H) and 1.36 (s, 3H). LCMS (M⁺): 295.1.

### 1-((3aR,4R,6R,6aR)-6-(((Bis(((S)-1-methoxy-1-oxopropan-2-yl)amino)phosphoryl)oxy)methyl)-2,2-dimethyltetrahydrofuro[3,4-d][1,3]dioxol-4-yl)-3-carboxamidepyridin-1-ium Ammonium Acetate (5)

### Method a Using Tert-Butyl Magnesium Chloride and p-Nitrophenyl bis(methyl L-alaninyl)phosphate

To a well stirred solution of compound 4 (520 mg, 1.7 mmol) in dry THF (10 mL) was added tert-butyl magnesium chloride (7 mL, 7 mmol, 1 M in THF) under nitrogen atmosphere, and the resulting solution was stirred at room temperature for 10 minutes. To the reaction mixture was added p-nitrophenyl bis(methyl L-alaninyl)phosphate (1 g, 2.6 mmol, prepared using standard literature procedure) in dry THF (5 mL), and the reaction mixture was stirred at ambient temperature for 2 hr. Excess reagent was then quenched with MeOH (5 mL) and saturated ammonium chloride solution (2 mL). The reaction mixture was concentrated under reduced pressure to get a crude residue. The crude residue was purified by reverse-phase prep HPLC (eluting with 10 mM ammonium acetate in acetonitrile as solvent A and water as solvent B), and column fractions containing compound 5 furnished compound 5 (15 mg, 1.5%) as a colorless gum upon lyophilisation. LCMS (M⁺): 545.2.

### Method B Using Phosphorus Oxychloride and Methyl L-Alaninate

Into a 100 mL single-neck round-bottom flask containing a solution of compound 4 (2 g, 4.9 mmol) in anhydrous triethyl phosphate (TEP, 20 mL) was added phosphoryl chloride (1.67 mL, 18 mmol) at 0° C. under nitrogen atmosphere, and the resulting reaction mixture was stirred at 0° C. for 48 hr. The reaction mixture was cooled to −78° C., and methyl L-alaninate hydrochloride salt (3.14 g, 22 mmol) in anhydrous CH₂Cl₂ (20 mL) was added to the flask. Triethylamine (6.27 mL, 45 mmol) was added at −78° C. The reaction mixture was allowed to attain ambient temperature with stirring, and stirring continued at ambient temperature for 1 hr. Excess reagent was quenched with saturated sodium carbonate (10 mL), ammonium chloride (10 mL) and water (10 mL). Dichloromethane was removed under reduced pressure. The aqueous layer was washed with 50% diethyl ether/hexane (200 mL) to remove the excess triethyl phosphate, and then the aqueous layer was concentrated under reduced pressure. The residue was treated with 30% MeOH/CH₂Cl₂ (200 mL), the resulting suspension was filtered through a pad of Celite, and the filtrate was concentrated under reduced pressure to obtain a crude mass. Purification of the crude mass by reverse-phase prep HPLC with 10 mM ammonium acetate in water as solvent A and acetonitrile as solvent B and lyophilisation of the desired column fractions furnished compound 5 (200 mg, 6%) as a white solid. LCMS (M⁺): 545.2.

### 1-((2R,3R,4S,5R)-5-(((Bis(((S)-1-methoxy-1-oxopropan-2-yl)amino)phosphoryl)oxy)methyl)-3,4-dihydroxytetrahydrofuran-2-yl)-3-carboxamidepyridin-1-ium Trifluoroacetate (MP-17)

The isopropylidene 5 (10 mg) was treated with 80% aqueous trifluoroacetic acid (2 mL) at 0° C. for 0.5 hr, and then the reaction mixture was concentrated under reduced pressure to get a crude residue. The crude residue was purified by reverse-phase prep HPLC with 0.1% trifluoroacetic acid in water as solvent A and acetonitrile as solvent B, and lyophilisation of the desired column fractions yielded MP-17 (4.5 mg, 48%) as a white solid. ¹H NMR (400 MHz, D₂O): δ 9.38 (s, 1H), 9.13 (d, J=6.3 Hz, 1H), 8.92 (d, J=7.4 Hz, 1H), 8.21 (d, J=7.1 Hz, 1H), 6.16 (s, 1H), 4.29-4.35 (m, 3H), 4.19 (s, 1H), 4.06-4.07 (m, 1H), 3.73-3.84 (m, 2H), 3.60 (d, J=7.6 Hz, 6H) and 1.20 (d, J=8 Hz, 6H). LCMS (M⁺): 505.1.

### 1-((2R,3R,4R,5R)-5-(((Bis(((S)-1-methoxy-1-oxopropan-2-yl)amino)phosphoryl)oxy)methyl)-3,4-bis(propionyloxy)tetrahydrofuran-2-yl)-3-carboxamidepyridin-1-ium Trifluoroacetate (MP-20)

Into a 50 mL single-neck round-bottom flask containing MP-17 (40 mg) in a mixture of anhydrous pyridine (2 mL) and acetonitrile (10 mL) was added propanoic anhydride (0.4 mL) at ambient temperature under nitrogen atmosphere, and the reaction mixture was stirred at ambient temperature for 16 hr. The reaction mixture was concentrated under reduced pressure, and the crude mass was purified by reverse-phase prep HPLC with 0.1% trifluoroacetic acid in water as solvent A and acetonitrile as solvent B. Lyophilisation of the desired column fractions furnished MP-20 (12 mg, 50%) as a white solid. ¹H NMR (400 MHz, D₂O): δ 9.43 (s, 1H), 9.16 (d, J=6.2 Hz, 1H), 8.97 (d, J=8 Hz, 1H), 8.25 (t, J=6.5 Hz, 1H), 6.54 (d, J=4.7 Hz, 1H), 5.43-5.44 (m, 2H), 4.82 (s, 1H), 4.40 (t, J=1.5 Hz, 1H), 4.25-4.25 (m, 1H), 3.87 (t, J=7.4 Hz, 2H), 3.61 (d, J=19.5 Hz, 6H), 2.39-2.41 (m, 4H), 1.28 (d, J=7.04 Hz, 6H) and 1.00-1.02 (m, 6H). LCMS (M⁺): 617.3.

### Example 2. Synthesis of 1-((2R,3R,4S,5R)-5-(((L-Valyl)oxy)methyl)-3,4-dihydroxytetrahydrofuran-2-yl)-3-carboxamidepyridin-1-ium Trifluoroacetate (MP-41)

FIG. 10 shows the process for the synthesis of MP-41.

### 1-((3aR,4R,6R,6aR)-6-((((tert-Butoxycarbonyl)-L-valyl)oxy)methyl)-2,2-dimethyltetrahydrofuro[3,4-d][1,3]dioxol-4-yl)-3-carboxamidepyridin-1-ium Triflate (8)

To a well stirred solution of compound 4 (700 mg, 1.6 mmol, prepared according to Example 1) in anhydrous THF (10 mL) were added (tert-butoxycarbonyl)-L-valine (618.4 mg, 2.84 mmol) and triphenylphosphine (TPP, 932.9 mg, 3.558 mmol) at ambient temperature under nitrogen atmosphere. The reaction mixture was stirred for 5 min at ambient temperature, and then diisopropyl azodicarboxylate (DIAD, 719.4 mg, 3.558 mmol) was added. The reaction mixture was stirred for 16 hr at room temperature. After completion of the reaction (monitored by LCMS), the reaction mixture was concentrated under reduced pressure to get crude compound 8 (3 g) as a thick viscous orange liquid, which was used in the next step without further purification. LCMS (M⁺): 494.

### 1-((2R,3R,4S,5R)-5-(((L-Valyl)oxy)methyl)-3,4-dihydroxytetrahydrofuran-2-yl)-3-carboxamidepyridin-1-ium Trifluoroacetate (MP-41)

To a well stirred solution of compound 8 (750 mg, 1.518 mmol) in anhydrous CH₂Cl₂ (20 mL) was added 2 mL of trifluoroacetic acid under inert atmosphere at 0° C., and the reaction mixture was stirred at ambient temperature for 1 hr. After completion of the reaction (monitored by LCMS), the reaction mixture was concentrated under reduced pressure. The crude residue was purified by reverse-phase HPLC to afford MP-41 (150 mg) as an off-white solid. ¹H NMR (400 MHz, CD₃OD): δ9.52 (s, 1H), 9.20 (d, J=6.2 Hz, 1H), 9.09 (d, J=8.1 Hz, 1H), 8.31-8.34 (m, 1H), 6.25 (d, J=4.1 Hz, 1H), 4.75 (t, J=14 Hz, 1H), 4.63-4.68 (m, 2H), 4.39 (t, J=9 Hz, 1H), 4.27-4.32 (m, 2H), 2.29-3.37 (m, 1H) and 1.08-1.11 (m, 6H). LCMS (M⁺): 354.

### Example 3. Synthesis of 3-Carboxamide-1-((2R,3R,4S,5R)-5-(((3-carboxypropanoyl)oxy)methyl)-3,4-dihydroxytetrahydrofuran-2-yl)pyridin-1-ium Trifluoroacetate (MP-42)

### 3-Carboxamide-1-((3aR,4R,6R,6aR)-6-(((3-carboxypropanoyl)oxy)methyl)-2,2-dimethyltetrahydrofuro[3,4-d][1,3]dioxol-4-yl)pyridin-1-ium Formate (10)

Into a 100 mL single-neck round-bottom flask containing a well stirred solution of compound 4 (1 g, 2.25 mmol, prepared according to Example 1) in a mixture of anhydrous pyridine (15 mL) and CH₂Cl₂ (5 mL) was added succinic anhydride (3.37 g, 33.7 mmol) at ambient temperature under nitrogen atmosphere. The reaction mixture was stirred at room temperature overnight, and then was concentrated under reduced pressure. The crude mass was purified by reverse-phase HPLC with 0.1% formic acid in water as solvent A and acetonitrile as solvent B to yield compound 10 (500 mg, 41%) as a colorless syrupy liquid. ¹H NMR (400 MHz, D₂O): δ 9.33 (s, 1H), 9.14 (d, J=6.4 Hz, 1H), 8.94 (d, J=8 Hz, 1H), 8.25-8.21 (m, 1H), 6.43 (d, J=2 Hz, 1H), 5.26 (t, J=2.4 Hz, 1H), 4.99 (d, J=6 Hz, 2H), 4.41 (s, 2H), 3.61 (s, 4H), 2.46-2.42 (m, 2H), 2.32-2.26 (m, 1H), 2.10-2.03 (m, 1H), 1.59 (s, 3H) and 1.38 (s, 3H). LCMS (M⁺): 395.1.

### 3-Carboxamide-1-((2R,3R,4S,5R)-5-(((3-carboxypropanoyl)oxy)methyl)-3,4-dihydroxytetrahydrofuran-2-yl)pyridin-1-ium Trifluoroacetate (MP-42)

Into a 25 mL single-neck round-bottom flask containing a well stirred solution of compound 10 (100 mg, 0.18 mmol) in CH₂Cl₂ (5 mL) was added 80% aqueous trifluoroacetic acid (5 mL) at 0° C. The reaction mixture was stirred at ambient temperature for 2 hr, and then was concentrated under reduced pressure. The crude product was purified by reverse-phase HPLC with 0.1% trifluoroacetic acid in water as solvent A and acetonitrile as solvent B to afford MP-42 (20 mg, 23%) as a colorless syrup. ¹H NMR (400 MHz, D₂O): δ 9.53 (s, 1H), 9.26 (d, J=6.4 Hz, 1H), 9.08-9.05 (m, 1H), 8.34-8.31 (m, 1H), 6.20 (d, J=5.2 Hz, 1H), 4.63-4.61 (m, 1H), 4.53 (t, J=3.6 Hz, 2H), 4.37 (t, J=5.2 Hz, 1H), 4.29-4.27 (m, 1H) and 2.63 (s, 4H). LCMS (M⁺): 355.1.

### Example 4. Synthesis of 3-Carboxamide-1-((2R,3R,4S,5R)-3,4-dihydroxy-5-(((4-methoxy-4-oxobutanoyl)oxy)methyl)tetrahydrofuran-2-yl)pyridin-1-ium Trifluoroacetate (MP-43)

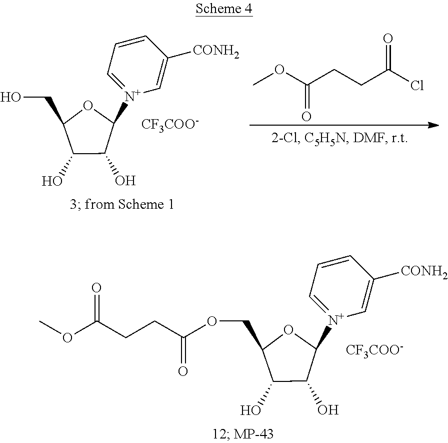

### 3-Carboxamide-1-((2R,3R,4S,5R)-3,4-dihydroxy-5-(((4-methoxy-4-oxobutanoyl)oxy)methyl)tetrahydrofuran-2-yl)pyridin-1-ium Trifluoroacetate (MP-43)

Into a 25 mL single-neck round-bottom flask containing a well stirred solution of compound 3 (368 mg, 1 mmol, prepared according to Example 1) in anhydrous DMF (5 mL) were added 2-chloropyridine (1.76 g, 15.51 mmol) and methyl 4-chloro-4-oxobutanoate (1.09 g, 7.23 mmol) at ambient temperature under nitrogen atmosphere. The reaction mixture was stirred at ambient temperature for 0.5 hr, the acid chloride was quenched with excess MeOH, and the reaction mixture was concentrated under reduced pressure. The crude product was purified by reverse-phase HPLC with 0.1% trifluoroacetic acid in water as solvent A and acetonitrile as solvent B to yield MP-43 (20 mg, 4%) as a colorless syrup. ¹HNMR (400 MHz, D₂O): δ 9.54 (s, 1H), 9.26 (d, J=6 Hz, 1H), 9.08 (d, J=8 Hz, 1H), 8.35-8.32 (m, 1H), 6.21 (d, J=5.2 Hz, 1H), 4.62-4.61 (m, 1H), 4.54-4.51 (m, 2H), 4.38-4.36 (m, 1H), 4.28-4.26 (m, 1H), 3.65 (s, 3H) and 2.66 (s, 4H). LCMS (M⁺): 369.1.

### Example 5. Elevation of NAD⁺ Level and Reduction of Cytotoxicity after Acute NAD⁺ Depletion and Cytotoxicity Induced by DNA Damage

Test NR derivatives were evaluated for their ability to increase NAD⁺ level and reduce cellular toxicity after acute NAD⁺ depletion and cytotoxicity induced by DNA damage. HepG2 (liver), HEK (kidney) and Jurkat (T-cells) cell lines were obtained from ATCC. HepG2 and HEK cells were incubated in Dulbecco's Modified Eagle Medium (DMEM) (Thermo Fischer) with 5% fetal bovine serum (FBS), while Jurkat cells were incubated in RPMI 1640 medium (Gibco). DNA damage in the cells was induced using the DNA-alkylating mutagen N-methyl-N-nitroso-N′-nitroguanidine (MNNG). Dose-response relationships of concentrations of MNNG and the magnitude of NAD⁺ depletion and cytotoxicity were established in the three different cell lines. Briefly, cells were incubated with varying concentrations of MNNG-containing media for 30 min. The cells were washed, and then were incubated with varying concentrations of a test compound for 3.5 hr. As controls, different combinations of incubation of the cells with or without MNNG for 30 min, washing of the cells, and incubation of the cells with or without a test compound for 3.5 hr were performed. Total cellular NAD⁺ level was measured using the NAD/NADH Glom Assay (Promega). Cytotoxicity was assessed using the CellTiter-Blue® Cell Viability Assay (Promega). About 80,000 cells per well were utilized for the experiments.

### MP-17 Reduced MNNG-Induced NAD⁺ Depletion and Cytotoxicity in Jurkat Cells:

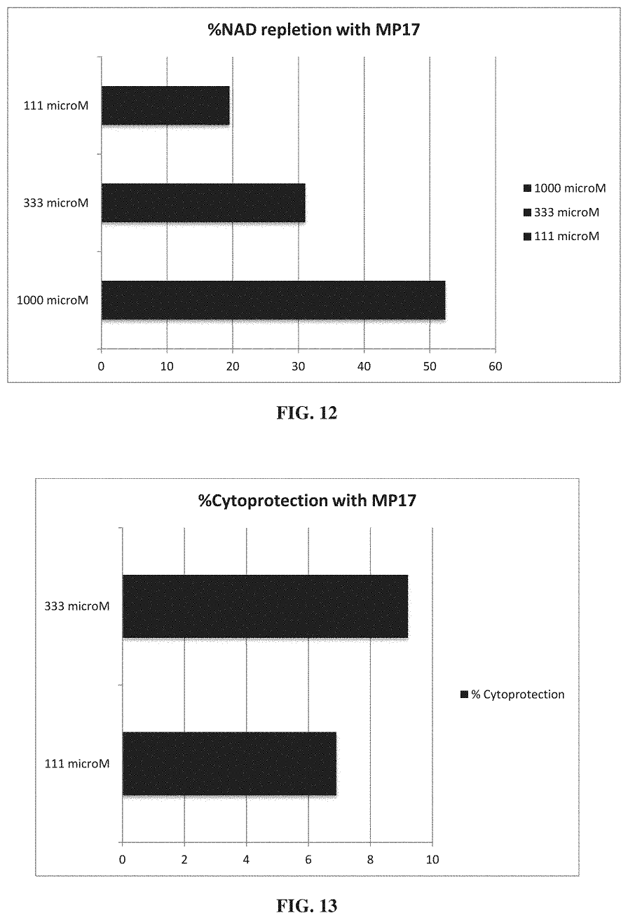

Jurkat cells were incubated with or without 100 μM of MNNG for 30 min. The cells were washed, and then were incubated with or without MP-17 (111-1000 μM) for 3.5 hr. MNNG treatment resulted in 76.9% depletion of NAD⁺ level at 4 hr. Depending on the concentration of MP-17, MP-17 induced 19.5% to 52.3% recovery of NAD⁺ level (FIG. 12).

Jurkat cells were incubated with or without 150 μM of MNNG for 30 min. The cells were washed, and then were incubated with or without MP-17 (111-1000 μM) for 3.5 hr. MNNG treatment resulted in 63.2% cytotoxicity at 4 hr. MP-17 provided cytoprotection (i.e., reduced cytotoxicity) by 6.9-9.2% (FIG. 13).

### MP-41 Reduced MNNG-Induced NAD⁺ Depletion and Cytotoxicity in Jurkat Cells:

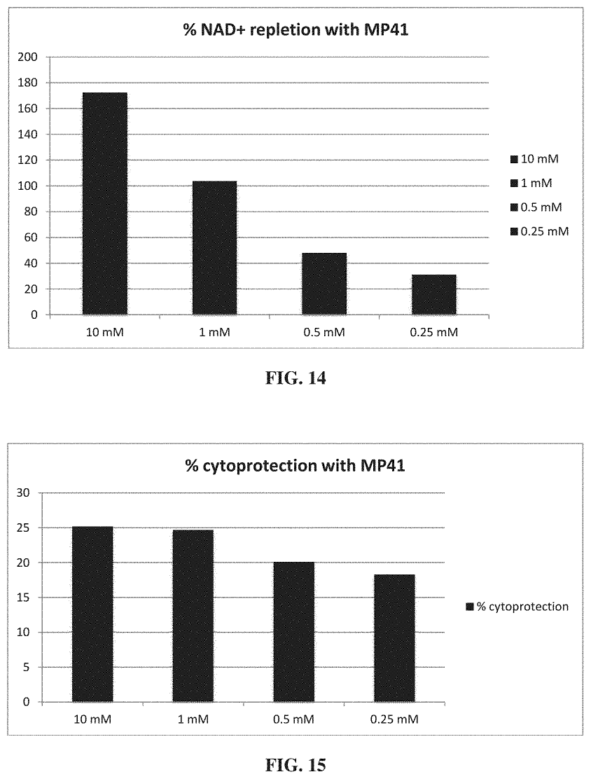

Jurkat cells were incubated with or without 75 μM of MNNG for 30 min. The cells were washed, and then were incubated with or without MP-41 (0.25-10 mM) for 3.5 hr. MNNG treatment resulted in 92.1% depletion of NAD⁺ level at 4 hr. Depending on the concentration of MP-41, MP-41 induced 31% to 172% recovery of NAD⁺ level (FIG. 14).

Jurkat cells were incubated with or without 75 μM of MNNG for 30 min. The cells were washed, and then were incubated with or without MP-41 (0.25-10 mM) for 3.5 hr. MNNG treatment resulted in 87% cytotoxicity at 4 hr. MP-41 provided cytoprotection (i.e., reduced cytotoxicity) by 18-25% (FIG. 15).

### MP-42 and MP-43 Reduced MNNG-Induced NAD⁺ Depletion and Cytotoxicity in HepG2 and Jurkat Cells:

Using similar procedures as described above for the assays of MP-17 and MP-41 in Jurkat cells, MP-42 and MP-43 reduced MNNG-induced NAD⁺ depletion and cytotoxicity in HepG2 and Jurkat cells, as indicated in Tables 1 and 2. The concentration of MNNG used in the cytotoxicity experiments with HepG2 cells (Table 2) was higher because these cells are more resistant to cytotoxicity despite a large reduction in NAD⁺ level.

TABLE 1

| | |
|---|---|
| MNNG Conc. | % NAD⁺ Repletion |

| Cell Line | (μM) | MP-42 (1 mM) | MP-43 (1 mM) |
|---|---|---|---|
| HepG2 | 100 | 23.7 | 31.1 |
| Jurkat | 100 | 8.2 | 12.9 |

TABLE 2

| | |
|---|---|
| MNNG Conc. | % Cytoprotection |

| Cell Line | (μM) | MP-42 (1 mM) | MP-43 (1 mM) |
|---|---|---|---|
| HepG2 | 600 | 9.9 | 5.7 |
| Jurkat | 100 | 4.5 | 6.3 |

### Example 6. Synergistic NAD⁺ Repletion and Cytoprotection by Combination of Nicotinamide Riboside and Very Low-Dose Olaparib

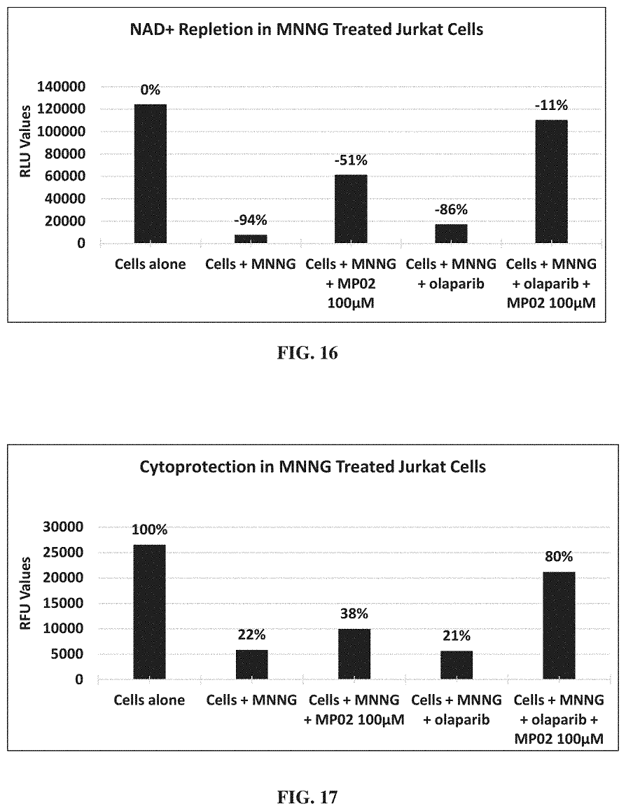

DNA damage was induced by MNNG in Jurkat cells, total cellular NAD⁺ level was measured, and cytotoxicity was assessed as described in Example 5. Briefly, Jurkat cells were incubated with or without 100 μM of MNNG for 30 min. The cells were washed, and then were incubated with or without 100 μM of nicotinamide riboside (NR) or 5 nM of olaparib, or both NR and olaparib, for 3.5 hr. MNNG treatment resulted in 94% depletion of NAD⁺ level at 4 hr. NR (100 μM) without olaparib increased NAD⁺ level by 43%, while olaparib (5 nM) without NR increased NAD⁺ level by 8%. However, the combination of both NR (100 PM) and olaparib (5 nM) synergistically increased NAD⁺ level by 75% to a similar level as in cells not treated with MNNG (FIG. 16). MP02 in FIGS. 16 and 17 is NR.

Jurkat cells were incubated with or without 200 μM of MNNG for 30 min. The cells were washed, and then were incubated with or without 100 μM of NR or 5 nM of olaparib, or both NR and olaparib, for 3.5 hr. MNNG treatment resulted in 78% cytotoxicity at 4 hr. NR (100 μM) without olaparib provided cytoprotection (i.e., reduced cytotoxicity) by 16%, while olaparib (5 nM) without NR provided no cytoprotection. However, the combination of both NR (100 μM) and olaparib (5 nM) synergistically enhanced cytoprotection by 58% to a cytotoxicity level of 20% relative to cells not treated with MNNG (FIG. 17).

### Example 7. Effects of Combinations of NR/NRH, Nitazoxanide or/and Olaparib on NAD⁺ Level, NAD⁺/NADH Ratio and ATP Level

### Cell Culture

### Conditions for Culture of Jurkat Cells:

Jurkat cells were revived for the NAD⁺ assay, NADH production assay and ATP assay. Cells were maintained in RPMI 1640 Media (Catalog No. A1049101, Thermo Fisher Scientific) supplemented with 10% FBS, 100 units/mL penicillin and 100 μg/mL streptomycin.

### Conditions for Culture of HepG2 Cells:

HepG2 cells were revived for the NAD⁺ assay, NADH production assay and ATP assay. Cells were maintained in DMEM GlutaMAX™ (Catalog No. 10569044, ThermoFisher Scientific) supplemented with 10% FBS, 100 units/mL penicillin and 100 μg/mL streptomycin.

### Treatment of Cells

Treatment without MNNG:

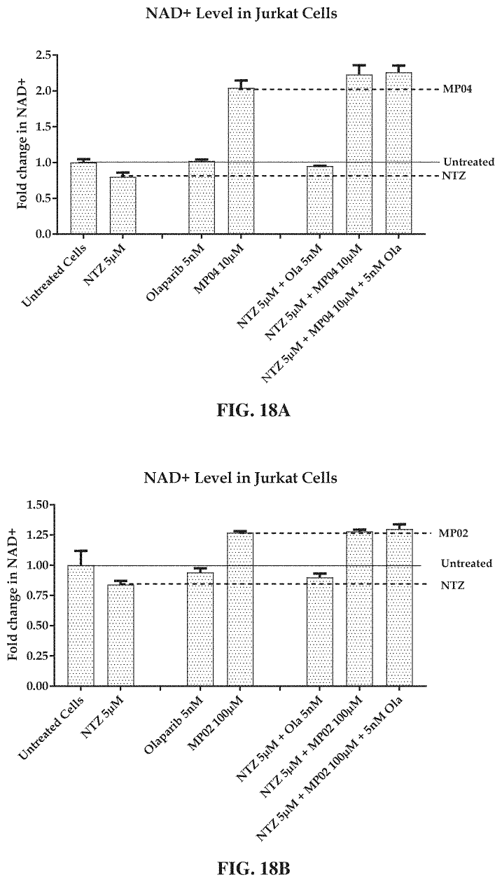

Cells were treated for 4 hr with NR (MP2), NRH (MP04), nitazoxanide (NTZ) or olaparib (Ola) alone, or combinations thereof, at the concentrations shown in FIGS. 18-25 at the start of the experiment. Cells were assayed for changes in NAD⁺ concentration, NADH production and ATP concentration at 4 hr. Three runs (n=3) were performed for each experiment.

Treatment with MNNG:

Cells were treated for 30 min with MNNG at the start of the experiment at a concentration in the range of 15-40 M for Jurkat cells and in the range of 25-600 μM for HepG2 cells. Cells were washed at 30 min and then were incubated for 4 hr with NR (MP2), NRH (MP04), nitazoxanide (NTZ) or olaparib (Ola) alone, or combinations thereof, at the concentrations shown in FIGS. 18-25. Cells were assayed for changes in NAD⁺ concentration, NADH production and ATP concentration at 4 hr. NAD⁺ and NADH values were standardized to untreated cells and NAD⁺/NADH ratios were calculated. Three runs (n=3) were performed for each experiment.

### Assays

### NAD⁺ Assay:

Total cellular NAD⁺ concentration was measured using HPLC/LCMS. Treated cells were pelleted and washed with ammonium bicarbonate buffer. Cells were lysed mechanically and extracted using a methanol/acetonitrile buffer. The extracted mixture was analyzed using an HPLC/LCMS instrument.

### NADH Production Assay:

NADH production was measured by the CellTiter-Blue® Assay (Promega). The assay measures the reduction of a redox dye (resazurin) to a fluorescent end product (resorufin) by NADH in cells, which thus depends on mitochondrial NADH production.

### ATP Assay:

Relative ATP levels were measured by the CellTiter-Glo® Assay (Promega). ATP levels were expressed in RLU (relative light units) to blank. Data interpretation was relative to control. The assay uses ATP in cells to create a chemiluminescent signal using luciferin and a thermostable luciferase.

FIG. 18A shows the effects of NRH (MP04), nitazoxanide or olaparib alone, and combinations thereof, on NAD⁺ level in Jurkat cells not treated with MNNG (n=3 for each experiment). The combination of NRH plus NTZ elicited a significantly higher NAD⁺ level than the sum of the results for NRH and NTZ alone. FIG. 18B shows the effects of NR (MP2), nitazoxanide or olaparib alone, and combinations thereof, on NAD⁺ level in Jurkat cells not treated with MNNG (n=3 for each experiment). The combination of NR plus NTZ elicited a significantly higher NAD⁺ level than the sum of the results for NR and NTZ alone. Even at a 10-fold lower concentration (10 M vs. 100 μM), NRH alone provided a markedly higher NAD⁺ level than NR alone.

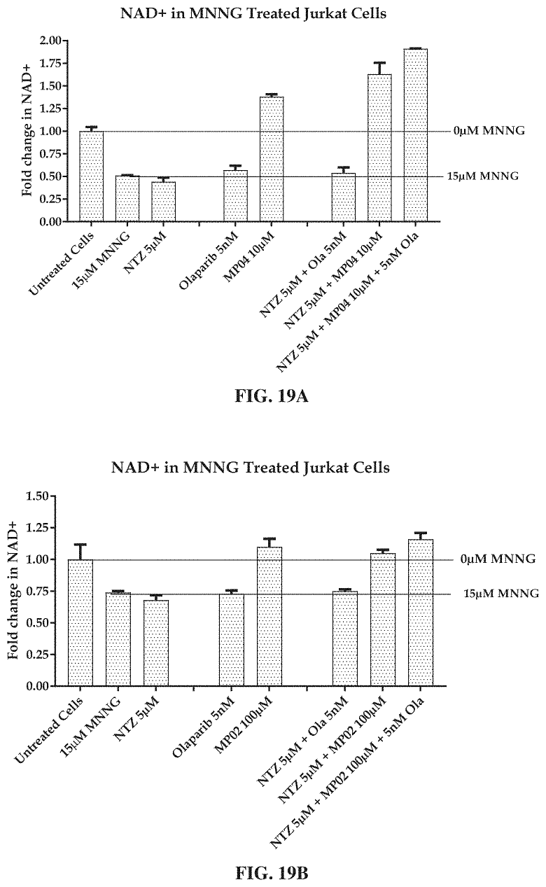

FIG. 19A shows the effects of NRH (MP04), nitazoxanide or olaparib alone, and combinations thereof, on NAD⁺ level in Jurkat cells treated with MNNG (n=3 for each experiment). MNNG treatment markedly reduced NAD⁺ level via stimulation of NAD⁺-consuming PARP. The combination of NRH plus NTZ elicited a significantly higher NAD⁺ level than the sum of the results for NRH and NTZ alone, and the combination of NRH plus NTZ and olaparib elicited a markedly higher NAD⁺ level than the sum of the results for NRH, NTZ and olaparib alone. FIG. 19B shows the effects of NR (MP2), nitazoxanide or olaparib alone, and combinations thereof, on NAD⁺ level in Jurkat cells treated with MNNG (n=3 for each experiment).

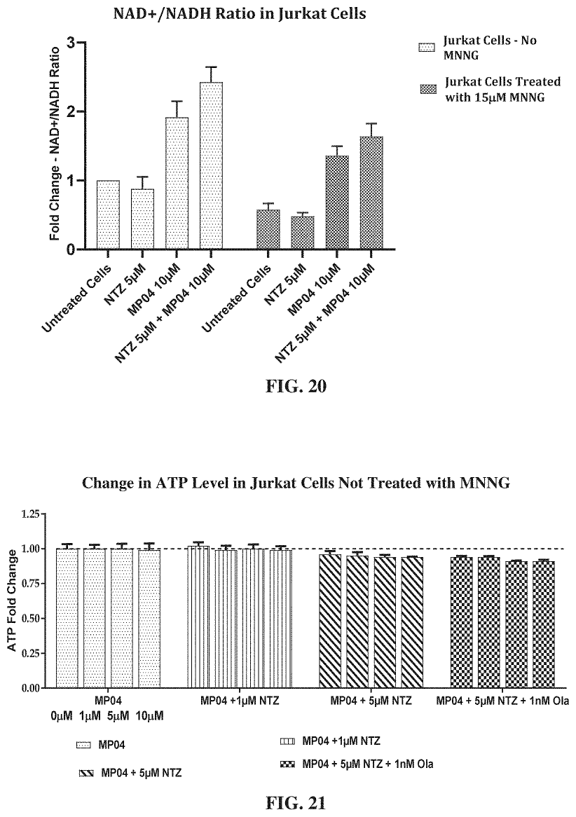

FIG. 20 shows the effects of NRH (MP04) or nitazoxanide alone, and a combination of both, on NAD⁺/NADH ratio in Jurkat cells not treated or treated with MNNG (n=3 for each experiment). MNNG treatment markedly reduced the NAD⁺/NADH ratio. The NAD⁺/NADH ratio is based on total cellular NAD⁺ level and mitochondrial NADH level.

FIG. 21 shows the effects of NRH (MP04) alone, and combinations of NRH plus nitazoxanide or NRH plus nitazoxanide and olaparib, on ATP level in Jurkat cells not treated with MNNG (n=3 for each experiment). FIG. 21 shows that a combination of NRH (5 or 10 μM) plus NTZ (5 μM) resulted in a modest reduction of ATP level. The combination of 10 μM NRH plus 5 μM NTZ also increased NAD⁺ level (FIG. 18A) and NAD⁺/NADH ratio (FIG. 20) in Jurkat cells not treated with MNNG. The results indicate that a combination of NRH (e.g., 10 μM) plus NTZ (e.g., 5 μM) can achieve healthy mitochondrial uncoupling in Jurkat cells not treated with MNNG. FIG. 21 also shows that a combination of NRH at various concentrations plus 5 μM NTZ and 1 nM olaparib resulted in a moderate reduction of ATP level, and a combination of 10 μM NRH plus 5 μM NTZ and 5 nM olaparib also increased NAD⁺ level in Jurkat cells not treated with MNNG (FIG. 18A).

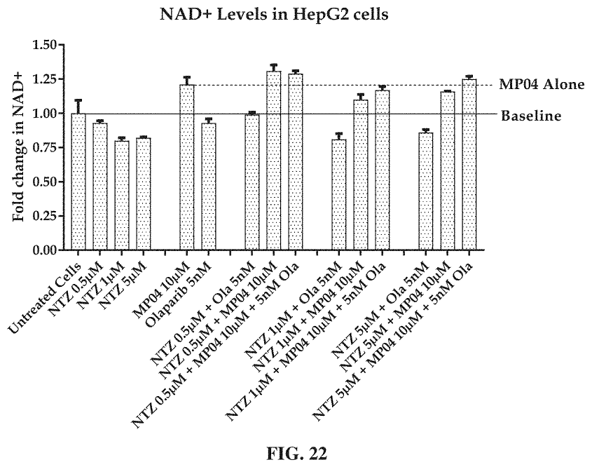

FIG. 22 shows the effects of NRH (MP04), nitazoxanide at various concentrations or olaparib alone, and combinations thereof, on NAD⁺ level in HepG2 cells not treated with MNNG (n=3 for each experiment). The combination of NRH plus 1 or 5 μM NTZ elicited a significantly higher NAD⁺ level than the sum of the results for NRH and NTZ alone, and the combination of NRH plus 1 or 5 μM NTZ and olaparib elicited a significantly higher NAD⁺ level than the sum of the results for NRH, NTZ and olaparib alone.

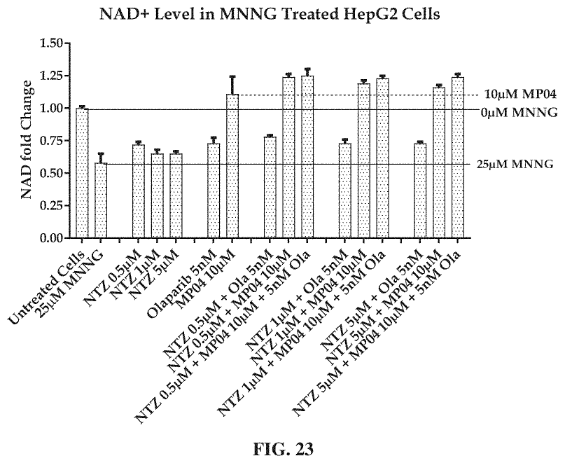

FIG. 23 shows the effects of NRH (MP04), nitazoxanide at various concentrations or olaparib alone, and combinations thereof, on NAD⁺ level in HepG2 cells treated with MNNG (n=3 for each experiment). MNNG treatment markedly reduced NAD⁺ level via stimulation of NAD⁺-consuming PARP.

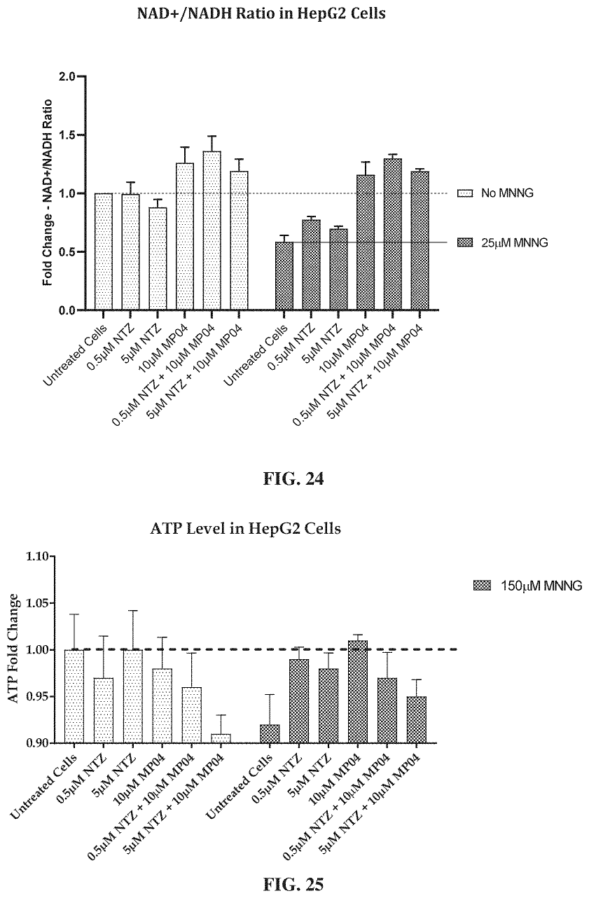

FIG. 24 shows the effects of NRH (MP04) or 0.5 or 5 μM nitazoxanide alone, and combinations of both, on NAD⁺/NADH ratio in HepG2 cells not treated or treated with MNNG (n=3 for each experiment). MNNG treatment markedly reduced the NAD⁺/NADH ratio. The NAD⁺/NADH ratio is based on total cellular NAD⁺ level and mitochondrial NADH level.

FIG. 25 shows the effects of NRH (MP04) or 0.5 or 5 μM nitazoxanide alone, and combinations of both, on ATP level in HepG2 cells not treated or treated with MNNG (n=3 for each experiment). MNNG treatment significantly reduced ATP level via stimulation of ATP-consuming PARP. FIG. 25 shows that a combination of 10 μM NRH plus 5 μM NTZ resulted in a modest or moderate reduction of ATP level in HepG2 cells not treated or treated with MNNG. The combination of 10 μM NRH plus 5 μM NTZ also increased NAD⁺ level (FIGS. 22 and 23) and NAD⁺/NADH ratio (FIG. 24) in HepG2 cells not treated or treated with MNNG. The results indicate that a combination of NRH (e.g., 10 μM) plus NTZ (e.g., 5 μM) can achieve healthy mitochondrial uncoupling in HepG2 cells not treated or treated with MNNG.

### Example 8. Combination of NRH and DNP Improved Viability and Metabolic Profile of HepG2 Cells

2,4-Dinitrophenol (commonly known as DNP) and NRH (MP04) were dissolved in DMSO to provide a final concentration of 50 μM DNP and 100 μM NRH in 5 mL of Dulbecco's Modified Eagle Medium (DMEM) media. Untreated cells were exposed to a similar volume of DMSO (vehicle only).

1×10⁵ HepG2 cells (a liver cancer cell line) were seeded in 25 mm³ culture plates and grown for 24 hr in high-glucose DMEM media using standard cell-culture procedures. The media in the culture plates was replaced with 5 mL of DMEM media containing DNP alone. NRH alone, or DNP plus NRH in the aforementioned concentrations. The cells were incubated at 37° C. under 6% CO₂ for 4 hr. The activity of lactate dehydrogenase (LDH) and the concentration of lactate released into the media, and the concentration of glucose in the media, after 4 hr were measured using an automated Roche e311 chemistry platform. After washing in trypsin EDTA, the cells were suspended and stained for viability with a 1:1 solution of 0.4% trypan blue. Live cells and dead cells were counted using a microscope (Olympus) and a hemocytometer. All experiments were performed in triplicate.

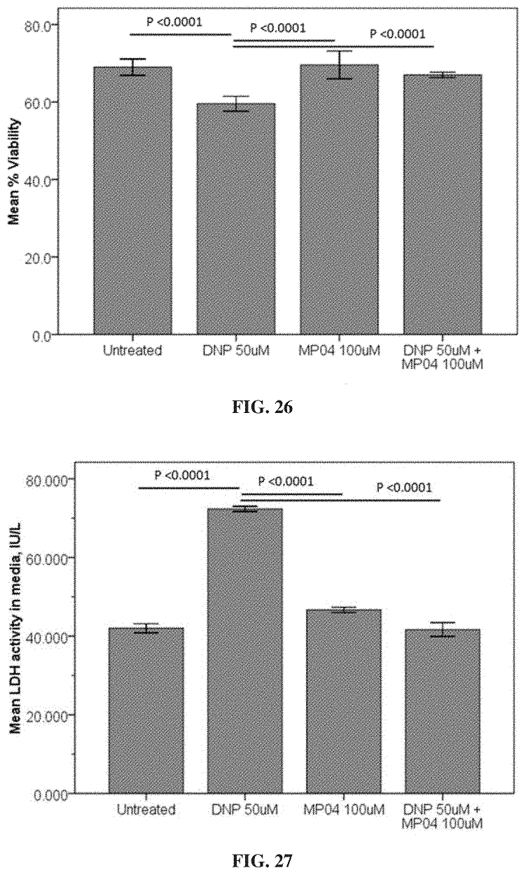

FIG. 26 shows the % viability of HepG2 cells untreated or treated with 50 μM DNP alone, 100 μM NRH (MP04) alone, or 50 μM DNP plus 100 μM NRH for 4 hr (the error bars represent +/− standard error of the mean [SEM]). 50 μM DNP alone significantly reduced % cell viability, suggesting that excessive uncoupling caused by a high concentration of DNP had cytotoxic effect. 100 μM NRH alone did not significantly affect % cell viability. HepG2 cells treated with 50 μM DNP plus 100 μM NRH had a similar % viability as untreated HepG2 cells, demonstrating that such a combination has cytoprotective effect.

FIG. 27 shows the activity (in international units per liter [IU/L]) of lactate dehydrogenase (LDH) released into the media from HepG2 cells untreated or treated with 50 μM DNP alone, 100 μM NRH (MP04) alone, or 50 μM DNP plus 100 μM NRH for 4 hr (the error bars represent +/−SEM). 50 μM DNP alone markedly increased LDH activity in the media. Release of LDH from cells and tissues is a marker of cell injury and tissue damage. 100 μM NRH alone modestly increased LDH activity in the media. HepG2 cells treated with 50 μM DNP plus 100 μM NRH had a similar LDH activity in the media as untreated HepG2 cells, demonstrating an additional cytoprotective effect of such a combination.

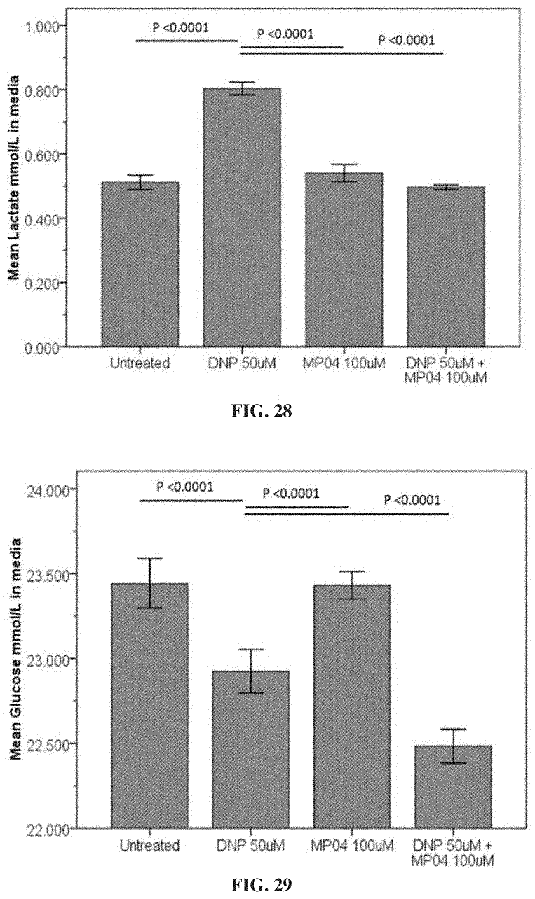

FIG. 28 shows the concentration (mmol/L) of lactate released into the media from HepG2 cells untreated or treated with 50 μM DNP alone, 100 μM NRH (MP04) alone, or 50 μM DNP plus 100 μM NRH for 4 hr (the error bars represent +/−SEM). 50 μM DNP alone markedly increased lactate concentration in the media, suggesting that excessive uncoupling caused by a high concentration of DNP increased glycolysis. 100 μM NRH alone did not significantly affect lactate concentration in the media, as was the case with 50 μM DNP plus 100 μM NRH, suggesting no increase in glycolysis.

FIG. 29 shows the concentration (mmol/L) of glucose in the media containing HepG2 cells untreated or treated with 50 μM DNP alone, 100 μM NRH (MP04) alone, or 50 μM DNP plus 100 μM NRH for 4 hr (the error bars represent +/−SEM). 50 μM DNP alone significantly reduced glucose concentration in the media, suggesting that excessive uncoupling caused by a high concentration of DNP increased glucose consumption by the cells via increased glycolysis to meet the energy need of the cells. 100 μM NRH alone did not significantly affect glucose concentration in the media. 50 μM DNP plus 100 μM NRH caused the greatest reduction of glucose concentration in the media, suggesting increased glucose consumption by the cells via increased TCA cycle flux. Without intending to be bound by theory, excessive uncoupling caused by a high concentration of DNP significantly reduces the efficiency of the TCA cycle and depletes ATP, and consequently the glycolytic HepG2 cancer cells increase glycolysis to meet their energy need. Addition of NRH leads to increased NAD⁺ level, which improves the redox state and enhances TCA cycle flux.

FIGS. 26-29 show that a combination of NRH and DNP can improve the health and metabolic profile of cells adversely affected by excessive uncoupling caused by a high concentration of DNP. The results show that use of a nicotinyl riboside compound such as NRH in combination with an uncoupler such as DNP can significantly increase the therapeutic index of the uncoupler, including medium-strength to strong uncouplers. Combination therapy with the nicotinyl riboside compound can lower the concentration of the uncoupler at which the uncoupler has therapeutic effect, and can increase the concentration of the uncoupler at which the uncoupler has toxic effect. Without intending to be bound by theory, a nicotinyl riboside compound can reverse deleterious effects due to excessive uncoupling by, e.g., providing increased NAD⁺ levels which maintain a redox state suitable for efficient TCA cycle flux and which support the activity of NAD⁺-dependent enzymes such as sirtuins that maintain or enhance cellular and mitochondrial health and function.

### Example 9. Effects of Combinations of Nicotinyl Riboside Compounds and Uncouplers on Cell Viability and Metabolic Profile of HepG2 Cells, Primary Human Hepatocytes, and Human Liver Organoids

Conditions of liver steatosis are mimicked in HepG2 cells, primary human hepatocytes, and human liver organoid models, such as by their exposure to palmitic acid or oleic acid for about 48-72 hours. The cells and organoids are then exposed to an uncoupler (e.g., DNP, BAM15, nitazoxanide or Example No. 134 in WO 2019/226490) alone at varying concentrations, to a nicotinyl riboside compound (e.g., NR, NRH, or a derivative thereof) alone at varying concentrations, or to an uncoupler plus a nicotinyl riboside compound at varying concentrations for, e.g., about 4, 8, 12, 24, 48 or 72 hours. As an illustrative example, the cells and organoids are exposed to about 10, 20, 30, 40 or 50 μM of DNP alone, to about 10, 20, 30, 40, 50 or 100 μM of NRH alone, and to 50 μM of DNP plus 100 μM of NRH and other combinations of concentrations of DNP plus NRH. Measurements performed for untreated cells and organoids and those treated with the uncoupler alone, the nicotinyl riboside compound alone, and the uncoupler plus the nicotinyl riboside compound include cell viability as assessed by, e.g., trypan blue dye exclusion; cellular oxygen consumption rate using, e.g., the Seahorse assay; cellular content of lipids (e.g., triglycerides); degree of de novo lipogenesis as assessed by, e.g., ¹⁴C incorporation into fatty acids; levels in the media of glucose, lactate, LDH, pro-inflammatory cytokines (e.g., IL-1α, -1β, -4 and -6, and TNF-α), and liver enzymes (e.g., ALT and AST).

### Example 10. Studies of Combinations of Nicotinyl Riboside Compounds and Uncouplers in Animal Models of Metabolic Disorders

Combinations of an uncoupler (e.g., controlled-release DNP, BAM15, nitazoxanide or Example No. 134 in WO 2019/226490) and a nicotinyl riboside compound (e.g., NR, NRH, or a derivative thereof) are evaluated for safety and efficacy in a high fat-diet Zucker Diabetic Fatty (ZDF) rat model of type 2 diabetes and NASH (adapted from, e.g., R. Perry et al., Science, 347:1253-1256 [2015]), a Western diet (WD) mouse model of obesity and insulin resistance (adapted from, e.g., S. Alexopoulos et al., Nature Commun.,11:2397 [2020]), a fatless AZIP/F-1 mouse model of severe lipodystrophy and diabetes (adapted from, e.g., A. Abulizi et al., FASEB J., 31:2916-2924 [2017]), and high-fat, fructose-fed cynomolgus macaques and spontaneously obese dysmetabolic rhesus macaques (adapted from, e.g., L. Goedeke et al., Sci. Transl. Med.,11, eaay0284 [2019]). Animals are untreated (treated with placebo) or treated with an uncoupler alone at varying doses, a nicotinyl riboside compound alone at varying doses, or a combination of the uncoupler and the nicotinyl riboside compound at varying doses. As an illustrative example, controlled-release DNP is given orally at about 5 mg/kg (e.g., once, or once daily or once every two days for about 1, 2, 4, 8 or 12 weeks) to achieve a steady-state plasma concentration of about 10 μM. NRH is given orally or intraperitoneally at about 100-250 mg/kg (e.g., once, or once daily for about 1, 2, 4, 8 or 12 weeks).

Measurements and analyses performed for untreated and treated animals include pharmacokinetics (PK) of the nicotinyl riboside compound and the uncoupler based on plasma/serum and liver concentrations; positional isotopomer nuclear magnetic resonance tracer analysis (PINTA) (which assesses, e.g., hepatic glucose production, pyruvate carboxylase flux [V_{PC}] and citrate synthase flux [V_{CS}]); histological analyses of the liver for steatosis (including the liver content of lipids [e.g., triglycerides and cholesterol]), inflammation and fibrosis; plasma/serum levels of lipids (e.g., triglycerides and cholesterol), LDL, VLDL, glucose, insulin, pro-inflammatory cytokines (e.g., IL-1α, -1β, -4 and -6, and TNF-α), liver enzymes (e.g., ALT and AST), urea nitrogen and creatinine; glucose tolerance test (GTT) and insulin tolerance test (ITT); cardiovascular measurements (e.g., heart rate); and body temperature. Safety is assessed based on, e.g., an implanted chip for continuous telemetry and temperature monitoring, and quantification of food intake, weight and activity.

Goals of the animal studies include determining whether co-administration of a nicotinyl riboside compound with an uncoupler reduces the lowest effective dose of the uncoupler compared to the lowest effective dose of the uncoupler given alone, and whether a particular dose of an uncoupler given alone and resulting in a significant adverse event or toxicity (e.g., a significant increase in body temperature, heart rate or the plasma/serum level of a liver enzyme) does not result in a significant adverse event or toxicity when co-administered with a nicotinyl riboside compound.

### Example 11. Studies of Combinations of NRH and Controlled-Release DNP in Animal Models of Metabolic Disorders

The safety and efficacy of combination regimens comprising an orally administered controlled-release formulation of DNP (CRDNP) and an orally administered formulation of NRH are evaluated in three high fat-diet animal models of NAFLD (including NASH) and insulin resistance—C57BJ6 mice, ZDF rats and cynomolgus monkeys.

In each of the three animal studies, about 54 animals are randomized to one of 9 treatment groups (n≈6 per group): (1) placebo; (2) CRDNP at about 5 mg/kg/day (to assess whether it is a minimally effective dose); (3) CRDNP at about 30 mg/kg/day (to assess whether it is a maximum tolerated dose); (4) CRDNP at about 100 mg/kg/day (to assess whether it is a toxic dose); (5) NRH at about 100 mg/kg/day; (6) NRH at about 250 mg/kg/day; (7) NRH at about 100 mg/kg/day plus CRDNP at about 5 mg/kg/day; (8) NRH at about 100 mg/kg/day plus CRDNP at about 30 mg/kg/day; and (9) NRH at about 100 mg/kg/day plus CRDNP at about 100 mg/kg/day. Each group undergoes treatment for about 6, 8 or 12 weeks.

All animals are fed with normal chow (control) or a high-fat diet for about 12 weeks. Blood samples are obtained on the first day of dosing and after the last dose. All animals are sacrificed at the end of the study. Measurements and analyses performed at baseline and at the end of the study for each of the 9 treatment groups include PK of NRH and DNP based on plasma/serum and liver concentrations; PINTA (which assesses, e.g., hepatic glucose production, V_{PC} and V_{CS}); histological analyses of the liver for steatosis (including the liver content of lipids [e.g., triglycerides and cholesterol]), inflammation and fibrosis; plasma/serum levels of lipids (e.g., triglycerides and cholesterol), LDL, VLDL, glucose, insulin, pro-inflammatory cytokines (e.g., IL-1α, -1β, -4 and -6, and TNF-α), liver enzymes (e.g., ALT and AST), urea nitrogen and creatinine; GTT and ITT; cardiovascular measurements (e.g., heart rate); and body temperature. Safety is assessed based on, e.g., an implanted chip for continuous telemetry and temperature monitoring, and quantification of food intake, weight and activity.

Goals of the animal studies include determining whether co-administration of NRH with CRDNP reduces the lowest effective dose of CRDNP compared to the lowest effective dose of CRDNP given alone, and whether a particular dose of CRDNP given alone and resulting in a significant adverse event or toxicity (e.g., a significant increase in body temperature, heart rate or the plasma/serum level of a liver enzyme) does not result in a significant adverse event or toxicity when co-administered with NRH.

### Example 12. Modulation of Immune Cells by Nicotinyl Riboside Compounds, Uncouplers and PARP Inhibitors

Peripheral blood mononuclear cells (e.g., T cells [including CD3-negative T cells], B cells and natural killer [NK] cells) are thawed and rested overnight in complete RPMI media. The cells are unstimulated or stimulated with CD3/CD28 for about 18 hr in the absence or presence of a nicotinyl riboside compound (e.g., NR, NRH, or a derivative thereof) alone at varying concentrations, an uncoupler (e.g., DNP, BAM15, nitazoxanide [NTZ] or Example No. 134 in WO 2019/226490) alone at varying concentrations, a PARP inhibitor (e.g., olaparib) alone at varying concentrations, or combinations thereof at varying concentrations. Non-limiting examples of test groups include: 1) unstimulated; 2) CD3/CD28; 3) NRH; 4) DNP or NTZ; 5) olaparib; 6) CD3/CD28+NRH; 7) CD3/CD28+DNP or NTZ; 8) CD3/CD28+olaparib; 9) CD3/CD28+NRH+DNP or NTZ; 10) CD3/CD28+NRH+olaparib; and 11) CD3/CD28+NRH+DNP or NTZ+olaparib. About 1 hr after initiation of the stimulation, GolgiStop and GolgiPlug are added to the culture media. About 18 hr afterward the cells are stained for intracellular cytokines (e.g., TNF-α, IFN-γ and IL-2) or the levels of cytokines released into the media are measured, and the cells are measured by flow cytometry. The number of CD4⁺, CD8+ and CD4⁺/CD8⁺ T cells, B cells and NK cells is counted.

### Example 13. Phase 1, Double-Blind, Placebo-Controlled Studies of Combinations of Nicotinyl Riboside Compounds and Uncouplers without or with PARP Inhibitors in Healthy Humans

The safety of combinations of a nicotinyl riboside compound (e.g., NR, NRH, or a derivative thereof) at varying doses and an uncoupler (e.g., controlled-release DNP, BAM15, nitazoxanide or Example No. 134 in WO 2019/226490) without or with a PARP inhibitor (e.g., olaparib) at varying doses is evaluated in Phase 1, double-blind, placebo-controlled, single- and multiple-ascending dose studies in healthy human subjects. An illustrative example is described for orally administered NRH, orally administered controlled-release DNP (CRDNP), and orally administered olaparib. The inclusion/exclusion criteria include healthy adults of 18-65 years of age and having a laboratory panel (e.g., blood chemistry, glucose and lipids) within normal limits.

Each group has N≈10, with about 8 active and about 2 placebo. For oral CRDNP, there are 4 single-ascending dose (SAD) cohorts taking about 10 mg, 30 mg, 100 mg and 300 mg per day, and 3 multiple-ascending dose (MAD) (7 days) cohorts taking about 10 mg, 30 mg and 100 mg per day. For oral NRH, there are 4 SAD/MAD (7 days) cohorts taking about 5 mg, 50 mg, 250 mg and 500 mg per day. For oral olaparib, there are 2 SAD/MAD (7 days) cohorts taking about 1 mg and 5 mg per day. In addition, there are 4 combination cohorts with 7 days of dosing in MAD (about 8 active and about 2 placebo): (1) about 30 mg/day CRDNP plus about 250 mg/day NRH; (2) about 100 mg/day CRDNP plus about 250 mg/day NRH; (3) about 30 mg/day CRDNP plus about 250 mg/day NRH plus about 5 mg/day olaparib; and (4) about 100 mg/day CRDNP plus about 250 mg/day NRH plus about 5 mg/day olaparib.

End points of the studies include body temperature, heart rate, adverse events, PINTA at baseline and about 12 hr after the first dose and the last dose, clinical chemistry and hematology, plasma/serum level of α-hydroxybutyrate (AHB), and whole-blood NADH/NAD⁺ ratio.

In addition to evaluating the safety of CRDNP alone, NRH alone and olaparib alone and combinations thereof, goals of the Phase 1 studies include determining whether a combination of CRDNP and NRH without or with olaparib improves PINTA measures (e.g., increases V_{c}s) and reduces AHB level and the NADH/NAD⁺ ratio.

### Example 14. Phase 2, Double-Blind, Placebo-Controlled Studies of Combinations of Nicotinyl Riboside Compounds and Uncouplers without or with PARP Inhibitors in Human Subjects with NASH

The efficacy and safety of an uncoupler (e.g., controlled-release DNP, BAM15, nitazoxanide or Example No. 134 in WO 2019/226490) alone at varying doses and in combination with a nicotinyl riboside compound (e.g., NR, NRH, or a derivative thereof) at varying doses without or with a PARP inhibitor (e.g., olaparib) at varying doses are evaluated in Phase 2, double-blind, placebo-controlled studies in human subjects with NASH but without cirrhosis. An illustrative example is described for orally administered NRH, orally administered controlled-release DNP (CRDNP), and orally administered olaparib. The inclusion/exclusion criteria include human subjects having NAFLD, insulin resistance, elevated ALT plasma/serum level, and an enhanced liver fibrosis (ELF) test score>9 and <11 or fibroscan>8 and kPa<14 (a measure of liver stiffness).

There are 7 treatment groups (N≈20 per group), with each treatment lasting about 12 weeks: (1) placebo; (2) about 30 mg/day CRDNP; (3) about 100 mg/day CRDNP; (4) about 30 mg/day CRDNP plus about 250 mg/day NRH; (5) about 100 mg/day CRDNP plus about 250 mg/day NRH; (6) about 30 mg/day CRDNP plus about 250 mg/day NRH plus about 5 mg/day olaparib; and (7) about 100 mg/day CRDNP plus about 250 mg/day NRH plus about 5 mg/day olaparib. End points of the studies include adverse events; clinical chemistry; proton density fat fraction in magnetic resonance imaging (MRI-PDFF); plasma/serum levels of lipids (e.g., triglycerides and cholesterol), LDL, lipoprotein(a) [Lp(a)], keratin 18 (CK18), AHB and liver enzymes (e.g., ALT and AST); whole-blood NADH/NAD⁺ ratio; ELF test score; and homeostatic model assessment for insulin resistance (HOMA-IR).

Goals of the Phase 2 studies include determining whether a combination of CRDNP and NRH without or with olaparib improves glycemic and lipid metabolic parameters, and reduces liver fat by MRI-PDFF, AHB level and the NADH/NAD⁺ ratio.

### Example 15. Measurement of Mitochondrial Function in Primary Cells

Mitochondrial function in different cell types (e.g., neural cells, liver cells, kidney cells, lymphoma cells and peripheral blood mononuclear cells) after exposure to NR/NAR derivatives of the disclosure or NR (positive control) is measured. Mitochondrial mass is measured using the Mitotracker assay (ThermoFisher Scientific). Mitochondrial super oxide production is measured using the Mitosox assay (ThermoFisher Scientific) assay. Mitochondrial membrane potential is measured using the JC-1 Dye assay (ThermoFisher Scientific).

### Example 16. In Vivo PK and Efficacy of NR and NAR Derivatives

In vivo pharmacokinetic studies and pharmacodynamic studies (e.g., NAD⁺ levels in the blood and in different cell types, such as neural cells, liver cells and kidney cells) of orally and parenterally (e.g., intravenously and subcutaneously) administered NR and NAR derivatives of the disclosure and NR (positive control) are performed in rats, and EC₅₀ values are calculated.

### Example 17. Stability of NR and NAR Derivatives

The stability of NR and NAR derivatives of the disclosure and NR (for comparison) in different types of media is determined using HPLC-based analytical methods. Examples of such media include: 1) phosphate buffers at pH 2, 4, 6, 7, 7.4, 8 and 9; 2) Cell Culture Media (CCM); 3) Dulbecco's Modified Eagle Medium (DMEM) supplemented with 10% fetal bovine serum (FBS); 4) rat plasma; and 5) human plasma.

### REFERENCES

The following citations are incorporated herein by reference in their entirety:
1. Adams and Victor's Principles of Neurology, 10th Ed. (2014)
2. Bieganowski, P. and Brenner, C., Discoveries of nicotinamide riboside as a nutrient and conserved NRK genes establish a Preiss-Handler independent route to NAD⁺ in fungi and humans, Cell, 117(4):495-502 (2004)
3. Bonkowski, M. and Sinclair, D., Slowing ageing by design: the rise of NAD and sirtuin-activating compounds, Nat. Rev. Mol. Cell Biol., 17(11):679-690 (2016)
4. Camacho-Pereira, J. et al., CD38 Dictates Age-Related NAD Decline and Mitochondrial Dysfunction through an SIRT3-Dependent Mechanism, Cell Metab., 23(6):1127-1139 (2016)
5. Chini, C. et al., NAD and the aging process: Role in life, death and everything in between, Mol. Cell Endocrinol., 455:62-74 (2017)
6. Croteau, D. et al., NAD in DNA repair and mitochondrial maintenance, Cell Cycle, 16(6):491-492 (2017)
7. de Picciotto, N. et al., Nicotinamide mononucleotide supplementation reverses vascular dysfunction and oxidative stress with aging in mice, Aging Cell, 15(3):522-530 (2016)
8. de Vita and Lawrence, Cancer: Principles and Practice of Oncology, 10^{th} Ed. (2015)
9. Fang, E. et al., NAD Replenishment Improves Lifespan and Healthspan in Ataxia Telangiectasia Models via Mitophagy and DNA Repair, Cell Metab., 24(4):566-581 (2016)
10. Fang, E. and Bohr, V., NAD: The convergence of DNA repair and mitophagy, Autophagy, 13(2):442-443 (2017)
11. Frederick, D. et al., Loss of NAD Homeostasis Leads to Progressive and Reversible Degeneration of Skeletal Muscle, Cell Metab., 24(2):269-282 (2016)
12. Imai, S. and Guarente, L., NAD⁺ and sirtuins in aging and disease, Trends Cell Biol., 24(8):464-471 (2014)
13. López-Otín, C. et al., The Hallmarks of Aging, Cell, 153(6):1194-1217 (2013)
14. Physician's Desk Reference, 70th Ed. (2016)
15. WO 2015/186068 A1
16. WO 2017/079195 A1
17. Mills, K. et al., Long-Term Administration of Nicotinamide Mononucleotide Mitigates Age-Associated Physiological Decline in Mice, Cell Metab., 24(6):795-806 (2016)
18. Ratajczak, J. et al., NRK1 controls nicotinamide mononucleotide and nicotinamide riboside metabolism in mammalian cells, Nat. Commun., 7:13103 (2016)
19. Stein, L. and Imai, S., The dynamic regulation of NAD metabolism in mitochondria, Trends Endocrinol. Metab., 23(9):420-428 (2012)
20. Stunkard and Wadden, Obesity: Theory and Therapy, 2nd Ed. (1993)
21. Trammell, S. et al., Nicotinamide riboside is uniquely and orally bioavailable in mice and humans, Nat. Commun., 7:12948 (2016)
22. U.S. Pat. No. 8,383,086
23. Yang, Y. and Sauve, A., NAD⁺ metabolism: Bioenergetics, signaling and manipulation for therapy, Biochim. Biophys. Acta,1864(12):1787-1800 (2016)
24. Zhang, H. et al., NAD⁺ repletion improves mitochondrialand stem cell function and enhances life span in mice, Science, 352(6292):1436-1443 (2016)

While various embodiments of the present disclosure have been described, such embodiments are provided by way of illustration and example only. Numerous variations thereof and modifications thereto will be apparent to those skilled in the art and are encompassed by the present disclosure. It is understood that various alternatives to the embodiments of the disclosure can be employed in practicing the disclosure and are encompassed by the disclosure.

What is claimed is:

1. A method of treating a mitochondria-related disease or condition, comprising administering to a subject in need of treatment a therapeutically effective amount of a nicotinyl riboside compound and a therapeutically effective amount of a mitochondrial uncoupler.

2. The method of claim 1, wherein the mitochondria-related disease or condition is a metabolic disorder, an obesity-associated condition, a disorder associated with oxidative stress, a neurological disorder, an inflammatory disorder, an immune-related disorder, a fibrotic disorder, an aging-related disorder, or a tumor or cancer.

3. The method of claim 2, wherein the metabolic disorder or the obesity-associated condition is a disorder associated with abnormal or ectopic lipid accumulation or storage, a lipid storage droplet disorder, lipodystrophy, obesity, a hyperphagia-associated disorder, metabolic syndrome, hypercholesterolemia, insulin resistance, diabetes, a cardiovascular disease, a liver disorder, a lysosomal storage disease, or a lipid storage disorder.

4. The method of claim 3, wherein:
the lipid storage droplet disorder is comparative gene identification-58 (CGI-58) deficiency (Chanarin-Dorfman syndrome), mitochondrial trifunctional protein (MTP) deficiency or apolipoprotein B (ApoB) deficiency;
the lipodystrophy is HIV-associated lipodystrophy or antiretroviral therapy (ART)-induced lipodystrophy;
the hyperphagia-associated disorder is Alstrom syndrome, Bardet-Biedl syndrome or Prader-Willi syndrome;
the hypercholesterolemia is familial hypercholesterolemia;
the diabetes is type 2 diabetes;
the cardiovascular disease is cardiomyopathy, coronary artery disease, stroke or ischemia-reperfusion injury;
the liver disorder is non-alcoholic fatty liver disease (NAFLD), non-alcoholic steatohepatitis (NASH), alcoholic liver disease (ALD), alcoholic steatohepatitis (ASH) or hepatotoxicity; and
the lipid storage disorder is lysosomal acid lipase (LAL) deficiency, Gaucher disease or Niemann-Pick disease.

5. The method of claim 2, wherein the disorder associated with oxidative stress is a metabolic disorder, a cardiovascular disorder, an ischemia-reperfusion injury, an inflammatory disorder, an autoimmune disorder, a degenerative disorder, a neurodegenerative disorder, a musculodegenerative disorder, a neuromuscular disorder, other muscle disorder, a neurodevelopmental disorder, a psychiatric disorder, a mitochondrial disease, a lysosomal storage disease, a disorder due to physical or body trauma, polycystic kidney disease, male infertility or an aging-related disorder.

6. The method of claim 2, wherein the neurological disorder is a neurodegenerative disorder, optic neuritis, Charcot-Marie-Tooth disease, epilepsy, ischemic stroke or traumatic brain injury.

7. The method of claim 2, wherein the inflammatory disorder or the fibrotic disorder is a liver disorder characterized by inflammation or/and fibrosis, a kidney disorder characterized by inflammation or/and fibrosis, a heart disorder characterized by inflammation or/and fibrosis, or a pancreas disorder characterized by inflammation or/and fibrosis.

8. The method of claim 2, wherein the immune-related disorder is an autoimmune disorder or a disorder associated with overactivation of the immune system.

9. The method of claim 2, wherein the tumor or cancer is associated with obesity or an activated class IA phosphoinositide 3-kinase p110α (PI3K-α).

10. The method of claim 1, wherein the nicotinyl riboside compound comprises at least one compound selected from nicotinamide riboside (NR), reduced NR (NRH), nicotinic acid riboside (NAR), reduced NAR (NARH), derivatives thereof, and pharmaceutically acceptable salts, solvates, hydrates, clathrates, polymorphs and stereoisomers thereof.

11. The method of claim 10, wherein the nicotinyl riboside compound comprises at least one compound selected from NR, NRH, NAR, NARH, and pharmaceutically acceptable salts, solvates, hydrates, clathrates, polymorphs and stereoisomers thereof.

12. The method of claim 10, wherein the nicotinyl riboside compound comprises at least one compound selected from nicotinamide riboside triacetate (NRTA), reduced NRTA (NRHTA), nicotinic acid riboside triacetate (NARTA), reduced NARTA (NARHTA), and pharmaceutically acceptable salts, solvates, hydrates, clathrates, polymorphs and stereoisomers thereof.

13. The method of claim 10, wherein the nicotinyl riboside compound comprises at least one compound selected from compounds of Formulas I and II: 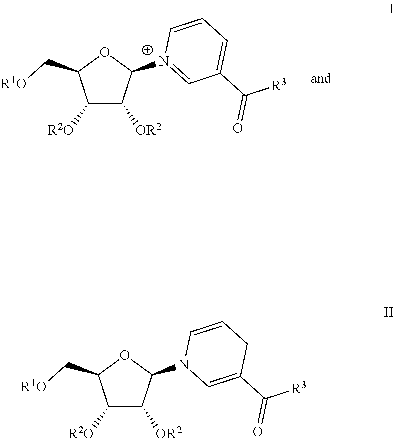
wherein:
R¹ is hydrogen, 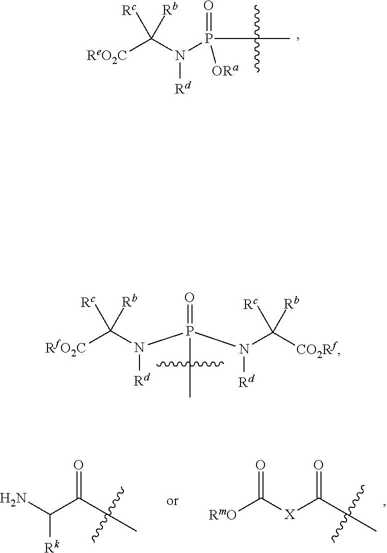
wherein:
R^{a} is hydrogen, a counterion, linear or branched C₁-C₆ alkyl, C₃-C₆ cycloalkyl, phenyl, 1-naphthyl or 2-naphthyl, wherein the phenyl is optionally substituted with F, Cl, —NO₂, linear or branched C₁-C₄ alkyl, —CF₃ or —O-(linear or branched C₁-C₄ alkyl);
R^{b} and R^{c} at each occurrence independently are hydrogen, linear or branched C₁-C₅ alkyl, —CH₂-phenyl, —CH₂-3-indole or —CH₂-5-imidazole, wherein the alkyl is optionally substituted with —OH, —OR^{j}, —SH, —SR^{j}, —NH₂, —NHR^{j}, —N(R^{j})₂, —NHC(═O)R^{j}, —NHC(═NH)NH₂, —C(═O)NH₂, —CO₂H or —C(═O)OR^{j}, and the phenyl is optionally substituted with —OH or —OR^{j}, wherein R^{j} at each occurrence independently is linear or branched C₁-C₄ alkyl;
R^{d} at each occurrence independently is hydrogen, methyl or linear or branched C₂-C₄ alkyl;
R^{e} and R^{f} at each occurrence independently are hydrogen, a counterion, linear or branched C₁-C₈ alkyl, C₃-C₆ cycloalkyl, —CH₂—(C₃-C₆ cycloalkyl), phenyl or —CH₂-phenyl, wherein the phenyl is optionally substituted with F, Cl, —NO₂, linear or branched C₁-C₄ alkyl, —CF₃ or —O-(linear or branched C₁-C₄ alkyl);
R^{k} is hydrogen, linear or branched C₁-C₆ alkyl, —CH₂-phenyl, —CH₂-3-indole or —CH₂-5-imidazole, wherein the alkyl is optionally substituted with —OH, —OR^{j}, —SH, —SR^{j}, —NH₂, —NHR^{j}, —N(R^{j})₂, —NHC(═O)R^{j}, —NHC(═NH)NH₂, —C(═O)NH₂, —CO₂H or —C(═O)OR^{j}, and the phenyl is optionally substituted with —OH or —OR^{j}, wherein R¹ at each occurrence independently is linear or branched C₁-C₄ alkyl;
R^{m} is hydrogen, a counterion, linear or branched C₁-C₆ alkyl, C₃-C₆ cycloalkyl, phenyl, —CH₂-phenyl or 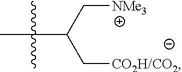
wherein the phenyl is optionally substituted with F, Cl, —NO₂, linear or branched C₁-C₄ alkyl, —CF₃ or —O-(linear or branched C₁-C₄ alkyl); and
X is cis or trans —HC═CH— or —(CH₂)ₙ— optionally substituted with —OH, —OR^{j} or —OC(═O)R^{j}, wherein R^{j} is linear or branched C₁-C₄ alkyl and n is 1, 2, 3, 4, 5 or 6;
R² at each occurrence independently is hydrogen, 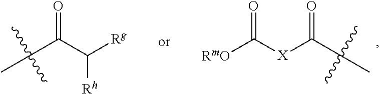
wherein:
R^{g} is hydrogen, linear or branched C₁-C₅ alkyl, —CH₂-phenyl, —CH₂-3-indole or —CH₂-5-imidazole, wherein the alkyl is optionally substituted with —OH, —OR^{j}, —SH, —SR^{j}, —NH₂, —NHR^{j}, —N(R^{j})₂, —NHC(═O)R^{j}, —NHC(═NH)NH₂, —C(═O)NH₂, —CO₂H or —C(═O)OR^{j}, and the phenyl is optionally substituted with —OH or —OR^{j}, wherein R^{j} at each occurrence independently is linear or branched C₁-C₄ alkyl;
R^{h} is hydrogen, methyl or —NH₂;
or R^{g} and R^{h} together with the carbon atom to which they are connected form a C₃-C₆ cycloalkyl or phenyl ring, wherein the phenyl ring is optionally substituted with F, Cl, —NO₂, linear or branched C₁-C₄ alkyl, —CF₃ or —O-(linear or branched C₁-C₄ alkyl); and
R^{m} and X are as defined above; and
R³ is —NH₂, —NHRⁿ, —N(Rⁿ)₂, —OH, —OR^{o} or 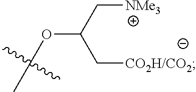
wherein:
Rⁿ at each occurrence independently is linear or branched C₁-C₆ alkyl or allyl, wherein the alkyl is optionally substituted with —OH or —O-(linear or branched C₁-C₃ alkyl), or both occurrences of Rⁿ and the nitrogen atom to which they are connected form a 3- to 6-membered heterocyclic ring; and
R^{o} is a counterion, linear or branched C₁-C₆ alkyl, C₃-C₆ cycloalkyl, phenyl or —CH₂-phenyl, wherein the phenyl is optionally substituted with F, Cl, —NO₂, linear or branched C₁-C₄ alkyl, —CF₃ or —O-(linear or branched C₁-C₄ alkyl);
or a pharmaceutically acceptable salt, solvate, hydrate, clathrate, polymorph or stereoisomer thereof.

14. The method of claim 1, wherein the therapeutically effective amount of the nicotinyl riboside compound is about 1-100 mg, 100-500 mg or 500-1000 mg per day.

15. The method of claim 1, wherein the uncoupler is OPC-163493, niclosamide, DK-520 (n-octanoyl ester of niclosamide), a niclosamide analog having —Cl or —CF₃ in place of the —NO₂ group, nitazoxanide, tizoxanide, oxyclozanide, BAM15, BHT, MitoBHT, DNP, DNP methyl ether, MP201, DNP-OC(═O)—N-piperidine, DNP-OC(═O)—N-morpholine, DNP-OC(═O)—N-piperazine, DNP-OC(═O)—N-piperazine-N-Me, 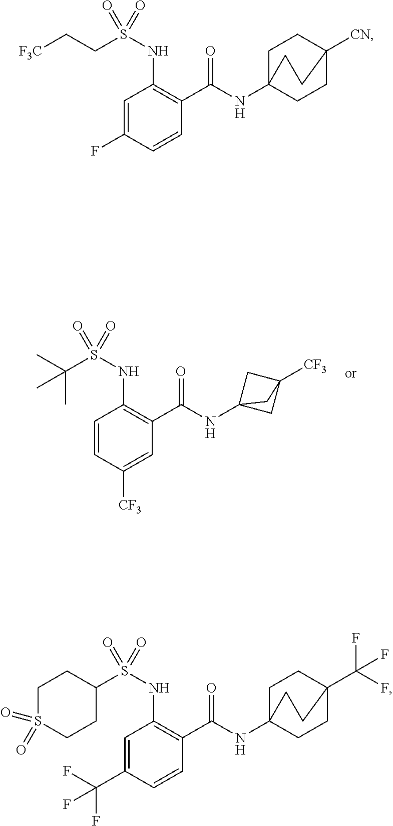 [SMILES: N#CC12CCC(NC(=O)c3ccc(F)cc3NS(=O)(=O)CCC(F)(F)F)(CC1)CC2 ; CC(C)(C)S(=O)(=O)Nc1ccc(C(F)(F)F)cc1C(=O)NC12CC(C(F)(F)F)(C1)C2 ; O=C(NC12CCC(C(F)(F)F)(CC1)CC2)c1ccc(C(F)(F)F)cc1NS(=O)(=O)C1CCS(=O)(=O)CC1]
or a pharmaceutically acceptable salt thereof, or a controlled-, slow- or sustained-release form thereof.

16. The method of claim 15, wherein the uncoupler is nitazoxanide, tizoxanide, BAM15, DNP or N-(4-cyanobicyclo[2.2.2]octan-1-yl)-4-fluoro-2-[(3,3,3-trifluoropropyl)sulfonamido]benzamide, or a pharmaceutically acceptable salt thereof, or a controlled-, slow- or sustained-release form thereof.

17. The method of claim 16, wherein the controlled-, slow- or sustained-release form of DNP is a pellet, particle, bead or sphere containing DNP and having a controlled-, slow- or sustained-release polymeric coating, or is a solid dosage form comprising a plurality of such pellets, particles, beads or spheres.

18. The method of claim 1, wherein the therapeutically effective amount of the uncoupler is about 1-100 mg, 100-300 mg or 300-500 mg per day.

19. The method of claim 1, wherein the nicotinyl riboside compound or/and the uncoupler is/are encapsulated in liposomes, micelles, cholestosomes, or lipid, polymeric or dendrimeric nano-/microparticles or nano-/microspheres.

20. The method of claim 1, wherein administration of the nicotinyl riboside compound in combination with the uncoupler provides mild, sustained mitochondrial uncoupling.

21. The method of claim 1, wherein administration of the nicotinyl riboside compound in combination with the uncoupler increases NAD⁺/NADH ratio by at least about 20%, 50% or 100%.

22. The method of claim 1, wherein administration of the nicotinyl riboside compound in combination with the uncoupler increases the safety or/and the efficacy of the uncoupler, or has synergistic effect(s).

23. The method of claim 1, wherein:
the mitochondria-related disease or condition is a metabolic disorder or an obesity-associated condition;
the nicotinyl riboside compound is NR, NRH, NRTA or NRHTA, or a pharmaceutically acceptable salt, solvate, hydrate, clathrate, polymorph or stereoisomer thereof; and
the uncoupler is nitazoxanide, tizoxanide, BAM15, DNP or N-(4-cyanobicyclo[2.2.2]octan-1-yl)-4-fluoro-2-[(3,3,3-trifluoropropyl)sulfonamido]benzamide, or a pharmaceutically acceptable salt, solvate, hydrate, clathrate, polymorph, stereoisomer, or controlled-, slow- or sustained-release form thereof.

24. The method of claim 1, further comprising administering one or more additional therapeutic agents.

25. The method of claim 24, wherein the one or more additional therapeutic agents are or comprise a poly(ADP-ribose) polymerase (PARP) inhibitor or/and a PI3K-α inhibitor.
